(19) United States
(12) Patent Application Publication
(10) Pub. No.: US 2026/0250423 A1
(43) Pub. Date: Aug. 27, 2026

(54) ANTIBODIES FOR USE IN AGRIN-DEFICIENT DISORDERS

(71) Applicant: argenx BV, Zwijnaarde (BE)

(72) Inventors: Roeland Vanhauwaert, Zwijnaarde (BE); Karen Silence, Zwijnaarde (BE); Julien Oury, Brooklyn, NY (US); Steven J. Burden, Sharon, CT (US); Sally Spendiff, Ottawa (CA); Hanns Lochmuller, Ottawa (CA)

(21) Appl. No.: 19/385,638

(22) Filed: Nov. 11, 2025

Related U.S. Application Data
(63) Continuation of application No. PCT/EP2024/062981, filed on May 10, 2024.
(60) Provisional application No. 63/516,303, filed on Jul. 28, 2023.

(30) Foreign Application Priority Data
May 15, 2023 (WO) PCT/EP2023/063007

(51) Int. Cl. C07K 16/40

(52) U.S. Cl. CPC C07K 16/40

(57) ABSTRACT

The present invention relates to a MuSK antibody-based molecule for use in the prevention or treatment of a disease or condition resulting from Agrin deficiency in a human subject.

### CROSS-REFERENCE TO RELATED APPLICATIONS

This application is a continuation of International Patent Application No. PCT/EP2024/062981, filed on May 10, 2024, which claims priority from International Patent Application No. PCT/EP2023/063007, filed on May 15, 2023, and U.S. Provisional Patent Application No. 63/516,303, filed on Jul. 28, 2023, the contents of each of which are incorporated herein by reference in their entirety.

### SEQUENCE LISTING

The content of the electronically submitted Sequence Listing XML (Name: 222980_SL.xml; Size: 378,407 bytes; Created on Nov. 7, 2025) is incorporated by reference herein in its entirety.

### FIELD OF THE INVENTION

The present invention relates to an antibody-based molecule for use in preventing, delaying, reversing or treating a disease or condition resulting from agrin deficiency in a human subject. In an embodiment, this antibody-based molecule is an anti-MuSK antibody.

### BACKGROUND

The development and maintenance of the neuromuscular junction (NMJ) critically depends on the agrin/LRP4/MuSK pathway, whereby an active neuronal form of agrin is secreted by the nerve terminal which binds to its receptor LRP4 on the muscle fibre surface. This induces phosphorylation of MuSK, which recruits DOK7 and causes the rapsyn mediated clustering of acetylcholine receptors (AChRs). Many neuromuscular disorders are hallmarked by impaired neuromuscular junctions (NMJs) and caused by genetic defects.

Agrin is a large proteoglycan which is encoded by AGRN gene in human and plays crucial role in the development of the neuromuscular junction and in the aggregation of acetylcholine receptors during synaptogenesis. Agrin functions by activating the MuSK (Muscle-Specific Kinase) protein, a receptor tyrosine kinase required for the formation and maintenance of the neuromuscular junction. Agrin deficiency can lead to congenital myasthenic syndromes and myasthenia gravis.

Congenital myasthenic syndromes (CMS) are a heterogeneous group of genetic disorders resulting from mutations that affect structures at the neuromuscular junction (NMJ). Characteristic features are fatigue and transient or permanent ocular, facial, bulbar, or limb muscle weakness with onset at birth or childhood, however the condition can also present during adolescence or adulthood.

There are no standardized treatment guidelines for CMS. Current treatments aim to improve symptoms, but dosing, duration, treatment combinations, and side effects tend to be poorly specified. The most common medications taken for CMS are acetylcholinesterase inhibitors, and 3,4-diaminopyridine (3,4-DAP), a potassium channel blocker, is the most common alternative or added pharmacologic treatment. However, acetylcholinesterase inhibitors or 3,4-DAP tend to be ineffective in patients with specific CMS subtypes. Other medications that may be used to manage symptoms include salbutamol, albuterol, ephedrine, and fluoxetine, depending on the specific CMS subtype.

Therefore, there is still a need for a new therapy for CMS and other diseases associated with agrin deficiency.

### BRIEF DESCRIPTION OF THE DRAWINGS

FIG. 1: Treatment and testing protocol for mice. Three groups of animals have been studied, an untreated wild type control group and 2 groups with Agrn^{nmf380} mice treated at days P5, P15, and P35 with an IP (intraperitoneal) injection of either the MuSK agonist antibody (3B2: represented by full length heavy chain SEQ ID NO: 282 and full length light chain SEQ ID NO: 283) or an isotope matched negative control antibody (Mota-hIgG1LALAdelk, represented by full length heavy chain SEQ ID NO:272 and full length light chain SEQ ID NO:273). Between P5 and P50 mice underwent several functional tests, at P50 mice were culled and tissues collected for further processing.

FIG. 2A-FIG. 2B: Survival and body weight of Agrn^{nmf380} mice. Survival (FIG. 2A) and body weight (FIG. 2B) of WT, Agrn^{nmf380} (Mot), and Agrn^{nmf380} (3B2) mice. Broken vertical lines indicate injections. Error bars indicate sd. WT n=6, Agrn^{nmf380} (Mot) n=6, Agrn^{nmf380} (3B2) n=6.

FIG. 3A-FIG. 3C: Tube test (hindlimb suspension test) in Agrn^{nmf380} mice at P7-9. Agrn^{nmf380} (3B2) mice held on significantly longer than Agrn^{nmf380} (Mot) mice (FIG. 3A). There were no significant differences in the number of pulls (FIG. 3B), or the hind limb suspension (HLS) score (FIG. 3C) between animals. Shapir-Wilk test for normality followed by kruskal-Wallis test with Dunn's correction for multiple comparisons (FIG. 3A) & (FIG. 3C), 1-way ANOVA with Tukey's correction (FIG. 3B). WT n=6, Agrn^{nmf380} (Mot) n=6, Agrn^{nmf380} (3B2) n=6.

FIG. 4A-FIG. 4D: Grip strength assessment of Agrn^{nmf380} mice. Animals were tested using a grip strength meter at days P23 and P48. For forelimb assessments (FIG. 4A) animals were held by their tails and allowed to grip the bar, for hindlimb assessments (FIG. 4B) restrained animals were held above the bar until they grabbed it with their hindlimbs. An average of three trials was taken. To determine forelimb ratio (FIG. 4C) and hindlimb ration (FIG. 4D) values were divided by body weight. 2-Way ANOVA with Tukey's multiple comparisons correction. At P23 WT n=6, Agrn^{nmf380} (Mot) n=3, Agrn^{nmf380} (3B2) n=6, at P42 WT n=6, Agrn^{nmf380} (Mot) n=1, Agrn^{nmf380} (3B2) n=6.

FIG. 5A-FIG. 5B: Inverted Hanging wire time for Agrn^{nmf380} mice. Animals were given a 30 second acclimatisation period on the grid and then inverted over a padded box. The time animals held on was recorded. This was repeated three times with at least a 15 min break in between attempts. WT animals held for significantly longer that Agrn^{nmf380} (Mot) and Agrn^{nmf380} (3B2) animals by P48 (FIG. 5A). When the hanging ratio was determined (FIG. 5B) WT animals also held on for significantly longer than treated and untreated Agrn^{nmf380} animals. Two-Way ANOVA with Tukey's multiple comparisons correction. At P23 WT n=6, Agrn^{nmf380} (Mot) n=3, Agrn^{nmf380} (3B2) n=6, at P42 WT n=6, Agrn^{nmf380} (Mot) n=1, Agrn^{nmf380} (3B2) n=6.

FIG. 6A-FIG. 6B: Muscle harvest weight of Agrn^{nmf380} mice. At the time of culling and tissue extraction muscles were blotted dry and weighed (FIG. 6A). To normalise the data the weight was also determined as a percentage of body weight (FIG. 6B). 2-Way ANOVA with Tukey's multiple comparisons correction. WT n=6, Agrn^{nmf380} (Mot) n=6, Agrn^{nmf380} (3B2) n=6. * p<0.05, ** p<0.005, *** p<0.001, **** p<0.0001.

FIG. 7A-1-FIG. 7D-2: Fibre size and MHC type, and MuSK, p-MuSK, and p-MuSK: MuSK in the gas muscle in Agrn^{nmf380} mice. Muscle cross sections were labelled and analysed for fibre size and type. (FIG. 7A-1) Haematoxylin and Eosin was used to label muscle cross sections from WT (left), Agrn^{nmf380} (Mot) (middle), and Agrn^{nmf380} (3B2) (right) mice quadriceps. (FIG. 7A-2) In quad muscle, Agrn^{nmf380} (Mot) mice had significantly decreased myofiber size compared to both WT mice and those treated with 3B2. (FIG. 7A-3) The same pattern existed in soleus muscle these changes were not significant. 1-way ANOVA with Tukey's multiple comparisons correction. Quad: WT n=6, Agrn^{nmf380} (Mot) n=4, Agrn^{nmf380} (3B2) n=6. Soleus: WT n=6, Agrn^{nmf380} (Mot) n=3 (note unequal sexes), Agrn^{nmf380} (3B2) n=4 *p<0.05, ** p<0.005, *** p<0.001, **** p<0.0001.

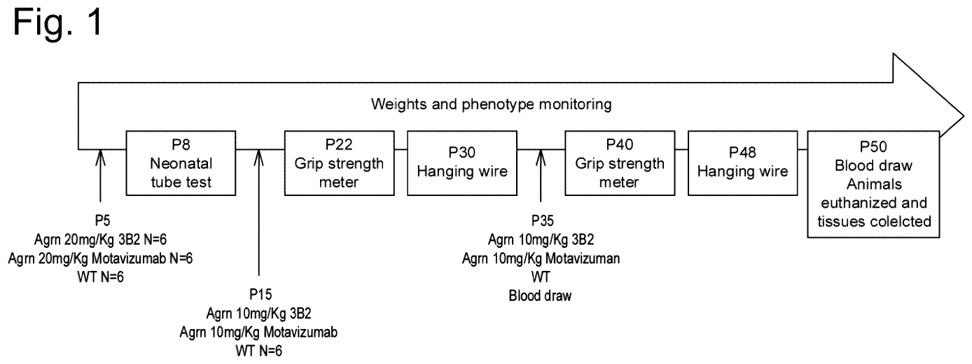

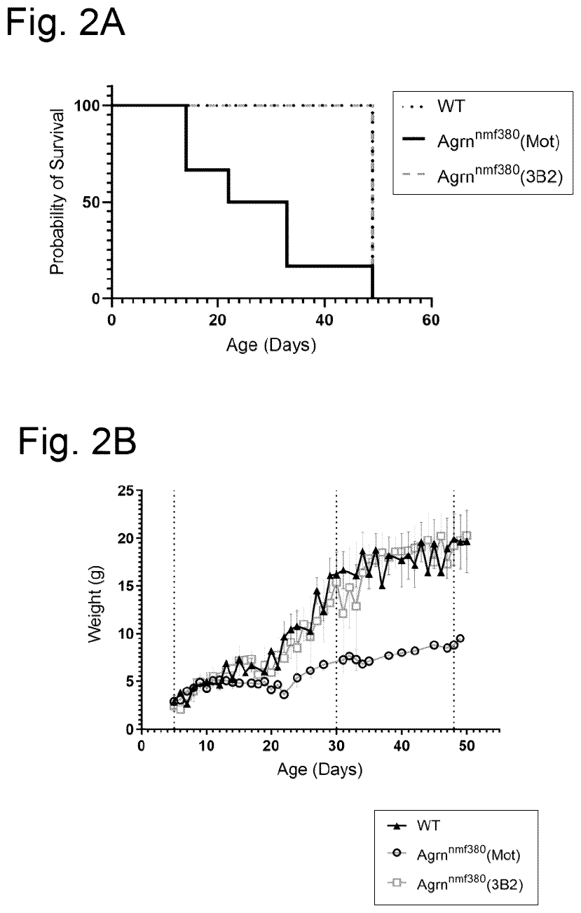

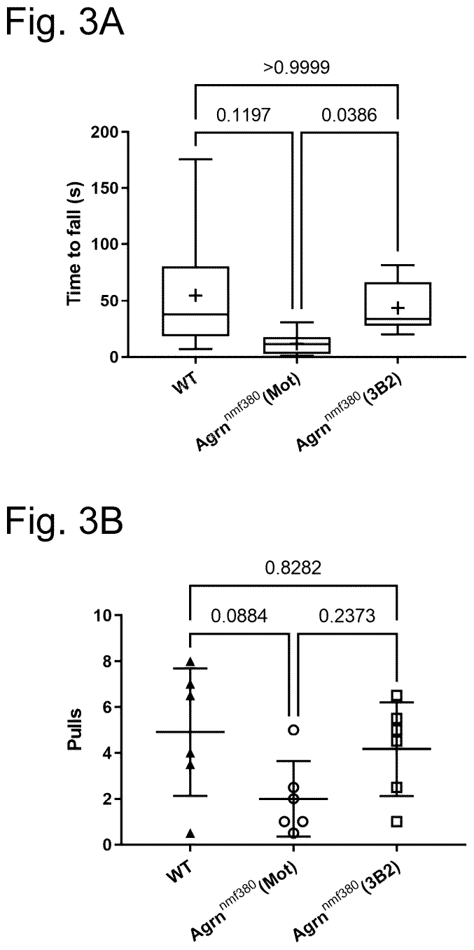

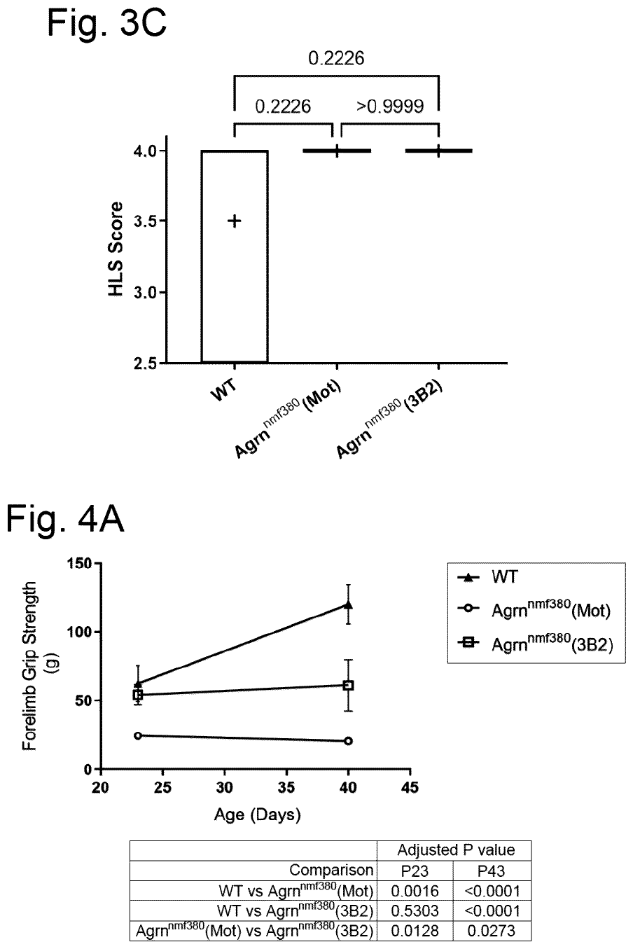

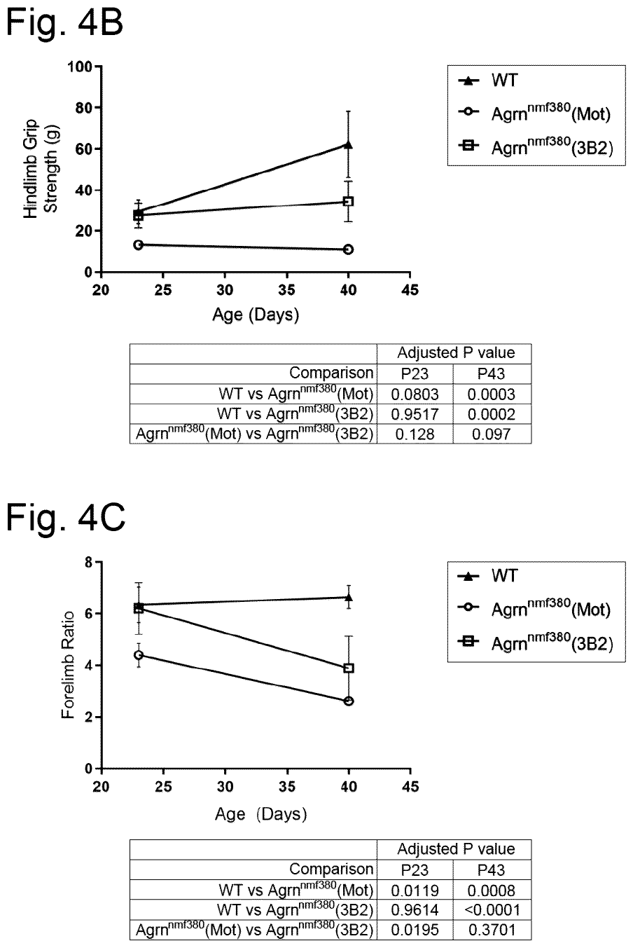

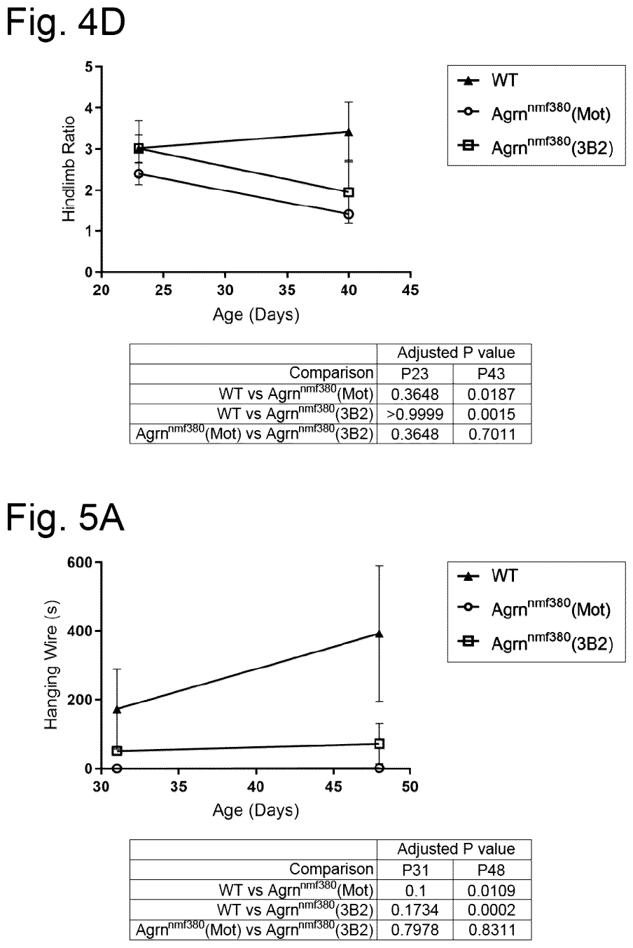

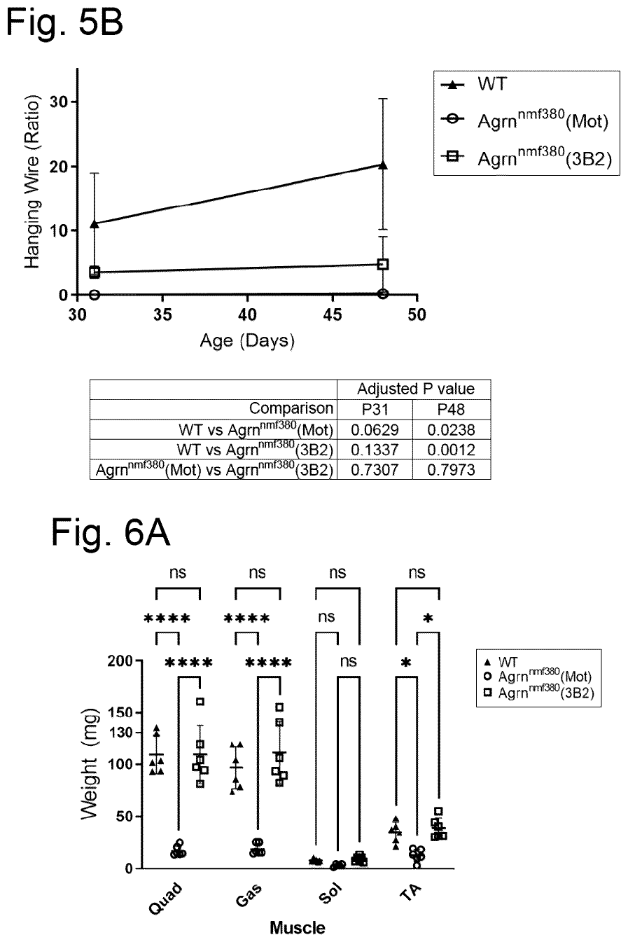

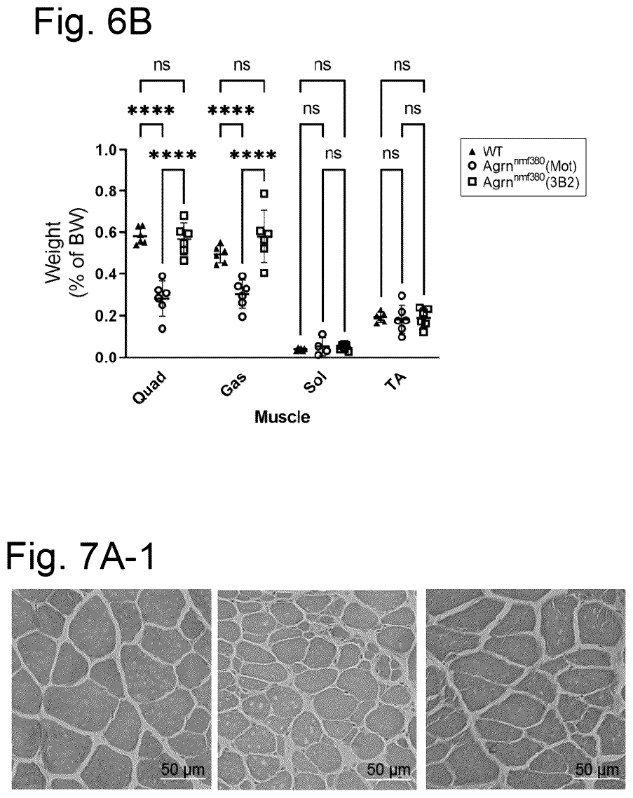

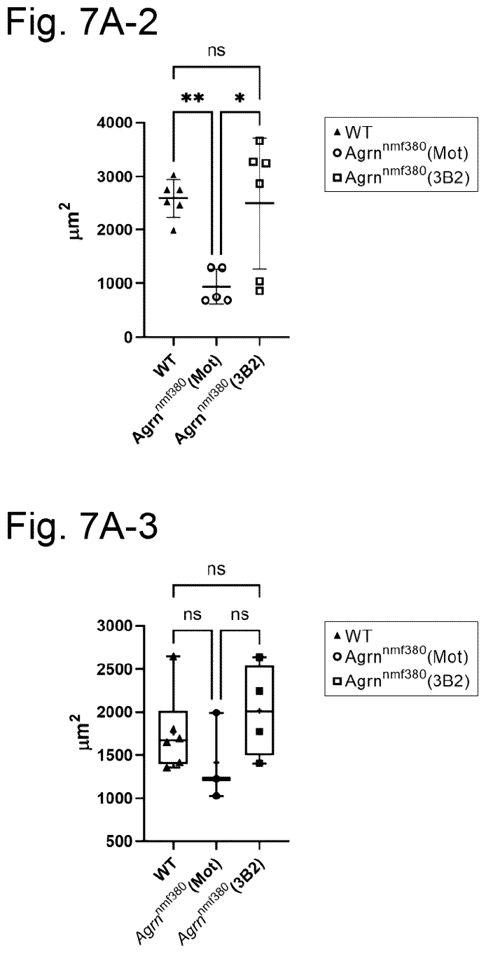

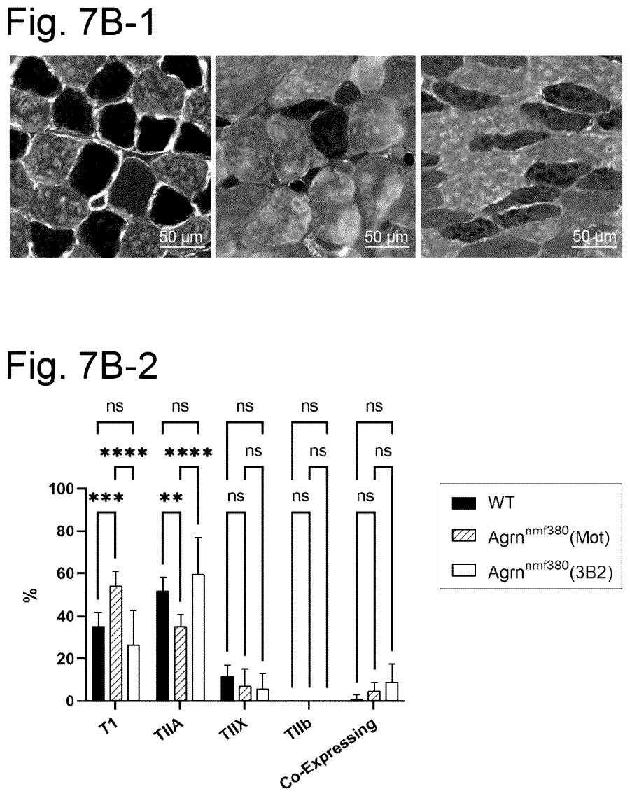

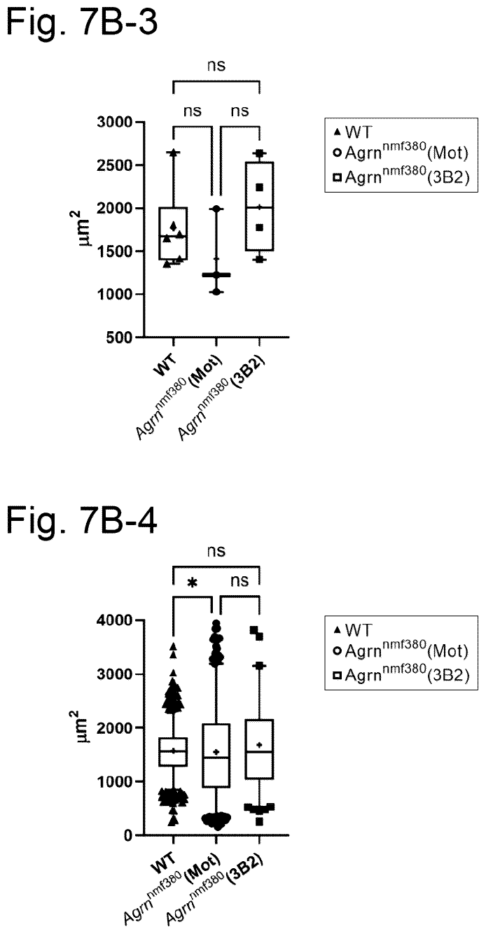

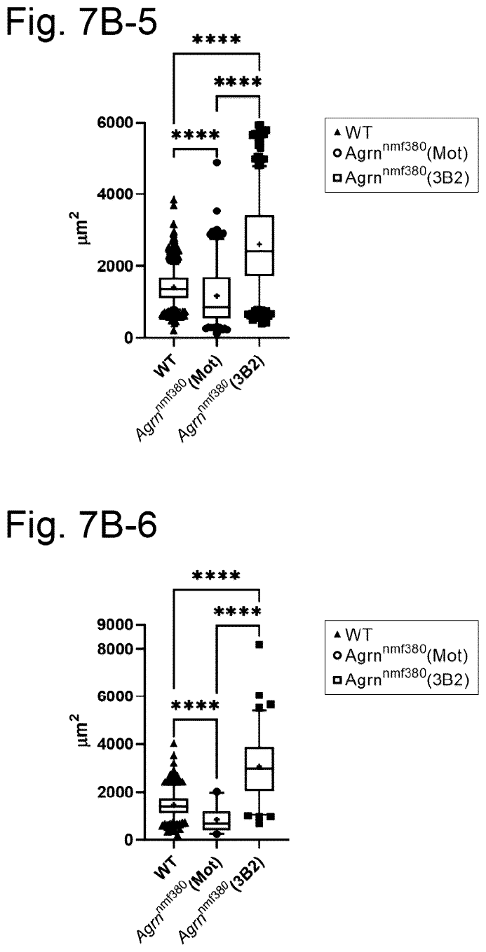

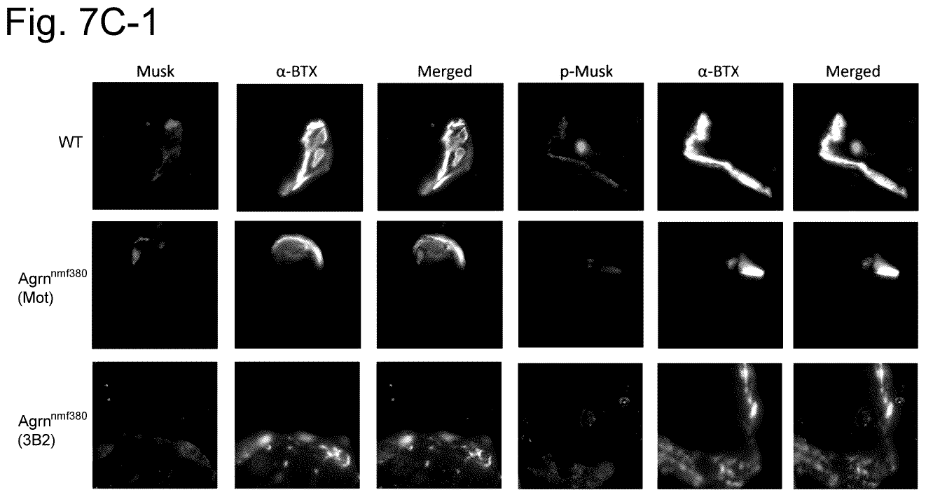

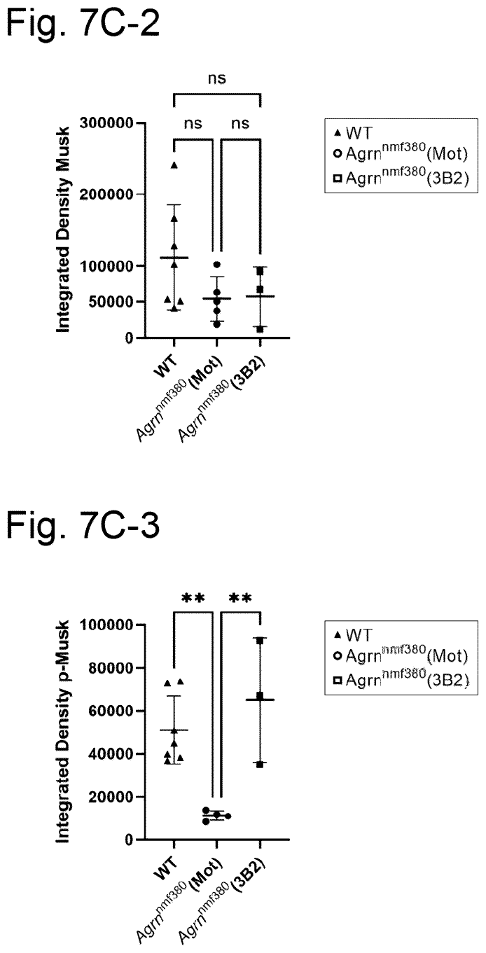

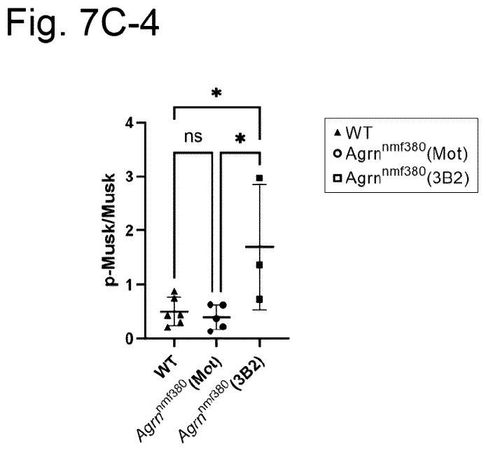

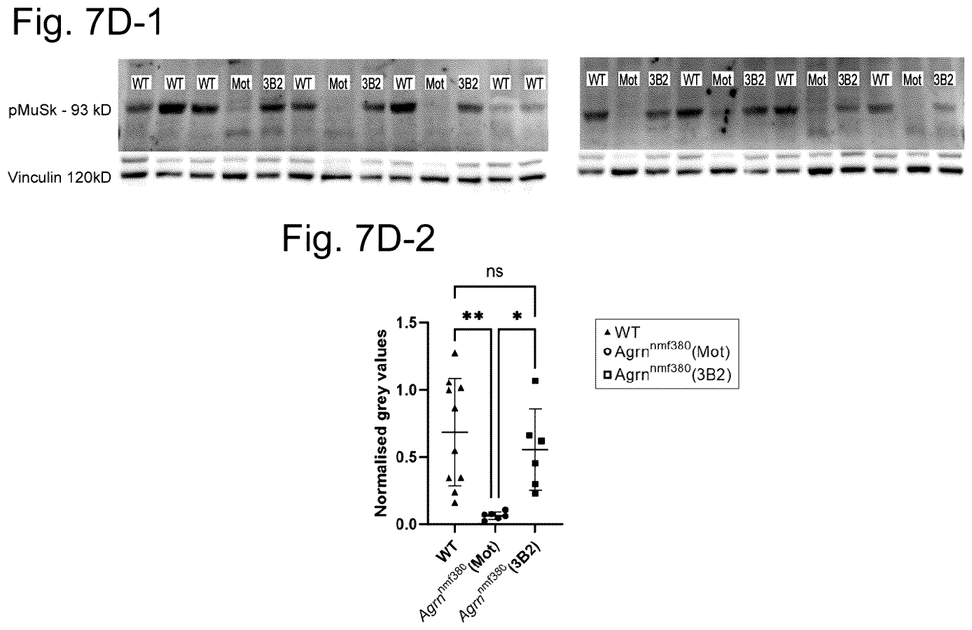

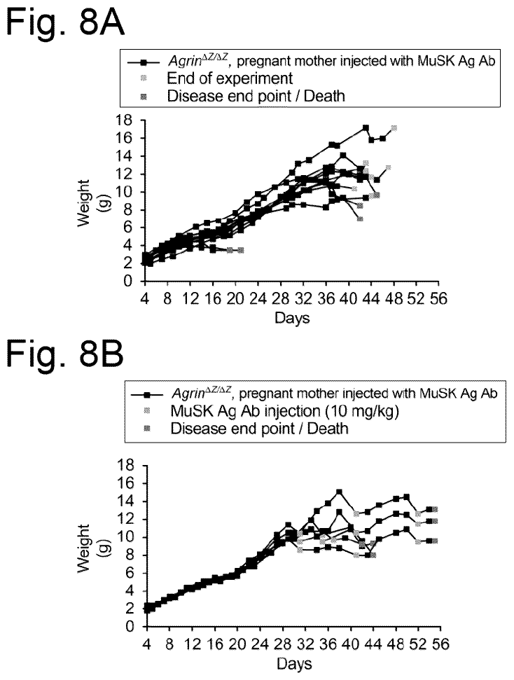

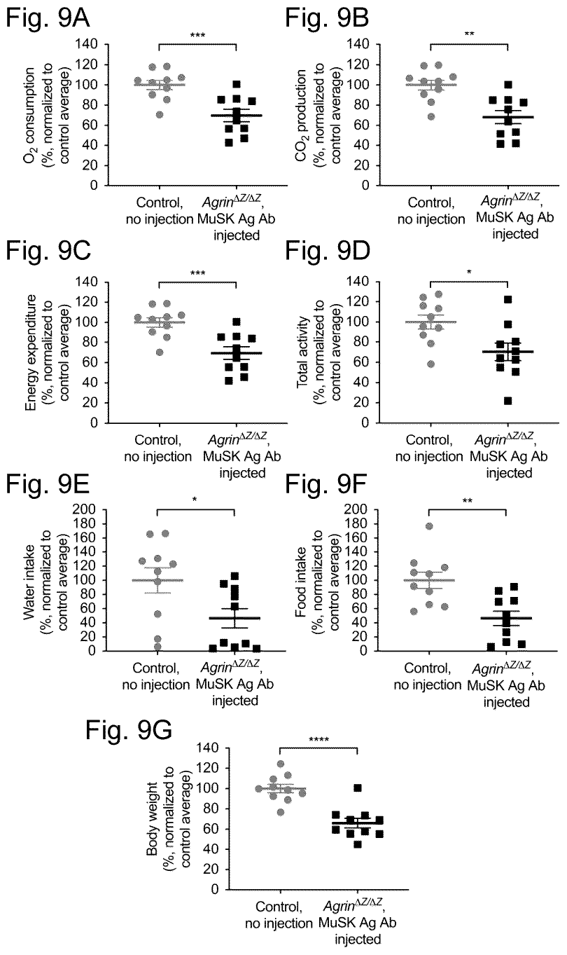

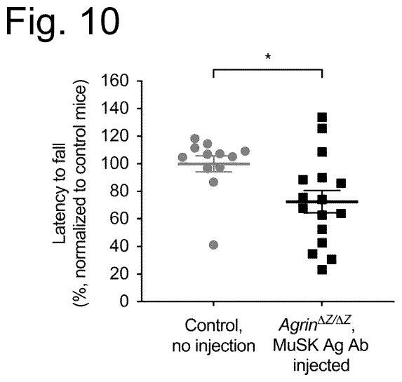

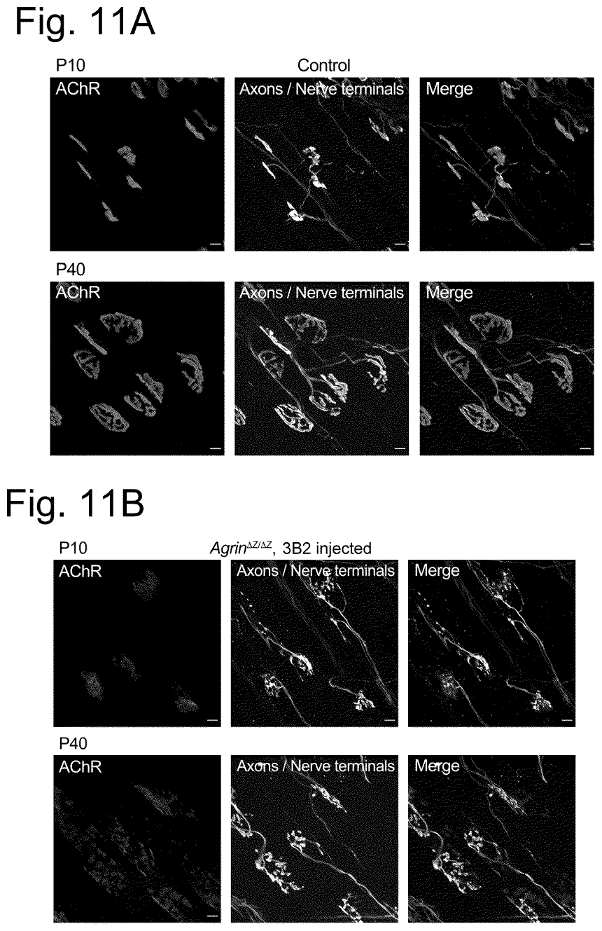

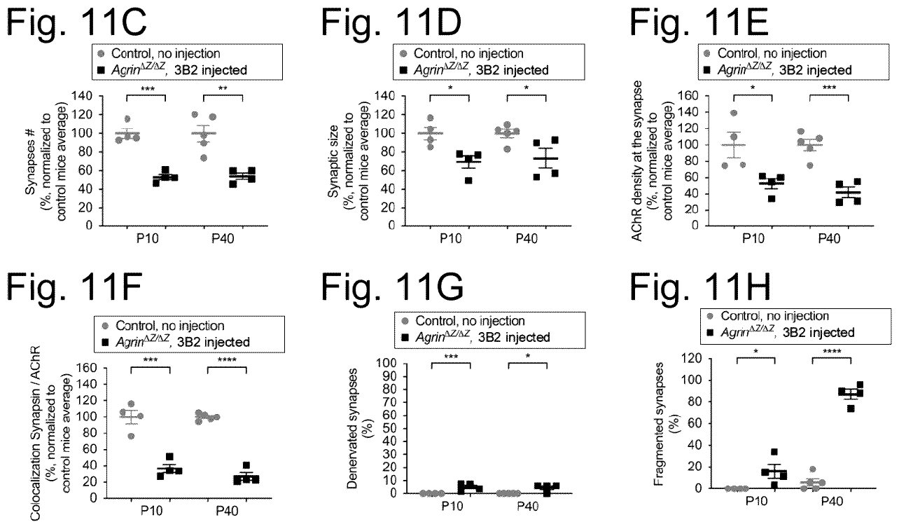

(FIG. 7B-1) Muscle cross sections were labelled and analysed for fibre and type, muscle was labelled and the numbers of fibres of each type recorded. (FIG. 7B-2) Soleus muscle was labelled and the numbers of fibres of each type recorded. Agrn^{nmf380} (3B2) mice demonstrated a full rescue of the fibre type switching observed in Agrn^{nmf380} (Mot) mice. 2-way ANOVA with Tukey's multiple comparisons correction. WT n=6, Agrn^{nmf380} (Mot) n=4, Agrn^{nmf380} (3B2) n=6. (FIG. 7B-3) WT mice had significantly larger slow type 1 fibres than Agrn^{nmf380} (Mot) mice. The difference between WT and Agrn^{nmf380} (Mot) mice and the rescue in Agrn^{nmf380} (3B2) mice was more pronounced in the fast type IIa (FIG. 7B-4), IIx (FIG. 7B-5) and IIb (FIG. 7B-6) fibres. * p<0.05, ** p<0.005, *** p<0.001, **** p<0.0001.

(FIG. 7C-1) Muscle cross sections were labelled and analysed for area and mean intensity values which was used to generate an integrated density value. (FIG. 7C-2) WT animals had significantly more MuSK than Agrn^{nmf380} (Mot) or Agrn^{nmf380} (3B2) animals. (FIG. 7C-3) However, addition of 3B2 restored p-MuSK levels in Agrn^{nmf380} (3B2) animals. (FIG. 7C-4) When the ratio of MuSK to p-MuSK was calculated Agrn^{nmf380} (3B2) animals had significantly more p-MuSK that Agrn^{nmf380} (Mot) or WT animals. 1-way ANOVA with Tukey's multiple comparisons correction. * p<0.05, ** p<0.005, *** p<0.001, **** p<0.0001.

(FIG. 7D-1) Muscles were run on a WB blot and the grey bands quantified. (FIG. 7D-2) Agrn^{nmf380} (Mot) animals had significantly less p-MuSK than WT animals. However, this was completely reversed with 3B2 treatment. 1-way ANOVA with Tukey's multiple comparisons correction. * p<0.05, ** p<0.005.

FIG. 8A-FIG. 8B: Postnatal injection of MuSK agonist antibodies 3B2 shows a moderate effect on survival or weight gain of Agrin^{ΔZ/ΔZ} mice. FIG. 8A. Agrin^{ΔZ/ΔZ} C57BL/6 progeny, who were born from 3B2-injected Agrin^{ΔZ/+}C57BL/6 female mice during pregnancy ultimately shows disease relapse around P30. Dark grey squares indicate natural death or sacrifice at disease end point (~20% weight loss); light grey squares indicate the end of experiment. FIG. 8B. Agrin^{ΔZ/ΔZ} C57BL/6 progeny, who were born from an 3B2-injected Agrin^{ΔZ/+}C57BL/6 female mouse during pregnancy ultimately shows disease relapse around P30. When they lost weight (~10% weight loss), they were re-injected with 3B2 (10 mg/kg), and monitored. Plots show the values for each individual mouse overtime. Dark grey squares indicate natural death or sacrifice at disease end point (~20% weight loss).

FIG. 9A-FIG. 9G: Agrin^{ΔZ/ΔZ} mice display respiratory, energy expenditure, activity, water intake, food intake, and body weight deficits compared to wildtype mice when disease relapses. P40 Agrin^{ΔZ/ΔZ} C57BL/6 mice, who were born from 3B2-injected Agrin^{ΔZ/+}C57BL/6 female mice during pregnancy, were placed in metabolic chambers and monitored 24 hours a day for 4 days. O₂ consumption, CO₂ production, energy expenditure, total activity, water intake, food intake, and body weight measurements were taken. The scatter plots show the values for 10 control (wildtype or Agrin^{ΔZ/+}) mice and 10 Agrin^{ΔZ/ΔZ} mice and the mean in percentage normalized to wildtype mice average ±SEM values. Two-sided Student's t-test (p, *<0.05, p, **<0.005, p, ***<0.0005, ****<0.00005).

FIG. 10: Agrin^{ΔZ/ΔZ} mice display motor deficits when disease relapses. AgrinΔZ/42 mice in a C57BL/6 background, who were born from 3B2-injected Agrin^{ΔZ/+}C57BL/6 female mice during pregnancy, display motor deficits at disease onset (10% weight loss) as assessed by the latency to fall from a rotating rotarod compared to non-injected wildtype mice. Plots show individual data points and mean in percentage normalized to non-injected mice average+SEM. Two-sided Student's t-test (p, *<0.05).

FIG. 11A-FIG. 11H: 3B2 partially restores synapse development in Agrin^{ΔZ/ΔZ} Progeny born from Agrin^{ΔZ/+}C57BL/6 female, injected with 3B2 during pregnancy. Diaphragm muscles from P10 and P40 mice were stained with Alexa 488-x-BGT to label AChRs (red) and antibodies to β-III Tubulin/Synapsin to label motor axons/nerve terminals (green). Scale bar, 10 μm.

### SUMMARY OF THE INVENTION

Provided herein is an antibody-based molecule for use in preventing or treating a disease or condition resulting from agrin deficiency in a human subject. In some embodiments, the antibody-based molecule is an anti-MuSK antibody. In some embodiments, the antibody-based molecule binds the MuSK Frizzled (Fz)-like domain sequence of SEQ ID NO: 130.

In some embodiments, the disease or condition resulting from agrin deficiency is such that the deficient agrin can no longer bind its receptor LRP4.

In some embodiments, the disease or condition resulting from agrin deficiency is such that the deficient agrin is still able to bind its natural receptor, LRP4, but with a deteriorated/decreased affinity and/or with a lower capacity to activate MuSK phosphorylation than its counterpart wild type agrin. In an embodiment, said antibody-based molecule may elicit a higher capacity to activate MuSK phosphorylation than deficient agrin.

In some embodiments, the disease or condition resulting from agrin deficiency is such that the expression level of MuSK is lower than the expression level of MuSK in a subject who is not agrin deficient. In an embodiment, wherein the antibody-based molecule may elicit a higher capacity to activate MuSK phosphorylation than deficient agrin.

In some embodiments, the disease or condition resulting from agrin deficiency is such that the binding of LRP4 to MuSK is impaired/antagonized, which in its turn leads to an impaired activation of MuSK. In an embodiment, the antibody-based molecule may elicit a higher capacity to activate MuSK phosphorylation than deficient agrin.

In some embodiments, the antibody-based molecule is an agonist MuSK antibody and/or has reduced or eliminated effector function. In some embodiments, the antibody-based molecule induces MuSK phosphorylation. In some embodiments, the antibody-based molecule comprises a heavy chain variable region (VH) and a light chain variable region (VL) as identified in table 3 and/or a CDR as identified in table 1 or 2.

In some embodiments, the antibody-based molecule comprises a heavy chain variable domain (VH) and a light chain variable domain (VL), wherein:
the VH comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156, and the VL comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In some embodiments, the antibody-based molecule comprises:
a heavy chain variable domain (VH) comprising an amino acid sequence that is at least 80% identical or similar to SEQ ID NO: 202, and
a light chain variable domain (VL) comprising an amino acid sequence that is at least 80% identical or similar to SEQ ID NO: 203 (3B2).

In some embodiments, the antibody-based molecule comprises:
a heavy chain variable domain (VH) comprising an amino acid sequence that is at least 80% identical or similar to SEQ ID NO: 202, and
a light chain variable domain (VL) comprising an amino acid sequence that is at least 80% identical or similar to SEQ ID NO: 203, and wherein:
   the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 150 or having 1, 2, 3, 4 or 5 amino acid alterations relative to SEQ ID NO: 150, and
a CDR-H3 amino acid sequence comprising SEQ ID NO: 156 or having 1, 2, 3, 4 or 5 amino acid alterations relative to SEQ ID NO:156, and
   the light chain variable domain comprises:
a CDR-L1 amino acid sequence comprising SEQ ID NO: 159 or having 1, 2, 3, 4 or 5 amino acid alterations relative to SEQ ID NO: 159,
a CDR-L2 amino acid sequence comprising SEQ ID NO: 172 or having 1, 2, 3, 4 or 5 amino acid alterations relative to SEQ ID NO: 172, and
a CDR-L3 amino acid sequence comprising SEQ ID NO: 183 or having 1, 2, 3, 4 or 5 amino acid alterations relative to SEQ ID NO:183 (3B2).

In some embodiments, the antibody-based molecule comprises:
a full length heavy chain comprising SEQ ID NO: 282,
a full length light chain comprising SEQ ID NO: 283, and wherein:
the full length heavy chain comprises L234A and L235A mutations numbered according the EU numbering system.

In some embodiments, the antibody-based molecule comprises a heavy chain variable region (VH) and a light chain variable region (VL), wherein:
the VH comprises:
   a CDR-H1 amino acid sequence which comprises SEQ ID NO:147 or has 1, 2, 3, 4 or 5 amino acid changes relative to SEQ ID NO: 147,
   a CDR-H2 amino acid sequence which comprises SEQ ID NO: 153 or has 1, 2, 3, 4 or 5 amino acid changes relative to SEQ ID NO: 153, and
   a CDR-H3 amino acid sequence which comprises SEQ ID NO: 156 or has 1, 2, 3, 4 or 5 amino acid changes relative to SEQ ID NO: 156 (3B2g2m1), and the VL comprises:
   a CDR-L1 amino acid sequence which comprises SEQ ID NO: 159 or has 1, 2, 3, 4 or 5 amino acid changes relative to SEQ ID NO: 159,
   a CDR-L2 amino acid sequence which comprises SEQ ID NO: 172 or has 1, 2, 3, 4 or 5 amino acid changes relative to SEQ ID NO: 172, and
   a CDR-L3 amino acid sequence which comprises SEQ ID NO: 195 or has 1, 2, 3, 4 or 5 amino acid changes relative to SEQ ID NO:195 (3B2g2m1).

In some embodiments the antibody-based molecule comprises:
a heavy chain variable region (VH) comprising an amino acid sequence that is at least 80% identical or similar to SEQ ID NO: 234 and
a light chain variable region (VL) comprising an amino acid sequence that is at least 80% identical or similar to SEQ ID NO: 235.

In some embodiments, the antibody-based molecule comprises:
a heavy chain variable region (VH) comprising an amino acid sequence that is at least 80% identical or similar to SEQ ID NO: 234 and
a light chain variable region (VL) comprising an amino acid sequence that is at least 80% identical or similar to SEQ ID NO: 235, and wherein:
   the VH comprises:
a CDR-H1 amino acid sequence which comprises SEQ ID NO: 147 or has 1, 2, 3, 4 or 5 amino acid changes relative to SEQ ID NO: 147,
a CDR-H2 amino acid sequence which comprises SEQ ID NO: 153 or has 1, 2, 3, 4 or 5 amino acid changes relative to SEQ ID NO: 153, and
a CDR-H3 amino acid sequence which comprises SEQ ID NO: 156 or has 1, 2, 3, 4 or 5 amino acid changes relative to SEQ ID NO: 156 (3B2g2m1), and
   the VL comprises:
a CDR-L1 amino acid sequence which comprises SEQ ID NO: 159 or has 1, 2, 3, 4 or 5 amino acid changes relative to SEQ ID NO: 159,
a CDR-L2 amino acid sequence which comprises SEQ ID NO: 172 or has 1, 2, 3, 4 or 5 amino acid changes relative to SEQ ID NO: 172, and
a CDR-L3 amino acid sequence which comprises SEQ ID NO: 195 or has 1, 2, 3, 4 or 5 amino acid changes relative to SEQ ID NO:195 (3B2g2m1).

In some embodiments, the antibody-based molecule comprises:
a full length heavy chain comprising SEQ ID NO: 268 and
a full length light chain comprising SEQ ID NO: 269, and
wherein the full length heavy chain comprises L234A and L235A mutations numbered according the EU numbering system.

In a second aspect, provided herein is a polynucleotide for use in preventing, delaying, reversing or treating a disease or condition resulting from agrin deficiency in a human subject, said polynucleotide comprising a nucleotide sequence which encodes the antibody-based molecule of any of the preceding embodiments or a VH or VL or CDR thereof.

In a third aspect, provided herein is an expression vector for use in preventing or treating a disease or condition resulting from agrin deficiency in a human subject, comprising the polynucleotide of the second aspect of this invention, preferably operably linked to a regulatory region which allows expression of the antibody or antigen binding fragment thereof or VH or VL or CDR thereof in a host cell or cell-free expression system.

In a fourth aspect, provided herein is host cell or cell-free expression system for use in preventing, delaying, reversing or treating a disease or condition resulting from agrin deficiency in a human subject, containing the expression vector of the third aspect of this invention.

In a fifth aspect, provided herein is a composition for use in preventing or treating a disease or condition resulting from agrin deficiency in a human subject, comprising an antibody-based molecule as defined in any of the preceding embodiments, a polynucleotide of the second aspect of this invention, or an expression vector of the third aspect of this invention preferably said composition comprising at least one pharmaceutically acceptable carrier or excipient.

In some embodiments, the antibody-based molecule, the polynucleotide, the expression vector, the host cell, the cell-free expression system or the composition for use according to any of the preceding aspects and embodiments of this invention, wherein the disease or condition is characterized by an impaired neuromuscular transmission and/or a denervation at the NMJ (neuromuscular junction). In some embodiments, the disease is a neuromuscular disease. In some embodiments, the neuromuscular disease is selected from the group consisting of: amyotrophic lateral sclerosis (ALS), spinal muscular atrophy (SMA), myasthenia gravis (MG), congenital myasthenia, congenital myasthenic syndrome (CMS), Lambert-Eaton myasthenic syndrome (LEMS), Lyme disease, poliomyelitis, post-poliomyelitis, heavy metal intoxication, Kennedy syndrome, adult-onset Tay-Sachs disease, hereditary spastic paraplegia, multifocal neuropathy, cervical spondylosis, extramedullary tumor with compressive radiculopathy and myelopathy, inclusion body myositis, progressive bulbar palsy, progressive muscular atrophy, motor neuron syndrome and thyrotoxic myopathy. In some embodiments, the disease is CMS, preferably the disease is presynaptic CMS or agrin induced CMS. In some embodiments, the disease is ALS or SMA.

In some embodiments, the subject has at least one of the symptoms selected from the group consisting of: early postnatal disease onset, impaired neuromuscular performance, muscle weakness, fatigue, a preponderance of slow muscle fibers, a disaggregation of NMJs resulting in remodeling, functional denervation, enlarged subsynaptic folds and premature lethality.

In some embodiments, the administration of said antibody-based molecule, polynucleotide, expression vector, or pharmaceutical composition to said human subject results in one or more of the following therapeutic effects:
an increase and maintenance of the number or percentage of fully innervated NMJ in the subject,
an improvement of the motor performance and/or grip strength of the subject,
an improvement of the contractile properties of a muscle at the NMJ of the subject,
an improvement of the resistance to fatigue of a muscle at the NMJ of the subject,
an induction of an increase of the muscle weight at the NMJ of the subject,
an increase in muscle mass of the subject,
an increase in muscle size of the subject,
an increase of nerve terminal area and complexity, acetylcholine perimeter, and/or endplate area, and/or
a stabilization of said disorder.

### DETAILED DESCRIPTION OF THE INVENTION

### General Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms “a”, “an”, and “the” include both singular and plural referents unless the context clearly dictates otherwise. The terms ‘disorder’ and ‘disease’ are used herein interchangeably. The terms “comprising”, “comprises” and “comprised of” as used herein are synonymous with ‘including’, ‘includes’ or ‘containing’, ‘contains’, and are inclusive or open-ended and do not exclude additional, non-recited members, compounds, products, elements or method steps. The expression “essentially consists of” used in the context of a product or a composition (“a product essentially consisting of” or “a composition essentially consisting of”) means that additional molecules may be present but that such molecule does not change/alter the characteristic/activity/functionality of said product or composition. For example, a composition may essentially consist of an antibody or an antibody fragment if the composition as such would exhibit similar characteristic/activity/functionality as one of the antibodies or as the one of the antibody fragments. The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, amino acid residues will be indicated either by their full name or according to the standard three-letter or one-letter amino acid code. As used herein, the terms “polypeptide” or “protein” are used interchangeably, and refer to a polymeric form of amino acids of any length, which can include coded and non-coded amino acids, chemically or biochemically modified or derivatized amino acids, and polypeptides having modified peptide backbones. A “peptide” is also a polymer of amino acids with a length which is usually of up to 50 amino acids. A polypeptide or peptide is represented by an amino acid sequence.

As used herein, the terms “nucleic acid molecule”, “polynucleotide”, “polynucleic acid”, “nucleic acid” are used interchangeably and refer to polymeric form of nucleotides of any length, either deoxyribonucleotides or ribonucleotides, or analogs thereof. A nucleic acid molecule is represented by a nucleic acid sequence, which is primarily characterized by its base sequence. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural identity or similarity between two macromolecules, particularly between two polypeptides or polynucleotides, from same or different taxons, wherein said similarity is due to shared ancestry. Hence, the term ‘homologues’ denotes so-related macromolecules having said secondary and optionally tertiary structural similarity. For comparing two or more nucleotide sequences, the ‘(percentage of) sequence identity’ between a first nucleotide sequence and a second nucleotide sequence may be calculated using methods known by the person skilled in the art, e.g. by dividing the number of nucleotides in the first nucleotide sequence that are identical to the nucleotides at the corresponding positions in the second nucleotide sequence by the total number of nucleotides in the first nucleotide sequence and multiplying by 100% or by using a known computer algorithm for sequence alignment such as NCBI Blast. In determining the degree of sequence similarity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions have been exemplified herein. Amino acid sequences and nucleic acid sequences are said to be ‘exactly the same’ if they have 100% sequence identity over their entire length.

Throughout this application, each time one refers to a specific amino acid sequence SEQ ID NO (take SEQ ID NO: Y as example), one may replace it by: a polypeptide comprising an amino acid sequence that has at least 80% sequence identity or similarity with amino acid sequence SEQ ID NO: Y. Throughout this application, the wording “a sequence is at least X % identical with another sequence” may be replaced by “a sequence has at least X % sequence identity with another sequence”.

Each amino acid sequence described herein by virtue of its identity percentage (at least 80%) with a given amino acid sequence respectively has in a further preferred embodiment an identity of at least 80%, 81%, 82%, 83%, 84%, 85%, 86%, 87%, 88%, 89%, 90%, 91%, 92%, 93%, 94%, 95%, 96%, 97%, 98%, 99% or more identity with the given amino acid sequence respectively. In a preferred embodiment, sequence identity is determined by comparing the whole length of the sequences as identified herein. Each amino acid sequence described herein by virtue of its similarity percentage (at least 80%) with a given amino acid sequence respectively has in a further preferred embodiment a similarity of at least 80%, 81%, 82%, 83%, 84%, 85%, 86%, 87%, 88%, 89%, 90%, 91%, 92%, 93%, 94%, 95%, 96%, 97%, 98%, 99% qwerty or more similarity with the given amino acid sequence respectively. In a preferred embodiment, sequence similarity is determined by comparing the whole length of the sequences as identified herein. Unless otherwise indicated herein, identity or similarity with a given SEQ ID NO means identity or similarity based on the full length of said sequence (i.e. over its whole length or as a whole).

“Sequence identity” is herein defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, FASTA, BLASTN, and BLASTP (Altschul, S. F. et al., J. Mol. Biol. 215:403-410 (1990)), EMBOSS Needle (Madeira, F., et al., Nucleic Acids Research 47 (W1): W636-W641 (2019)). The BLAST program is publicly available from NCBI and other sources (BLAST Manual, Altschul, S., et al., NCBI NLM NIH Bethesda, MD 20894; Altschul, S., et al., J. Mol. Biol. 215:403-410 (1990)). The EMBOSS program is publicly available from EMBL-EBI. The well-known Smith Waterman algorithm may also be used to determine identity. The EMBOSS Needle program is the preferred program used.

Preferred parameters for polypeptide sequence comparison include the following: Algorithm: Needleman and Wunsch, J. Mol. Biol. 48 (3): 443-453 (1970); Comparison matrix: BLOSUM62 from Henikoff and Henikoff, Proc. Natl. Acad. Sci. USA. 89:10915-10919 (1992); Gap Open Penalty: 10; and Gap Extend Penalty: 0.5. A program useful with these parameters is publicly available as the EMBOSS Needle program from EMBL-EBI. The aforementioned parameters are the default parameters for a Global Pairwise Sequence alignment of proteins (along with no penalty for end gaps).

Preferred parameters for nucleic acid comparison include the following: Algorithm: Needleman and Wunsch, J. Mol. Biol. 48:443-453 (1970); Comparison matrix: DNAfull; Gap Open Penalty: 10; Gap Extend Penalty: 0.5. A program useful with these parameters is publicly available as the EMBOSS Needle program from EMBL-EBI. The aforementioned parameters are the default parameters for a Global Pairwise Sequence alignment of nucleotide sequences (along with no penalty for end gaps).

Also provided herein are embodiments wherein any embodiment described herein may be combined with any one or more other embodiments, provided the combination is not mutually exclusive.

### Antibody-Based Molecule

The present invention is based on the surprising discovery that agrin deficiency can be rescued by treating with MuSK antibodies. These treated agrin deficient animals as exemplified in the examples showed improvements in at least one of the following parameters and/or symptoms: the number or percentage of fully innervated NMJ in the subject, the motor performance and/or grip strength, the contractile properties of a muscle at the NMJ, the resistance to fatigue of a muscle at the NMJ, the muscle weight at the NMJ, muscle mass of the subject, muscle size of the subject, nerve terminal area and complexity, acetylcholine perimeter, endplate area, the stabilization of said disorder, myofiber size, NMJ morphology, muscle strength of the subject, body/muscle weight and survival of the subject.

All antibody-based molecule defined herein are encompassed as such in the present invention. In the context of the present application, the wording “antibody-based molecule” may be replaced by the word “antibody” or by the expression “antibody or a functional fragment thereof” or by the expression “antibody or antigen binding fragment”.

In a first aspect of this invention, there is provided an antibody-based molecule for use in preventing, delaying, reversing or treating a disease or condition resulting from agrin deficiency in a human subject.

All these features have been further defined herein. Antibody-based molecules include, without limitation antibodies, full antibodies, epitope binding fragments of whole antibodies, antigen binding fragment of whole antibodies and antibody derivatives. An epitope binding fragment of an antibody can be obtained through the actual fragmenting of a parental antibody (for example, a Fab or (Fab) 2 fragment). Alternatively, the epitope binding fragment is an amino acid sequence that comprises a portion of the amino acid sequence of such parental antibody. As used herein, a molecule is said to be a “derivative” of an antibody (or relevant portion thereof) if it is obtained through the actual chemical modification of a parent antibody or portion thereof, or if it comprises an amino acid sequence that is substantially similar to the amino acid sequence of such parental antibody or relevant portion thereof (for example, differing by less than 30%, less than 20%, less than 10%, or less than 5% from such parental molecule or such relevant portion thereof, or by 10 amino acid residues, or by fewer than 10, 9, 8, 7, 6, 5, 4, 3 or 2 amino acid residues from such parental molecule or relevant portion thereof).

In an embodiment, an antibody-based molecule of the present invention is an intact immunoglobulin or a molecule having an epitope-binding 333 acids encoding such fragments in recombinant cells (see e.g., Evans et al. “Rapid Expression Of An Anti-Human C5 Chimeric Fab Utilizing A Vector That Replicates In COS And 293 Cells,” J. Immunol. Meth. 184:123-38 (1995), which is hereby incorporated by reference in its entirety). For example, a chimeric gene encoding a portion of a F(ab′) 2 fragment could include DNA sequences encoding the CH1 domain and hinge region of the heavy chain, followed by a translational stop codon to yield such a truncated antibody fragment molecule. Suitable fragments capable of binding to a desired epitope may be readily screened for utility in the same manner as an intact antibody.

Antibody derivatives include those molecules that contain at least one epitope-binding domain of an antibody, and are typically formed using recombinant techniques. One exemplary antibody derivative includes a single chain Fv (scFv). A scFv is formed from the two domains of the Fv fragment, the VL and the VH, which may be encoded by separate genes. Such gene sequences or their encoding cDNA are joined, using recombinant methods, by a flexible linker (typically of about 10, 12, 15 or more amino acid residues) that enables them to be made as a single protein chain in which the VL and VH associate to form monovalent epitope-binding molecules (see e.g., Bird et al. “Single-Chain Antigen-Binding Proteins,” Science 242:423-426 (1988); and Huston et al. “Protein Engineering Of Antibody Binding Sites: Recovery Of Specific Activity In An Anti-Digoxin Single-Chain Fv Analogue Produced In Escherichia coli,” Proc. Natl. Acad. Sci. (U.S.A.) 85:5879-5883 (1988), which are hereby incorporated by reference in their entirety). Alternatively, by employing a flexible linker that is not too short (e.g., not less than about 9 residues) to enable the VL and VH of different single polypeptide chains to associate together, one can form a bispecific antibody, having binding specificity for two different epitopes.

In another embodiment, the antibody derivative is a divalent or bivalent single-chain variable fragment, engineered by linking two scFvs together either in tandem (i.e., tandem scFv), or such that they dimerize to form a diabody (Holliger et al. “Diabodies′: Small Bivalent And Bispecific Antibody Fragments,” Proc. Natl. Acad. Sci. (U.S.A.) 90 (14), 6444-8 (1993), which is hereby incorporated by reference in its entirety). In yet another embodiment, the antibody is a triabody, i.e., a trivalent single chain variable fragment, engineered by linking three scFvs together, either in tandem or in a trimer formation to form a triabody. In another embodiment, the antibody is a tetrabody of four single chain variable fragments. In another embodiment, the antibody is a “linear antibody” which is an antibody comprising a pair of tandem Fd segments (VH-CH1-VH-CH1) that form a pair of antigen binding regions (see Zapata et al. Protein Eng. 8 (10): 1057-1062 (1995), which is hereby incorporated by reference in its entirety). In another embodiment, the antibody derivative is a minibody, consisting of the single-chain Fv regions coupled to the CH3 region (i.e., scFv-CH3).

These and other useful antibody fragments and derivatives in the context of the present invention are discussed further herein. It also should be understood that the term antibody-based molecule, unless specified otherwise, also includes antibody-like polypeptides, such as chimeric antibodies and humanized antibodies, antigen binding fragments and antibody fragments retaining the ability to specifically bind to the antigen (epitope-binding fragments, antigen binding fragments or functional fragments) provided by any known technique, such as enzymatic cleavage, peptide synthesis, and recombinant techniques.

An antibody as generated herein may be of any isotype. As used herein, “isotype” refers to the immunoglobulin class (for instance IgG1, IgG2, IgG3, IgG4, IgD, IgA, IgE, or IgM) that is encoded by heavy chain constant region genes. The choice of isotype typically will be guided by the desired effector functions, such as antibody-dependent cellular cytotoxicity (ADCC) induction. Exemplary isotypes are IgG1, IgG2, IgG3, and IgG4. Particularly useful isotypes of the MuSK antibodies disclosed herein include IgG1 and IgG2. Either of the human light chain constant regions, kappa or lambda, may be used. If desired, the class of a MuSK antibody of the present invention may be switched by known methods. For example, an antibody of the present invention that was originally IgM may be class switched to an IgG antibody of the present invention. Further, class switching techniques may be used to convert one IgG subclass to another, for instance from IgG1 to IgG2. Thus, the effector function of the antibodies of the present invention may be changed by isotype switching to, e.g., an IgG1, IgG2, IgG3, IgG4, IgD, IgA, IgE, or IgM antibody for various therapeutic uses.

In an embodiment, one, two, or more amino acid substitutions are introduced into an IgG constant region Fc region to alter the effector function(s) of the antibody-based molecule. For example, one or more amino acids selected from amino acid residues 234, 235, 236, 237, 238, 239, 243, 265, 267, 268, 292, 297, 300, 318, 320, 322, 327, 328, 329, 330, 331, 332, and 396, numbered according to the EU numbering system (www.imgt.org/IMGTScientificChart/Numbering/Hu_IGHGnber.html #notes, and Edelman, G. M. et al., Proc. Natl. Acad. USA, 63, 78-85 (1969). PMID: 5257969), can be replaced with a different amino acid residue such that the antibody-based molecule has an altered affinity for an effector ligand but retains the antigen-binding ability. In an embodiment, the amino acid 234 or 235 has been replaced. In another embodiment, the amino acids 234 and 235 have been replaced. In this context, a preferred amino acid sequence of a human IgG constant Fc region comprises SEQ ID NO:266 or 267. In this context, for example, the amino acids 234 and 235 numbered according to the EU numbering system correspond to amino acids 7 and 8 in SEQ ID NO:266 and 267 (i.e. a human IgG constant Fc region of an antibody-based molecule disclosed herein), or the amino acids 234 and 235 numbered according the EU numbering system correspond to amino acids 238 and 239 in SEQ ID NO: 268 and 270 and 282 (i.e. a human full length heavy chain of an antibody-based molecule disclosed herein). The positions typically differ, because variable regions vary in length, which introduces a “delta” between the numberings. In the case depicted above, that delta is 4. Accordingly, the same holds for other amino acid positions identified above (i.e. 236, 237, 238, 239, 243, 265, 267, 268, 292, 297, 300, 318, 320, 322, 327, 328, 329, 330, 331, 332, and 396) numbered according to the EU numbering system when identifying the corresponding positions in SEQ ID NO: 266 or 267 or 268 or 270 or 282. Within the application as filed, one can either refer to the position of an amino acid using the EU numbering system or using the actual position in a given Fc region (for example SEQ ID NO: 266 or 267) or in a full length heavy chain (for example SEQ ID NO: 268 or 270 or 282).

Accordingly, in an embodiment, 1, 2, 3, 4, 5, 6, 7, 8, 9, 10, 11, 12, 13, 14, 15, 16 or 17 amino acid substitutions are introduced into SEQ ID NO: 266 or 267. In an embodiment, 1, 2, 3, 4 amino acid substitutions are introduced into SEQ ID NO:266 or 267. In an embodiment, 1 or 2 amino acid substitutions are introduced into SEQ ID NO:266 or 267. Accordingly, in an embodiment, 1, 2, 3, 4, 5, 6, 7, 8, 9, 10, 11, 12, 13, 14, 15, 16 or 17 amino acid substitutions are introduced into SEQ ID NO: 266 or 267 and said substitutions are introduced at amino acid positions selected from amino acid residues 234, 235, 236, 237, 239, 243, 267, 292, 297, 300, 318, 320, 322, 328, 330, 332, and 396 numbered according the EU numbering system of said sequence. In an embodiment, 1 or 2 amino acid substitutions are introduced into SEQ ID NO:266 or 267. In an embodiment, the amino acid 234 or 235 numbered according to the EU numbering system of SEQ ID NO: 266 or 267 has been replaced. In another embodiment, the amino acids 234 and 235 numbered according to the EU numbering system of SEQ ID NO: 266 or 267 have been replaced.

The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In an embodiment, one or more amino acid substitutions may be introduced into the Fc region of the antibody-based molecule described herein to remove potential glycosylation sites on the Fc region, which may reduce Fc receptor binding (see, e.g., Shields R L et al., (2001) J Biol Chem 276:6591-604, which is herein incorporated by reference in its entirety). In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

In a first embodiment, one or more of the following mutations have been introduced into the constant region of the antibody-based molecule described herein (all numbered according to the EU numbering system): an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In a second embodiment, one or more of the following mutations have been introduced into the constant region of the antibody-based molecule described herein (all numbered according to the EU numbering system): an L234A and/or an L235A substitution; an L234A and an L235A substitution; an L234A, an L235A and a P329G substitution; an L234A, an L235A and a G236K substitution; an L234A, an L235A and a G236E substitution; an L234A, an L235A and a G236R substitution; an L234A and a G236R substitution; an L234A, L235S and a G236R substitution; an L234A, L235T and a G236R substitution; an L234D, L235H and a G236R substitution; an L234D, L235K and a G236R substitution; an L234D and a G236R substitution; an L234D, L235Q and a G236R substitution; an L234D, L235S and a G236R substitution; an L234E, L235D and a G236R substitution; an L234E, L235H and a G236R substitution; an L234E, L2351 and a G236R substitution; an L234G, L235H and a G236R substitution; an L234G, L235Q and a G236R substitution; an L234G, L235S and a G236R substitution; an L234H, L2351 and a G236R substitution; an L234H, L235S and a G236R substitution; an L234K, L235Q and a G236R substitution; an L234K, L235R and a G236R substitution; an L234K, L235S and a G236R substitution; an L234K, L235T and a G236R substitution; an L234K, L235V and a G236R substitution; an L234Q, L235A and a G236R substitution; an L234Q, L235D and a G236R substitution; an L234Q, L235H and a G236R substitution; an L234Q and a G236R substitution; an L234Q, L235Q and a G236R substitution; an L234Q, L235R and a G236R substitution; an L234Q, L235S and a G236R substitution; an L234Q, L235T and a G236R substitution; an L234Q, L235V and a G236R substitution; an L234R, L235D and a G236R substitution; an L234R, L235E and a G236R substitution; an L234R, L235H and a G236R substitution; an L234R, L2351 and a G236R substitution; an L234R, L235K and a G236R substitution; an L234R and a G236R substitution; an L234R, L235Q and a G236R substitution; an L234R, L235R and a G236R substitution; an L234R, L235T and a G236R substitution; an L234S, L235E and a G236R substitution; an L234S, L235G and a G236R substitution; an L234S, L235H and a G236R substitution; an L234S, L2351 and a G236R substitution; an L234S and a G236R substitution; an L234S, L235R and a G236R substitution; L234S, L235T and a G236R substitution; L234S, L235V and a G236R substitution; an L234T, L235A and a G236R substitution; an L234T, L235D and a G236R, an L234T, L235H and a G236R substitution; an L234T, L2351 and a G236R substitution; an L234T, L235K and a G236R substitution; an L234T, L235Q and a G236R substitution; an L234T, L235R and a G236R substitution; an L234T, L235S and a G236R substitution; an L234T, L235T and a G236R substitution; an L234T, L235V and a G236R substitution; a G236R and an L328R substitution; an L234A, an L235A, a G237A, a P238S, an H268A, an A330S and a P331S substitution; an E233P, an L234V, an L235A, a G326 deletion, an A327G, an A330S and a P331S substitution; an L235A and a G236R substitution; an L235S and a G236R substitution.

In a third embodiment, one or more of the following mutations have been introduced into the Fc region SEQ ID NO: 266 or SEQ ID NO: 267 of the antibody-based molecule described herein (all numbered according to the EU numbering system): an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In a fourth embodiment, one or more of the following mutations have been introduced into the Fc region SEQ ID NO: 266 or SEQ ID NO: 267 of the antibody-based molecule described herein (all numbered according to the EU numbering system): an L234A and/or an L235A substitution; an L234A and an L235A substitution; an L234A, an L235A and a P329G substitution; an L234A, an L235A and a G236K substitution; an L234A, an L235A and a G236E substitution; an L234A, an L235A and a G236R substitution; an L234A and a G236R substitution; an L234A, L235S and a G236R substitution; an L234A, L235T and a G236R substitution; an L234D, L235H and a G236R substitution; an L234D, L235K and a G236R substitution; an L234D and a G236R substitution; an L234D, L235Q and a G236R substitution; an L234D, L235S and a G236R substitution; an L234E, L235D and a G236R substitution; an L234E, L235H and a G236R substitution; an L234E, L2351 and a G236R substitution; an L234G, L235H and a G236R substitution; an L234G, L235Q and a G236R substitution; an L234G, L235S and a G236R substitution; an L234H, L2351 and a G236R substitution; an L234H, L235S and a G236R substitution; an L234K, L235Q and a G236R substitution; an L234K, L235R and a G236R substitution; an L234K, L235S and a G236R substitution; an L234K, L235T and a G236R substitution; an L234K, L235V and a G236R substitution; an L234Q, L235A and a G236R substitution; an L234Q, L235D and a G236R substitution; an L234Q, L235H and a G236R substitution; an L234Q and a G236R substitution; an L234Q, L235Q and a G236R substitution; an L234Q, L235R and a G236R substitution; an L234Q, L235S and a G236R substitution; an L234Q, L235T and a G236R substitution; an L234Q, L235V and a G236R substitution; an L234R, L235D and a G236R substitution; an L234R, L235E and a G236R substitution; an L234R, L235H and a G236R substitution; an L234R, L2351 and a G236R substitution; an L234R, L235K and a G236R substitution; an L234R and a G236R substitution; an L234R, L235Q and a G236R substitution; an L234R, L235R and a G236R substitution; an L234R, L235T and a G236R substitution; an L234S, L235E and a G236R substitution; an L234S, L235G and a G236R substitution; an L234S, L235H and a G236R substitution; an L234S, L2351 and a G236R substitution; an L234S and a G236R substitution; an L234S, L235R and a G236R substitution; L234S, L235T and a G236R substitution; L234S, L235V and a G236R substitution; an L234T, L235A and a G236R substitution; an L234T, L235D and a G236R, an L234T, L235H and a G236R substitution; an L234T, L2351 and a G236R substitution; an L234T, L235K and a G236R substitution; an L234T, L235Q and a G236R substitution; an L234T, L235R and a G236R substitution; an L234T, L235S and a G236R substitution; an L234T, L235T and a G236R substitution; an L234T, L235V and a G236R substitution; a G236R and an L328R substitution; an L234A, an L235A, a G237A, a P238S, an H268A, an A330S and a P331S substitution; an E233P, an L234V, an L235A, a G326 deletion, an A327G, an A330S and a P331S substitution; an L235A and a G236R substitution; an L235S and a G236R substitution.

In an embodiment, one or more of the following mutations are introduced into the Fc region SEQ ID NO: 266 or SEQ ID NO: 267 of the antibody-based molecules described herein: an L234A and/or an L235A substitution (numbered according to the EU numbering system). In an embodiment, the following mutations are introduced into the Fc region SEQ ID NO: 266 or SEQ ID NO: 267 of the antibody-based molecule described herein: an L234A and an L235A substitutions numbered according to the EU numbering system. This embodiment results in an antibody-based molecule with a heavy chain represented by SEQ ID NO:268 or 270 or 282. Such an antibody with altered, diminished even abolished effector function is attractive in the context of the invention.

### MuSK Antibody Based Molecules

The present invention also relates to anti-MuSK antibody-based molecules, including anti-MuSK antibodies, epitope-binding domains thereof, antigen binding fragments thereof and antibody derivatives that are for use in preventing, delaying, reversing or treating a disease or condition resulting from agrin deficiency in a human subject. The term “anti-MuSK antibody” may be replaced by the term “MuSK antibody”. Any anti-MuSK antibody-based molecule including anti-MuSK antibodies, epitope-binding domains thereof, antigen binding fragments thereof and antibody derivatives that is capable of binding muscle-specific tyrosine protein kinase (MuSK) is encompassed within the present invention.

The invention provides the insight that such antibody-based molecules are useful for the prevention or treatment of diseases or conditions where a subject is in need of increased MuSK signaling or MuSK phosphorylation, such as neuromuscular disease or conditions associated with agrin deficiency. In an embodiment, such anti-MuSK antibody is able to activate or induce MuSK signalling and/or MuSK phosphorylation.

MuSK is a receptor tyrosine kinase that is expressed in skeletal muscle and has a crucial, master role in forming and maintaining neuromuscular synapses (Burden et al., “The Role of MuSK in Synapse Formation and Neuromuscular Disease,” Cold Spring Harb. Perspect. Biol. 5: a009167 (2013), which is hereby incorporated by reference in its entirety). MuSK is a single pass, 120 kDa transmembrane protein, composed of an extracellular region containing three Ig-like domains and a Frizzled (Fz)-like domain, and an intracellular region containing a juxtamembrane region, a kinase domain and a short cytoplasmic tail (Jennings et al., “Muscle-Specific trk-Related Receptor with a Kringle Domain Defines a Distinct Class of Receptor Tyrosine Kinases,” Proc. Natl. Acad. Sci. USA 90:2895-2899 (1993) and Valenzuela et al., “Receptor Tyrosine Kinase Specific for the Skeletal Muscle Lineage: Expression in Embryonic muscle, at the Neuromuscular Junction, and After Injury,” Neuron 15:573-584 (1995), which are hereby incorporated by reference in their entirety). MuSK phosphorylation is stimulated by agrin, a signal provided by motor neurons. Once activated, MuSK stimulates pathways that (1) cluster and anchor AChRs and additional muscle proteins critical for synaptic transmission, (2) enhance transcription of genes encoding synaptic proteins in muscle ‘synaptic nuclei’ and (3) promote the production of retrograde signals that promote presynaptic differentiation and attachment of motor nerve terminals to muscle. In the absence of MuSK, neuromuscular synapses fail to form (Burden et al., “The Role of MuSK in Synapse Formation and Neuromuscular Disease,” Cold Spring Harb. Perspect. Biol. 5: a009167 (2013), which is hereby incorporated by reference in its entirety). In addition to its role during synapse formation, MuSK is also required to maintain adult synapses, as inhibition of MuSK expression in adult muscle leads to profound defects in presynaptic and postsynaptic differentiation (Kong et al., “Inhibition of Synapse Assembly in Mammalian Muscle in vivo by RNA Interference,” EMBO Rep 5:183-188 (2004) and Hesser et al., “Synapse Disassembly and Formation of New Synapses in Postnatal Muscle Upon Conditional Inactivation of MuSK,” Mol. Cell. Neurosci. 31:470-480 (2006), which are hereby incorporated by reference in their entirety). Consistent with these findings in mice, mutations that impair MuSK kinase activity or inhibit signaling steps downstream from MuSK cause myasthenia (CM), characterized by structurally and functionally defective synapses, leading to muscle weakness and fatigue (Beeson et al., “Dok-7 Mutations Underlie a Neuromuscular Junction Synaptopathy,” Science 313:1975-1978 (2006); Muller et al., “Phenotypical Spectrum of DOK7 Mutations in Congenital Myasthenic Syndromes,” Brain 130:1497-1506 (2007); and Selcen et al., “A Compensatory Subpopulation of Motor Neurons in a Mouse Model of Amyotrophic Lateral Sclerosis,” J. Comp. Neurol. 490:209-219 (2008), which are hereby incorporated by reference in their entirely).

The amino acid sequence of human MuSK has the amino acid sequence of SEQ ID NO: 129 below.

(SEQ ID NO: 129)
MRELVNIPLVHILTLVAFSGTEKLPKAPVITTPLETVDALVEEVATFMCAVESYPQPEISWTRNKILIKLF
DTRYSIRENGQLLTILSVEDSDDGIYCCTANNGVGGAVESCGALQVKMKPKITRPPINVKIIEGLKAVLP
CTTMGNPKPSVSWIKGDSPLRENSRIAVLESGSLRIHNVQKEDAGQYRCVAKNSLGTAYSKVVKLEV
EVFARILRAPESHNVTFGSFVTLHCTATGIPVPTITWIENGNAVSSGSIQESVKDRVIDSRLQLFITKPGL
YTCIATNKHGEKFSTAKAAATISIAEWSKPQKDNKGYCAQYRGEVCNAVLAKDALVFLNTSYADPEEA
QELLVHTAWNELKVVSPVCRPAAEALLCNHIFQECSPGVVPTPIPICREYCLAVKELFCAKEWLVMEE
KTHRGLYRSEMHLLSVPECSKLPSMHWDPTACARLPHLDYNKENLKTFPPMTSSKPSVDIPNLPSSS
SSSFSVSPTYSMTVIISIMSSFAIFVLLTITTLYCCRRRKQWKNKKRESAAVTLTTLPSELLLDRLHPNPM
YQRMPLLLNPKLLSLEYPRNNIEYVRDIGEGAFGRVFQARAPGLLPYEPFTMVAVKMLKEEASADMQA
DFQREAALMAEFDNPNIVKLLGVCAVGKPMCLLFEYMAYGDLNEFLRSMSPHTVCSLSHSDLSMRAQ
VSSPGPPPLSCAEQLCIARQVAAGMAYLSERKFVHRDLATRNCLVGENMVVKIADFGLSRNIYSADYY
KANENDAIPIRWMPPESIFYNRYTTESDWWAYGVVLWEIFSYGLQPYYGMAHEEVIYYVRDGNILSCP
ENCPVELYNLMRLCWSKLPADRPSFTSIHRILERMCERAEGTVSV.

In some embodiments, the antibody-based molecule of the present invention binds the MuSK Frizzled (Fz)-like domain sequence of SEQ ID NO: 130.

In accordance with the present invention, the MuSK antibody-based molecules described herein bind to an epitope within the Frizzled (Fz)-like domain of the MuSK protein. The Fz-like domain of MuSK has the amino acid sequence of SEQ ID NO: 130 as shown below.

(SEQ ID NO: 130)
DNKGYCAQYRGEVCNAVLAKDALVFLNTSYADPEEAQELLVHTAWNELKVVSPVCRPAAEALLCNHI
FQECSPGVVPTPIPICREYCLAVKELFCAKEWLVMEEKTHRGLYRSEMHLLSVPECSKLPSMHWDPT
ACARL

The MuSK antibody-based molecules bind the human Fz-like domain of MuSK.

The term “epitope” as used herein refers to an antigenic determinant capable of being bound to an antibody. Epitopes usually comprise surface groupings of molecules such as amino acids or sugar side chains and usually have specific three-dimensional structural characteristics, as well as specific charge characteristics. Conformational and non-conformational epitopes are distinguished in that the binding to the former, but not the latter, is lost in the presence of denaturing solvents. An epitope may comprise amino acid residues directly involved in the binding (also called the immunodominant component of the epitope) and other amino acid residues, which are not directly involved in the binding, such as amino acid residues that are effectively blocked by the specific antigen-binding peptide (in other words, the amino acid residue is within the footprint of the specific antigen-binding peptide). An epitope typically includes at least 3, and more usually, at least 5, 6, 7, 8, 9, 10, 11, 12, 13, 14, 15, 16, 17, 18, 19, 20 or more amino acids in a unique spatial conformation.

In an embodiment, the MuSK antibody-based molecules, for use according to the invention binds the MuSK Frizzled (Fz) like domain. In an embodiment, the MuSK antibody or antigen binding fragment immunospecifically bind an epitope within the MuSK Fz-like domain sequence of SEQ ID NO: 130 more frequently, more rapidly, with greater duration and/or with greater affinity or avidity than an alternative epitope. In an embodiment, the MuSK antibody-based molecules described herein bind immunospecifically to any 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues of SEQ ID NO: 130. The term “affinity”, “specific binding”, “binding”, “immunospecific binding”, “binding activity” or “specific binding activity”, as used herein, refers to the degree to which an antibody or an antibody fragment as defined herein binds to an epitope within the MuSK-Fz-like domain sequence of SEQ ID NO:130.

In an embodiment, the MuSK antibody-based molecules as disclosed herein bind to the MuSK Fz-like domain with an affinity corresponding to a KD of about 10⁻⁷ M or less. For example, the MuSK antibody-based molecules disclosed herein bind to the MuSK Fz-like domain with an affinity corresponding to a KD of about 10⁻⁸ M, of about 10⁻⁹ M, of about 10⁻¹⁰ M, of about 10⁻¹¹ M, of about 10⁻¹² M or less when determined by, for instance, surface plasmon resonance (SPR) technology in a Biacore 3000 instrument (preferably using the antibody as the ligand and MuSK as the analyte). The MuSK antibody-based molecules as disclosed herein bind to the MuSK Fz-like domain with an affinity corresponding to a KD that is at least ten-fold lower, such as at least 100-fold lower, for instance at least 1,000-fold lower, such as at least 10,000-fold lower, for instance at least 100,000-fold lower than its affinity for binding to a non-specific antigen (e.g., bovine serum albumin, casein, etc.). The amount with which the affinity is lower is dependent on the KD of the antibody, so that when the KD of the antibody is very low (that is, the antibody is highly specific), then the amount with which the affinity for the antigen is lower than the affinity for a non-specific antigen may be at least 10,000 fold. The term “kd” (sec-1 or 1/s), as used herein, refers to the dissociation rate constant of a particular antibody-antigen interaction. The value is also referred to as the koff value and reflects the avidity of the antibody for its target. The term “ka” (M−1×sec−1 or 1/Ms), as used herein, refers to the association rate constant of a particular antibody-antigen interaction. The term “KD” (M), as used herein, refers to the dissociation equilibrium constant of a particular antibody-antigen interaction and is obtained by dividing the kd by the ka. The term “KA” (M−1 or 1/M), as used herein, refers to the association equilibrium constant of a particular antibody-antigen interaction and is obtained by dividing the ka by the kd.

In an embodiment, the MuSK antibody-based molecules described herein have a pH-dependent binding affinity for MuSK that allows for antibody recycling to enhance antigen binding. For example, in an embodiment, the association rate constant or dissociation rate constant may differ under acidic vs. neutral vs. basic pH conditions. In one embodiment, the MuSK antibody-based molecules described herein have a higher dissociation rate constant under acidic pH conditions, e.g., pH of <7.0, compared to neutral pH conditions, e.g., pH of ~7.0-7.9. In some embodiments, the MuSK antibody-based molecules described herein have a 2-fold to 3-fold higher dissociation rate constant (i.e., decreased binding affinity) at an acidic pH (e.g., pH ~5.5) as compared to a neutral pH. (pH ~7.4). In an embodiment, the MuSK antibody-based molecules bind the MuSK Fz-like domain with a higher affinity at neutral pH conditions than at acidic pH conditions. In other words, in an embodiment, the MuSK antibody-based molecules bind the MuSK Fz-like domain with a higher dissociation rate at acidic pH conditions than under neutral pH conditions. Neutral pH conditions may be defined as being a pH comprised from 7.0 to 7.9. Acidic pH conditions may be defined as being a pH being less than 7.0. Higher may mean at least 10%, 20%, 30%, 40%, 50%, 60%, 70%, 80%, 90%, 100%, 150%, 200%, 250%, 300% higher. Antibodies having this pH dependent dissociation characteristic dissociate from the antigen after binding and activation but before lysosomal degradation. Once dissociated, the antibody is transported via the neonatal Fc receptor back into circulation and is released to bind more antigen.

In some embodiments, the MuSK antibody-based molecule induces or activates MuSK phosphorylation. In some embodiments, binding of the MuSK antibodies of the present invention to their respective epitopes activated MuSK signaling. Preferably, the epitope is within the Fz-like domain and the binding to said epitope activates MuSK signalling. In particular, when the MuSK antibodies of the present invention bind their respective epitope of the MuSK Fz-like domain, this binding induces MuSK phosphorylation and activation. The MuSK antibodies of the present invention induce MuSK phosphorylation by about 50% to about 100% relative to MuSK phosphorylation induced by agrin activation (as measured, e.g., in a C2C12 phosphorylation assay). In this embodiment, the MuSK phosphorylation induced by the MuSK antibodies of the present invention is compared to the one induced by wild type agrin. In an embodiment, the MuSK antibodies of the present invention induce about 55%, 60%, 65%, 70%, 75%, 80%, 85%, 90%, 95% or 100% 95% MuSK phosphorylation (relative to MuSK phosphorylation induced by agrin activation without agrin deficiency). In an embodiment, the MuSK antibody-based molecules of the present invention induce about 90% to about 100% MuSK phosphorylation (relative to MuSK phosphorylation induced by agrin activation without agrin deficiency), upon MuSK binding. In this context the MuSK phosphorylation is assessed in a subject that is not deficient for agrin to be able to assess the level of MuSK phosphorylation properly. Phosphorylation of MuSK may be assessed using techniques known to the skilled person such as western blotting or a C2C12 myotube phosphorylation assay. Such antibodies activating MuSK signalling (i.e. induction of the dimerization of MuSK, induction of the tyrosine phosphorylation of MuSK) are agonist antibodies.

In some embodiments, the MuSK antibodies of the present invention, preferably MuSK antibodies that bind to the Fz-domain of MuSK, do not interfere (i.e., do not block, impede, inhibit, or reduce) with natural ligand binding and stimulation of MuSK. Some known MuSK binding proteins (or ligands) include Irp4/agrin and ColQ. In some embodiments, the MuSK antibodies of the present invention potentiate natural MuSK activation, i.e., phosphorylation, induced by natural ligand binding. In the context of the present invention, agrin may be seen as a natural ligand for MuSK activation via binding to LRP4. Such MuSK antibodies are agonist antibodies. In some embodiments, the antibodies of the invention, in combination with the natural ligand, activate MuSK (i.e., MuSK phosphorylation) to >20% of endogenous activation levels such as at least 30%, 40%, 50%, 60%, 70%, 80%, 90%, 100%, 110%, 130%, 150%, 200% of endogenous activation levels. MuSK phosphorylation may be assessed using techniques known to the skilled person. Such techniques may be western blotting.

Accordingly, in an embodiment, activities of the MuSK antibody-based molecules of the invention include: (i) binding to an epitope of human muscle-specific tyrosine-protein kinase (MuSK), preferably said epitope present in the MuSK Frizzled (Fz)-like domain sequence of SEQ ID NO: 130, wherein said antibody-based molecule induces MuSK phosphorylation upon binding to its epitope, and/or (ii) binding to its epitope, preferably the MuSK Fz-like domain does not block, impede, or inhibit natural or endogenous MuSK ligand induced phosphorylation, and may potentiate said natural or endogenous MuSK ligand induced phosphorylation, and (iii) binding to its epitope (preferably the MuSK Fz-like domain) occurs with a higher affinity at neutral pH conditions than at acidic pH conditions. In an embodiment, the MuSK antibody-based molecules bind to human Fz-like domain (SEQ ID NO: 130) of human MuSK.

In an embodiment, the MuSK antibody-based molecule for use in preventing, delaying, reversing or treating a disease or condition resulting from agrin deficiency in a human subject is an agonist MuSK antibody and/or has reduced or eliminated effector function.

In a first embodiment, the disease or condition resulting from agrin deficiency is such that the deficient agrin can no longer bind its receptor LRP4. In an embodiment, the MuSK antibody-based molecule for use in preventing, delaying, reversing or treating a disease or condition resulting from agrin deficiency: (i) binds to an epitope of human MuSK, preferably said epitope being preferably present in the MuSK Frizzled (Fz)-like domain sequence of SEQ ID NO: 130, and (ii) induces MuSK phosphorylation upon binding to its epitope. In such embodiment, the MuSK antibody-based molecule for use in preventing, delaying, reversing or treating a disease or condition resulting from agrin deficiency: (i) binds to an epitope of human MuSK, preferably said epitope being preferably present in the MuSK Frizzled (Fz)-like domain sequence of SEQ ID NO: 130, (ii) induces MuSK phosphorylation upon binding to its epitope and (iii) wherein the binding simulates the binding of the natural ligand of MuSK, being agrin or LRP/agrin.

In a second embodiment, the disease or condition resulting from agrin deficiency is such that the deficient agrin is still able to bind its natural receptor, LRP4 but with a deteriorated/decreased affinity and/or with a lower capacity to activate MuSK phosphorylation than its counterpart wild type agrin. In this embodiment, the MuSK antibody-based molecule for use in preventing, delaying, reversing or treating a disease or condition resulting from agrin deficiency may elicit a higher capacity to activate MuSK phosphorylation than deficient agrin.

In this context, affinity may be assessed using techniques known to the skilled person as earlier defined herein. Techniques for assessing the phosphorylation of MuSK are also known to the skilled person and have also been defined earlier herein.

In this embodiment, the deteriorated/decreased affinity of the deficient agrin to LRP4 may be at least 10%, 20%, 30%, 40%, 50%, 60%, 70%, 80%, 90% lower than the corresponding affinity of wild type agrin to LRP4.

In this embodiment, the capacity to activate MuSK phosphorylation by the deficient agrin is at least 10%, 20%, 30%, 40%, 50%, 60%, 70%, 80%, 90% less than the corresponding capacity of wild type agrin.

In this embodiment, the higher capacity to activate MuSK phosphorylation of the MuSK antibody-based molecule is at least 10%, 20%, 30%, 40%, 50%, 60%, 70%, 80%, 90%, 100%, 200%, 500%, 1000% higher than the corresponding capacity of deficient agrin.

In a third embodiment, the disease or condition resulting from agrin deficiency is such that the expression level of MuSK is lower than the expression level of MuSK in a subject who is not agrin deficient (as exemplified in FIG. 7C-2). In this embodiment, the MuSK antibody-based molecule for use in preventing, delaying, reversing or treating a disease or condition resulting from agrin deficiency may elicit a higher capacity to activate MuSK phosphorylation than deficient agrin (as exemplified in FIG. 7C-4).

Such properties allow to compensate for the lower expression level of MuSK.

Each of these features have been defined earlier herein.

In this embodiment, the lower expression of MuSK may at least 10%, 20%, 30%, 40%, 50%, 60%, 70%, 80%, 90% less than the corresponding expression of MuSK in a subject expressing wild type agrin.

In a fourth embodiment, the disease or condition resulting from agrin deficiency is such that the binding of LRP4 to MuSK is impaired/antagonized, which in its turn leads to an impaired activation of MuSK. A binding of LRP4 to MuSK may be considered to be impaired/antagonized when the binding affinity of LRP4 to MuSK is lower than the binding affinity of LRP4 to MuSK in subjects who are not deficient for agrin. In this embodiment, the MuSK antibody-based molecule for use in preventing, delaying, reversing or treating a disease or condition resulting from agrin deficiency may elicit a higher capacity to activate MuSK phosphorylation than deficient agrin.

Such properties allow to compensate for the impaired/antagonized binding of LRP4 to MuSK and/or for the impaired activation of MuSK.

Each of these features have been defined earlier herein.

In this embodiment, a lower binding affinity means the binding affinity is at least 10%, 20%, 30%, 40%, 50%, 60%, 70%, 80%, 90% lower than the corresponding binding affinity in a subject expressing wild type agrin. In this embodiment, the impaired activation of MuSK may be at least 10%, 20%, 30%, 40%, 50%, 60%, 70%, 80%, 90% lower than the corresponding activation of MuSK in a subject expressing wild type agrin.

Each of the first, third and/or fourth embodiments defined above may be combined. Each of the second, third and/or fourth embodiments defined above may be combined.

We hypothesized that agrin deficiency may cause less or no activation of MuSK and therefore less activation of the MuSK signalling pathway may occur. As a result, the NMJ may be impaired which may in turn have an impact on MuSK expression level. It is therefore surprising and unexpected that using a MuSK antibody-based molecule such therapeutic results could be obtained when MuSK expression level is decreased.

The MuSK antibody-based molecule may have reduced or eliminated effector function. Reduced or eliminated effector function may be obtained as earlier described herein by introducing mutation in the human IgG constant Fc region. Preferably, at least 1, 2, 3, 4, 5, 6, 7, 8, 9, 10, 11, 12, 13, 14, 15, 16 or 17 amino acid substitutions are introduced into said Fc region. Preferably, at least 1, 2, 3, 4, amino acid substitutions are introduced into said Fc region. Said Fc region may comprise SEQ ID NO: 266 or 267 and said substitutions are introduced at amino acid positions selected from amino acid residues 234, 235, 236, 237, 238, 239, 243, 265, 267, 268, 292, 297, 300, 318, 320, 322, 327, 328, 329, 330, 331, 332, and 396 numbered according to the EU numbering system of said sequence.

In an embodiment, said Fc region may comprise SEQ ID NO: 266 or 267 and said substitutions are introduced at amino acid positions selected from amino acid residues 234 or 235 of said sequence numbered according to the EU numbering system. In an embodiment, said Fc region may comprise SEQ ID NO: 266 or 267 and said substitutions are introduced at amino acid positions selected from amino acid residues 234 and 235 numbered according to the EU numbering system of said sequence.

In an embodiment, one or more of the following mutations (all numbered according to the EU numbering system) are introduced into the human IgG constant Fc region SEQ ID NO: 266 or SEQ ID NO: 267 of the antibody-based molecule described herein: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; or a P396L substitution.

In one embodiment, each of the combinations of mutations described earlier in the fourth embodiment of this application in the human IgG constant Fc region of the antibody-based molecule described herein may be made.

In a preferred embodiment, L234A or L235A substitution is introduced into the human IgG constant Fc region of the antibody-based molecule described herein. In a more preferred embodiment, L234A and L235A substitutions are introduced into the human IgG constant Fc region of the antibody-based molecule described herein. This embodiment results in an antibody-based molecule with a heavy chain represented by SEQ ID NO:268 or 270 or 282.

In an even more preferred embodiment, said anti-MuSK antibody or antigen binding fragment thereof, comprises:
a) a heavy chain variable domain (VH) comprising an amino acid sequence that is at least 80% identical or similar to SEQ ID NO: 234 and
b) a light chain variable domain (VL) comprising an amino acid sequence that is at least 80% identical or similar to SEQ ID NO: 235.

Within this context, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

Preferred anti-MuSK antibody or antigen binding fragment thereof, comprises:
a) a full length heavy chain comprising SEQ ID NO: 268 and
b) a full length light chain comprising SEQ ID NO: 269.

Preferred anti-MuSK antibody or antigen binding fragment thereof, comprises:
a) a full length heavy chain comprising SEQ ID NO: 270 and
b) a full length light chain comprising SEQ ID NO: 271.

In another even more preferred embodiment, said anti-MuSK antibody or antigen binding fragment thereof, comprises:
a) a heavy chain variable domain (VH) comprising an amino acid sequence that is at least 80% identical or similar to SEQ ID NO: 202 and
b) a light chain variable domain (VL) comprising an amino acid sequence that is at least 80% identical or similar to SEQ ID NO: 203.

Within this context, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

Preferred anti-MuSK antibody or antigen binding fragment thereof, comprises:
a) a full length heavy chain comprising SEQ ID NO: 282 and
b) a full length light chain comprising SEQ ID NO: 283.

In an embodiment, the antibody-based molecules of the present invention are “humanized,” particularly if they are to be employed for therapeutic purposes. The term “humanized” refers to a chimeric molecule, generally prepared using recombinant techniques, having an antigen-binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) of such variable domains grafted to appropriate human framework regions of human variable domains. The framework residues of such humanized molecules may be wild-type (e.g., fully human) or they may be modified to contain one or more amino acid substitutions not found in the human antibody whose sequence has served as the basis for humanization. Humanization lessens or eliminates the likelihood that a constant region of the molecule will act as an immunogen in human individuals, but the possibility of an immune response to the foreign variable region remains (LoBuglio, A.F. et al. “Mouse/Human Chimeric Monoclonal Antibody In Man: Kinetics And Immune Response,” Proc. Natl. Acad. Sci. USA 86:4220-4224 (1989), which is hereby incorporated by reference in its entirety). Another approach focuses not only on providing human-derived constant regions, but modifying the variable regions so as to reshape them as closely as possible to human form. The variable regions of both heavy and light chains contain three complementarity-determining regions (CDRs) which vary in response to the antigens in question and determine binding capability. The CDRs are flanked by four framework regions (FRs) which are relatively conserved in a given species and which putatively provide a scaffolding for the CDRs. When non-human antibodies are prepared with respect to a particular antigen, the variable regions can be “reshaped” or “humanized” by grafting CDRs derived from non-human antibody onto the FRs present in the human antibody to be modified. Suitable methods for humanizing the non-human antibody described herein are known in the art see e.g., Sato, K. et al., Cancer Res 53:851-856 (1993); Riechmann, L. et al., “Reshaping Human Antibodies for Therapy,” Nature 332:323-327 (1988); Verhoeyen, M. et al., “Reshaping Human Antibodies: Grafting An Antilysozyme Activity,” Science 239:1534-1536 (1988); Kettleborough, C. A. et al., “Humanization Of A Mouse Monoclonal Antibody By CDR-Grafting: The Importance Of Framework Residues On Loop Conformation,” Protein Engineering 4:773-3783 (1991); Maeda, H. et al., “Construction Of Reshaped Human Antibodies With HIV-Neutralizing Activity,” Human Antibodies Hybridoma 2:124-134 (1991); Gorman, S. D. et al., “Reshaping A Therapeutic CD4 Antibody,” Proc. Natl. Acad. Sci. USA 88:4181-4185 (1991); Tempest, P. R. et al., “Reshaping A Human Monoclonal Antibody To Inhibit Human Respiratory Syncytial Virus Infection In Vivo,” Bio/Technology 9:266-271 (1991); Co, M. S. et al., “Humanized Antibodies For Antiviral Therapy,” Proc. Natl. Acad. Sci. USA 88:2869-2873 (1991); Carter, P. et al., “Humanization Of An Anti-p185her2 Antibody For Human Cancer Therapy,” Proc. Natl. Acad. Sci. USA 89:4285-4289 (1992); and Co, M.S. et al., “Chimeric And Humanized Antibodies With Specificity For The CD33 Antigen,” J. Immunol. 148:1149-1154 (1992), which are hereby incorporated by reference in their entirety. In some embodiments, humanized MuSK antibodies of the present invention preserve all CDR sequences (for example, a humanized antibody containing all six CDRs from the llama or mouse antibody). In other embodiments, humanized MuSK antibodies of the present invention have one or more CDRs (one, two, three, four, five, six) which are altered with respect to the original antibody. Methods of humanizing an antibody are well-known in the art and suitable for humanizing the antibodies disclosed herein (see, e.g., U.S. Pat. No. 5,225,539 to Winter; U.S. Pat. Nos. 5,530,101 and 5,585,089 to Queen and Selick; U.S. Pat. No. 5,859,205 to Robert et al.; U.S. Pat. No. 6,407,213 to Carter; and U.S. Pat. No. 6,881,557 to Foote, which are hereby incorporated by reference in their entirety).

In some antibodies only part of a CDR, namely the subset of CDR residues required for binding termed the “specificity determining residues” (“SDRs”), are needed to retain binding of the antibody. CDR residues not contacting antigen and not in the SDRs can be identified based on previous studies from regions of Kabat CDRs lying outside Chothia hypervariable loops (see, Kabat et al., SEQUENCES OF PROTEINS OF IMMUNOLOGICAL INTEREST, National Institutes of Health Publication No. 91-3242 (1992); Chothia, C. et al., “Canonical Structures For The Hypervariable Regions Of Immunoglobulins,” J. Mol. Biol. 196:901-917 (1987), which are hereby incorporated by reference in their entirety), by molecular modeling and/or empirically, or as described in Gonzales, N. R. et al., “SDR Grafting Of A Murine Antibody Using Multiple Human Germline Templates To Minimize Its Immunogenicity,” Mol. Immunol. 41:863-872 (2004), which is hereby incorporated by reference in its entirety. In such humanized antibodies, at positions in which one or more donor CDR residues is absent or in which an entire donor CDR is omitted, the amino acid residue occupying the position can be an amino acid residue occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of non-human amino acids in a humanized antibody and consequently decreasing potential immunogenicity. However, substitutions can also cause changes of affinity, and significant reductions in affinity are preferably avoided. Substitutions may also cause changes of activity. Such substitutions causing a significant reduction in activity are also preferably avoided. In this context, the antibody or antibody fragment should still exhibit a detectable activity of the antibody as earlier defined herein or an activity of the antibody at least to some extent. Positions for substitution within CDRs and amino acids to substitute can also be selected empirically.

Phage display technology can alternatively be used to increase (or decrease) CDR affinity of the antibody-based molecules of the present invention. This technology, referred to as affinity maturation, employs mutagenesis or “CDR walking” and re-selection using the target antigen or an antigenic fragment thereof to identify antibodies having CDRs that bind with higher (or lower) affinity to the antigen when compared with the initial or parental antibody (see, e.g. Glaser et al., “Antibody Engineering By Codon-Based Mutagenesis In A Filamentous Phage Vector System,” J. Immunology 149:3903-3913 (1992), which is hereby incorporated by reference in its entirety). Mutagenizing entire codons rather than single nucleotides results in a semi-randomized repertoire of amino acid mutations. Libraries can be constructed consisting of a pool of variant clones each of which differs by a single amino acid alteration in a single CDR from another member of such library and which contain variants potentially representing each possible amino acid substitution for each CDR residue. Mutants with increased (or decreased) binding affinity for the antigen can be screened by contacting the immobilized mutants with labeled antigen. Any screening method known in the art can be used to identify variant antibody-based binding molecules with increased or decreased affinity to the antigen (e.g., ELISA) (See Wu, H. et al., “Stepwise In Vitro Affinity Maturation Of Vitaxin, An Alphav Beta3-Specific Humanized mAb,” Proc. Natl. Acad. Sci. USA 95:6037-6042 (1998); Yelton et al., “Affinity Maturation Of The BR96 Anti-Carcinoma Antibody By Codon-Based Mutagenesis,” J. Immunology 155:1994 (1995), which are hereby incorporated by reference in their entirety). CDR walking, which randomizes the light chain may be used (see, Schier, R. et al., “Isolation Of Picomolar Affinity Anti-c-erbB-2 Single-Chain Fv By Molecular Evolution Of The Complementarity Determining Regions In The Center Of The Antibody Binding Site,” J. Mol. Biol. 263:551-567 (1996), which is hereby incorporated by reference in its entirety).

Methods for affinity maturation of the MuSK antibody molecule are described herein and disclosed for example, in Krause, J. C. et al., “An Insertion Mutation That Distorts Antibody Binding Site Architecture Enhances Function of a Human Antibody,” MBio. 2 (1): e00345-10 (2011); Kuan, C.T. et al., “Affinity-Matured Anti-Glycoprotein NMB Recombinant Immunotoxins Targeting Malignant Gliomas And Melanomas,” Int. J. Cancer 10.1002/ijc.25645 (2010); Hackel, B. J. et al., “Stability And CDR Composition Biases Enrich Binder Functionality Landscapes,” J. Mol. Biol. 401 (1): 84-96 (2010); Montgomery, D. L. et al., “Affinity Maturation And Characterization Of A Human Monoclonal Antibody Against HIV-1 gp41,” MAbs 1 (5): 462-474 (2009); Gustchina, E. et al., “Affinity Maturation By Targeted Diversification Of The CDR-H2 Loop Of A Monoclonal Fab Derived From A Synthetic Naïve Human Antibody Library And Directed Against The Internal Trimeric Coiled-Coil Of Gp41 Yields A Set Of Fabs With Improved HIV-1 Neutralization Potency And Breadth,” Virology 393 (1): 112-119 (2009); Finlay, W. J. et al., “Affinity Maturation Of A Humanized Rat Antibody For Anti-RAGE Therapy: Comprehensive Mutagenesis Reveals A High Level Of Mutational Plasticity Both Inside And Outside The Complementarity-Determining Regions,” J. Mol. Biol. 388 (3): 541-558 (2009); Bostrom, J. et al., “Improving Antibody Binding Affinity And Specificity For Therapeutic Development,” Methods Mol. Biol. 525:353-376 (2009); Steidl, S. et al., “In Vitro Affinity Maturation Of Human GM-CSF Antibodies By Targeted CDR-Diversification,” Mol. Immunol. 46 (1): 135-144 (2008); and Barderas, R. et al., “Affinity Maturation Of Antibodies Assisted By In Silico Modeling,” Proc. Natl. Acad. Sci. USA 105 (26): 9029-9034 (2008), which are hereby incorporated by reference in their entirety.

In the context of this application, an amino acid alteration (change or modification) may be an amino acid substitution, addition, deletion or chemical modification.

In an embodiment, the MuSK-antibody based molecule as described herein comprises the amino acid sequence of any one, any two, any three, any four, any five, or any six CDRs as provided in Tables 1 and 2 herein.

In one embodiment, the antibody-based molecule that binds to human muscle-specific tyrosine-protein kinase (MuSK) comprises a heavy chain variable domain, where the heavy chain variable domain comprises:
(i) a complementarity-determining region 1 (CDR-H1) comprising an amino acid sequence of any one of SEQ ID NOS: 1-16, 135, 136, 147-149 or a modified amino acid sequence of any one of SEQ ID NOS: 1-16, 135, 136, or 147-149 said modified sequence having 1, 2, 3, 4 or 5 amino acid alterations relative to SEQ ID NOs: 1-16, 135, 136 or 147-149;
(ii) a complementarity-determining region 2 (CDR-H2) comprising an amino acid sequence of any one of SEQ ID NOS: 17-32, 137, 138, 150-155 or a modified amino acid sequence of any one of SEQ ID NOs: 17-32, 137, 138, or 150-155 said modified sequences having 1, 2, 3, 4 or 5 amino acid alterations relative to SEQ ID NOs: 17-32, 137, 138, or 150-155; and
(iii) a complementarity-determining region 3 (CDR-H3) comprising an amino acid sequence of any one of SEQ ID NOs: 33-48, 139, 140, 156-158, 240-251, or a modified amino acid sequence of any one of SEQ ID NO: 33-48, 139, 140, 156-158, or 240-251, said modified sequence having 1, 2, 3, 4 or 5 amino acid alterations relative to SEQ ID NOs: 33-48, 139, 140, 156-158, or 240-251.

In an embodiment, the antibody-based molecule that binds to human muscle-specific tyrosine-protein kinase (MuSK) comprises:
(i) a heavy chain variable domain comprising the CDR-H1 of SEQ ID NO: 1 or having 1, 2, 3, 4 or 5 amino acid alterations relative to SEQ ID NO: 1, the CDR-H2 of SEQ ID NO: 17 or having 1, 2, 3, 4 or 5 amino acid alterations relative to SEQ ID NO:17, and the CDR-H3 of SEQ ID NO: 33 or having 1, 2, 3, 4 or 5 amino acid alterations relative to SEQ ID NO:33;
(ii) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 34 or having 1, 2, 3, 4 or 5 amino acid alterations relative to SEQ ID NO:34;
(iii) a heavy chain variable domain comprising the CDR-H1 of SEQ ID NO: 3 or having 1, 2, 3, 4 or 5 amino acid alterations relative to SEQ ID NO:3, the CDR-H2 of SEQ ID NO: 19 or having 1, 2, 3, 4 or 5 amino acid alterations relative to SEQ ID NO:19, and the CDR-H3 of SEQ ID NO: 35 or having 1, 2, 3, 4 or 5 amino acid alterations relative to SEQ ID NO:35;
(iv) a heavy chain variable domain comprising the CDR-H1 of SEQ ID NO: 4 or having 1, 2, 3, 4 or 5 amino acid alterations relative to SEQ ID NO:4, the CDR-H2 of SEQ ID NO: 20 or having 1, 2, 3, 4 or 5 amino acid alterations relative to SEQ ID NO:20, and the CDR-H3 of SEQ ID NO: 36 or having 1, 2, 3, 4 or 5 amino acid alterations relative to SEQ ID NO:36;
(v) a heavy chain variable domain comprising the CDR-H1 of SEQ ID NO: 5 or having 1, 2, 3, 4 or 5 amino acid alterations relative to SEQ ID NO:5, the CDR-H2 of SEQ ID NO: 21 or having 1, 2, 3, 4 or 5 amino acid alterations relative to SEQ ID NO:21, and the CDR-H3 of SEQ ID NO: 37 or having 1, 2, 3, 4 or 5 amino acid alterations relative to SEQ ID NO:37;
(vi) a heavy chain variable domain comprising the CDR-H1 of SEQ ID NO: 6 or having 1, 2, 3, 4 or 5 amino acid alterations relative to SEQ ID NO:6, the CDR-H2 of SEQ ID NO: 22 or having 1, 2, 3, 4 or 5 amino acid alterations relative to SEQ ID NO:22, and the CDR-H3 of SEQ ID NO: 38 or having 1, 2, 3, 4 or 5 amino acid alterations relative to SEQ ID NO:38;
(vii) a heavy chain variable domain comprising the CDR-H1 of SEQ ID NO: 7 or having 1, 2, 3, 4 or 5 amino acid alterations relative to SEQ ID NO:7, the CDR-H2 of SEQ ID NO: 23 or having 1, 2, 3, 4 or 5 amino acid alterations relative to SEQ ID NO:23, and the CDR-H3 of SEQ ID NO: 39 or having 1, 2, 3, 4 or 5 amino acid alterations relative to SEQ ID NO:39;
(viii) a heavy chain variable domain comprising the CDR-H1 of SEQ ID NO: 8 or having 1, 2, 3, 4 or 5 amino acid alterations relative to SEQ ID NO:8, the CDR-H2 of SEQ ID NO: 24 or having 1, 2, 3, 4 or 5 amino acid alterations relative to SEQ ID NO:24, and the CDR-H3 of SEQ ID NO: 40 or having 1, 2, 3, 4 or 5 amino acid alterations relative to SEQ ID NO:40;
(ix) a heavy chain variable domain comprising the CDR-H1 of SEQ ID NO: 9 or having 1, 2, 3, 4 or 5 amino acid alterations relative to SEQ ID NO:9, the CDR-H2 of SEQ ID NO: 25 or having 1, 2, 3, 4 or 5 amino acid alterations relative to SEQ ID NO:25, and the CDR-H3 of SEQ ID NO: 41 or having 1, 2, 3, 4 or 5 amino acid alterations relative to SEQ ID NO:41;
(x) a heavy chain variable domain comprising the CDR-H1 of SEQ ID NO: 10 or having 1, 2, 3, 4 or 5 amino acid alterations relative to SEQ ID NO:10, the CDR-H2 of SEQ ID NO: 26 or having 1, 2, 3, 4 or 5 amino acid alterations relative to SEQ ID NO:26, and the CDR-H3 of SEQ ID NO: 42 or having 1, 2, 3, 4 or 5 amino acid alterations relative to SEQ ID NO:42;
(xi) a heavy chain variable domain comprising the CDR-H1 of SEQ ID NO: 11 or having 1, 2, 3, 4 or 5 amino acid alterations relative to SEQ ID NO:11, the CDR-H2 of SEQ ID NO: 27 or having 1, 2, 3, 4 or 5 amino acid alterations relative to SEQ ID NO:27, and the CDR-H3 of SEQ ID NO: 43 or having 1, 2, 3, 4 or 5 amino acid alterations relative to SEQ ID NO:43;
(xii) a heavy chain variable domain comprising the CDR-H1 of SEQ ID NO: 12 or having 1, 2, 3, 4 or 5 amino acid alterations relative to SEQ ID NO:12, the CDR-H2 of SEQ ID NO: 28 or having 1, 2, 3, 4 or 5 amino acid alterations relative to SEQ ID NO:28, and the CDR-H3 of SEQ ID NO: 44 or having 1, 2, 3, 4 or 5 amino acid alterations relative to SEQ ID NO:44;
(xiii) a heavy chain variable domain comprising the CDR-H1 of SEQ ID NO: 13 or having 1, 2, 3, 4 or 5 amino acid alterations relative to SEQ ID NO:13, the CDR-H2 of SEQ ID NO: 29 or having 1, 2, 3, 4 or 5 amino acid alterations relative to SEQ ID NO:29, and the CDR-H3 of SEQ ID NO: 45 or having 1, 2, 3, 4 or 5 amino acid alterations relative to SEQ ID NO:45;
(xiv) a heavy chain variable domain comprising the CDR-H1 of SEQ ID NO: 14 or having 1, 2, 3, 4 or 5 amino acid alterations relative to SEQ ID NO:14, the CDR-H2 of SEQ ID NO: 30 or having 1, 2, 3, 4 or 5 amino acid alterations relative to SEQ ID NO:30, and the CDR-H3 of SEQ ID NO: 46 or having 1, 2, 3, 4 or 5 amino acid alterations relative to SEQ ID NO:46;
(xv) a heavy chain variable domain comprising the CDR-H1 of SEQ ID NO: 15 or having 1, 2, 3, 4 or 5 amino acid alterations relative to SEQ ID NO:15, the CDR-H2 of SEQ ID NO: 31 or having 1, 2, 3, 4 or 5 amino acid alterations relative to SEQ ID NO:31, and the CDR-H3 of SEQ ID NO: 47 or having 1, 2, 3, 4 or 5 amino acid alterations relative to SEQ ID NO:47;
(xvi) a heavy chain variable domain comprising the CDR-H1 of SEQ ID NO: 16 or having 1, 2, 3, 4 or 5 amino acid alterations relative to SEQ ID NO: 16, the CDR-H2 of SEQ ID NO: 32 or having 1, 2, 3, 4 or 5 amino acid alterations relative to SEQ ID NO:32, and the CDR-H3 of SEQ ID NO: 48 or having 1, 2, 3, 4 or 5 amino acid alterations relative to SEQ ID NO:48;
(xvii) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 139 or having 1, 2, 3, 4 or 5 amino acid alterations relative to SEQ ID NO: 139; or
(xviii) a heavy chain variable domain comprising the CDR-H1 of SEQ ID NO: 136 or having 1, 2, 3, 4 or 5 amino acid alterations relative to SEQ ID NO:136, the CDR-H2 of SEQ ID NO: 138 or having 1, 2, 3, 4 or 5 amino acid alterations relative to SEQ ID NO:138, and the CDR-H3 of SEQ ID NO: 140 or having 1, 2, 3, 4 or 5 amino acid alterations relative to SEQ ID NO:140. The sequences of the heavy chain CDRs are provided in Table 1.

In an embodiment, the antibody-based molecule that binds to human muscle-specific tyrosine-protein kinase (MuSK) comprises:
(ii.a) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 240 (X2m1) or having 1, 2, 3, 4 or 5 amino acid alterations relative to SEQ ID NO:240;
(ii.b) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 241 (X2m2) or having 1, 2, 3, 4 or 5 amino acid alterations relative to SEQ ID NO:241;
(ii.c) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO: 18, and the CDR-H3 of SEQ ID NO: 242 (X2m3) or having 1, 2, 3, 4 or 5 amino acid alterations relative to SEQ ID NO:242;
(ii.d) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 243 (X2m4) or having 1, 2, 3, 4 or 5 amino acid alterations relative to SEQ ID NO:243;
(ii.e) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 244 (X2m5) or having 1, 2, 3, 4 or 5 amino acid alterations relative to SEQ ID NO:244;
(ii.f) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 245 (X2m6) or having 1, 2, 3, 4 or 5 amino acid alterations relative to SEQ ID NO:245;
(ii.g) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO:18, and the CDR-H3 of SEQ ID NO: 246 (X2m7) or having 1, 2, 3, 4 or 5 amino acid alterations relative to SEQ ID NO:246; or (ii.h) a heavy chain variable domain comprising the CDR-H1 of SEQ ID NO: 2 or having 1, 2, 3, 4 or 5 amino acid alterations relative to SEQ ID NO:2, the CDR-H2 of SEQ ID NO: 18 or having 1, 2, 3, 4 or 5 amino acid alterations relative to SEQ ID NO: 18, and the CDR-H3 of SEQ ID NO: 247 (X2m8) or having 1, 2, 3, 4 or 5 amino acid alterations relative to SEQ ID NO:247.

In an embodiment, the antibody-based molecule that binds to human muscle-specific tyrosine-protein kinase (MuSK) comprises:
(xvii.a) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 248 (X17m1) or having 1, 2, 3, 4 or 5 amino acid alterations relative to SEQ ID NO:248;
(xvii.b) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO: 137, and the CDR-H3 of SEQ ID NO: 249 (X17m2) or having 1, 2, 3, 4 or 5 amino acid alterations relative to SEQ ID NO:249;
(xvii.c) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 250 (X17m3) or having 1, 2, 3, 4 or 5 amino acid alterations relative to SEQ ID NO:250; or
(xvii.d) a heavy chain variable domain comprising the CDR-H1 of SEQ ID NO: 135 or having 1, 2, 3, 4 or 5 amino acid alterations relative to SEQ ID NO:135, the CDR-H2 of SEQ ID NO: 137 or having 1, 2, 3, 4 or 5 amino acid alterations relative to SEQ ID NO:137, and the CDR-H3 of SEQ ID NO: 251 (X17m6) or having 1, 2, 3, 4 or 5 amino acid alterations relative to SEQ ID NO:251.

In an embodiment, the antibody-based molecule that binds to human muscle-specific tyrosine-protein kinase (MuSK) comprises a heavy chain variable domain, where the heavy chain variable domain comprises:
(xix) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 150 or having 1, 2, 3, 4 or 5 amino acid alterations relative to SEQ ID NO: 150, and the CDR-H3 of SEQ ID NO: 156 or having 1, 2, 3, 4 or 5 amino acid alterations relative to SEQ ID NO: 156;
(xx) a heavy chain variable domain comprising the CDR-H1 of SEQ ID NO: 148 or having 1, 2, 3, 4 or 5 amino acid alterations relative to SEQ ID NO:148, the CDR-H2 of SEQ ID NO: 151 or having 1, 2, 3, 4 or 5 amino acid alterations relative to SEQ ID NO: 151 and the CDR-H3 of SEQ ID NO: 157 or having 1, 2, 3, 4 or 5 amino acid alterations relative to SEQ ID NO:157; or
(xxi) a heavy chain variable domain comprising the CDR-H1 of SEQ ID NO: 149 or having 1, 2, 3, 4 or 5 amino acid alterations relative to SEQ ID NO:149, the CDR-H2 of SEQ ID NO: 152 or having 1, 2, 3, 4 or 5 amino acid alterations relative to SEQ ID NO: 152, and the CDR-H3 of SEQ ID NO: 158 or having 1, 2, 3, 4 or 5 amino acid alterations relative to SEQ ID NO:158.

In an embodiment, the antibody-based molecule that binds to human muscle-specific tyrosine-protein kinase (MuSK) comprises a heavy chain variable domain, where the heavy chain variable domain comprises:
(xxii) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 153 or having 1, 2, 3, 4 or 5 amino acid alterations relative to SEQ ID NO:153, and the CDR-H3 of SEQ ID NO:156 (3B2g1m1/3B2g2m1) or having 1, 2, 3, 4 or 5 amino acid alterations relative to SEQ ID NO: 156;
(xxiii) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 154 or having 1, 2, 3, 4 or 5 amino acid alterations relative to SEQ ID NO:154, and the CDR-H3 of SEQ ID NO: 156 (3B2g1m2/3B2g2m2) or having 1, 2, 3, 4 or 5 amino acid alterations relative to SEQ ID NO:156; or
(xxiv) a heavy chain variable domain comprising the CDR-H1 of SEQ ID NO: 147 or having 1, 2, 3, 4 or 5 amino acid alterations relative to SEQ ID NO:147, the CDR-H2 of SEQ ID NO: 155 or having 1, 2, 3, 4 or 5 amino acid alterations relative to SEQ ID NO:155, and the CDR-H3 of SEQ ID NO: 156 (3B2g1m4/3B2g2m4) or having 1, 2, 3, 4 or 5 amino acid alterations relative to SEQ ID NO:156. The sequences of the heavy chain CDRs are provided in Table 1.

In an embodiment, the antibody-based molecule that binds to human muscle-specific tyrosine-protein kinase (MuSK) comprises:
(i) a heavy chain variable domain comprising the CDR-H1 of SEQ ID NO: 1, the CDR-H2 of SEQ ID NO: 17, and the CDR-H3 of SEQ ID NO: 33;
(ii) a heavy chain variable domain comprising the CDR-H1 of SEQ ID NO: 2, the CDR-H2 of SEQ ID NO: 18, and the CDR-H3 of SEQ ID NO: 34;
(iii) a heavy chain variable domain comprising the CDR-H1 of SEQ ID NO: 3, the CDR-H2 of SEQ ID NO: 19, and the CDR-H3 of SEQ ID NO: 35;
(iv) a heavy chain variable domain comprising the CDR-H1 of SEQ ID NO: 4, the CDR-H2 of SEQ ID NO: 20, and the CDR-H3 of SEQ ID NO: 36;
(v) a heavy chain variable domain comprising the CDR-H1 of SEQ ID NO: 5, the CDR-H2 of SEQ ID NO: 21, and the CDR-H3 of SEQ ID NO: 37;
(vi) a heavy chain variable domain comprising the CDR-H1 of SEQ ID NO: 6, the CDR-H2 of SEQ ID NO: 22, and the CDR-H3 of SEQ ID NO: 38;
(vii) a heavy chain variable domain comprising the CDR-H1 of SEQ ID NO: 7, the CDR-H2 of SEQ ID NO: 23, and the CDR-H3 of SEQ ID NO: 39;
(viii) a heavy chain variable domain comprising the CDR-H1 of SEQ ID NO: 8, the CDR-H2 of SEQ ID NO: 24, and the CDR-H3 of SEQ ID NO: 40;
(ix) a heavy chain variable domain comprising the CDR-H1 of SEQ ID NO: 9, the CDR-H2 of SEQ ID NO: 25, and the CDR-H3 of SEQ ID NO: 41;
(x) a heavy chain variable domain comprising the CDR-H1 of SEQ ID NO: 10, the CDR-H2 of SEQ ID NO: 26, and the CDR-H3 of SEQ ID NO: 42;
(xi) a heavy chain variable domain comprising the CDR-H1 of SEQ ID NO: 11, the CDR-H2 of SEQ ID NO: 27, and the CDR-H3 of SEQ ID NO: 43;
(xii) a heavy chain variable domain comprising the CDR-H1 of SEQ ID NO: 12, the CDR-H2 of SEQ ID NO: 28, and the CDR-H3 of SEQ ID NO: 44;
(xiii) a heavy chain variable domain comprising the CDR-H1 of SEQ ID NO: 13, the CDR-H2 of SEQ ID NO: 29, and the CDR-H3 of SEQ ID NO: 45;
(xiv) a heavy chain variable domain comprising the CDR-H1 of SEQ ID NO: 14, the CDR-H2 of SEQ ID NO: 30, and the CDR-H3 of SEQ ID NO: 46;
(xv) a heavy chain variable domain comprising the CDR-H1 of SEQ ID NO: 15, the CDR-H2 of SEQ ID NO: 31, and the CDR-H3 of SEQ ID NO: 47;
(xvi) a heavy chain variable domain comprising the CDR-H1 of SEQ ID NO: 16, the CDR-H2 of SEQ ID NO: 32, and the CDR-H3 of SEQ ID NO: 48;
(xvii) a heavy chain variable domain comprising the CDR-H1 of SEQ ID NO: 135, the CDR-H2 of SEQ ID NO: 137, and the CDR-H3 of SEQ ID NO: 139; or
(xviii) a heavy chain variable domain comprising the CDR-H1 of SEQ ID NO: 136, the CDR-H2 of SEQ ID NO: 138, and the CDR-H3 of SEQ ID NO: 140. The sequences of the heavy chain CDR sequences are provided in Table 1 below.

In an embodiment, the antibody-based molecule that binds to human muscle-specific tyrosine-protein kinase (MuSK) comprises:
(ii.a) a heavy chain variable domain comprising the CDR-H1 of SEQ ID NO: 2, the CDR-H2 of SEQ ID NO: 18, and the CDR-H3 of SEQ ID NO: 240 (X2m1);
(ii.b) a heavy chain variable domain comprising the CDR-H1 of SEQ ID NO: 2, the CDR-H2 of SEQ ID NO: 18, and the CDR-H3 of SEQ ID NO: 241 (X2m2);
(ii.c) a heavy chain variable domain comprising the CDR-H1 of SEQ ID NO: 2, the CDR-H2 of SEQ ID NO: 18, and the CDR-H3 of SEQ ID NO: 242 (X2m3);
(ii.d) a heavy chain variable domain comprising the CDR-H1 of SEQ ID NO: 2, the CDR-H2 of SEQ ID NO: 18, and the CDR-H3 of SEQ ID NO: 243 (X2m4);
(ii.e) a heavy chain variable domain comprising the CDR-H1 of SEQ ID NO: 2, the CDR-H2 of SEQ ID NO: 18, and the CDR-H3 of SEQ ID NO: 244 (X2m5);
(ii.f) a heavy chain variable domain comprising the CDR-H1 of SEQ ID NO: 2, the CDR-H2 of SEQ ID NO: 18, and the CDR-H3 of SEQ ID NO: 245 (X2m6);
(ii.g) a heavy chain variable domain comprising the CDR-H1 of SEQ ID NO: 2, the CDR-H2 of SEQ ID NO: 18, and the CDR-H3 of SEQ ID NO: 246 (X2m7); or (ii.h) a heavy chain variable domain comprising the CDR-H1 of SEQ ID NO: 2, the CDR-H2 of SEQ ID NO: 18, and the CDR-H3 of SEQ ID NO: 247 (X2m8).

In an embodiment, the antibody-based molecule that binds to human muscle-specific tyrosine-protein kinase (MuSK) comprises:
(xvii.a) a heavy chain variable domain comprising the CDR-H1 of SEQ ID NO: 135, the CDR-H2 of SEQ ID NO: 137, and the CDR-H3 of SEQ ID NO: 248 (X17m1);
(xvii.b) a heavy chain variable domain comprising the CDR-H1 of SEQ ID NO: 135, the CDR-H2 of SEQ ID NO: 137, and the CDR-H3 of SEQ ID NO: 249 (X17m2);
(xvii.c) a heavy chain variable domain comprising the CDR-H1 of SEQ ID NO: 135, the CDR-H2 of SEQ ID NO: 137, and the CDR-H3 of SEQ ID NO: 250 (X17m3); or (xvii.d) a heavy chain variable domain comprising the CDR-H1 of SEQ ID NO: 135, the CDR-H2 of SEQ ID NO: 137, and the CDR-H3 of SEQ ID NO: 251 (X17m6).

In an embodiment, the antibody-based molecule that binds to human muscle-specific tyrosine-protein kinase (MuSK) comprises:
(xix) a heavy chain variable domain comprising the CDR-H1 of SEQ ID NO: 147, the CDR-H2 of SEQ ID NO: 150, and the CDR-H3 of SEQ ID NO: 156;
(xx) a heavy chain variable domain comprising the CDR-H1 of SEQ ID NO: 148, the CDR-H2 of SEQ ID NO: 151, and the CDR-H3 of SEQ ID NO: 157; or
(xxi) a heavy chain variable domain comprising the CDR-H1 of SEQ ID NO: 149, the CDR-H2 of SEQ ID NO: 152, and the CDR-H3 of SEQ ID NO: 158.

In an embodiment, the antibody-based molecule that binds to human muscle-specific tyrosine-protein kinase (MuSK) comprises:
(xxii) a heavy chain variable domain comprising the CDR-H1 of SEQ ID NO: 147, the CDR-H2 of SEQ ID NO: 153, and the CDR-H3 of SEQ ID NO:156 (3B2g1m1/3B2g2m1);
(xxiii) a heavy chain variable domain comprising the CDR-H1 of SEQ ID NO: 147, the CDR-H2 of SEQ ID NO: 154, and the CDR-H3 of SEQ ID NO: 156 (3B2g1m2/3B2g2m2); or
(xxiv) a heavy chain variable domain comprising the CDR-H1 of SEQ ID NO: 147, the CDR-H2 of SEQ ID NO: 155, and the CDR-H3 of SEQ ID NO: 156 (3B2g1m4/3B2g2m4). The sequences of the heavy chain CDR sequences are provided in Table 1 below.

In an embodiment, the antibody-based molecule that binds to human muscle-specific tyrosine-protein kinase (MuSK) comprises a heavy chain variable domain, where the heavy chain variable domain comprises the CDR-H1 of SEQ ID NO: 147, CDR-H2 of SEQ ID NO: 153 or a CDR-H2 amino acid sequence having at least 0,1,2,3,4, or 5 alterations relative to SEQ ID NO: 153, and the CDR-H3 of SEQ ID NO:156 (3B2g2m1). In an embodiment, the CDR-H2 amino acid sequence has at least 0,1,2,3,4, or 5 alterations relative to SEQ ID NO: 153. In accordance with this embodiment, the CDR-H2 amino acid sequence has at least 0,1,2,3,4, or 5 alterations relative to SEQ ID NO: 153, wherein said alterations are present at residues 1, 2, 6, 7, 8, 9, 10, 11, 12, 13, 14, 15, 16, 17, or any combination thereof.

In an embodiment, the antibody-based molecule that binds to human muscle-specific tyrosine-protein kinase (MuSK) comprises a heavy chain variable domain, where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 150 or having 1, 2, 3, 4 or 5 amino acid alterations relative to SEQ ID NO: 150, and
a CDR-H3 amino acid sequence comprising SEQ ID NO: 156 or having 1, 2, 3, 4 or 5 amino acid alterations relative to SEQ ID NO:156.

In an embodiment, the CDR-H2 of the antibody comprises a proline (P) at position 3, a tryptophan (W) 30 at position 4, and a serine(S) or asparagine (N) at position 5. In a preferred embodiment, the CDR-H2 of the antibody comprises a proline (P) at position 3, a tryptophan (W) at position 4, and an asparagine (N) at position 5.

In an embodiment, the antibody-based molecule that binds to human muscle-specific tyrosine-protein kinase (MuSK) comprises a heavy chain variable domain, where the heavy chain variable domain comprises:
a CDR-H1 amino acid sequence comprising or consisting of SEQ ID NO: 147,
a CDR-H2 amino acid sequence comprising or consisting of SEQ ID NO: 150, and
a CDR-H3 amino acid sequence comprising or consisting of SEQ ID NO:156 (3B2).

In an embodiment, the antibody-based molecule that binds to human muscle-specific tyrosine-protein kinase (MuSK) comprises a heavy chain variable domain, where the heavy chain variable domain comprises:
a CDR-H1 amino acid sequence comprising SEQ ID NO: 147 or having 1, 2, 3, 4 or 5 amino acid alterations relative to SEQ ID NO: 147,
a CDR-H2 amino acid sequence comprising SEQ ID NO: 153 or having 1, 2, 3, 4 or 5 amino acid alterations relative to SEQ ID NO: 153, and
a CDR-H3 amino acid sequence comprising SEQ ID NO: 156 or having 1, 2, 3, 4 or 5 amino acid alterations relative to SEQ ID NO:156 (3B2g2m1).

In an embodiment, the CDR-H2 of the antibody comprises a proline (P) at position 3, a tryptophan (W) at position 4, and a serine(S) or asparagine (N) at position 5.

In an embodiment, the antibody-based molecule that binds to human muscle-specific tyrosine-protein kinase (MuSK) comprises a heavy chain variable domain, where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO:156 (3B2g2m1).

The sequences of the heavy chain CDR sequences are provided in Table 1 below.

| | HCDR1 | | HCDR2 | | HCDR3 | |
|---|---|---|---|---|---|---|
| mAb/Fab | | SEQ ID | | SEQ ID | | SEQ ID |
| name | Sequence | NO: | Sequence | NO: | Sequence | NO: |
| X1 | SSSIH | 1 | SISSSSGSTSYADSVKG | 17 | KYWSQYYWAHYYGGLDY | 33 |
| X2 | SSSIH | 2 | SISSSYGSTSYADSVKG | 18 | SEGDRYVSGYMGMDY | 34 |
| X2m1 | SSSIH | 2 | SISSSYGSTSYADSVKG | 18 | SEGDRYVSGYFGFDY | 240 |
| X2m2 | SSSIH | 2 | SISSSYGSTSYADSVKG | 18 | SEGDRYVSGYFGLDY | 241 |
| X2m3 | SSSIH | 2 | SISSSYGSTSYADSVKG | 18 | SEGDRYVSGYSGFDY | 242 |
| X2m4 | SSSIH | 2 | SISSSYGSTSYADSVKG | 18 | SEGDRYVSGYSGLDY | 243 |
| X2m5 | SSSIH | 2 | SISSSYGSTSYADSVKG | 18 | SEGDRYVSGYFGMDY | 244 |
| X2m6 | SSSIH | 2 | SISSSYGSTSYADSVKG | 18 | SEGDRYVSGYSGMDY | 245 |
| X2m7 | SSSIH | 2 | SISSSYGSTSYADSVKG | 18 | SEGDRYVSGYMGFDY | 246 |
| X2m8 | SSSIH | 2 | SISSSYGSTSYADSVKG | 18 | SEGDRYVSGYMGLDY | 247 |
| X3 | SSSIH | 3 | SISSSSGYTYYADSVKG | 19 | SWYEMWMSGYFGFDY | 35 |
| X4 | SSSIH | 4 | SISSSSGSTYYADSVKG | 20 | GEHDYYVFGYLGMDY | 36 |
| X5 | SSSIH | 5 | SISSSSGSTSYADSVKG | 21 | SYTMFYYGGWYGSGYFGM | 37 |
| | | | | | DY | |
| X6 | SSSIH | 6 | SISSYSGYTYYADSVKG | 22 | TYGSYYVSSYTGMDY | 38 |
| X7 | SSSIH | 7 | SISSSYSSTYYADSVKG | 23 | LAGLYHYPGYLGLDY | 39 |
| X8 | SSSIH | 8 | SISSSSGSTSYADSVKG | 24 | SWSYHPWYYHVGWYTGLD | 40 |
| | | | | | Y | |
| X9 | SSSIH | 9 | SIYSSSGSTYYADSVKG | 25 | SGGEFYITSYYGMDY | 41 |
| X10 | SSSIH | 10 | SISSSYSSTSYADSVKG | 26 | KYYRWRHNKYQGFDY | 42 |
| X11 | SSSIH | 11 | SISSYSGSTYYADSVKG | 27 | SWGSYYVSGFVGFDY | 43 |
| X12 | SSSIH | 12 | YISPSSGYTSYADSVKG | 28 | QYWVPQWWITQYFGMDY | 44 |
| X13 | SSSIH | 13 | SISSSSGSTSYADSVKG | 29 | SSEHWYTIGYYGIDY | 45 |
| X14 | SSSIH | 14 | SISSSSGYTYYADSVKG | 30 | GSHHWFLWIYSGLDY | 46 |
| X15 | SSSIH | 15 | SISSSYGSTSYADSVKG | 31 | SEGDRYVSGYMGMDY | 47 |
| X16 | SSSIH | 16 | SIYSSYGYTSYADSVKG | 32 | NWGYYMYWGWYYALDY | 48 |
| X17 | YSSIH | 135 | SIYSSSGSTYYADSVKG | 137 | GDHGYYVFGYLGMDY | 139 |
| X17m1 | YSSIH | 135 | SIYSSSGSTYYADSVKG | 137 | GDHGYYVSGYLGMDY | 248 |
| X17m2 | YSSIH | 135 | SIYSSSGSTYYADSVKG | 137 | GDHGYYVYGYLGMDY | 249 |
| X17m3 | YSSIH | 135 | SIYSSSGSTYYADSVKG | 137 | GDHGYYVSGYLGFDY | 250 |
| X17m6 | YSSIH | 135 | SIYSSSGSTYYADSVKG | 137 | GEHGYYVSGYLGFDY | 251 |
| X18 | SSSIH | 136 | SISSSSGYTSYADSVKG | 138 | KYSKRAYPDYYWRGLDY | 140 |
| 14D10 | DYGMS | 147 | AIPWNGGSTYYKESVKG | 150 | RSGRIAFGALDA | 156 |
| 7G4 | DYGMS | 147 | AIPWNGGSTYYKESVKG | 150 | RSGRIAFGALDA | 156 |
| 3C4 | DYGMS | 147 | AIPWNGGSTYYKESVKG | 150 | RSGRIAFGALDA | 156 |
| 3B2 | DYGMS | 147 | AIPWNGGSTYYKESVKG | 150 | RSGRIAFGALDA | 156 |
| 3G3 | DYGMS | 147 | AIPWNGGSTYYKESVKG | 150 | RSGRIAFGALDA | 156 |
| 31G2 | DYGMS | 147 | AIPWNGGSTYYKESVKG | 150 | RSGRIAFGALDA | 156 |
| 31B7 | DYGMS | 147 | AIPWNGGSTYYKESVKG | 150 | RSGRIAFGALDA | 156 |
| 17H10 | ARYYS | 148 | VIAYDGSTYYSPSLKS | 151 | GSSRVAAAFDS | 157 |
| | WS | | | | | |
| 23B6 | ARYYS | 148 | VIAYDGSTYYSPSLKS | 151 | GSSRVAAAFDS | 157 |
| | WS | | | | | |
| 30E1 | ARYYS | 148 | VIAYDGSTYYSPSLKS | 151 | GSSRVAAAFDS | 157 |
| | WS | | | | | |
| 30A11 | ARYYS | 148 | VIAYDGSTYYSPSLKS | 151 | GSSRVAAAFDS | 157 |
| | WS | | | | | |
| 16F11 | LYYMN | 149 | VIDTHSIAYYADSVKG | 152 | GRTALVR | 158 |
| 4C11 | LYYMN | 149 | VIDTHSIAYYADSVKG | 152 | GRTALVR | 158 |
| 7A12 | LYYMN | 149 | VIDTHSIAYYADSVKG | 152 | GRTALVR | 158 |
| 7G12 | LYYMN | 149 | VIDTHSIAYYADSVKG | 152 | GRTALVR | 158 |
| 7B8 | LYYMN | 149 | VIDTHSIAYYADSVKG | 152 | GRTALVR | 158 |
| 3B2g1m1 | DYGMS | 147 | AIPWSGGSTYYKESVKG | 153 | RSGRIAFGALDA | 156 |
| 3B2g1m2 | DYGMS | 147 | AIPGSGGSTYYKESVKG | 154 | RSGRIAFGALDA | 156 |
| 3B2g1m4 | DYGMS | 147 | AIPWQGGSTYYKESVKG | 155 | RSGRIAFGALDA | 156 |
| 3B2g2m1 | DYGMS | 147 | AIPWSGGSTYYKESVKG | 153 | RSGRIAFGALDA | 156 |
| 3B2g2m2 | DYGMS | 147 | AIPGSGGSTYYKESVKG | 154 | RSGRIAFGALDA | 156 |
| 3B2g2m4 | DYGMS | 147 | AIPWQGGSTYYKESVKG | 155 | RSGRIAFGALDA | 156 |

In some embodiments, the MuSK antibody-based molecules as disclosed herein further comprise a light chain variable domain. The light chain variable domain comprises:
(i) a complementarity-determining region 1 (CDR-L1) having an amino acid sequence of any one of SEQ ID NOs: 49-64, 141, 142, 159-169, or a modified amino acid sequence of any one of SEQ ID NO: 49-64, 141, 142, or 159-169, said modified sequence having at least 80% sequence identity to any one of SEQ ID NO: 49-64, 141, 142, or 159-169;
(ii) a complementarity-determining region 2 (CDR-L2) having an amino acid sequence of any one of SEQ ID NOs: 65-80, 143, 144, 170-179, or a modified amino acid sequence of any one of SEQ ID NO: 65-80, 143, 144 or 170-179, said modified sequence having at least 80% sequence identity to any one of SEQ ID NO: 65-80, 143, 144 or 170-179; and
(iii) a complementarity-determining region 3 (CDR-L3) having an amino acid sequence of any one of SEQ ID NOs: 81-96, 145, 146, 180-195, or a modified amino acid sequence of any one of SEQ ID NO: 81-96, 145, 146, or 180-195, said modified sequence having at least 80% sequence identity to any one of SEQ ID NO: 81-96, 145, 146 or 180-195.

In some embodiments, the MuSK antibody-based molecules as disclosed herein further comprise a light chain variable domain. The light chain variable domain comprises:
(iv) a complementarity-determining region 1 (CDR-L1) having an amino acid sequence of any one of SEQ ID NOs: 49-64, 141, 142, 159-169, or a modified amino acid sequence of any one of SEQ ID NO: 49-64, 141, 142, or 159-169, said modified sequence having 1, 2, 3, 4 or 5 amino acid alterations relative to to any one of SEQ ID NO: 49-64, 141, 142, or 159-169;
(v) a complementarity-determining region 2 (CDR-L2) having an amino acid sequence of any one of SEQ ID NOs: 65-80, 143, 144, 170-179, or a modified amino acid sequence of any one of SEQ ID NO: 65-80, 143, 144 or 170-179, said modified sequence having 1, 2, 3, 4 or 5 amino acid alterations relative to any one of SEQ ID NO: 65-80, 143, 144 or 170-179; and
(vi) a complementarity-determining region 3 (CDR-L3) having an amino acid sequence of any one of SEQ ID NOs: 81-96, 145, 146, 180-195, or a modified amino acid sequence of any one of SEQ ID NO: 81-96, 145, 146, or 180-195, said modified sequence having 1, 2, 3, 4 or 5 amino acid alterations relative to any one of SEQ ID NO: 81-96, 145, 146 or 180-195.

In an embodiment, the light chain variable domain of the MuSK antibody-based molecule disclosed herein comprises:
(i) a light chain variable domain comprising the CDR-L1 of SEQ ID NO: 49 or having 1, 2, 3, 4 or 5 amino acid alterations relative to SEQ ID NO:49, the CDR-L2 of SEQ ID NO: 65 or having 1, 2, 3, 4 or 5 amino acid alterations relative to SEQ ID NO:65, and the CDR-L3 of SEQ ID NO: 81 or having 1, 2, 3, 4 or 5 amino acid alterations relative to SEQ ID NO:81;
(ii) a light chain variable domain comprising the CDR-L1 of SEQ ID NO: 50 or having 1, 2, 3, 4 or 5 amino acid alterations relative to SEQ ID NO:50, the CDR-L2 of SEQ ID NO: 66 or having 1, 2, 3, 4 or 5 amino acid alterations relative to SEQ ID NO:66, and the CDR-L3 of SEQ ID NO: 82 or having 1, 2, 3, 4 or 5 amino acid alterations relative to SEQ ID NO:82;
(iii) a light chain variable domain comprising the CDR-L1 of SEQ ID NO: 51 or having 1, 2, 3, 4 or 5 amino acid alterations relative to SEQ ID NO:51, the CDR-L2 of SEQ ID NO: 67 or having 1, 2, 3, 4 or 5 amino acid alterations relative to SEQ ID NO:67, and the CDR-L3 of SEQ ID NO: 83 or having 1, 2, 3, 4 or 5 amino acid alterations relative to SEQ ID NO:83;
(iv) a light chain variable domain comprising the CDR-L1 of SEQ ID NO: 52 or having 1, 2, 3, 4 or 5 amino acid alterations relative to SEQ ID NO:52, the CDR-L2 of SEQ ID NO: 68 or having 1, 2, 3, 4 or 5 amino acid alterations relative to SEQ ID NO:68, and the CDR-L3 of SEQ ID NO: 84 or having 1, 2, 3, 4 or 5 amino acid alterations relative to SEQ ID NO:84;
(v) a light chain variable domain comprising the CDR-L1 of SEQ ID NO: 53 or having 1, 2, 3, 4 or 5 amino acid alterations relative to SEQ ID NO:53, the CDR-L2 of SEQ ID NO: 69 or having 1, 2, 3, 4 or 5 amino acid alterations relative to SEQ ID NO:69, and the CDR-L3 of SEQ ID NO: 85 or having 1, 2, 3, 4 or 5 amino acid alterations relative to SEQ ID NO:85;
(vi) a light chain variable domain comprising the CDR-L1 of SEQ ID NO: 54 or having 1, 2, 3, 4 or 5 amino acid alterations relative to SEQ ID NO:54, the CDR-L2 of SEQ ID NO: 70 or having 1, 2, 3, 4 or 5 amino acid alterations relative to SEQ ID NO:70, and the CDR-L3 of SEQ ID NO: 86 or having 1, 2, 3, 4 or 5 amino acid alterations relative to SEQ ID NO:86;
(vii) a light chain variable domain comprising the CDR-L1 of SEQ ID NO: 55 or having 1, 2, 3, 4 or 5 amino acid alterations relative to SEQ ID NO:55, the CDR-L2 of SEQ ID NO: 71 or having 1, 2, 3, 4 or 5 amino acid alterations relative to SEQ ID NO:71, and the CDR-L3 of SEQ ID NO: 87 or having 1, 2, 3, 4 or 5 amino acid alterations relative to SEQ ID NO:87;
(viii) a light chain variable domain comprising the CDR-L1 of SEQ ID NO: 56 or having 1, 2, 3, 4 or 5 amino acid alterations relative to SEQ ID NO:56, the CDR-L2 of SEQ ID NO: 72 or having 1, 2, 3, 4 or 5 amino acid alterations relative to SEQ ID NO:72, and the CDR-L3 of SEQ ID NO: 88 or having 1, 2, 3, 4 or 5 amino acid alterations relative to SEQ ID NO:88;
(ix) a light chain variable domain comprising the CDR-L1 of SEQ ID NO: 57 or having 1, 2, 3, 4 or 5 amino acid alterations relative to SEQ ID NO:57, the CDR-L2 of SEQ ID NO: 73 or having 1, 2, 3, 4 or 5 amino acid alterations relative to SEQ ID NO:73, and the CDR-L3 of SEQ ID NO: 89 or having 1, 2, 3, 4 or 5 amino acid alterations relative to SEQ ID NO:89;
(x) a light chain variable domain comprising the CDR-L1 of SEQ ID NO: 58 or having 1, 2, 3, 4 or 5 amino acid alterations relative to SEQ ID NO:58, the CDR-L2 of SEQ ID NO: 74 or having 1, 2, 3, 4 or 5 amino acid alterations relative to SEQ ID NO:74, and the CDR-L3 of SEQ ID NO: 90 or having 1, 2, 3, 4 or 5 amino acid alterations relative to SEQ ID NO:90;
(xi) a light chain variable domain comprising the CDR-L1 of SEQ ID NO: 59 or having 1, 2, 3, 4 or 5 amino acid alterations relative to SEQ ID NO:59, the CDR-L2 of SEQ ID NO: 75 or having 1, 2, 3, 4 or 5 amino acid alterations relative to SEQ ID NO:75, and the CDR-L3 of SEQ ID NO: 91 or having 1, 2, 3, 4 or 5 amino acid alterations relative to SEQ ID NO:91;
(xii) a light chain variable domain comprising the CDR-L1 of SEQ ID NO: 60 or having 1, 2, 3, 4 or 5 amino acid alterations relative to SEQ ID NO:60, the CDR-L2 of SEQ ID NO: 76 or having 1, 2, 3, 4 or 5 amino acid alterations relative to SEQ ID NO:76, and the CDR-L3 of SEQ ID NO: 92 or having 1, 2, 3, 4 or 5 amino acid alterations relative to SEQ ID NO:92;
(xiii) a light chain variable domain comprising the CDR-L1 of SEQ ID NO: 61 or having 1, 2, 3, 4 or 5 amino acid alterations relative to SEQ ID NO:61, the CDR-L2 of SEQ ID NO: 77 or having 1, 2, 3, 4 or 5 amino acid alterations relative to SEQ ID NO:77, and the CDR-L3 of SEQ ID NO: 93 or having 1, 2, 3, 4 or 5 amino acid alterations relative to SEQ ID NO:93;
(xiv) a light chain variable domain comprising the CDR-L1 of SEQ ID NO: 62 or having 1, 2, 3, 4 or 5 amino acid alterations relative to SEQ ID NO:62, the CDR-L2 of SEQ ID NO: 78 or having 1, 2, 3, 4 or 5 amino acid alterations relative to SEQ ID NO:78, and the CDR-L3 of SEQ ID NO: 94 or having 1, 2, 3, 4 or 5 amino acid alterations relative to SEQ ID NO:94;
(xv) a light chain variable domain comprising the CDR-L1 of SEQ ID NO: 63 or having 1, 2, 3, 4 or 5 amino acid alterations relative to SEQ ID NO:63, the CDR-L2 of SEQ ID NO: 79 or having 1, 2, 3, 4 or 5 amino acid alterations relative to SEQ ID NO:79, and the CDR-L3 of SEQ ID NO: 95 or having 1, 2, 3, 4 or 5 amino acid alterations relative to SEQ ID NO:95;
(xvi) a light chain variable domain comprising the CDR-L1 of SEQ ID NO: 64 or having 1, 2, 3, 4 or 5 amino acid alterations relative to SEQ ID NO:64, the CDR-L2 of SEQ ID NO: 80 or having 1, 2, 3, 4 or 5 amino acid alterations relative to SEQ ID NO:80, and the CDR-L3 of SEQ ID NO: 96 or having 1, 2, 3, 4 or 5 amino acid alterations relative to SEQ ID NO:96;
(xvii) a light chain variable domain comprising the CDR-L1 of SEQ ID NO: 141 or having 1, 2, 3, 4 or 5 amino acid alterations relative to SEQ ID NO:141, the CDR-L2 of SEQ ID NO: 143 or having 1, 2, 3, 4 or 5 amino acid alterations relative to SEQ ID NO:143, and the CDR-L3 of SEQ ID NO: 145 or having 1, 2, 3, 4 or 5 amino acid alterations relative to SEQ ID NO: 145; or
(xviii) a light chain variable domain comprising the CDR-L1 of SEQ ID NO: 142 or having 1, 2, 3, 4 or 5 amino acid alterations relative to SEQ ID NO:142, the CDR-L2 of SEQ ID NO: 144 or having 1, 2, 3, 4 or 5 amino acid alterations relative to SEQ ID NO:144, and the CDR-L3 of SEQ ID NO: 146 or having 1, 2, 3, 4 or 5 amino acid alterations relative to SEQ ID NO:146. The sequences of the light chain CDRs are provided in Table 2 below.

In an embodiment, the light chain variable domain of the MuSK antibody-based molecule disclosed herein comprises:
(xix) a light chain variable domain comprising the CDR-L1 of SEQ ID NO: 159 or having 1, 2, 3, 4 or 5 amino acid alterations relative to SEQ ID NO: 159, the CDR-L2 of SEQ ID NO: 170 or having 1, 2, 3, 4 or 5 amino acid alterations relative to SEQ ID NO:170, and the CDR-L3 of SEQ ID NO: 180 or having 1, 2, 3, 4 or 5 amino acid alterations relative to SEQ ID NO:180;
(xx) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 171 or having 1, 2, 3, 4 or 5 amino acid alterations relative to SEQ ID NO:171, and the CDR-L3 of SEQ ID NO: 181 or having 1, 2, 3, 4 or 5 amino acid alterations relative to SEQ ID NO:181;
(xxi) a light chain variable domain comprising the CDR-L1 of SEQ ID NO: 160 or having 1, 2, 3, 4 or 5 amino acid alterations relative to SEQ ID NO:160, the CDR-L2 of SEQ ID NO: 172 or having 1, 2, 3, 4 or 5 amino acid alterations relative to SEQ ID NO:172, and the CDR-L3 of SEQ ID NO: 182 or having 1, 2, 3, 4 or 5 amino acid alterations relative to SEQ ID NO:182;
(xxii) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 172 or having 1, 2, 3, 4 or 5 amino acid alterations relative to SEQ ID NO:172, and the CDR-L3 of SEQ ID NO: 183 or having 1, 2, 3, 4 or 5 amino acid alterations relative to SEQ ID NO:183;
(xxiii) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 171 or having 1, 2, 3, 4 or 5 amino acid alterations relative to SEQ ID NO:171, and the CDR-L3 of SEQ ID NO: 184 or having 1, 2, 3, 4 or 5 amino acid alterations relative to SEQ ID NO:184;
(xxiv) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 173 or having 1, 2, 3, 4 or 5 amino acid alterations relative to SEQ ID NO:173, and the CDR-L3 of SEQ ID NO: 185 or having 1, 2, 3, 4 or 5 amino acid alterations relative to SEQ ID NO:185;
(xxv) a light chain variable domain comprising the CDR-L1 of SEQ ID NO: 159 or having 1, 2, 3, 4 or 5 amino acid alterations relative to SEQ ID NO:159, the CDR-L2 of SEQ ID NO: 173 or having 1, 2, 3, 4 or 5 amino acid alterations relative to SEQ ID NO:173, and the CDR-L3 of SEQ ID NO: 186 or having 1, 2, 3, 4 or 5 amino acid alterations relative to SEQ ID NO:186;
(xxvi) a light chain variable domain comprising the CDR-L1 of SEQ ID NO: 161 or having 1, 2, 3, 4 or 5 amino acid alterations relative to SEQ ID NO:161, the CDR-L2 of SEQ ID NO: 174 or having 1, 2, 3, 4 or 5 amino acid alterations relative to SEQ ID NO:174, and the CDR-L3 of SEQ ID NO: 187 or having 1, 2, 3, 4 or 5 amino acid alterations relative to SEQ ID NO:187;
(xxvii) a light chain variable domain comprising the CDR-L1 of SEQ ID NO: 162 or having 1, 2, 3, 4 or 5 amino acid alterations relative to SEQ ID NO: 162, the CDR-L2 of SEQ ID NO: 174 or having 1, 2, 3, 4 or 5 amino acid alterations relative to SEQ ID NO:174, and the CDR-L3 of SEQ ID NO: 188 or having 1, 2, 3, 4 or 5 amino acid alterations relative to SEQ ID NO: 188;
(xxviii) a light chain variable domain comprising the CDR-L1 of SEQ ID NO: 163 or having 1, 2, 3, 4 or 5 amino acid alterations relative to SEQ ID NO: 163, the CDR-L2 of SEQ ID NO: 174 or having 1, 2, 3, 4 or 5 amino acid alterations relative to SEQ ID NO:174, and the CDR-L3 of SEQ ID NO: 188 or having 1, 2, 3, 4 or 5 amino acid alterations relative to SEQ ID NO:188;
(xxix) a light chain variable domain comprising the CDR-L1 of SEQ ID NO: 164 or having 1, 2, 3, 4 or 5 amino acid alterations relative to SEQ ID NO: 164, the CDR-L2 of SEQ ID NO: 174 or having 1, 2, 3, 4 or 5 amino acid alterations relative to SEQ ID NO:174, and the CDR-L3 of SEQ ID NO: 189 or having 1, 2, 3, 4 or 5 amino acid alterations relative to SEQ ID NO:189;
(xxx) a light chain variable domain comprising the CDR-L1 of SEQ ID NO: 165 or having 1, 2, 3, 4 or 5 amino acid alterations relative to SEQ ID NO:165, the CDR-L2 of SEQ ID NO: 175 or having 1, 2, 3, 4 or 5 amino acid alterations relative to SEQ ID NO:175, and the CDR-L3 of SEQ ID NO: 190 or having 1, 2, 3, 4 or 5 amino acid alterations relative to SEQ ID NO:190;
(xxxi) a light chain variable domain comprising the CDR-L1 of SEQ ID NO: 166 or having 1, 2, 3, 4 or 5 amino acid alterations relative to SEQ ID NO:166, the CDR-L2 of SEQ ID NO: 176 or having 1, 2, 3, 4 or 5 amino acid alterations relative to SEQ ID NO:176, and the CDR-L3 of SEQ ID NO: 191 or having 1, 2, 3, 4 or 5 amino acid alterations relative to SEQ ID NO:191;
(xxxii) a light chain variable domain comprising the CDR-L1 of SEQ ID NO: 167 or having 1, 2, 3, 4 or 5 amino acid alterations relative to SEQ ID NO:167, the CDR-L2 of SEQ ID NO: 177 or having 1, 2, 3, 4 or 5 amino acid alterations relative to SEQ ID NO:177, and the CDR-L3 of SEQ ID NO: 192 or having 1, 2, 3, 4 or 5 amino acid alterations relative to SEQ ID NO:192;
(xxxiii) a light chain variable domain comprising the CDR-L1 of SEQ ID NO: 168 or having 1, 2, 3, 4 or 5 amino acid alterations relative to SEQ ID NO: 168, the CDR-L2 of SEQ ID NO: 178 or having 1, 2, 3, 4 or 5 amino acid alterations relative to SEQ ID NO:178, and the CDR-L3 of SEQ ID NO: 193 or having 1, 2, 3, 4 or 5 amino acid alterations relative to SEQ ID NO: 193; or
(xxxiv) a light chain variable domain comprising the CDR-L1 of SEQ ID NO: 169 or having 1, 2, 3, 4 or 5 amino acid alterations relative to SEQ ID NO:169, the CDR-L2 of SEQ ID NO: 179 or having 1, 2, 3, 4 or 5 amino acid alterations relative to SEQ ID NO:179, and the CDR-L3 of SEQ ID NO: 194 or having 1, 2, 3, 4 or 5 amino acid alterations relative to SEQ ID NO: 194.

In an embodiment, the light chain variable domain of the MuSK antibody based molecule disclosed herein comprises:
(i) a light chain variable domain comprising the CDR-L1 of SEQ ID NO: 49, the CDR-L2 of SEQ ID NO: 65, and the CDR-L3 of SEQ ID NO: 81;
(ii) a light chain variable domain comprising the CDR-L1 of SEQ ID NO: 50, the CDR-L2 of SEQ ID NO: 66, and the CDR-L3 of SEQ ID NO: 82;
(iii) a light chain variable domain comprising the CDR-L1 of SEQ ID NO: 51, the CDR-L2 of SEQ ID NO: 67, and the CDR-L3 of SEQ ID NO: 83;
(iv) a light chain variable domain comprising the CDR-L1 of SEQ ID NO: 52, the CDR-L2 of SEQ ID NO: 68, and the CDR-L3 of SEQ ID NO: 84;
(v) a light chain variable domain comprising the CDR-L1 of SEQ ID NO: 53, the CDR-L2 of SEQ ID NO: 69, and the CDR-L3 of SEQ ID NO: 85;
(vi) a light chain variable domain comprising the CDR-L1 of SEQ ID NO: 54, the CDR-L2 of SEQ ID NO: 70, and the CDR-L3 of SEQ ID NO: 86;
(vii) a light chain variable domain comprising the CDR-L1 of SEQ ID NO: 55, the CDR-L2 of SEQ ID NO: 71, and the CDR-L3 of SEQ ID NO: 87;
(viii) a light chain variable domain comprising the CDR-L1 of SEQ ID NO: 56, the CDR-L2 of SEQ ID NO: 72, and the CDR-L3 of SEQ ID NO: 88;
(ix) a light chain variable domain comprising the CDR-L1 of SEQ ID NO: 57, the CDR-L2 of SEQ ID NO: 73, and the CDR-L3 of SEQ ID NO: 89;
(x) a light chain variable domain comprising the CDR-L1 of SEQ ID NO: 58, the CDR-L2 of SEQ ID NO: 74, and the CDR-L3 of SEQ ID NO: 90;
(xi) a light chain variable domain comprising the CDR-L1 of SEQ ID NO: 59, the CDR-L2 of SEQ ID NO: 75, and the CDR-L3 of SEQ ID NO: 91;
(xii) a light chain variable domain comprising the CDR-L1 of SEQ ID NO: 60, the CDR-L2 of SEQ ID NO: 76, and the CDR-L3 of SEQ ID NO: 92;
(xiii) a light chain variable domain comprising the CDR-L1 of SEQ ID NO: 61, the CDR-L2 of SEQ ID NO: 77, and the CDR-L3 of SEQ ID NO: 93;
(xiv) a light chain variable domain comprising the CDR-L1 of SEQ ID NO: 62, the CDR-L2 of SEQ ID NO: 78, and the CDR-L3 of SEQ ID NO: 94;
(xv) a light chain variable domain comprising the CDR-L1 of SEQ ID NO: 63, the CDR-L2 of SEQ ID NO: 79, and the CDR-L3 of SEQ ID NO: 95;
(xvi) a light chain variable domain comprising the CDR-L1 of SEQ ID NO: 64, the CDR-L2 of SEQ ID NO: 80, and the CDR-L3 of SEQ ID NO: 96;
(xvii) a light chain variable domain comprising the CDR-L1 of SEQ ID NO: 141, the CDR-L2 of SEQ ID NO: 143, and the CDR-L3 of SEQ ID NO: 145; or
(xviii) a light chain variable domain comprising the CDR-L1 of SEQ ID NO: 142, the CDR-L2 of SEQ ID NO: 144, and the CDR-L3 of SEQ ID NO: 146. The sequences of the light chain CDRs are provided in Table 2 below.

In an embodiment, the light chain variable domain of the MuSK antibody based molecule disclosed herein comprises:
(xix) a light chain variable domain comprising the CDR-L1 of SEQ ID NO: 159, the CDR-L2 of SEQ ID NO: 170, and the CDR-L3 of SEQ ID NO: 180;
(xx) a light chain variable domain comprising the CDR-L1 of SEQ ID NO: 159, the CDR-L2 of SEQ ID NO: 171, and the CDR-L3 of SEQ ID NO: 181;
(xxi) a light chain variable domain comprising the CDR-L1 of SEQ ID NO: 160, the CDR-L2 of SEQ ID NO: 172, and the CDR-L3 of SEQ ID NO: 182;
(xxii) a light chain variable domain comprising the CDR-L1 of SEQ ID NO: 159, the CDR-L2 of SEQ ID NO: 172, and the CDR-L3 of SEQ ID NO: 183;
(xxiii) a light chain variable domain comprising the CDR-L1 of SEQ ID NO: 159, the CDR-L2 of SEQ ID NO: 171, and the CDR-L3 of SEQ ID NO: 184;
(xxiv) a light chain variable domain comprising the CDR-L1 of SEQ ID NO: 159, the CDR-L2 of SEQ ID NO: 173, and the CDR-L3 of SEQ ID NO: 185;
(xxv) a light chain variable domain comprising the CDR-L1 of SEQ ID NO: 159, the CDR-L2 of SEQ ID NO: 173, and the CDR-L3 of SEQ ID NO: 186;
(xxvi) a light chain variable domain comprising the CDR-L1 of SEQ ID NO: 161, the CDR-L2 of SEQ ID NO: 174, and the CDR-L3 of SEQ ID NO: 187;
(xxvii) a light chain variable domain comprising the CDR-L1 of SEQ ID NO: 162, the CDR-L2 of SEQ ID NO: 174, and the CDR-L3 of SEQ ID NO: 188;
(xxviii) a light chain variable domain comprising the CDR-L1 of SEQ ID NO: 163, the CDR-L2 of SEQ ID NO: 174, and the CDR-L3 of SEQ ID NO: 188;
(xxix) a light chain variable domain comprising the CDR-L1 of SEQ ID NO: 164, the CDR-L2 of SEQ ID NO: 174, and the CDR-L3 of SEQ ID NO: 189;
(xxx) a light chain variable domain comprising the CDR-L1 of SEQ ID NO: 165, the CDR-L2 of SEQ ID NO: 175, and the CDR-L3 of SEQ ID NO: 190;
(xxxi) a light chain variable domain comprising the CDR-L1 of SEQ ID NO: 166, the CDR-L2 of SEQ ID NO: 176, and the CDR-L3 of SEQ ID NO: 191;
(xxxii) a light chain variable domain comprising the CDR-L1 of SEQ ID NO: 167, the CDR-L2 of SEQ ID NO: 177, and the CDR-L3 of SEQ ID NO: 192;
(xxxiii) a light chain variable domain comprising the CDR-L1 of SEQ ID NO: 168, the CDR-L2 of SEQ ID NO: 178, and the CDR-L3 of SEQ ID NO: 193; or
(xxxiv) a light chain variable domain comprising the CDR-L1 of SEQ ID NO: 169, the CDR-L2 of SEQ ID NO: 179, and the CDR-L3 of SEQ ID NO: 194.

In an embodiment, the antibody-based molecule that binds to human muscle-specific tyrosine-protein kinase (MuSK) comprises a light chain variable domain, where the light chain variable domain comprises:
a CDR-L1 amino acid sequence comprising SEQ ID NO:159 or having 1, 2, 3, 4 or 5 amino acid alternations relative to SEQ ID NO: 159,
a CDR-L2 amino acid sequence comprising SEQ ID NO: 172 or having 1, 2, 3, 4 or 5 amino acid alternations relative to SEQ ID NO: 172, and
a CDR-L3 amino acid sequence comprising SEQ ID NO: 183 or having 1, 2, 3, 4 or 5 amino acid alternations relative SEQ ID NO:183 (3B2).

In an embodiment, the antibody-based molecule that binds to human muscle-specific tyrosine-protein kinase (MuSK) comprises a light chain variable domain, where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:183 (3B2).

In an embodiment, the light chain variable domain of the MuSK antibody based molecule disclosed herein comprises the CDR-L1 of SEQ ID NO: 159, the CDR-L2 of SEQ ID NO: 172, and the CDR-L3 of SEQ ID NO: 195 or a CDR-L3 having 1, 2, 3, 4 or 5 amino acid alterations relative to the amino acid sequence of SEQ ID NO: 195, wherein said alteration is present at residue 1, 2, 6, 7, 8, 9, 10, 11, 12, 13, 14, 15, 16, 17, or any combination thereof.

In an embodiment, the antibody-based molecule that binds to human muscle-specific tyrosine-protein kinase (MuSK) comprises a light chain variable domain, where the light chain variable domain comprises:
a CDR-L1 amino acid sequence comprising SEQ ID NO:159 or having 1, 2, 3, 4 or 5 amino acid alternations relative to SEQ ID NO: 159,
a CDR-L2 amino acid sequence comprising SEQ ID NO: 172 or having 1, 2, 3, 4 or 5 amino acid alternations relative to SEQ ID NO: 172, and
a CDR-L3 amino acid sequence comprising SEQ ID NO: 195 or having 1, 2, 3, 4 or 5 amino acid alternations relative SEQ ID NO:195 (3B2g2m1).

In an embodiment, the CDR-L1, CDR-L2, CDR-L3 amino acid sequence has at least 0, 1, 2, 3, 4 or 5 amino acid alternations relative to SEQ ID NO: 159, 172 or 195 (respectively).

In an embodiment, the antibody-based molecule that binds to human muscle-specific tyrosine-protein kinase (MuSK) comprises a light chain variable domain, where the light chain variable domain comprises:
a CDR-L1 amino acid sequence comprising or consisting of SEQ ID NO: 159,
a CDR-L2 amino acid sequence comprising or consisting of SEQ ID NO: 172, and a CDR-L3 amino acid sequence comprising or consisting of SEQ ID NO: 195 (3B2g2m1).

The sequences of the light chain CDR sequences are provided in Table 2 below.

| | LCDR1 | | LCDR2 | | LCDR3 | |
|---|---|---|---|---|---|---|
| mAb/Fab | | SEQ ID | | SEQ ID | | SEQ ID |
| name | Sequence | NO: | Sequence | NO: | Sequence | NO: |
| X1 | RASQSVSSAVA | 49 | SASSLYS | 65 | QQSSSSLIT | 81 |
| X2 | RASQSVSSAVA | 50 | SASSLYS | 66 | QQSGVWLIT | 82 |
| X3 | RASQSVSSAVA | 51 | SASSLYS | 67 | QQSSSSLIT | 83 |
| X4 | RASQSVSSAVA | 52 | SASSLYS | 68 | QQSYKPGALIT | 84 |
| X5 | RASQSVSSAVA | 53 | SASSLYS | 69 | QQSSSSLIT | 85 |
| X6 | RASQSVSSAVA | 54 | SASSLYS | 70 | QQSSSSLIT | 86 |
| X7 | RASQSVSSAVA | 55 | SASSLYS | 71 | QQSSRSSLLT | 87 |
| X8 | RASQSVSSAVA | 56 | SASSLYS | 72 | QQSSSSLIT | 88 |
| X9 | RASQSVSSAVA | 57 | SASSLYS | 73 | QQSSSSLIT | 89 |
| X10 | RASQSVSSAVA | 58 | SASSLYS | 74 | QQSLWYPVT | 90 |
| X11 | RASQSVSSAVA | 59 | SASSLYS | 75 | QQNSYYLIT | 91 |
| X12 | RASQSVSSAVA | 60 | SASSLYS | 76 | QQSSSSLIT | 92 |
| X13 | RASQSVSSAVA | 61 | SASSLYS | 77 | QQSYGSFSLIT | 93 |
| X14 | RASQSVSSAVA | 62 | SASSLYS | 78 | QQGSYHLIT | 94 |
| X15 | RASQSVSSAVA | 63 | SASSLYS | 79 | QQSGVWLIT | 95 |
| X16 | RASQSVSSAVA | 64 | SASSLYS | 80 | QQWSSAQALIT | 96 |
| X17 | RASQSVSSAVA | 141 | SASSLYS | 143 | QQSYKPGALIT | 145 |
| X18 | RASQSVSSAVA | 142 | SASSLYS | 144 | QQSYWWPIT | 146 |
| 14D10 | GLSSGSVTSSNYPD | 159 | TTNSRHS | 170 | ALYMGGGSNVYV | 180 |
| 7G4 | GLSSGSVTSSNYPD | 159 | STNSRHS | 171 | ALYMGRGSNKDYV | 181 |
| 3C4 | GLSSGSVTASNYPD | 160 | STDSRHS | 172 | ALYMYSDSKLYV | 182 |
| 3B2 | GLSSGSVTSSNYPD | 159 | STDSRHS | 172 | GLYMYSGSKNYV | 183 |
| 3G3 | GLSSGSVTSSNYPD | 159 | STNSRHS | 171 | ALYMGSDIRNYV | 184 |
| 31G2 | GLSSGSVTSSNYPD | 159 | STNSRLS | 173 | ALYMGSGSRNYV | 185 |
| 31B7 | GLSSGSVTSSNYPD | 159 | STNSRLS | 173 | ALYMGSESRNYV | 186 |
| 17H10 | GGNRIGGKSVQ | 161 | ADSRRPS | 174 | HVWGSTASAD | 187 |
| 23B6 | GGDNIGSKNAQ | 162 | ADSRRPS | 174 | HVWDSSTNAW | 188 |
| 30E1 | GGDNIGSKNTQ | 163 | ADSRRPS | 174 | HVWDSSTNAW | 188 |
| 30A11 | GGDNIASKNVQ | 164 | ADSRRPS | 174 | QVWDSSTNVAV | 189 |
| 16F11 | KSSQSVVFGSNQKSY | 165 | YASTQES | 175 | QQAYSAPT | 190 |
| | LN | | | | | |
| 4C11 | RSSQSVLYSSNQKNY | 166 | WASARES | 176 | QQSYKPPYG | 191 |
| | LN | | | | | |
| 7A12 | ESSQSVLYNQKNYLN | 167 | WASTRQS | 177 | QQAYNAPLT | 192 |
| 7G12 | KSSQRVQLGSNQKSY | 168 | YASTQQS | 178 | QQGYSAPFT | 193 |
| | LN | | | | | |
| 7B8 | KSSQSVLYNQKNYLA | 169 | WASTRES | 179 | QQGYSVPYT | 194 |
| 3B2g1m1 | GLSSGSVTSSNYPD | 159 | STDSRHS | 172 | GLYMYSGSKNYV | 183 |
| 3B2g1m2 | GLSSGSVTSSNYPD | 159 | STDSRHS | 172 | GLYMYSGSKNYV | 183 |
| 3B2g1m4 | GLSSGSVTSSNYPD | 159 | STDSRHS | 172 | GLYMYSGSKNYV | 183 |
| 3B2g2m1 | GLSSGSVTSSNYPD | 159 | STDSRHS | 172 | GLYSYSGSKNYV | 195 |
| 3B2g2m2 | GLSSGSVTSSNYPD | 159 | STDSRHS | 172 | GLYSYSGSKNYV | 195 |
| 3B2g2m4 | GLSSGSVTSSNYPD | 159 | STDSRHS | 172 | GLYSYSGSKNYV | 195 |

Suitable amino acid modifications to the heavy chain CDR sequences and/or the light chain CDR sequences of the MuSK antibody-based molecule disclosed herein include, for example, conservative substitutions or functionally equivalent amino acid residue substitutions that result in variant CDR sequences having similar or enhanced binding characteristics to those of the CDR sequences disclosed herein as described above. Encompassed by the present invention are CDRs of Tables 1 and 2 containing 1, 2, 3, 4, 5, or more amino acid alterations (depending on the length of the CDR) that maintain or enhance MuSK binding of the antibody. Suitable amino acid modifications to the heavy chain CDR sequences of Table 1 and/or the light chain CDR sequences of Tables 1 and 2 include, for example, conservative substitutions or functionally equivalent amino acid residue substitutions that result in variant CDR sequences having similar or enhanced binding characteristics to those of the CDR sequences of Table 1 and Table 2. Conservative substitutions are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine histidine), (3) aliphatic (glycine, alanine, valine, leucine, isoleucine, serine, threonine), with serine and threonine optionally grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, W H Freeman and Co., 1981, which is hereby incorporated by reference in its entirety). Non-conservative substitutions can also be made to the heavy chain CDR sequences of Table 1 and the light chain CDR sequences of Table 2. Non-conservative substitutions involve substituting one or more amino acid residues of the CDR with one or more amino acid residues from a different class of amino acids to improve or enhance the binding properties of CDR. The amino acid sequences of the heavy chain CDRs of Table 1 and/or the light chain CDRs of Table 2 may further comprise one or more internal neutral amino acid insertions or deletions that maintain or enhance MuSK binding.

In an embodiment, the MuSK antibody-based molecule comprises:
(i) a heavy chain variable domain comprising the CDR-H1 of SEQ ID NO: 1, the CDR-H2 of SEQ ID NO: 17, and the CDR-H3 of SEQ ID NO: 33, and a light chain variable domain comprising the CDR-L1 of SEQ ID NO: 49, the CDR-L2 of SEQ ID NO: 65, and the CDR-L3 of SEQ ID NO: 81;
(ii) a heavy chain variable domain comprising the CDR-H1 of SEQ ID NO: 2, the CDR-H2 of SEQ ID NO: 18, and the CDR-H3 of SEQ ID NO: 34, and a light chain variable domain comprising the CDR-L1 of SEQ ID NO: 50, the CDR-L2 of SEQ ID NO: 66, and the CDR-L3 of SEQ ID NO: 82;
(iii) a heavy chain variable domain comprising the CDR-H1 of SEQ ID NO: 3, the CDR-H2 of SEQ ID NO: 19, and the CDR-H3 of SEQ ID NO: 35, and a light chain variable domain comprising the CDR-L1 of SEQ ID NO: 51, the CDR-L2 of SEQ ID NO: 67, and the CDR-L3 of SEQ ID NO: 83;
(iv) a heavy chain variable domain comprising the CDR-H1 of SEQ ID NO: 4, the CDR-H2 of SEQ ID NO: 20, and the CDR-H3 of SEQ ID NO: 36, and a light chain variable domain comprising the CDR-L1 of SEQ ID NO: 52, the CDR-L2 of SEQ ID NO: 68, and the CDR-L3 of SEQ ID NO: 84;
(v) a heavy chain variable domain comprising the CDR-H1 of SEQ ID NO: 5, the CDR-H2 of SEQ ID NO: 21, and the CDR-H3 of SEQ ID NO: 37, and a light chain variable domain comprising the CDR-L1 of SEQ ID NO: 53, the CDR-L2 of SEQ ID NO: 69, and the CDR-L3 of SEQ ID NO: 85;
(vi) a heavy chain variable domain comprising the CDR-H1 of SEQ ID NO: 6, the CDR-H2 of SEQ ID NO: 22, and the CDR-H3 of SEQ ID NO: 38, and a light chain variable domain comprising the CDR-L1 of SEQ ID NO: 54, the CDR-L2 of SEQ ID NO: 70, and the CDR-L3 of SEQ ID NO: 86;
(vii) a heavy chain variable domain comprising the CDR-H1 of SEQ ID NO: 7, the CDR-H2 of SEQ ID NO: 23, and the CDR-H3 of SEQ ID NO: 39, and a light chain variable domain comprising the CDR-L1 of SEQ ID NO: 55, the CDR-L2 of SEQ ID NO:71, and the CDR-L3 of SEQ ID NO: 87;
(viii) a heavy chain variable domain comprising the CDR-H1 of SEQ ID NO: 8, the CDR-H2 of SEQ ID NO: 24, and the CDR-H3 of SEQ ID NO: 40, and a light chain variable domain comprising the CDR-L1 of SEQ ID NO: 56, the CDR-L2 of SEQ ID NO: 72, and the CDR-L3 of SEQ ID NO: 88;
(ix) a heavy chain variable domain comprising the CDR-H1 of SEQ ID NO: 9, the CDR-H2 of SEQ ID NO: 25, and the CDR-H3 of SEQ ID NO: 41, and a light chain variable domain comprising the CDR-L1 of SEQ ID NO: 57, the CDR-L2 of SEQ ID NO: 73, and the CDR-L3 of SEQ ID NO: 89;
(x) a heavy chain variable domain comprising the CDR-H1 of SEQ ID NO: 10, the CDR-H2 of SEQ ID NO: 26, and the CDR-H3 of SEQ ID NO: 42, and a light chain variable domain comprising the CDR-L1 of SEQ ID NO: 58, the CDR-L2 of SEQ ID NO: 74, and the CDR-L3 of SEQ ID NO: 90;
(xi) a heavy chain variable domain comprising the CDR-H1 of SEQ ID NO: 11, the CDR-H2 of SEQ ID NO: 27, and the CDR-H3 of SEQ ID NO: 43, and a light chain variable domain comprising the CDR-L1 of SEQ ID NO: 59, the CDR-L2 of SEQ ID NO: 75, and the CDR-L3 of SEQ ID NO: 91;
(xii) a heavy chain variable domain comprising the CDR-H1 of SEQ ID NO: 12, the CDR-H2 of SEQ ID NO: 28, and the CDR-H3 of SEQ ID NO: 44, and a light chain variable domain comprising the CDR-L1 of SEQ ID NO: 60, the CDR-L2 of SEQ ID NO: 76, and the CDR-L3 of SEQ ID NO: 92;
(xiii) a heavy chain variable domain comprising the CDR-H1 of SEQ ID NO: 13, the CDR-H2 of SEQ ID NO: 29, and the CDR-H3 of SEQ ID NO: 45, and a light chain variable domain comprising the CDR-L1 of SEQ ID NO: 61, the CDR-L2 of SEQ ID NO: 77, and the CDR-L3 of SEQ ID NO: 93;
(xiv) a heavy chain variable domain comprising the CDR-H1 of SEQ ID NO: 14, the CDR-H2 of SEQ ID NO: 30, and the CDR-H3 of SEQ ID NO: 46, and a light chain variable domain comprising the CDR-L1 of SEQ ID NO: 62, the CDR-L2 of SEQ ID NO: 78, and the CDR-L3 of SEQ ID NO: 94;
(xv) a heavy chain variable domain comprising the CDR-H1 of SEQ ID NO: 15, the CDR-H2 of SEQ ID NO: 31, and the CDR-H3 of SEQ ID NO: 47, and a light chain variable domain comprising the CDR-L1 of SEQ ID NO: 63, the CDR-L2 of SEQ ID NO: 79, and the CDR-L3 of SEQ ID NO: 95;
(xvi) a heavy chain variable domain comprising the CDR-H1 of SEQ ID NO: 16, the CDR-H2 of SEQ ID NO: 32, and the CDR-H3 of SEQ ID NO: 48, and a light chain variable domain comprising the CDR-L1 of SEQ ID NO: 64, the CDR-L2 of SEQ ID NO: 80, and the CDR-L3 of SEQ ID NO: 96;
(xvii) a heavy chain variable domain comprising the CDR-H1 of SEQ ID NO: 135, the CDR-H2 of SEQ ID NO: 137, and the CDR-H3 of SEQ ID NO: 139, and a light chain variable domain comprising the CDR-L1 of SEQ ID NO: 141, the CDR-L2 of SEQ ID NO: 143, and the CDR-L3 of SEQ ID NO: 145; or
(xviii) a heavy chain variable domain comprising the CDR-H1 of SEQ ID NO: 136, the CDR-H2 of SEQ ID NO: 138, and the CDR-H3 of SEQ ID NO: 140, and a light chain variable domain comprising the CDR-L1 of SEQ ID NO: 142, the CDR-L2 of SEQ ID NO: 144, and the CDR-L3 of SEQ ID NO: 146.

In an embodiment, the MuSK antibody-based molecule comprises:
(ii.a) a heavy chain variable domain comprising the CDR-H1 of SEQ ID NO: 2, the CDR-H2 of SEQ ID NO: 18, and the CDR-H3 of SEQ ID NO: 240, and a light chain variable domain comprising the CDR-L1 of SEQ ID NO: 50, the CDR-L2 of SEQ ID NO: 66, and the CDR-L3 of SEQ ID NO: 82 (X2m1);
(ii.b) a heavy chain variable domain comprising the CDR-H1 of SEQ ID NO: 2, the CDR-H2 of SEQ ID NO: 18, and the CDR-H3 of SEQ ID NO: 241, and a light chain variable domain comprising the CDR-L1 of SEQ ID NO: 50, the CDR-L2 of SEQ ID NO: 66, and the CDR-L3 of SEQ ID NO: 82 (X2m2);
(ii.c) a heavy chain variable domain comprising the CDR-H1 of SEQ ID NO: 2, the CDR-H2 of SEQ ID NO: 18, and the CDR-H3 of SEQ ID NO: 242, and a light chain variable domain comprising the CDR-L1 of SEQ ID NO: 50, the CDR-L2 of SEQ ID NO: 66, and the CDR-L3 of SEQ ID NO: 82 (X2m3);
(ii.d) a heavy chain variable domain comprising the CDR-H1 of SEQ ID NO: 2, the CDR-H2 of SEQ ID NO: 18, and the CDR-H3 of SEQ ID NO: 243, and a light chain variable domain comprising the CDR-L1 of SEQ ID NO: 50, the CDR-L2 of SEQ ID NO: 66, and the CDR-L3 of SEQ ID NO: 82 (X2m4);
(ii.e) a heavy chain variable domain comprising the CDR-H1 of SEQ ID NO: 2, the CDR-H2 of SEQ ID NO: 18, and the CDR-H3 of SEQ ID NO: 244, and a light chain variable domain comprising the CDR-L1 of SEQ ID NO: 50, the CDR-L2 of SEQ ID NO: 66, and the CDR-L3 of SEQ ID NO: 82 (X2m5);
(ii.f) a heavy chain variable domain comprising the CDR-H1 of SEQ ID NO: 2, the CDR-H2 of SEQ ID NO: 18, and the CDR-H3 of SEQ ID NO: 245, and a light chain variable domain comprising the CDR-L1 of SEQ ID NO: 50, the CDR-L2 of SEQ ID NO: 66, and the CDR-L3 of SEQ ID NO: 82 (X2m6);
(ii.g) a heavy chain variable domain comprising the CDR-H1 of SEQ ID NO: 2, the CDR-H2 of SEQ ID NO: 18, and the CDR-H3 of SEQ ID NO: 246, and a light chain variable domain comprising the CDR-L1 of SEQ ID NO: 50, the CDR-L2 of SEQ ID NO: 66, and the CDR-L3 of SEQ ID NO: 82 (X2m7); or
(ii.h) a heavy chain variable domain comprising the CDR-H1 of SEQ ID NO: 2, the CDR-H2 of SEQ ID NO: 18, and the CDR-H3 of SEQ ID NO: 247, and a light chain variable domain comprising the CDR-L1 of SEQ ID NO: 50, the CDR-L2 of SEQ ID NO: 66, and the CDR-L3 of SEQ ID NO: 82 (X2m8).

In an embodiment, the MuSK antibody-based molecule comprises:
(xvii.a) a heavy chain variable domain comprising the CDR-H1 of SEQ ID NO: 135, the CDR-H2 of SEQ ID NO: 137, and the CDR-H3 of SEQ ID NO: 248, and a light chain variable domain comprising the CDR-L1 of SEQ ID NO: 141, the CDR-L2 of SEQ ID NO: 143, and the CDR-L3 of SEQ ID NO: 145 (X17m1);
(xvii.b) a heavy chain variable domain comprising the CDR-H1 of SEQ ID NO: 135, the CDR-H2 of SEQ ID NO: 137, and the CDR-H3 of SEQ ID NO: 249, and a light chain variable domain comprising the CDR-L1 of SEQ ID NO: 141, the CDR-L2 of SEQ ID NO: 143, and the CDR-L3 of SEQ ID NO: 145 (X17m2);
(xvii.c) a heavy chain variable domain comprising the CDR-H1 of SEQ ID NO: 135, the CDR-H2 of SEQ ID NO: 137, and the CDR-H3 of SEQ ID NO: 250, and a light chain variable domain comprising the CDR-L1 of SEQ ID NO: 141, the CDR-L2 of SEQ ID NO: 143, and the CDR-L3 of SEQ ID NO: 145 (X17m3); or
(xvii.d) a heavy chain variable domain comprising the CDR-H1 of SEQ ID NO: 135, the CDR-H2 of SEQ ID NO: 137, and the CDR-H3 of SEQ ID NO: 251, and a light chain variable domain comprising the CDR-L1 of SEQ ID NO: 141, the CDR-L2 of SEQ ID NO: 143, and the CDR-L3 of SEQ ID NO: 145 (X17m6).

In an embodiment, the MuSK antibody-based molecule comprises:
(i) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 170, and the CDR-L3 of SEQ ID NO: 180 (14D10);
(ii) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 171, and the CDR-L3 of SEQ ID NO: 181 (7G4);
(iii) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 160, the CDR-L2 of SEQ ID NO: 172, and the CDR-L3 of SEQ ID NO: 182 (3C4);
(iv) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2);
(v) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 171, and the CDR-L3 of SEQ ID NO: 184 (3G3);
(vi) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 173, and the CDR-L3 of SEQ ID NO: 185 (31G2);
(vii) a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 173, and the CDR-L3 of SEQ ID NO: 186 (31B7);
(viii) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 161, the CDR-L2 of SEQ ID NO: 174, and the CDR-L3 of SEQ ID NO: 187 (17H10);
(ix) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 162, the CDR-L2 of SEQ ID NO: 174, and the CDR-L3 of SEQ ID NO: 188 (23B6);
(x) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 163, the CDR-L2 of SEQ ID NO: 174, and the CDR-L3 of SEQ ID NO: 188 (30E1);
(xi) a heavy chain variable domain comprising the CDR-H1 of SEQ ID NO: 148, the CDR-H2 of SEQ ID NO: 151, and the CDR-H3 of SEQ ID NO: 157, and the light chain variable domain comprising the CDR-L1 of SEQ ID NO: 164, the CDR-L2 of SEQ ID NO: 174, and the CDR-L3 of SEQ ID NO: 189 (30A11);
(xii) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 165, the CDR-L2 of SEQ ID NO: 175, and the CDR-L3 of SEQ ID NO: 190 (16F11);
(xiii) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 166, the CDR-L2 of SEQ ID NO: 176, and the CDR-L3 of SEQ ID NO: 191 (4C11);
(xiv) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 167, the CDR-L2 of SEQ ID NO: 177, and the CDR-L3 of SEQ ID NO: 192 (7A12);
(xv) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 168, the CDR-L2 of SEQ ID NO: 178, and the CDR-L3 of SEQ ID NO: 193 (7G12);
(xvi) a heavy chain variable domain comprising the CDR-H1 of SEQ ID NO: 149, the CDR-H2 of SEQ ID NO: 152, and the CDR-H3 of SEQ ID NO: 158, and the light chain variable domain comprising the CDR-L1 of SEQ ID NO: 169, the CDR-L2 of SEQ ID NO: 179, and the CDR-L3 of SEQ ID NO: 194 (7B8);
(xvii) a heavy chain variable domain comprising the CDR-H1 of SEQ ID NO: 147, the CDR-H2 of SEQ ID NO: 153, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m1);
(xviii) a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m2);
(xvix) a heavy chain variable domain comprising the CDR-H1 of SEQ ID NO: 147, the CDR-H2 of SEQ ID NO: 155, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m4);
(xx) a heavy chain variable domain comprising the CDR-H1 of SEQ ID NO: 147, the CDR-H2 of SEQ ID NO: 153, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m1);
(xxi) a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m2); or
(xxii) a heavy chain variable domain comprising the CDR-H1 of SEQ ID NO: 147, the CDR-H2 of SEQ ID NO: 155, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m4)

In a preferred embodiment, the antibody-based molecule that binds to human muscle-specific tyrosine-protein kinase (MuSK) comprises a heavy chain variable domain (VH) and a light chain variable domain (VL), wherein:
the VH comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156, and
the VL comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the CDR-H2 of the antibody comprises a proline (P) at position 3, a tryptophan (W) 30 at position 4, and a serine(S) or asparagine (N) at position 5. In a preferred embodiment, the CDR-H2 of the antibody comprises a proline (P) at position 3, a tryptophan (W) at position 4, and an asparagine (N) at position 5.

In a more preferred embodiment, the antibody-based molecule that binds to human muscle-specific tyrosine-protein kinase (MuSK) comprises a heavy chain variable domain and a light chain variable domain, where the heavy chain variable domain comprises:
a CDR-H1 amino acid sequence comprising or consisting of SEQ ID NO: 147,
a CDR-H2 amino acid sequence comprising or consisting of SEQ ID NO: 150, and
a CDR-H3 amino acid sequence comprising or consisting of SEQ ID NO:156 (3B2) and where the light chain variable domain comprises:
a CDR-L1 amino acid sequence comprising or consisting of SEQ ID NO: 159,
a CDR-L2 amino acid sequence comprising or consisting of SEQ ID NO: 172, and
a CDR-L3 amino acid sequence comprising or consisting of SEQ ID NO:183 (3B2).

In a preferred embodiment, the antibody-based molecule that binds to human muscle-specific tyrosine-protein kinase (MuSK) comprises a heavy chain variable domain and a light chain variable domain,
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO:156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the CDR-H2 of the antibody comprises a proline (P) at position 3, a tryptophan (W) at position 4, and a serine(S) or asparagine (N) at position 5.

In a more preferred embodiment, the antibody-based molecule that binds to human muscle-specific tyrosine-protein kinase (MuSK) comprises a heavy chain variable domain and a light chain variable domain, where the heavy chain variable domain comprises:
a CDR-H1 amino acid sequence comprising or consisting of SEQ ID NO: 147,
a CDR-H2 amino acid sequence comprising or consisting of SEQ ID NO: 153, and
a CDR-H3 amino acid sequence comprising or consisting of SEQ ID NO: 156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

The MuSK antibody-based molecule as described herein may comprise a variable light (VL) chain, a variable heavy (VH) chain, or a combination of VL and VH chains. In some embodiments, the VH chain of the MuSK antibody-based molecule comprises any one of the VH amino acid sequences provided in Table 3 below, or an amino acid sequence that is at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, identical or similar to any one of the VH amino acid sequences listed in Table 3. In some embodiments, the VL chain of the MuSK antibody-based molecule comprises any one of the VL amino acid sequences provided in Table 3 below, or an amino acid sequence that is at least 60%, identical or similar to any one of the VL amino acid sequences listed in Table 3. In an embodiment, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%.

| mAb/Fab | | | SEQ ID |
|---|---|---|---|
| name | Domain | Sequence | NO: |
| X1 | VH | EVQLVESGGGLVQPGGSLRLSCAASGFTFSSSSIHWVRQAPGKGLEWVA | 97 |
| | | SISSSSGSTSYADSVKGRFTISADTSKNTAYLQMNSLRAEDTAVYYCARKY | |
| | | WSQYYWAHYYGGLDYWGQGTLVTVSS | |
| | VL | DIQMTQSPSSLSASVGDRVTITCRASQSVSSAVAWYQQKPGKAPKLLIYSA | 98 |
| | | SSLYSGVPSRFSGSRSGTDFTLTISSLQPEDFATYYCQQSSSSLITFGQGT | |
| | | KVEIK | |
| X2 | VH | EVQLVESGGGLVQPGGSLRLSCAASGFTVSSSSIHWVRQAPGKGLEWVA | 99 |
| | | SISSSYGSTSYADSVKGRFTISADTSKNTAYLQMNSLRAEDTAVYYCARSE | |
| | | GDRYVSGYMGMDYWGQGTLVTVSS | |
| X2m1 | VH | EVQLVESGGGLVQPGGSLRLSCAASGFTVSSSSIHWVRQAPGKGLEWVA | 252 |
| | | SISSSYGSTSYADSVKGRFTISADTSKNTAYLQMNSLRAEDTAVYYCARSE | |
| | | GDRYVSGYFGFDYWGQGTLVTVSS | |
| X2m2 | VH | EVQLVESGGGLVQPGGSLRLSCAASGFTVSSSSIHWVRQAPGKGLEWVA | 253 |
| | | SISSSYGSTSYADSVKGRFTISADTSKNTAYLQMNSLRAEDTAVYYCARSE | |
| | | GDRYVSGYFGLDYWGQGTLVTVSS | |
| X2m3 | VH | EVQLVESGGGLVQPGGSLRLSCAASGFTVSSSSIHWVRQAPGKGLEWVA | 254 |
| | | SISSSYGSTSYADSVKGRFTISADTSKNTAYLQMNSLRAEDTAVYYCARSE | |
| | | GDRYVSGYSGFDYWGQGTLVTVSS | |
| X2m4 | VH | EVQLVESGGGLVQPGGSLRLSCAASGFTVSSSSIHWVRQAPGKGLEWVA | 255 |
| | | SISSSYGSTSYADSVKGRFTISADTSKNTAYLQMNSLRAEDTAVYYCARSE | |
| | | GDRYVSGYSGLDYWGQGTLVTVSS | |
| X2m5 | VH | EVQLVESGGGLVQPGGSLRLSCAASGFTVSSSSIHWVRQAPGKGLEWVA | 256 |
| | | SISSSYGSTSYADSVKGRFTISADTSKNTAYLQMNSLRAEDTAVYYCARSE | |
| | | GDRYVSGYFGMDYWGQGTLVTVSS | |
| X2m6 | VH | EVQLVESGGGLVQPGGSLRLSCAASGFTVSSSSIHWVRQAPGKGLEWVA | 257 |
| | | SISSSYGSTSYADSVKGRFTISADTSKNTAYLQMNSLRAEDTAVYYCARSE | |
| | | GDRYVSGYSGMDYWGQGTLVTVSS | |
| X2m7 | VH | EVQLVESGGGLVQPGGSLRLSCAASGFTVSSSSIHWVRQAPGKGLEWVA | 258 |
| | | SISSSYGSTSYADSVKGRFTISADTSKNTAYLQMNSLRAEDTAVYYCARSE | |
| | | GDRYVSGYMGFDYWGQGTLVTVSS | |
| X2m8 | VH | EVQLVESGGGLVQPGGSLRLSCAASGFTVSSSSIHWVRQAPGKGLEWVA | 259 |
| | | SISSSYGSTSYADSVKGRFTISADTSKNTAYLQMNSLRAEDTAVYYCARSE | |
| | | GDRYVSGYMGLDYWGQGTLVTVSS | |
| X2 | VL | DIQMTQSPSSLSASVGDRVTITCRASQSVSSAVAWYQQKPGKAPKLLIYSA | 100 |
| | | SSLYSGVPSRFSGSRSGTDFTLTISSLQPEDFATYYCQQSGVWLITFGQGT | |
| | | KVEIK | |
| X3 | VH | EVQLVESGGGLVQPGGSLRLSCAASGFTISSSSIHWVRQAPGKGLEWVAS | 101 |
| | | ISSSSGYTYYADSVKGRFTISADTSKNTAYLQMNSLRAEDTAVYYCARSWY | |
| | | EMWMSGYFGFDYWGQGTLVTVSS | |
| | VL | DIQMTQSPSSLSASVGDRVTITCRASQSVSSAVAWYQQKPGKAPKLLIYSA | 102 |
| | | SSLYSGVPSRFSGSRSGTDFTLTISSLQPEDFATYYCQQSSSSLITFGQGT | |
| | | KVEIK | |
| X4 | VH | EVQLVESGGGLVQPGGSLRLSCAASGFTVSSSSIHWVRQAPGKGLEWVA | 103 |
| | | SISSSSGSTYYADSVKGRFTISADTSKNTAYLQMNSLRAEDTAVYYCARGE | |
| | | HDYYVFGYLGMDYWGQGTLVTVSS | |
| | VL | DIQMTQSPSSLSASVGDRVTITCRASQSVSSAVAWYQQKPGKAPKLLIYSA | 104 |
| | | SSLYSGVPSRFSGSRSGTDFTLTISSLQPEDFATYYCQQSYKPGALITFGQ | |
| | | GTKVEIK | |
| X5 | VH | EVQLVESGGGLVQPGGSLRLSCAASGFTFYSSSIHWVRQAPGKGLEWVA | 105 |
| | | SISSSSGSTSYADSVKGRFTISADTSKNTAYLQMNSLRAEDTAVYYCARSY | |
| | | TMFYYGGWYGSGYFGMDYWGQGTLVTVSS | |
| | VL | DIQMTQSPSSLSASVGDRVTITCRASQSVSSAVAWYQQKPGKAPKLLIYSA | 106 |
| | | SSLYSGVPSRFSGSRSGTDFTLTISSLQPEDFATYYCQQSSSSLITFGQGT | |
| | | KVEIK | |
| X6 | VH | EVQLVESGGGLVQPGGSLRLSCAASGFTFSSSSIHWVRQAPGKGLEWVA | 107 |
| | | SISSYSGYTYYADSVKGRFTISADTSKNTAYLQMNSLRAEDTAVYYCARTY | |
| | | GSYYVSSYTGMDYWGQGTLVTVSS | |
| | VL | DIQMTQSPSSLSASVGDRVTITCRASQSVSSAVAWYQQKPGKAPKLLIYSA | 108 |
| | | SSLYSGVPSRFSGSRSGTDFTLTISSLQPEDFATYYCQQSSSSLITFGQGT | |
| | | KVEIK | |
| X7 | VH | EVQLVESGGGLVQPGGSLRLSCAASGFTLYSSSIHWVRQAPGKGLEWVA | 109 |
| | | SISSSYSSTYYADSVKGRFTISADTSKNTAYLQMNSLRAEDTAVYYCARLA | |
| | | GLYHYPGYLGLDYWGQGTLVTVSS | |
| | VL | DIQMTQSPSSLSASVGDRVTITCRASQSVSSAVAWYQQKPGKAPKLLIYSA | 110 |
| | | SSLYSGVPSRFSGSRSGTDFTLTISSLQPEDFATYYCQQSSRSSLLTFGQG | |
| | | TKVEIK | |
| X8 | VH | EVQLVESGGGLVQPGGSLRLSCAASGFTVSSSSIHWVRQAPGKGLEWVA | 111 |
| | | SISSSSGSTSYADSVKGRFTISADTSKNTAYLQMNSLRAEDTAVYYCARSW | |
| | | SYHPWYYHVGWYTGLDYWGQGTLVTVSS | |
| | VL | DIQMTQSPSSLSASVGDRVTITCRASQSVSSAVAWYQQKPGKAPKLLIYSA | 112 |
| | | SSLYSGVPSRFSGSRSGTDFTLTISSLQPEDFATYYCQQSSSSLITFGQGT | |
| | | KVEIK | |
| X9 | VH | EVQLVESGGGLVQPGGSLRLSCAASGFTFSSSSIHWVRQAPGKGLEWVA | 113 |
| | | SIYSSSGSTYYADSVKGRFTISADTSKNTAYLQMNSLRAEDTAVYYCARSG | |
| | | GEFYITSYYGMDYWGQGTLVTVSS | |
| | VL | DIQMTQSPSSLSASVGDRVTITCRASQSVSSAVAWYQQKPGKAPKLLIYSA | 114 |
| | | SSLYSGVPSRFSGSRSGTDFTLTISSLQPEDFATYYCQQSSSSLITFGQGT | |
| | | KVEIK | |
| X10 | VH | EVQLVESGGGLVQPGGSLRLSCAASGFTFSSSSIHWVRQAPGKGLEWVA | 115 |
| | | SISSSYSSTSYADSVKGRFTISADTSKNTAYLQMNSLRAEDTAVYYCARKY | |
| | | YRWRHNKYQGFDYWGQGTLVTVSS | |
| | VL | DIQMTQSPSSLSASVGDRVTITCRASQSVSSAVAWYQQKPGKAPKLLIYSA | 116 |
| | | SSLYSGVPSRFSGSRSGTDFTLTISSLQPEDFATYYCQQSLWYPVTFGQG | |
| | | TKVEIK | |
| X11 | VH | EVQLVESGGGLVQPGGSLRLSCAASGFTISSSSIHWVRQAPGKGLEWVAS | 117 |
| | | ISSYSGSTYYADSVKGRFTISADTSKNTAYLQMNSLRAEDTAVYYCARSWG | |
| | | SYYVSGFVGFDYWGQGTLVTVSS | |
| | VL | DIQMTQSPSSLSASVGDRVTITCRASQSVSSAVAWYQQKPGKAPKLLIYSA | 118 |
| | | SSLYSGVPSRFSGSRSGTDFTLTISSLQPEDFATYYCQQNSYYLITFGQGT | |
| | | KVEIK | |
| X12 | VH | EVQLVESGGGLVQPGGSLRLSCAASGFTISSSSIHWVRQAPGKGLEWVAY | 119 |
| | | ISPSSGYTSYADSVKGRFTISADTSKNTAYLQMNSLRAEDTAVYYCARQYW | |
| | | VPQWWITQYFGMDYWGQGTLVTVSS | |
| | VL | DIQMTQSPSSLSASVGDRVTITCRASQSVSSAVAWYQQKPGKAPKLLIYSA | 120 |
| | | SSLYSGVPSRFSGSRSGTDFTLTISSLQPEDFATYYCQQSSSSLITFGQGT | |
| | | KVEIK | |
| X13 | VH | EVQLVESGGGLVQPGGSLRLSCAASGFTISSSSIHWVRQAPGKGLEWVAS | 121 |
| | | ISSSSGSTSYADSVKGRFTISADTSKNTAYLQMNSLRAEDTAVYYCARSSE | |
| | | HWYTIGYYGIDYWGQGTLVTVSS | |
| | VL | DIQMTQSPSSLSASVGDRVTITCRASQSVSSAVAWYQQKPGKAPKLLIYSA | 122 |
| | | SSLYSGVPSRFSGSRSGTDFTLTISSLQPEDFATYYCQQSYGSFSLITFGQ | |
| | | GTKVEIK | |
| X14 | VH | EVQLVESGGGLVQPGGSLRLSCAASGFTFSSSSIHWVRQAPGKGLEWVA | 123 |
| | | SISSSSGYTYYADSVKGRFTISADTSKNTAYLQMNSLRAEDTAVYYCARGS | |
| | | HHWFLWIYSGLDYWGQGTLVTVSS | |
| | VL | DIQMTQSPSSLSASVGDRVTITCRASQSVSSAVAWYQQKPGKAPKLLIYSA | 124 |
| | | SSLYSGVPSRFSGSRSGTDFTLTISSLQPEDFATYYCQQGSYHLITFGQGT | |
| | | KVEIK | |
| X15 | VH | EVQLVESGGGLVQPGGSLRLSCAASGFTVSSSSIHWVRQAPGKGLEWVA | 125 |
| | | SISSSYGSTSYADSVKGRFTISADTSKNTAYLQMNSLRAEDTAVYYCARSE | |
| | | GDRYVSGYMGMDYWGQGTLVTVSS | |
| | VL | DIQMTQSPSSLSASVGDRVTITCRASQSVSSAVAWYQQKPGKAPKLLIYSA | 126 |
| | | SSLYSGVPSRFSGSRSGTDFTLTISSLQPEDFATYYCQQSGVWLITFGQGT | |
| | | KVEIK | |
| X16 | VH | EVQLVESGGGLVQPGGSLRLSCAASGFTFSSSSIHWVRQAPGKGLEWVA | 127 |
| | | SIYSSYGYTSYADSVKGRFTISADTSKNTAYLQMNSLRAEDTAVYYCARNW | |
| | | GYYMYWGWYYALDYWGQGTLVTVSS | |
| | VL | DIQMTQSPSSLSASVGDRVTITCRASQSVSSAVAWYQQKPGKAPKLLIYSA | 128 |
| | | SSLYSGVPSRFSGSRSGTDFTLTISSLQPEDFATYYCQQWSSAQALITFGQ | |
| | | GTKVEIK | |
| X17 | VH | EVQLVESGGGLVQPGGSLRLSCAASGFTISYSSIHWVRQAPGKGLEWVAS | 131 |
| | | IYSSSGSTYYADSVKGRFTISADTSKNTAYLQMNSLRAEDTAVYYCARGDH | |
| | | GYYVFGYLGMDYWGQGTLVTVSS | |
| X17m1 | VH | EVQLVESGGGLVQPGGSLRLSCAASGFTISYSSIHWVRQAPGKGLEWVAS | 260 |
| | | IYSSSGSTYYADSVKGRFTISADTSKNTAYLQMNSLRAEDTAVYYCARGDH | |
| | | GYYVSGYLGMDYWGQGTLVTVSS | |
| X17m2 | VH | EVQLVESGGGLVQPGGSLRLSCAASGFTISYSSIHWVRQAPGKGLEWVAS | 261 |
| | | IYSSSGSTYYADSVKGRFTISADTSKNTAYLQMNSLRAEDTAVYYCARGDH | |
| | | GYYVYGYLGMDYWGQGTLVTVSS | |
| X17m3 | VH | EVQLVESGGGLVQPGGSLRLSCAASGFTISYSSIHWVRQAPGKGLEWVAS | 262 |
| | | IYSSSGSTYYADSVKGRFTISADTSKNTAYLQMNSLRAEDTAVYYCARGDH | |
| | | GYYVSGYLGFDYWGQGTLVTVSS | |
| X17m6 | VH | EVQLVESGGGLVQPGGSLRLSCAASGFTISYSSIHWVRQAPGKGLEWVAS | 263 |
| | | IYSSSGSTYYADSVKGRFTISADTSKNTAYLQMNSLRAEDTAVYYCARGEH | |
| | | GYYVSGYLGFDYWGQGTLVTVSS | |
| X17 | VL | DIQMTQSPSSLSASVGDRVTITCRASQSVSSAVAWYQQKPGKAPKLLIYSA | 132 |
| | | SSLYSGVPSRFSGSRSGTDFTLTISSLQPEDFATYYCQQSYKPGALITFGQ | |
| | | GTKVEIK | |
| X18 | VH | EVQLVESGGGLVQPGGSLRLSCAASGFTISSSSIHWVRQAPGKGLEWVAS | 133 |
| | | ISSSSGYTSYADSVKGRFTISADTSKNTAYLQMNSLRAEDTAVYYCARKYS | |
| | | KRAYPDYYWRGLDYWGQGTLVTVSS | |
| | VL | DIQMTQSPSSLSASVGDRVTITCRASQSVSSAVAWYQQKPGKAPKLLIYSA | 134 |
| | | SSLYSGVPSRFSGSRSGTDFTLTISSLQPEDFATYYCQQSYWWPITFGQGT | |
| | | KVEIK | |
| 14D10 | VH | ELQLVESGGGLVQPGGSLRLSCAASGFTFDDYGMSWVRQAPGKGLEWV | 196 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRTLI | 197 |
| | | YTTNSRHSGVPSRFSGSISGNKAALTITGAQPEDEADYYCALYMGGGSNV | |
| | | YVFGGGTKLTVL | |
| 7G4 | VH | ELQLVESGGGLVQPGGSLRLSCAASGFTFDDYGMSWVRQAPGKGLEWV | 198 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRALI | 199 |
| | | YSTNSRHSGVPSRFSGSISGNKAALTITGAQPEDEADYYCALYMGRGSNK | |
| | | DYVFGGGTKLTVL | |
| 3C4 | VH | ELQLVESGGGLVQPGGSLRLSCAASGFTFDDYGMSWVRQAPGKGLEWV | 200 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTASNYPDWYQQTPGQAPRGLI | 201 |
| | | YSTDSRHSGVPSRFSGSISGNKAALTITGAQPEDEADYYCALYMYSDSKLY | |
| | | VFGGGTKLTVL | |
| 3B2 | VH | ELQLVESGGGLVQPGGSLRLSCAASGFTFDDYGMSWVRQAPGKGLEWV | 202 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRGLI | 203 |
| | | YSTDSRHSGVPSRFSGSISGNKAALTITGAQSEDEADYYCGLYMYSGSKN | |
| | | YVFGGGTKLTVL | |
| 3G3 | VH | ELQLVESGGGLVQPGGSLRLSCAASGFTFDDYGMSWVRQAPGKGLEWV | 204 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QTVVTQEPSLSVSPGGTVTLTCGLSSGSVTSSNYPDWYQQTPGQAPRALI | 205 |
| | | YSTNSRHSGVPSRFSGSTSGNKAALTITGAQPEDEADYYCALYMGSDIRN | |
| | | YVFGGGTKLTVL | |
| 31G2 | VH | ELQLVESGGGLVQPGGSLRLSCAASGFTFDDYGMSWVRQAPGKGLEWV | 206 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRALI | 207 |
| | | YSTNSRLSGVPSRFSGSFSGNKAALTITGAQPEDEADYYCALYMGSGSRN | |
| | | YVFGGGTKLTVL | |
| 31B7 | VH | ELQLVESGGGLVQPGGSLRLSCAASGFTFDDYGMSWVRQAPGKGLEWV | 208 |
| | | SAIPWNGGSTYYKESVKGRFTISRDNAKKTLYLQMNSLKSEDTAVYYCAKR | |
| | | SGRIAFGALDAWGQGTLVTVSS | |
| | VL | QAVVTQEPSLSVSPGGTVTLTCGLSSGSVTSSNYPDWYQQTPGQAPRALI | 209 |
| | | YSTNSRLSGVPSRFSGSFSGNKAALTITGAQPEDEADYYCALYMGSESRN | |
| | | YVFGGGTKLTVL | |
| 17H10 | VH | QVQVQESGPGLVKPSQTLSLTCTVSGGSITARYYSWSWIRQPPGKGLEW | 210 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVSVALRQTAKITCGGNRIGGKSVQWYQQKPGQAPMLVIYAD | 211 |
| | | SRRPSGIPERFTGSNSGNTATLTITGAQAEDEADYYCHVWGSTASADFGG | |
| | | GTHLTVL | |
| 23B6 | VH | QVQVQESGPGLVKPSQTLSLTCTVSGGSITARYYSWSWIRQPPGKGLEW | 212 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVSVALRQTAKITCGGDNIGSKNAQWYQQKPGQAPVMVLYAD | 213 |
| | | SRRPSGIPERFSGSNSGNTATLTISGAQAEDEADYYCHVWDSSTNAWFGG | |
| | | GTHLTVL | |
| 30E1 | VH | QVQVQESGPGLVKPSQTLSLTCTVSGGSITARYYSWSWIRQPPGKGLEW | 214 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVSVALRRTAKITCGGDNIGSKNTQWYQQKPGQAPVLVIYADS | 215 |
| | | RRPSGIPERFSGSNSGNTATLTISGAQAEDEADYYCHVWDSSTNAWFGGG | |
| | | THLTVL | |
| 30A11 | VH | QVQVQESGPGLVKPSQTLSLTCTVSGGSITARYYSWSWIRQPPGKGLEW | 216 |
| | | MGVIAYDGSTYYSPSLKSRTSISRDTSKNQFSLHLSSVTPDDTAVYYCARG | |
| | | SSRVAAAFDSWGQGTQVTVSS | |
| | VL | SYELTQSPSVTVALRQTAKITCGGDNIASKNVQWYQQKPGQAPSLVIWAD | 217 |
| | | SRRPSGIPVRFSGSNFGNTATLTISGAQAEDEADYYCQVWDSSTNVAVFG | |
| | | GGTHLTVL | |
| 16F11 | VH | EVQLVESGGGLVQPGGSLSLSCVASGFTFSLYYMNWVRQAPGKGLEWLS | 218 |
| | | VIDTHSIAYYADSVKGRFTISRDNVKNTLYLQLNNLKPEDTALYYCVLGRTAL | |
| | | VRWGQGTQVTVSS | |
| | VL | DIVMTQSPSSVTASVGEKVTINCKSSQSVVFGSNQKSYLNWYQQRPGQSP | 219 |
| | | RLLIYYASTQESGIPDRFSGSGSTTDFTLTISSVQPEDAAVYYCQQAYSAPT | |
| | | FGSGTRLEIK | |
| 4C11 | VH | EVQLVESGGGLVQPGGSLSLSCVASGFTFSLYYMNWVRQAPGKGLEWLS | 220 |
| | | VIDTHSIAYYADSVKGRFTISRDNVKNTLYLQLNNLKPEDTALYYCVLGRTAL | |
| | | VRWGQGTQVTVSS | |
| | VL | DIVMTQSPSSVTASAGERVTINCRSSQSVLYSSNQKNYLNWYQQRLGQSP | 221 |
| | | RLLIYWASARESGVPDRFSGSGSTTNFTLTISSFQPEDAAVYYCQQSYKPP | |
| | | YGFGSGTRLEIK | |
| 7A12 | VH | EVQLVESGGGLVQPGGSLSLSCVASGFTFSLYYMNWVRQAPGKGLEWLS | 222 |
| | | VIDTHSIAYYADSVKGRFTISRDNVKNTLYLQLNNLKPEDTALYYCVLGRTAL | |
| | | VRWGQGTQVTVSS | |
| | VL | EIVLTQSPSSVTASIGEKVTINCESSQSVLYNQKNYLNWYQQRPGQSPRLLI | 223 |
| | | YWASTRQSGVPDRFSGSGSGSTTDFTLTISSFQPEDVAVYYCQQAYNAPL | |
| | | TFGPGTKVELK | |
| 7G12 | VH | EVQLVESGGGLVQPGGSLSLSCVASGFTFSLYYMNWVRQAPGKGLEWLS | 224 |
| | | VIDTHSIAYYADSVKGRFTISRDNVKNTLYLQLNNLKPEDTALYYCVLGRTAL | |
| | | VRWGQGTQVTVSS | |
| | VL | EIVLTQSPNSVTASVGEKVTINCKSSQRVQLGSNQKSYLNWYQQRPGQSP | 225 |
| | | RLLIYYASTQQSGIPDRFSGSGSATDFTLTINSVQPEDAAVYYCQQGYSAP | |
| | | FTFGQGTKVELK | |
| 7B8 | VH | EVQLVESGGGLVQPGGSLSLSCVASGFTFSLYYMNWVRQAPGKGLEWLS | 226 |
| | | VIDTHSIAYYADSVKGRFTISRDNVKNTLYLQLNNLKPEDTALYYCVLGRTAL | |
| | | VRWGQGTQVTVSS | |
| | VL | EIVLTQSPSSVTASAGEKVTINCKSSQSVLYNQKNYLAWYQQRPGQSPRLL | 227 |
| | | IYWASTRESGVPDRFSGSGSTTDFTLTISSFQPEDVAVYYCQQGYSVPYTF | |
| | | GSGTRLEIK | |
| 3B2g1m1 | VH | EVQLLESGGGLVQPGGSLRLSCAASGFTFSDYGMSWVRQAPGKGLEWVS | 228 |
| | | AIPWSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 229 |
| | | YSTDSRHSGVPDRFSGSILGNKAALTITGAQADDESDYYCGLYMYSGSKN | |
| | | YVFGGGTKLTVL | |
| 3B2g1m2 | VH | EVQLLESGGGLVQPGGSLRLSCAASGFTFSDYGMSWVRQAPGKGLEWVS | 230 |
| | | AIPGSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 231 |
| | | YSTDSRHSGVPDRFSGSILGNKAALTITGAQADDESDYYCGLYMYSGSKN | |
| | | YVFGGGTKLTVL | |
| 3B2g1m4 | VH | EVQLLESGGGLVQPGGSLRLSCAASGFTFSDYGMSWVRQAPGKGLEWVS | 232 |
| | | AIPWQGGSTYYKESVKGRFTISRDNSKNTLYLQMNSLRAEDTAVYYCAKR | |
| | | SGRIAFGALDAWGQGTLVTVSS | |
| | VL | QTVVTQEPSFSVSPGGTVTLTCGLSSGSVTSSNYPDWYQQTPGQAPRTLI | 233 |
| | | YSTDSRHSGVPDRFSGSILGNKAALTITGAQADDESDYYCGLYMYSGSKN | |
| | | YVFGGGTKLTVL | |
| 3B2g2m1 | VH | EVQLLESGGGLVQPGGSLRLSCAASGFTFSDYGMSWVRQAPGKGLEWVS | 234 |
| | | AIPWSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 235 |
| | | YSTDSRHSGVPDRFSGSILGNKAALTITGAQADDESDYYCGLYSYSGSKNY | |
| | | VFGGGTKLTVL | |
| 3B2g2m2 | VH | EVQLLESGGGLVQPGGSLRLSCAASGFTFSDYGMSWVRQAPGKGLEWVS | 236 |
| | | AIPGSGGSTYYKESVKGRFTISRDNSKNTLYLQMNSLRAEDTAVYYCAKRS | |
| | | GRIAFGALDAWGQGTLVTVSS | |
| | VL | QTVVTQEPSFSVSPGGTVTLTCGLSSGSVTSSNYPDWYQQTPGQAPRTLI | 237 |
| | | YSTDSRHSGVPDRFSGSILGNKAALTITGAQADDESDYYCGLYSYSGSKNY | |
| | | VFGGGTKLTVL | |
| 3B2g2m4 | VH | EVQLLESGGGLVQPGGSLRLSCAASGFTFSDYGMSWVRQAPGKGLEWVS | 238 |
| | | AIPWQGGSTYYKESVKGRFTISRDNSKNTLYLQMNSLRAEDTAVYYCAKR | |
| | | SGRIAFGALDAWGQGTLVTVSS | |
| | VL | QTVVTQEPSFSVSPGGTVTLTCGLSSGSVTSSNYPDWYQQTPGQAPRTLI | 239 |
| | | YSTDSRHSGVPDRFSGSILGNKAALTITGAQADDESDYYCGLYSYSGSKNY | |
| | | VFGGGTKLTVL | |

In an embodiment, the MuSK antibody-based molecule disclosed herein comprises:
(i) a heavy chain variable domain comprising an amino acid sequence that is at least 80% identical to SEQ ID NO: 97 and a light chain variable domain comprising an amino acid sequence that is at least 80% identical to SEQ ID NO: 98;
(ii) a heavy chain variable domain comprising an amino acid sequence that is at least 80% identical to any one of SEQ ID NOS: 99 and 252-259 and a light chain variable domain comprising an amino acid sequence that is at least 80% identical to SEQ ID NO: 100;
(iii) a heavy chain variable domain comprising an amino acid sequence that is at least 80% identical to SEQ ID NO: 101 and a light chain variable domain comprising an amino acid sequence that is at least 80% identical to SEQ ID NO: 102;
(iv) a heavy chain variable domain comprising an amino acid sequence that is at least 80% identical to SEQ ID NO: 103 and a light chain variable domain comprising an amino acid sequence that is at least 80% identical to SEQ ID NO: 104;
(v) a heavy chain variable domain comprising an amino acid sequence that is at least 80% identical to SEQ ID NO: 105 and a light chain variable domain comprising an amino acid sequence that is at least 80% identical to SEQ ID NO: 106;
(vi) a heavy chain variable domain comprising an amino acid sequence that is at least 80% identical to SEQ ID NO: 107 and a light chain variable domain comprising an amino acid sequence that is at least 80% identical to SEQ ID NO: 108;
(vii) a heavy chain variable domain comprising an amino acid sequence that is at least 80% identical to SEQ ID NO: 109 and a light chain variable domain comprising an amino acid sequence that is at least 80% identical to SEQ ID NO: 110;
(viii) a heavy chain variable domain comprising an amino acid sequence that is at least 80% identical to SEQ ID NO: 111 and a light chain variable domain comprising an amino acid sequence that is at least 80% identical to SEQ ID NO: 112;
(ix) a heavy chain variable domain comprising an amino acid sequence that is at least 80% identical to SEQ ID NO: 113 and a light chain variable domain comprising an amino acid sequence that is at least 80% identical to SEQ ID NO: 114;
(x) a heavy chain variable domain comprising an amino acid sequence that is at least 80% identical to SEQ ID NO: 115 and a light chain variable domain comprising an amino acid sequence that is at least 80% identical to SEQ ID NO: 116;
(xi) a heavy chain variable domain comprising an amino acid sequence that is at least 80% identical to SEQ ID NO: 117 and a light chain variable domain comprising an amino acid sequence that is at least 80% identical to SEQ ID NO: 118;
(xii) a heavy chain variable domain comprising an amino acid sequence that is at least 80% identical to SEQ ID NO: 119 and a light chain variable domain comprising an amino acid sequence that is at least 80% identical to SEQ ID NO: 120;
(xiii) a heavy chain variable domain comprising an amino acid sequence that is at least 80% identical to SEQ ID NO: 121 and a light chain variable domain comprising an amino acid sequence that is at least 80% identical to SEQ ID NO: 122;
(xiv) a heavy chain variable domain comprising an amino acid sequence that is at least 80% identical to SEQ ID NO: 123 and a light chain variable domain comprising an amino acid sequence that is at least 80% identical to SEQ ID NO: 124;
(xv) a heavy chain variable domain comprising an amino acid sequence that is at least 80% identical to SEQ ID NO: 125 and a light chain variable domain comprising an amino acid sequence that is at least 80% identical to SEQ ID NO: 126;
(xvi) a heavy chain variable domain comprising an amino acid sequence that is at least 80% identical to SEQ ID NO: 127 and a light chain variable domain comprising an amino acid sequence that is at least 80% identical to SEQ ID NO: 128;
(xvii) a heavy chain variable domain comprising an amino acid sequence that is at least 80% identical to any one of SEQ ID NOS: 131 and 260-263 and a light chain variable domain comprising an amino acid sequence that is at least 80% identical to SEQ ID NO: 132; or
(xviii) a heavy chain variable domain comprising an amino acid sequence that is at least 80% identical to SEQ ID NO: 133 and a light chain variable domain comprising an amino acid sequence that is at least 80% identical to SEQ ID NO: 134.

In some embodiments, the MuSK antibody-based molecule disclosed herein comprises:
(i) a heavy chain variable domain comprising an amino acid sequence that is at least 80% identical to SEQ ID NO: 196 and a light chain variable domain comprising an amino acid sequence that is at least 80% identical to SEQ ID NO: 197;
(ii) a heavy chain variable domain comprising an amino acid sequence that is at least 80% identical to SEQ ID NO: 198 and a light chain variable domain comprising an amino acid sequence that is at least 80% identical to SEQ ID NO: 199;
(iii) a heavy chain variable domain comprising an amino acid sequence that is at least 80% identical to SEQ ID NO: 200 and a light chain variable domain comprising an amino acid sequence that is at least 80% identical to SEQ ID NO: 201;
(iv) a heavy chain variable domain comprising an amino acid sequence that is at least 80% identical to SEQ ID NO: 202 and a light chain variable domain comprising an amino acid sequence that is at least 80% identical to SEQ ID NO: 203;
(v) a heavy chain variable domain comprising an amino acid sequence that is at least 80% identical to SEQ ID NO: 204 and a light chain variable domain comprising an amino acid sequence that is at least 80% identical to SEQ ID NO: 205;
(vi) a heavy chain variable domain comprising an amino acid sequence that is at least 80% identical to SEQ ID NO: 206 and a light chain variable domain comprising an amino acid sequence that is at least 80% identical to SEQ ID NO: 207;
(vii) a heavy chain variable domain comprising an amino acid sequence that is at least 80% identical to SEQ ID NO: 208 and a light chain variable domain comprising an amino acid sequence that is at least 80% identical to SEQ ID NO: 209;
(viii) a heavy chain variable domain comprising an amino acid sequence that is at least 80% identical to SEQ ID NO: 210 and a light chain variable domain comprising an amino acid sequence that is at least 80% identical to SEQ ID NO: 211;
(ix) a heavy chain variable domain comprising an amino acid sequence that is at least 80% identical to SEQ ID NO: 212 and a light chain variable domain comprising an amino acid sequence that is at least 80% identical to SEQ ID NO: 213;
(x) a heavy chain variable domain comprising an amino acid sequence that is at least 80% identical to SEQ ID NO: 214 and a light chain variable domain comprising an amino acid sequence that is at least 80% identical to SEQ ID NO: 215;
(xi) a heavy chain variable domain comprising an amino acid sequence that is at least 80% identical to SEQ ID NO: 216 and a light chain variable domain comprising an amino acid sequence that is at least 80% identical to SEQ ID NO: 217;
(xii) a heavy chain variable domain comprising an amino acid sequence that is at least 80% identical to SEQ ID NO: 218 and a light chain variable domain comprising an amino acid sequence that is at least 80% identical to SEQ ID NO: 219;
(xiii) a heavy chain variable domain comprising an amino acid sequence that is at least 80% identical to SEQ ID NO: 220 and a light chain variable domain comprising an amino acid sequence that is at least 80% identical to SEQ ID NO: 221;
(xiv) a heavy chain variable domain comprising an amino acid sequence that is at least 80% identical to SEQ ID NO: 222 and a light chain variable domain comprising an amino acid sequence that is at least 80% identical to SEQ ID NO: 223;
(xv) a heavy chain variable domain comprising an amino acid sequence that is at least 80% identical to SEQ ID NO: 224 and a light chain variable domain comprising an amino acid sequence that is at least 80% identical to SEQ ID NO: 225;
(xvi) a heavy chain variable domain comprising an amino acid sequence that is at least 80% identical to SEQ ID NO: 226 and a light chain variable domain comprising an amino acid sequence that is at least 80% identical to SEQ ID NO: 227;
(xvii) a heavy chain variable domain comprising an amino acid sequence that is at least 80% identical to SEQ ID NO: 228 and a light chain variable domain comprising an amino acid sequence that is at least 80% identical to SEQ ID NO: 229;
(xviii) a heavy chain variable domain comprising an amino acid sequence that is at least 80% identical to SEQ ID NO: 230 and a light chain variable domain comprising an amino acid sequence that is at least 80% identical to SEQ ID NO: 231;
(xix) a heavy chain variable domain comprising an amino acid sequence that is at least 80% identical to SEQ ID NO: 232 and a light chain variable domain comprising an amino acid sequence that is at least 80% identical to SEQ ID NO: 233;
(xx) a heavy chain variable domain comprising an amino acid sequence that is at least 80% identical to SEQ ID NO: 234 and a light chain variable domain comprising an amino acid sequence that is at least 80% identical to SEQ ID NO: 235;
(xxi) a heavy chain variable domain comprising an amino acid sequence that is at least 80% identical to SEQ ID NO: 236 and a light chain variable domain comprising an amino acid sequence that is at least 80% identical to SEQ ID NO: 237; or
(xxii) a heavy chain variable domain comprising an amino acid sequence that is at least 80% identical to SEQ ID NO: 238 and a light chain variable domain comprising an amino acid sequence that is at least 80% identical to SEQ ID NO: 239.

In a preferred embodiment, the MuSK antibody-based molecule (or the anti-MuSK antibody or antigen binding fragment thereof) disclosed herein comprises:
a heavy chain variable domain (VH) comprising an amino acid sequence that is at least 80% identical or similar to SEQ ID NO: 234 and a light chain variable domain (VL) comprising an amino acid sequence that is at least 80% identical or similar to SEQ ID NO: 235.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the MuSK antibody-based molecule disclosed herein comprises:
a heavy chain variable domain comprising amino acid sequence SEQ ID NO: 234 and a light chain variable domain comprising amino acid sequence SEQ ID NO: 235.

In a preferred embodiment, the antibody-based molecule that binds to human muscle-specific tyrosine-protein kinase (MuSK) comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In a more preferred embodiment, the antibody-based molecule that binds to human muscle-specific tyrosine-protein kinase (MuSK) comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO: 156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising at least 80% sequence identity to SEQ ID NO: 266 or 267, where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the CDR-H2 of the antibody comprises a proline (P) at position 3, a tryptophan (W) at position 4, and a serine(S) or asparagine (N) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO: 156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO: 195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the CDR-H2 of the antibody comprises a proline (P) at position 3, a tryptophan (W) at position 4, and a serine(S) or asparagine (N) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprising SEQ ID NO: 234 and the light chain variable domain comprising SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO: 195 (3B2g2m1).

In an embodiment, the CDR-H2 of the antibody comprises a proline (P) at position 3, a tryptophan (W) at position 4, and a serine(S) or asparagine (N) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprising SEQ ID NO: 234 and the light chain variable domain comprising SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO: 156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).
   a CDR-L3 amino acid sequence comprising SEQ ID NO: 195 or having 1, 2, 3, 4 or 5 amino acid alterations relative to SEQ ID NO: 195 (3B2g2m1).

In an embodiment, the antibody-based molecule that binds to human muscle-specific tyrosine-protein kinase (MuSK) comprises wild-type human IgG constant Fc region, a heavy chain variable domain and a light chain variable domain,
where the wild-type human IgG constant Fc region comprising at least 80% sequence identity to SEQ ID NO: 266 or 267, wherein a), wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A, and
where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO: 156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, wherein L234A and/or L235A substitution(s) is (are) numbered according to the EU numbering system introduced into said Fc region, and where the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, wherein L234A and L235A substitutions numbered according the EU numbering system are introduced into said Fc region, and
where the heavy chain variable domain comprising SEQ ID NO: 234 and the light chain variable domain comprising SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO: 156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).
   a CDR-L3 amino acid sequence comprising SEQ ID NO: 195 or having 1, 2, 3, 4 or 5 amino acid alterations relative to SEQ ID NO:195 (3B2g2m1).

In an embodiment, the anti-MuSK antibody or antigen binding fragment thereof, comprises:
a) A full length heavy chain comprising an amino acid sequence that is at least 80% identical or similar to SEQ ID NO: 268 and
b) A full length light chain comprising an amino acid sequence that is at least 80% identical or similar to SEQ ID NO: 269, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the anti-MuSK antibody or antigen binding fragment thereof, comprises:
a) a full length heavy chain comprising SEQ ID NO: 268 and
b) a full length light chain comprising SEQ ID NO: 269, and
c) wherein the full length heavy chain comprises L234A and L235A mutations numbered according the EU numbering system.

In an embodiment, the anti-MuSK antibody or antigen binding fragment thereof, comprises:
a) a full length heavy chain comprising SEQ ID NO: 270 and
b) a full length light chain comprising SEQ ID NO: 271, and
c) wherein the full length heavy chain comprises L234A and L235A mutations numbered according the EU numbering system.

In an embodiment, the anti-MuSK antibody or antigen binding fragment thereof, comprises:
a) a full length heavy chain comprising an amino acid sequence that is at least 80% identical or similar to SEQ ID NO: 270 and
b) a full length light chain comprising an amino acid sequence that is at least 80% identical or similar to SEQ ID NO: 271, and
c) wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the anti-MuSK antibody or antigen binding fragment thereof, comprises:
a) A full length heavy chain comprising SEQ ID NO: 270 and
b) A full length light chain comprising SEQ ID NO: 271, and
c) Wherein the full length heavy chain comprises L234A and L235A mutations numbered according the EU numbering system.

In a preferred embodiment, the MuSK antibody-based molecule (or the anti-MuSK antibody or antigen binding fragment thereof) disclosed herein comprises:
a heavy chain variable domain (VH) comprising an amino acid sequence that is at least 80% identical or similar to SEQ ID NO: 202 and a light chain variable domain (VL) comprising an amino acid sequence that is at least 80% identical or similar to SEQ ID NO: 203.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the MuSK antibody-based molecule disclosed herein comprises a heavy chain variable domain comprising amino acid sequence SEQ ID NO: 202 and a light chain variable domain comprising amino acid sequence SEQ ID NO: 203.

In a preferred embodiment, the antibody-based molecule that binds to human muscle-specific tyrosine-protein kinase (MuSK) comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156, and
   the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO: 183 (3B2).

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising at least 80% sequence identity to SEQ ID NO: 266 or 267, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and a CDR-H3 amino acid sequence comprising SEQ ID NO: 156 or having 1, 2, 3, 4 or 5 amino acid alterations relative to SEQ ID NO: 156, and
   the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the CDR-H2 of the antibody comprises a proline (P) at position 3, a tryptophan (W) 30 at position 4, and a serine(S) or asparagine (N) at position 5. In a preferred embodiment, the CDR-H2 of the antibody comprises a proline (P) at position 3, a tryptophan (W) at position 4, and an asparagine (N) at position 5.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, where the heavy chain variable domain comprising SEQ ID NO: 202 and the light chain variable domain comprising SEQ ID NO: 203, and wherein:
the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156, and
the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO: 183 (3B2).

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising at least 80% sequence identity to SEQ ID NO: 266 or 267, wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A, and
where the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and wherein:
the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156, and
the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the antibody-based molecule that binds to human muscle-specific tyrosine-protein kinase (MuSK) comprises wild-type human IgG constant Fc region, a heavy chain variable domain and a light chain variable domain, where the wild-type human IgG constant Fc region comprising SEQ ID NO: 266 or 267, wherein L234A and L235A substitutions numbered according the EU numbering system are introduced into said Fc region, and
where the heavy chain variable domain comprising SEQ ID NO: 202 and the light chain variable domain comprising SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO: 156 (3B2) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the anti-MuSK antibody or antigen binding fragment thereof, comprises:
a) a full length heavy chain comprising an amino acid sequence that is at least 80% identical or similar to SEQ ID NO: 282 and
b) a full length light chain comprising an amino acid sequence that is at least 80% identical or similar to SEQ ID NO: 283, and
c) wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the anti-MuSK antibody or antigen binding fragment thereof, comprises:
a) a full length heavy chain comprising SEQ ID NO: 282 and
b) a full length light chain comprising SEQ ID NO: 283, and
c) wherein the full length heavy chain comprises L234A and L235A mutations numbered according the EU numbering system.

### Polynucleotide

Another aspect of the present invention is directed to isolated polynucleotides encoding the MuSK antibody-based molecules described herein. In one embodiment, the polynucleotide comprises a nucleotide sequence which encodes the antibody-based molecule comprising a heavy chain variable region (VH) and a light chain variable region (VL) as identified in table 3 and/or a CDR as identified in table 1 or 2. Accordingly, the invention provides a polynucleotide for use in preventing, delaying, reversing or treating a disease or condition resulting from agrin deficiency in a human subject, wherein the polynucleotide comprises a nucleotide sequence encoding the anti-MuSK antibody or antigen binding fragment or VH, VL or CDRs domain thereof as identified in previous embodiments. In one embodiment, the polynucleotide encoding the MuSK antibody of the present invention comprises a nucleotide sequence encoding any one, any two, any three, any four, any five, or any six of the CDRs described supra, including the heavy chain CDRs of SEQ ID NOS: 1-48, 135-140, 147-158, 240-251 and the light chain CDRs of SEQ ID NOS: 49-96, 141-146, and 159-195.

In an embodiment, the polynucleotide comprises a nucleotide sequence encoding a VH domain, where the VH domain comprises:
(i) a heavy chain variable domain comprising the CDR-H1 of SEQ ID NO: 1, the CDR-H2 of SEQ ID NO: 17, and the CDR-H3 of SEQ ID NO: 33;
(ii) a heavy chain variable domain comprising the CDR-H1 of SEQ ID NO: 2, the CDR-H2 of SEQ ID NO: 18, and the CDR-H3 of SEQ ID NO: 34;
(iii) a heavy chain variable domain comprising the CDR-H1 of SEQ ID NO: 3, the CDR-H2 of SEQ ID NO: 19, and the CDR-H3 of SEQ ID NO: 35;
(iv) a heavy chain variable domain comprising the CDR-H1 of SEQ ID NO: 4, the CDR-H2 of SEQ ID NO: 20, and the CDR-H3 of SEQ ID NO: 36;
(v) a heavy chain variable domain comprising the CDR-H1 of SEQ ID NO: 5, the CDR-H2 of SEQ ID NO: 21, and the CDR-H3 of SEQ ID NO: 37;
(vi) a heavy chain variable domain comprising the CDR-H1 of SEQ ID NO: 6, the CDR-H2 of SEQ ID NO: 22, and the CDR-H3 of SEQ ID NO: 38;
(vii) a heavy chain variable domain comprising the CDR-H1 of SEQ ID NO: 7, the CDR-H2 of SEQ ID NO: 23, and the CDR-H3 of SEQ ID NO: 39;
(viii) a heavy chain variable domain comprising the CDR-H1 of SEQ ID NO: 8, the CDR-H2 of SEQ ID NO: 24, and the CDR-H3 of SEQ ID NO: 40;
(ix) a heavy chain variable domain comprising the CDR-H1 of SEQ ID NO: 9, the CDR-H2 of SEQ ID NO: 25, and the CDR-H3 of SEQ ID NO: 41;
(x) a heavy chain variable domain comprising the CDR-H1 of SEQ ID NO: 10, the CDR-H2 of SEQ ID NO: 26, and the CDR-H3 of SEQ ID NO: 42;
(xi) a heavy chain variable domain comprising the CDR-H1 of SEQ ID NO: 11, the CDR-H2 of SEQ ID NO: 27, and the CDR-H3 of SEQ ID NO: 43;
(xii) a heavy chain variable domain comprising the CDR-H1 of SEQ ID NO: 12, the CDR-H2 of SEQ ID NO: 28, and the CDR-H3 of SEQ ID NO: 44;
(xiii) a heavy chain variable domain comprising the CDR-H1 of SEQ ID NO: 13, the CDR-H2 of SEQ ID NO: 29, and the CDR-H3 of SEQ ID NO: 45;
(xiv) a heavy chain variable domain comprising the CDR-H1 of SEQ ID NO: 14, the CDR-H2 of SEQ ID NO: 30, and the CDR-H3 of SEQ ID NO: 46;
(xv) a heavy chain variable domain comprising the CDR-H1 of SEQ ID NO: 15, the CDR-H2 of SEQ ID NO: 31, and the CDR-H3 of SEQ ID NO: 47;
(xvi) a heavy chain variable domain comprising the CDR-H1 of SEQ ID NO: 16, the CDR-H2 of SEQ ID NO: 32, and the CDR-H3 of SEQ ID NO: 48;
(xvii) a heavy chain variable domain comprising the CDR-H1 of SEQ ID NO: 135, the CDR-H2 of SEQ ID NO: 137, and the CDR-H3 of SEQ ID NO: 139; or
(xviii) a heavy chain variable domain comprising the CDR-H1 of SEQ ID NO: 136, the CDR-H2 of SEQ ID NO: 138, and the CDR-H3 of SEQ ID NO: 140.

In some embodiments, the polynucleotide comprises a nucleotide sequence encoding a VH domain, where the VH domain comprises:
(ii.a) a heavy chain variable domain comprising the CDR-H1 of SEQ ID NO: 2, the CDR-H2 of SEQ ID NO: 18, and the CDR-H3 of SEQ ID NO: 240 (X2m1);
(ii.b) a heavy chain variable domain comprising the CDR-H1 of SEQ ID NO: 2, the CDR-H2 of SEQ ID NO: 18, and the CDR-H3 of SEQ ID NO: 241 (X2m2);
(ii.c) a heavy chain variable domain comprising the CDR-H1 of SEQ ID NO: 2, the CDR-H2 of SEQ ID NO: 18, and the CDR-H3 of SEQ ID NO: 242 (X2m3);
(ii.d) a heavy chain variable domain comprising the CDR-H1 of SEQ ID NO: 2, the CDR-H2 of SEQ ID NO: 18, and the CDR-H3 of SEQ ID NO: 243 (X2m4);
(ii.e) a heavy chain variable domain comprising the CDR-H1 of SEQ ID NO: 2, the CDR-H2 of SEQ ID NO: 18, and the CDR-H3 of SEQ ID NO: 244 (X2m5);
(ii.f) a heavy chain variable domain comprising the CDR-H1 of SEQ ID NO: 2, the CDR-H2 of SEQ ID NO: 18, and the CDR-H3 of SEQ ID NO: 245 (X2m6);
(ii.g) a heavy chain variable domain comprising the CDR-H1 of SEQ ID NO: 2, the CDR-H2 of SEQ ID NO: 18, and the CDR-H3 of SEQ ID NO: 246 (X2m7); or (ii.h) a heavy chain variable domain comprising the CDR-H1 of SEQ ID NO: 2, the CDR-H2 of SEQ ID NO: 18, and the CDR-H3 of SEQ ID NO: 247 (X2m8).

In some embodiments, the polynucleotide comprises a nucleotide sequence encoding a VH domain, where the VH domain comprises:
(xvii.a) a heavy chain variable domain comprising the CDR-H1 of SEQ ID NO: 135, the CDR-H2 of SEQ ID NO: 137, and the CDR-H3 of SEQ ID NO: 248 (X17m1);
(xvii.b) a heavy chain variable domain comprising the CDR-H1 of SEQ ID NO: 135, the CDR-H2 of SEQ ID NO: 137, and the CDR-H3 of SEQ ID NO: 249 (X17m2);
(xvii.c) a heavy chain variable domain comprising the CDR-H1 of SEQ ID NO: 135, the CDR-H2 of SEQ ID NO: 137, and the CDR-H3 of SEQ ID NO: 250 (X17m3); or (xvii.d) a heavy chain variable domain comprising the CDR-H1 of SEQ ID NO: 135, the CDR-H2 of SEQ ID NO: 137, and the CDR-H3 of SEQ ID NO: 251 (X17m6).

In an embodiment, the polynucleotide comprises a nucleotide sequence encoding a VH domain, where the VH domain comprises:
(xix) a heavy chain variable domain comprising the CDR-H1 of SEQ ID NO: 147, the CDR-H2 of SEQ ID NO: 150, and the CDR-H3 of SEQ ID NO: 156;
(xx) a heavy chain variable domain comprising the CDR-H1 of SEQ ID NO: 148, the CDR-H2 of SEQ ID NO: 151, and the CDR-H3 of SEQ ID NO: 157; or
(xxi) a heavy chain variable domain comprising the CDR-H1 of SEQ ID NO: 149, the CDR-H2 of SEQ ID NO: 152, and the CDR-H3 of SEQ ID NO: 158.

In an embodiment, the polynucleotide comprises a nucleotide sequence encoding a VH domain, where the VH domain comprises:
(xxii) a heavy chain variable domain comprising the CDR-H1 of SEQ ID NO: 147, the CDR-H2 of SEQ ID NO: 153, and the CDR-H3 of SEQ ID NO:156;
(xxiii) a heavy chain variable domain comprising the CDR-H1 of SEQ ID NO: 147, the CDR-H2 of SEQ ID NO: 154, and the CDR-H3 of SEQ ID NO: 156; or
(xxiv) a heavy chain variable domain comprising the CDR-H1 of SEQ ID NO: 147, the CDR-H2 of SEQ ID NO: 155, and the CDR-H3 of SEQ ID NO: 156.

In an embodiment, the polynucleotide comprises a nucleotide sequence encoding a VL domain, where the VL domain comprises:
(i) a light chain variable domain comprising the CDR-L1 of SEQ ID NO: 49, the CDR-L2 of SEQ ID NO: 65, and the CDR-L3 of SEQ ID NO: 81;
(ii) a light chain variable domain comprising the CDR-L1 of SEQ ID NO: 50, the CDR-L2 of SEQ ID NO: 66, and the CDR-L3 of SEQ ID NO: 82;
(iii) a light chain variable domain comprising the CDR-L1 of SEQ ID NO: 51, the CDR-L2 of SEQ ID NO: 67, and the CDR-L3 of SEQ ID NO: 83;
(iv) a light chain variable domain comprising the CDR-L1 of SEQ ID NO: 52, the CDR-L2 of SEQ ID NO: 68, and the CDR-L3 of SEQ ID NO: 84;
(v) a light chain variable domain comprising the CDR-L1 of SEQ ID NO: 53, the CDR-L2 of SEQ ID NO: 69, and the CDR-L3 of SEQ ID NO: 85;
(vi) a light chain variable domain comprising the CDR-L1 of SEQ ID NO: 54, the CDR-L2 of SEQ ID NO: 70, and the CDR-L3 of SEQ ID NO: 86;
(vii) a light chain variable domain comprising the CDR-L1 of SEQ ID NO: 55, the CDR-L2 of SEQ ID NO: 71, and the CDR-L3 of SEQ ID NO: 87;
(viii) a light chain variable domain comprising the CDR-L1 of SEQ ID NO: 56, the CDR-L2 of SEQ ID NO: 72, and the CDR-L3 of SEQ ID NO: 88;
(ix) a light chain variable domain comprising the CDR-L1 of SEQ ID NO: 57, the CDR-L2 of SEQ ID NO: 73, and the CDR-L3 of SEQ ID NO: 89;
(x) a light chain variable domain comprising the CDR-L1 of SEQ ID NO: 58, the CDR-L2 of SEQ ID NO: 74, and the CDR-L3 of SEQ ID NO: 90;
(xi) a light chain variable domain comprising the CDR-L1 of SEQ ID NO: 59, the CDR-L2 of SEQ ID NO: 75, and the CDR-L3 of SEQ ID NO: 91;
(xii) a light chain variable domain comprising the CDR-L1 of SEQ ID NO: 60, the CDR-L2 of SEQ ID NO: 76, and the CDR-L3 of SEQ ID NO: 92;
(xiii) a light chain variable domain comprising the CDR-L1 of SEQ ID NO: 61, the CDR-L2 of SEQ ID NO: 77, and the CDR-L3 of SEQ ID NO: 93;
(xiv) a light chain variable domain comprising the CDR-L1 of SEQ ID NO: 62, the CDR-L2 of SEQ ID NO: 78, and the CDR-L3 of SEQ ID NO: 94;
(xv) a light chain variable domain comprising the CDR-L1 of SEQ ID NO: 63, the CDR-L2 of SEQ ID NO: 79, and the CDR-L3 of SEQ ID NO: 95;
(xvi) a light chain variable domain comprising the CDR-L1 of SEQ ID NO: 64, the CDR-L2 of SEQ ID NO: 80, and the CDR-L3 of SEQ ID NO: 96;
(xvii) a light chain variable domain comprising the CDR-L1 of SEQ ID NO: 141, the CDR-L2 of SEQ ID NO: 143, and the CDR-L3 of SEQ ID NO: 145; or
(xviii) a light chain variable domain comprising the CDR-L1 of SEQ ID NO: 142, the CDR-L2 of SEQ ID NO: 144, and the CDR-L3 of SEQ ID NO: 146.

In an embodiment, the polynucleotide comprises a nucleotide sequence encoding a VL domain, where the VL domain comprises:
(xix) a light chain variable domain comprising the CDR-L1 of SEQ ID NO: 159, the CDR-L2 of SEQ ID NO: 170, and the CDR-L3 of SEQ ID NO: 180;
(xx) a light chain variable domain comprising the CDR-L1 of SEQ ID NO: 159, the CDR-L2 of SEQ ID NO: 171, and the CDR-L3 of SEQ ID NO: 181;
(xxi) a light chain variable domain comprising the CDR-L1 of SEQ ID NO: 160, the CDR-L2 of SEQ ID NO: 172, and the CDR-L3 of SEQ ID NO: 182;
(xxii) a light chain variable domain comprising the CDR-L1 of SEQ ID NO: 159, the CDR-L2 of SEQ ID NO: 172, and the CDR-L3 of SEQ ID NO: 183;
(xxiii) a light chain variable domain comprising the CDR-L1 of SEQ ID NO: 159, the CDR-L2 of SEQ ID NO: 171, and the CDR-L3 of SEQ ID NO: 184;
(xxiv) a light chain variable domain comprising the CDR-L1 of SEQ ID NO: 159, the CDR-L2 of SEQ ID NO: 173, and the CDR-L3 of SEQ ID NO: 185;
(xxv) a light chain variable domain comprising the CDR-L1 of SEQ ID NO: 159, the CDR-L2 of SEQ ID NO: 173, and the CDR-L3 of SEQ ID NO: 186;
(xxvi) a light chain variable domain comprising the CDR-L1 of SEQ ID NO: 161, the CDR-L2 of SEQ ID NO: 174, and the CDR-L3 of SEQ ID NO: 187;
(xxvii) a light chain variable domain comprising the CDR-L1 of SEQ ID NO: 162, the CDR-L2 of SEQ ID NO: 174, and the CDR-L3 of SEQ ID NO: 188;
(xxviii) a light chain variable domain comprising the CDR-L1 of SEQ ID NO: 163, the CDR-L2 of SEQ ID NO: 174, and the CDR-L3 of SEQ ID NO: 188;
(xxix) a light chain variable domain comprising the CDR-L1 of SEQ ID NO: 164, the CDR-L2 of SEQ ID NO: 174, and the CDR-L3 of SEQ ID NO: 189;
(xxx) a light chain variable domain comprising the CDR-L1 of SEQ ID NO: 165, the CDR-L2 of SEQ ID NO: 175, and the CDR-L3 of SEQ ID NO: 190;
(xxxi) a light chain variable domain comprising the CDR-L1 of SEQ ID NO: 166, the CDR-L2 of SEQ ID NO: 176, and the CDR-L3 of SEQ ID NO: 191;
(xxxii) a light chain variable domain comprising the CDR-L1 of SEQ ID NO: 167, the CDR-L2 of SEQ ID NO: 177, and the CDR-L3 of SEQ ID NO: 192;
(xxxiii) a light chain variable domain comprising the CDR-L1 of SEQ ID NO: 168, the CDR-L2 of SEQ ID NO: 178, and the CDR-L3 of SEQ ID NO: 193; or
(xxxiv) a light chain variable domain comprising the CDR-L1 of SEQ ID NO: 169, the CDR-L2 of SEQ ID NO: 179, and the CDR-L3 of SEQ ID NO: 194.

In an embodiment, the polynucleotide comprises a nucleotide sequence encoding a VL domain, where the VL domain comprises:
(xxxv) a light chain variable domain comprising the CDR-L1 of SEQ ID NO: 159, the CDR-L2 of SEQ ID NO: 172, and the CDR-L3 of SEQ ID NO: 183; or
(xxxvi) a light chain variable domain comprising the CDR-L1 of SEQ ID NO: 159, the CDR-L2 of SEQ ID NO: 172, and the CDR-L3 of SEQ ID NO: 195.

In an embodiment, the isolated polynucleotide encoding the MuSK antibody-based molecule encodes any one of the VH and/or VL domain sequences as provided in Table 3. The nucleic acid molecules described herein include isolated polynucleotides, portions of expression vectors or portions of linear DNA sequences, including linear DNA sequences used for in vitro transcription/translation, and vectors compatible with prokaryotic, eukaryotic or filamentous phage expression, secretion, and/or display of the antibodies or binding fragments thereof described herein.

In a preferred embodiment, the polynucleotide comprises a nucleotide sequence encoding a VH that comprises an amino acid sequence that is at least 80% identical or similar to SEQ ID NO: 234. In another preferred embodiment, the polynucleotide comprises a nucleotide sequence encoding a VL that comprises an amino acid sequence that is at least 80% identical or similar to SEQ ID NO: 235.

In a preferred embodiment, the polynucleotide comprises a nucleotide sequence encoding an antibody-based molecule that binds to human muscle-specific tyrosine-protein kinase (MuSK), said molecule comprising a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO: 156 (3B2g2m1) and where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the polynucleotide comprises or consists of a nucleotide sequence that is at least 80% 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 264. In an even more preferred embodiment, the polynucleotide comprises or consists of SEQ ID NO:264.

In another more preferred embodiment, the polynucleotide comprises or consists of a nucleotide sequence that is at least 80% 81%, 82%, 83%, 84%, 85%, 86%, 87%, 88%, 89%, 90%, 91%, 92%, 93%, 94%, 95%, 96%, 97%, 98%, 99% or 100% identical to SEQ ID NO: 265. In an even more preferred embodiment, the polynucleotide comprises or consists of SEQ ID NO:265.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain is encoded by a nucleotide sequence that is at least 80% identical to SEQ ID NO: 264 and the light chain variable domain is encoded by a nucleotide sequence that is at least 80% identical to SEQ ID NO: 265. In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:276 encoding the full length heavy chain, and
b) a nucleotide sequence that is at least 80% identical to SEQ ID NO:278 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:277 encoding the heavy chain variable domain, and
b) a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:276 encoding the full length heavy chain, wherein said nucleotide sequence comprising a nucleotide sequence that is at least 80% identical to SEQ ID NO: 277 encoding the heavy chain variable domain, and
b) a nucleotide sequence that is at least 80% identical to SEQ ID NO:278 encoding the full length light chain, wherein said nucleotide sequence comprising a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence SEQ ID NO:276 and
b) a nucleotide sequence SEQ ID NO:278.

In a preferred embodiment, the polynucleotide comprises a nucleotide sequence encoding a VH that comprises an amino acid sequence that is at least 80% identical or similar to SEQ ID NO: 202. In another preferred embodiment, the polynucleotide comprises a nucleotide sequence encoding a VL that comprises an amino acid sequence that is at least 80% identical or similar to SEQ ID NO: 203

In a preferred embodiment, the polynucleotide comprises a nucleotide sequence encoding an antibody-based molecule that binds to human muscle-specific tyrosine-protein kinase (MuSK), said molecule comprising a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO: 183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (3B2) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the polynucleotide comprises a nucleotide sequence encoding an antibody-based molecule that binds to human muscle-specific tyrosine-protein kinase (MuSK), that comprises a heavy chain variable domain and a light chain variable domain, wherein the heavy chain variable domain is encoded by a nucleotide sequence that is at least 80% identical to SEQ ID NO: 284 and the light chain variable domain is encoded by a nucleotide sequence that is at least 80% identical to SEQ ID NO: 285.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:288 encoding the full length heavy chain, and
b) a nucleotide sequence that is at least 80% identical to SEQ ID NO:289 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:284 encoding the heavy chain variable domain, and
b) a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence that is at least 80% identical to SEQ ID NO:288 encoding the full length heavy chain, wherein said nucleotide sequence comprising a nucleotide sequence that is at least 80% identical to SEQ ID NO: 284 encoding the heavy chain variable domain, and
b) a nucleotide sequence that is at least 80% identical to SEQ ID NO:289 encoding the full length light chain, wherein said nucleotide sequence comprising a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the polynucleotide comprises:
a) a nucleotide sequence SEQ ID NO:288 and
b) a nucleotide sequence SEQ ID NO:289.

The polynucleotides of the invention may be produced by chemical synthesis such as solid phase polynucleotide synthesis on an automated polynucleotide synthesizer and assembled into complete single or double stranded molecules. Alternatively, the polynucleotides may be produced by other techniques such a PCR followed by routine cloning. Techniques for producing or obtaining polynucleotides of a given sequence are well known in the art.

The polynucleotides may comprise at least one non-coding sequence, such as a promoter or enhancer sequence, intron, polyadenylation signal, a cis sequence facilitating RepA binding, and the like. The polynucleotide sequences may also comprise additional sequences encoding for example a linker sequence, a marker or a tag sequence, such as a histidine tag or an HA tag to facilitate purification or detection of the protein, a signal sequence, a fusion protein partner such as RepA, Fc portion, or bacteriophage coat protein such as plX or plll.

### Vector

In another aspect, there is provided a vector (preferably an expression vector) for producing the MuSK antibody-based molecule for use in preventing, delaying, reversing or treating a disease or condition resulting from agrin deficiency in a human subject comprising the polynucleotide encoding the MuSK antibody-based molecule (or anti-MuSK antibody or antigen binding fragment thereof) as described herein.

Such vectors include, without limitation, plasmid vectors, viral vectors, including without limitation, vaccina vector, lentiviral vector, adenoviral vector, adeno-associated viral vector, vectors for baculovirus expression, transposon based vectors or any other vector suitable for introduction of the polynucleotides described herein into a given organism or genetic background by any means to facilitate expression of the encoded antibody polypeptide. In one embodiment, the polynucleotide encoding the heavy chain variable domain, alone or together with the polynucleotide encoding the light chain variable domain as described herein, are combined with sequences of a promoter, a translation initiation segment (e.g., a ribosomal binding sequence and start codon), a 3′ untranslated region, polyadenylation signal, a termination codon, and transcription termination to form one or more expression vector constructs.

In one embodiment, the vector is an adenoviral-associated viral (AAV) vector. A number of therapeutic AAV vectors suitable for delivery of the polynucleotides encoding antibodies described herein to the central nervous system are known in the art. See e.g., Deverman et al., “Gene Therapy for Neurological Disorders: Progress and Prospects,” Nature Rev. 17:641-659 (2018), which in hereby incorporated by reference in its entirety. Suitable AAV vectors include serotypes AAV1, AAV2, AAV3, AAV4, AAV5, AAV6, AAV7, AAV8, AAV9, AAV10, or AAV11 in their native form or engineered for enhanced tropism. AAV vectors known to have tropism for the CNS that are particularly suited for therapeutic expression of the MuSK antibodies described herein include, AAV1, AAV2, AAV4, AAV5, AAV8 and AAV9 in their native form or engineered for enhanced tropism. In one embodiment, the AAV vector is an AAV2 vector. In another embodiment, the AAV vector is an AAV5 vector (Vitale et al., “Anti-tau Conformational scFv MC1 Antibody Efficiently Reduces Pathological Tau Species in Adult JNPL3 Mice,” Acta Neuropathol. Commun. 6:82 (2018), which is hereby incorporate by reference in its entirety). In another embodiment, the AAV vector is an AAV9 vector (Haiyan et al., “Targeting Root Cause by Systemic scAAV9-hIDS Gene Delivery: Functional Correction and Reversal of Severe MPSII in Mice,” Mol. Ther. Methods Clin. Dev. 10:327-340 (2018), which is hereby incorporated by reference in its entirety). In another embodiment, the AAV vector is an AAVrh10 vector (Liu et al., “Vectored Intracerebral Immunizations with the Anti-Tau Monoclonal Antibody PHF1 Markedly Reduces Tau Pathology in Mutant Transgenic Mice,” J. Neurosci. 36 (49): 12425-35 (2016), which is hereby incorporated by reference in its entirety).

In another embodiment the AAV vector is a hybrid vector comprising the genome of one serotype, e.g., AAV2, and the capsid protein of another serotype, e.g., AAV1 or AAV3-9 to control tropism. See e.g., Broekman et al., “Adeno-associated Virus Vectors Serotyped with AAV8 Capsid are More Efficient than AAV-1 or-2 Serotypes for Widespread Gene Delivery to the Neonatal Mouse Brain,” Neuroscience 138:501-510 (2006), which is hereby incorporated by reference in its entirety. In one embodiment, the AAV vector is an AAV2/8 hybrid vector (Ising et al., “AAV-mediated Expression of Anti-Tau ScFv Decreases Tau Accumulation in a Mouse Model of Tauopathy,” J. Exp. Med. 214 (5): 1227 (2017), which is hereby incorporated by reference in its entirety). In another embodiment the AAV vector is an AAV2/9 hybrid vector (Simon et al., “A Rapid Gene Delivery-Based Mouse Model for Early-Stage Alzheimer Disease-Type Tauopathy,” J. Neuropath. Exp. Neurol. 72 (11): 1062-71 (2013), which is hereby incorporated by reference in its entirety).

In another embodiment, the AAV vector is one that has been engineered or selected for its enhanced CNS transduction after intraparenchymal administration, e.g., AAV-DJ (Grimm et al., J. Viol. 82:5887-5911 (2008), which is hereby incorporated by reference in its entirety); increased transduction of neural stem and progenitor cells, e.g., SCH9 and AAV4.18 (Murlidharan et al., J. Virol. 89:3976-3987 (2015) and Ojala et al., Mol. Ther. 26:304-319 (2018), which are hereby incorporated by reference in their entirety); enhanced retrograde transduction, e.g., rAAV2-retro (Muller et al., Nat. Biotechnol. 21:1040-1046 (2003), which is hereby incorporated by reference in its entirety); selective transduction into brain endothelial cells, e.g., AAV-BRI (Korbelin et al., EMBO Mol. Med. 8:609-625 (2016), which is hereby incorporated by reference in its entirety); or enhanced transduction of the adult CNS after IV administration, e.g., AAV-PHP.B and AAVPHP.eB (Deverman et al., Nat. Biotechnol. 34:204-209 (2016) and Chan et al., Nat. Neurosci. 20:1172-1179 (2017), which are hereby incorporated by reference in their entirety.

In accordance with this embodiment, the expression vector construct encoding the MuSK antibody-based molecule includes the polynucleotide encoding the heavy chain polypeptide, a functional fragment thereof, a variant thereof, or combinations thereof. The expression construct can alternatively include a nucleic acid sequence encoding the light chain polypeptide, a functional fragment thereof, a variant thereof, or combinations thereof. In an embodiment, the expression vector construct includes a nucleic acid sequence encoding the heavy chain polypeptide, a functional fragment thereof, or a variant thereof, and the light chain polypeptide, a functional fragment thereof, or a variant thereof.

In an embodiment, the expression construct further comprises a promoter sequence suitable for driving expression of the MuSK antibody-based molecule. Suitable promoter sequences include, without limitation, the elongation factor 1-alpha promoter (EF1a) promoter, a phosphoglycerate kinase-1 promoter (PGK) promoter, a cytomegalovirus immediate early gene promoter (CMV), a chimeric liver-specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter. Other promoters suitable for driving gene expression in mammalian cells that are known in the art are also suitable for incorporation into the expression constructs disclosed herein.

In an embodiment, the expression construct (or expression vector) further encodes a linker sequence. The linker sequence can encode an amino acid sequence that spatially separates and/or links the one or more components of the expression construct (heavy chain and light chain components of the encoded antibody).

In a preferred embodiment, the expression vector comprises a polynucleotide that encodes an antibody-based molecule that binds to human muscle-specific tyrosine-protein kinase (MuSK) and that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the expression vector comprises a nucleotide encoding an antibody-based molecule that binds to human muscle-specific tyrosine-protein kinase (MuSK) and comprising a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:276 encoding the full length heavy chain, and
b) a nucleotide sequence that is at least 80% identical to SEQ ID NO:278 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:277 encoding the heavy chain variable domain, and
b) a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:276 encoding the full length heavy chain, wherein said nucleotide sequence comprising a nucleotide sequence that is at least 80% identical to SEQ ID NO: 277 encoding the heavy chain variable domain, and
b) a nucleotide sequence that is at least 80% identical to SEQ ID NO:278 encoding the full length light chain, wherein said nucleotide sequence comprising a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence SEQ ID NO:276 and
b) a nucleotide sequence SEQ ID NO:278.

In a preferred embodiment, the expression vector comprises a polynucleotide that encodes an antibody-based molecule that binds to human muscle-specific tyrosine-protein kinase (MuSK) and that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the expression vector comprises a polynucleotide encoding an antibody-based molecule that binds to human muscle-specific tyrosine-protein kinase (MuSK) and comprising a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (3B2) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:288 encoding the full length heavy chain, and
b) a nucleotide sequence that is at least 80% identical to SEQ ID NO:289 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:284 encoding the heavy chain variable domain, and
b) a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence that is at least 80% identical to SEQ ID NO:288 encoding the full length heavy chain, wherein said nucleotide sequence comprising a nucleotide sequence that is at least 80% identical to SEQ ID NO: 284 encoding the heavy chain variable domain, and
b) a nucleotide sequence that is at least 80% identical to SEQ ID NO:289 encoding the full length light chain, wherein said nucleotide sequence comprising a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the expression vector comprises a polynucleotide encoding an antibody-based molecule that binds to human muscle-specific tyrosine-protein kinase (MuSK) and comprising:
a) a nucleotide sequence SEQ ID NO:288 and
b) a nucleotide sequence SEQ ID NO:289.

### Host Cell

Another aspect of the present invention is a host cell or cell-free expression system for producing the MuSK antibody-based molecule for use in preventing, delaying, reversing or treating a disease or condition resulting from agrin deficiency in a human subject wherein the cell contains the expression vector encoding the MuSK antibodies (or antigen binding fragment thereof) and optionally producing said MuSK antibodies as described herein.

The MuSK antibody-based molecules described herein can optionally be produced by a cell line, a mixed cell line, an immortalized cell or clonal population of immortalized cells, as well known in the art (see e.g., Ausubel et al., ed., Current Protocols in Molecular Biology, John Wiley & Sons, Inc., NY, N.Y. (1987-2001); Sambrook et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan et al., eds., Current Protocols in Immunology, John Wiley & Sons, Inc., NY (1994-2001); Colligan et al., Current Protocols in Protein Science, John Wiley & Sons, NY, N.Y., (1997-2001), which are hereby incorporated by reference in their entirety).

In some embodiments, the host cell chosen for expression may be of mammalian origin. Suitable mammalian host cells include, without limitation, COS-1 cells, COS-7 cells, HEK293 cells, BHK21 cells, CHO cells, BSC-1 cells, HeG2 cells, SP2/0 cells, Hela cells, mammalian myeloma cells, mammalian lymphoma cells, or any derivative, immortalized or transformed cell thereof. Other suitable host cells include, without limitation, yeast cells, insect cells, and plant cells. Alternatively, the host cell may be selected from a species or organism incapable of glycosylating polypeptides, e.g., a prokaryotic cell or organism, such as BL21, BL21 (DE3), BL21-GOLD (DE3), XL1-Blue, JM109, HMS174, HMS174 (DE3), and any of the natural or engineered E. coli spp, Klebsiella spp., or Pseudomonas spp strains.

The MuSK antibody-based molecules described herein can be prepared by any of a variety of techniques using the isolated polynucleotides, vectors, and host cells described supra. In general, antibodies can be produced by cell culture techniques, including the generation of monoclonal antibodies via conventional techniques, or via transfection of antibody genes, heavy chains and/or light chains into suitable bacterial or mammalian cell hosts, in order to allow for the production of antibodies, wherein the antibodies may be recombinant. In an embodiment, the MuSK antibody-based molecule described herein is a monoclonal antibody or functional binding fragment thereof. Standard molecular biology techniques are used to prepare the recombinant expression vector, transfect the host cells, select for transformants, culture the host cells and recover the antibody from the culture medium. Transfecting the host cell can be carried out using a variety of techniques commonly used for the introduction of exogenous DNA into a prokaryotic or eukaryotic host cell, e.g., by electroporation, calcium-phosphate precipitation, DEAE-dextran transfection and the like. Although it is possible to express the antibodies described herein in either prokaryotic or eukaryotic host cells, expression of antibodies in eukaryotic cells, in particular mammalian cells is sometimes preferable, because such eukaryotic cells (and in particular mammalian cells) are more likely than prokaryotic cells to assemble and secrete a properly folded and immunologically active antibody.

As noted above, exemplary mammalian host cells for expressing the recombinant antibodies of the invention include Chinese Hamster Ovary (CHO cells) (including dhfr-CHO cells, described in Urlaub and Chasin, Proc. Natl. Acad. Sci. USA, 77:4216-4220 (1980), which is hereby incorporated by reference in its entirety). Other suitable mammalian host cells include, without limitation, NSO myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown.

Host cells can also be used to produce functional antibody fragments, such as Fab fragments or scFv molecules. It is understood that variations on the above procedure are within the scope of the present invention. For example, it may be desirable to transfect a host cell with DNA encoding functional fragments of either the light chain and/or the heavy chain of an antibody described herein. Recombinant DNA technology may also be used to remove some or all of the DNA encoding either or both of the light and heavy chains that is not necessary for binding to the antigens of interest. The molecules expressed from such truncated DNA molecules are also encompassed by the antibodies described herein.

The antibodies and antibody binding fragments are recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

In a preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO: 195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:276 encoding the full length heavy chain, and
b) a nucleotide sequence that is at least 80% identical to SEQ ID NO:278 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:277 encoding the heavy chain variable domain, and
b) a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:276 encoding the full length heavy chain, wherein said nucleotide sequence comprising a nucleotide sequence that is at least 80% identical to SEQ ID NO: 277 encoding the heavy chain variable domain, and
b) a nucleotide sequence that is at least 80% identical to SEQ ID NO:278 encoding the full length light chain, wherein said nucleotide sequence comprising a nucleotide sequence that is at least 80% identical to SEQ ID NO:279 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence SEQ ID NO:276 and
b) a nucleotide sequence SEQ ID NO:278.

In a preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO: 156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the host cell expresses an antibody-based molecule that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO: 156 (3B2) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:288 encoding the full length heavy chain, and
b) a nucleotide sequence that is at least 80% identical to SEQ ID NO:289 encoding the full length light chain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:284 encoding the heavy chain variable domain, and
b) a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence that is at least 80% identical to SEQ ID NO:288 encoding the full length heavy chain, wherein said nucleotide sequence comprising a nucleotide sequence that is at least 80% identical to SEQ ID NO: 284 encoding the heavy chain variable domain, and
b) a nucleotide sequence that is at least 80% identical to SEQ ID NO:289 encoding the full length light chain, wherein said nucleotide sequence comprising a nucleotide sequence that is at least 80% identical to SEQ ID NO:285 encoding the light chain variable domain.

In an embodiment, the identity is at least 81%, 82%, 83%, 84%, 85%, 86%, 87%, 88%, 89%, 90%, 91%, 92%, 93%, 94%, 95%, 96%, 97%, 98%, 99%.

In an embodiment, the host cell expresses an antibody-based molecule that binds to human muscle-specific tyrosine-protein kinase (MuSK), that comprises:
a) a nucleotide sequence SEQ ID NO:288 and
b) a nucleotide sequence SEQ ID NO:289.

### Compositions Comprising MuSK Antibody-Based Molecules

The MuSK antibody-based molecules or polynucleotide encoding the MuSK antibody-based molecules of the present invention may be advantageously administered as compositions.

Therefore in a further aspect, there is provided a composition for use in preventing, delaying, reversing or treating a disease or condition resulting from agrin deficiency in a human subject, said composition comprising an antibody or antigen binding fragment, a polynucleotide, an expression vector as defined herein.

In an embodiment, said composition is a pharmaceutical composition. In an embodiment, said pharmaceutical composition comprising at least one pharmaceutically acceptable carrier or excipients.

In an embodiment, such compositions are pharmaceutical compositions comprising an active therapeutic agent (i.e., the MuSK antibody) and one or more of a variety of other pharmaceutically acceptable components. See REMINGTON: THE SCIENCE AND PRACTICE OF PHARMACY (21st Edition) (2005) (Troy, D. B. et al. (Eds.) Lippincott Williams & Wilkins (Publs.), Baltimore MD), which is hereby incorporated by reference in its entirety. The preferred form depends on the intended mode of administration and therapeutic application. The compositions can also include, depending on the formulation desired, pharmaceutically acceptable, non-toxic carriers, excipients, diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition, and which are vehicles commonly used to formulate pharmaceutical compositions for animal or human administration. The diluent is selected to not affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, or non-toxic, nontherapeutic, non-immunogenic stabilizers and the like. Examples of suitable aqueous and non-aqueous carriers which may be employed in the pharmaceutical compositions of the present invention include water, saline, phosphate-buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well-known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

The compositions may also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Suitability for carriers and other components of pharmaceutical compositions is determined based on the lack of significant negative impact on the desired biological properties of the active antibody-based molecule of the present invention (e.g., less than a substantial impact (e.g., 10% or less relative inhibition, 5% or less relative inhibition, etc.) on antigen binding).

The pharmaceutical compositions of the present invention may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present invention may also comprise isotonicity agents, such as sugars, polyalcohols, such as mannitol, sorbitol, glycerol or sodium chloride in the compositions.

The pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The antibodies of the present invention may be prepared with carriers that will protect the antibodies against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well-known in the art. Methods for the preparation of such formulations are generally known to those skilled in the art. See, e.g., SUSTAINED AND CONTROLLED RELEASE DRUG DELIVERY SYSTEMS, J. R. Robinson, ed., Marcel Dekker, Inc., New York, 1978.

In one embodiment, the antibodies of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art.

Pharmaceutical compositions for injection must typically be sterile and stable under the conditions of manufacture and storage. The composition may be formulated as a solution, microemulsion, liposome, or other ordered structure suitable to achieve high drug concentration. The carrier may be an aqueous or non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

For parenteral administration, agents of the present invention are typically formulated as injectable dosages of a solution or suspension of the substance in a physiologically acceptable diluent with a pharmaceutical carrier that can be a sterile liquid such as water, oil, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin. Peanut oil, soybean oil, and mineral oil are all examples of useful materials. In general, glycols, such as propylene glycol or polyethylene glycol, are preferred liquid carriers, particularly for injectable solutions. Agents of the invention can be administered in the form of a depot injection or implant preparation which can be formulated in such a manner as to permit a sustained release of the active ingredient.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles, such as polylactide, polyglycolide, or copolymer, for enhanced adjuvant effect (Langer, et al., Science 249:1527 (1990); Hanes, et al., Advanced Drug Delivery Reviews 28:97-119 (1997), which are hereby incorporated by reference in their entirety). Additional formulations suitable for other modes of administration include intranasal, and pulmonary formulations, suppositories, and transdermal applications.

In an embodiment, the composition comprises the anti-MuSK antibody (or antigen-binding fragment) which comprises a heavy chain variable domain comprising the CDR-H1 of SEQ ID NO: 147, the CDR-H2 of SEQ ID NO: 153, and the CDR-H3 of SEQ ID NO: 156, and a light chain variable domain comprising the CDR-L1 of SEQ ID NO: 159, the CDR-L2 of SEQ ID NO: 172, and the CDR-L3 of SEQ ID NO: 195 (3B2g2m1).

In an embodiment, the composition comprises the anti-MuSK antibody (or antigen-binding fragment) which comprises a heavy chain variable domain comprising the CDR-H1 of SEQ ID NO: 147, the CDR-H2 of SEQ ID NO: 153, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m1).

In an embodiment, the anti-MuSK antibody (or antigen-binding fragment) which comprises a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2g1m2).

In an embodiment, the anti-MuSK antibody (or antigen-binding fragment) which comprises a heavy chain variable domain comprising the CDR-H1 of SEQ ID NO: 147, the CDR-H2 of SEQ ID NO: 154, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 195 (3B2g2m2).

In an embodiment, the anti-MuSK antibody (or antigen-binding fragment) which comprises a heavy chain variable domain comprising the CDR-H1 of SEQ ID NO: 147, the CDR-H2 of SEQ ID NO: 150, and the CDR-H3 of SEQ ID NO: 156, and the light chain variable domain comprising the CDR-L1 of SEQ ID NO: 159, the CDR-L2 of SEQ ID NO: 172, and the CDR-L3 of SEQ ID NO: 183 (3B2).

In a preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO:156 or having 1, 2, 3, 4 or 5 amino acid alterations relative to SEQ ID NO:156 (3B2g2m1) and
where the light chain variable domain comprises:
   a CDR-L1 amino acid sequence comprises SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO: 195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, composition comprises an antibody or antigen-binding fragment that binds to human muscle-specific tyrosine-protein kinase (MuSK) that comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO: 195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises wild-type human IgG constant Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the composition comprises an antibody or antigen-binding fragment that binds to human muscle-specific tyrosine-protein kinase (MuSK) that comprises wild-type human IgG constant Fc region wherein L234A and/or L235A substitution(s) numbered according the EU numbering system is (are) introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 153 or having 1, 2, 3, 4 or 5 amino acid alterations relative to SEQ ID NO: 153, and
   a CDR-H3 amino acid sequence comprising SEQ ID NO: 156 or having 1, 2, 3, 4 or 5 amino acid alterations relative to SEQ ID NO:156 (3B2g2m1) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 195 or having 1, 2, 3, 4 or 5 amino acid alterations relative to SEQ ID NO: 195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises wild-type human IgG constant Fc region wherein L234A and/or L235A substitution(s) numbered according the EU numbering system is (are) introduced into said Fc region, a heavy chain variable domain and a light chain variable domain, where the wild-type human IgG constant Fc region comprising SEQ ID NO: 266 or 267, a heavy chain variable domain and a light chain variable domain, wherein the heavy chain variable domain comprises an amino acid sequence that is at least 80% identical or similar to SEQ ID NO: 234 and the light chain variable domain comprises an amino acid sequence that is at least 80% identical or similar to SEQ ID NO: 235, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 153, and
   a CDR-H3 amino acid sequence comprising or consisting of SEQ ID NO:156 (3B2g2m1) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:195 (3B2g2m1).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In an embodiment, the composition comprises an antibody or antigen-binding fragment that binds to human muscle-specific tyrosine-protein kinase (MuSK) that comprises:
a) A full length heavy chain comprising an amino acid sequence that is at least 80% identical or similar to SEQ ID NO: 268 and
b) A full length light chain comprising an amino acid sequence that is at least 80% identical or similar to SEQ ID NO: 269, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the composition comprises an antibody or antigen-binding fragment that binds to human muscle-specific tyrosine-protein kinase (MuSK) that comprises:
a) a full length heavy chain comprising SEQ ID NO: 268 and
b) a full length light chain comprising SEQ ID NO: 269, and
c) wherein the full length heavy chain comprises L234A and L235A mutations numbered according the EU numbering system.

In an embodiment, the anti-MuSK antibody or antigen binding fragment thereof, comprises:
a) a full length heavy chain comprising SEQ ID NO: 270 and
b) a full length light chain comprising SEQ ID NO: 271, and
c) wherein the full length heavy chain comprises L234A and L235A mutations numbered according the EU numbering system.

In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

In an embodiment, the composition comprises an antibody or antigen-binding fragment that binds to human muscle-specific tyrosine-protein kinase (MuSK) that comprises:
a) A full length heavy chain comprising an amino acid sequence that is at least 80% identical or similar to SEQ ID NO: 270 and
b) A full length light chain comprising an amino acid sequence that is at least 80% identical or similar to SEQ ID NO: 271, and
c) Wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the composition comprises an antibody or antigen-binding fragment that binds to human muscle-specific tyrosine-protein kinase (MuSK) that comprises:
a) A full length heavy chain comprising SEQ ID NO: 270 and
b) A full length light chain comprising SEQ ID NO: 271, and
c) Wherein the full length heavy chain comprises L234A and L235A mutations numbered according the EU numbering system.

In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

In a preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK), that comprises a heavy chain variable domain and a light chain variable domain, wherein the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (3B2) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises a heavy chain constant domain and light chain a constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, where the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO: 156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a preferred embodiment, the composition comprises an antibody or antigen-binding fragment that binds to human muscle-specific tyrosine-protein kinase (MuSK) that comprises a heavy chain constant domain and a light chain constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, wherein L234A and/or L235A substitution(s) (numbered according to the EU numbering system) is (are) introduced into said Fc region, and where the heavy chain variable domain comprises an amino acid sequence that is at least 80% identical or similar to SEQ ID NO: 202 and the light chain variable domain comprises an amino acid sequence that is at least 80% identical or similar to SEQ ID NO: 203, and
where the heavy chain variable domain comprises:
   a CDR-H1 amino acid sequence comprising SEQ ID NO: 147 or having 1, 2, 3, 4 or 5 amino acid alterations relative to SEQ ID NO: 147,
   a CDR-H2 amino acid sequence comprising SEQ ID NO: 150 or having 1, 2, 3, 4 or 5 amino acid alterations relative to SEQ ID NO: 150, and
   a CDR-H3 amino acid sequence comprising SEQ ID NO: 156 or having 1, 2, 3, 4 or 5 amino acid alterations relative to SEQ ID NO:156 (3B2) and
where the light chain variable domain comprises:
   a CDR-L1 amino acid sequence comprising SEQ ID NO: 159 or having 1, 2, 3, 4 or 5 amino acid alterations relative to SEQ ID NO: 159,
   a CDR-L2 amino acid sequence comprising SEQ ID NO: 172 or having 1, 2, 3, 4 or 5 amino acid alterations relative to SEQ ID NO: 172, and
   a CDR-L3 amino acid sequence comprising SEQ ID NO: 183 or having 1, 2, 3, 4 or 5 amino acid alterations relative to SEQ ID NO:183 (3B2).

In an embodiment, the identity or similarity is at least 81%, 82%, 83%, 84%, 85%, 86%, 87%, 88%, 89%, 90%, 91%, 92%, 93%, 94%, 95%, 96%, 97%, 98%, 99%.

In a more preferred embodiment, the composition comprises an antibody or antigen-binding fragment that binds to human muscle-specific tyrosine-protein kinase (MuSK) that comprises a heavy chain constant domain and light chain a constant domain (or human lambda constant domain), a heavy chain variable domain and a light chain variable domain, where the heavy chain constant domain comprising at least 80% sequence identity to SEQ ID NO: 280 and the light chain constant domain comprising at least 80% sequence identity to SEQ ID NO: 281, wherein L234A and L235A substitutions (numbered according to the EU numbering system) are introduced into said Fc region, and
where the heavy chain variable domain comprising SEQ ID NO: 202 and the light chain variable domain comprising SEQ ID NO: 203, and
where the heavy chain variable domain comprises:
   a CDR-H1 amino acid sequence comprising or consisting of SEQ ID NO: 147,
   a CDR-H2 amino acid sequence comprising or consisting of SEQ ID NO: 150, and
   a CDR-H3 amino acid sequence comprising or consisting of SEQ ID NO:156 (3B2) and
where the light chain variable domain comprises:
   a CDR-L1 amino acid sequence comprising or consisting of SEQ ID NO: 159,
   a CDR-L2 amino acid sequence comprising or consisting of SEQ ID NO: 172, and
   a CDR-L3 amino acid sequence comprising or consisting of SEQ ID NO:183 (3B2).

In an embodiment, the composition comprises an antibody or antigen-binding fragment that binds to human muscle-specific tyrosine-protein kinase (MuSK) that comprises:
a) a full length heavy chain comprising an amino acid sequence that is at least 80% identical or similar to SEQ ID NO: 282 and
b) a full length light chain comprising an amino acid sequence that is at least 80% identical or similar to SEQ ID NO: 283, and
c) wherein one or more of the following mutations (all numbered according to the EU numbering system) have been introduced into the full length heavy chain: an N297A substitution; an N297Q substitution; an L234A substitution; an L234D substitution; an L234E substitution; an L234G substitution; an L234H substitution; an L234F substitution; an L234K substitution; an L234Q substitution; an L234R substitution; an L234S substitution; an L234T substitution; an L235A substitution; an L235D substitution; an L235E substitution; an L235F substitution; an L235G substitution; an L235V substitution; an L235H substitution; an L2351 substitution; an L235K substitution; an L235R substitution; an L235S substitution; L235T substitution; an L235Q substitution; an L237A substitution; an S239D substitution; an E233P substitution; an L234V substitution; a C236 deletion; a G236E substitution; a G236R substitution; a G236K substitution; a G237A substitution; a P238A substitution; an F243L substitution; a D265A substitution; an S267E substitution; an H268A substitution; an R292P substitution; a Y300L substitution; a K322A substitution; a K322Q substitution; an A327Q substitution; an L328F substitution; an L328R substitution; a P329A substitution; a P329G substitution; an A330L substitution; an A330S substitution; a P331S substitution; an 1332E substitution; a P396L substitution; or each of the combinations of mutations described earlier in the fourth embodiment of this application, preferably the mutations is L234A or L235A, more preferably the mutations are L234A and L235A.

In an embodiment, the composition comprises an antibody or antigen-binding fragment that binds to human muscle-specific tyrosine-protein kinase (MuSK) that comprises:
a) a full length heavy chain comprising SEQ ID NO: 282 and
b) a full length light chain comprising SEQ ID NO: 283, and
c) wherein the full length heavy chain comprises L234A and L235A mutations numbered according the EU numbering system.

In an embodiment, the binding to an effector ligand is reduced of at least 10%, 20%, 30%, 40%, 50%, 60%, 70%, 80%, 90%, or is no longer detectable compared to the binding to the same ligand by the antibody not having any amino acid substitutions into its human IgG constant Fc region.

The polynucleotide compositions can result in the generation of the antibody-based molecule in the human subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the antibody-based molecule in the subject within at least about 1 day, 2 days, 3 days, 4 days, 5 days, 6 days, 7 days, 8 days, 9 days, or 10 days of administration of the composition to the subject. The composition can result in generation of the antibody-based molecule in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The polynucleotide composition, when administered to the subject in need thereof, can result in the persistent generation of the antibody-based molecule in the subject. The composition can result in the generation of the antibody-based molecule in the subject for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days.

The MuSK antibody-based molecules of the present invention can be administered by subcutaneous, parenteral, topical, or intranasal means for therapeutic treatment. Intramuscular injection (for example, into the arm or leg muscles), subcutaneous injection and intravenous infusion are preferred methods of administration of the molecules of the present invention. In some methods, such molecules are administered as a sustained release composition or device, such as a Medipad™ device (Elan Pharm. Technologies, Dublin, Ireland). In some methods, the antibodies disclosed herein are injected directly into a particular tissue, for example intracranial injection.

In one embodiment, a pharmaceutical composition of the present invention is administered parenterally. The phrases “parenteral administration” and “administered parenterally” as used herein denote modes of administration other than enteral and topical administration, usually by injection, and include epidermal, intravenous, intramuscular, intraarterial, intrathecal, intracapsular, intracranial, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection, subcutaneous and infusion. In one embodiment that pharmaceutical composition is administered by intravenous or subcutaneous injection or infusion.

### Agrin Deficiency

Five key genes act to form and maintain neuromuscular synapses. Neuronal isoforms of Agrin bind Lrp4 in muscle, leading to association between Lrp4 and MuSK (via Ig1 domain of MuSK) and stimulation of MuSK kinase activity. Once activated and phosphorylated, MuSK recruits Dok7, which stabilizes MuSK phosphorylation. MuSK activation ultimately leads to the anchoring of AChRs and other postsynaptic proteins at neuromuscular synapses, in a manner that is poorly understood but depends upon Rapsyn, an intracellular peripheral membrane protein that is bound to AChRs. The absence of only one of these genes cause neonatal lethality in mice due to a failure to form neuromuscular synapses. Mutations that reduce the activity of these genes in human, cause congenital myasthenic syndromes (CMS), a heterogeneous group of rare diseases, that lead to diminished anchoring of AChRs and structural abnormalities at neuromuscular synapses, and compromise synaptic transmission and causing muscle weakness and fatigue. These findings, as well as experimental evidence in mice, demonstrate that this pathway is essential not only for forming neuromuscular synapses during development but also for maintaining synapses in adults.

Agrin has a key role in synapse formation. The Agrin gene is expressed widely, but alternative splicing produces isoforms, encoded by exons 32 and 33 and including 8, 11 or 19 amino acids in the carboxy terminal region of Agrin, that are expressed only in neurons. Neuronal Agrin is a ~200kd heparin sulphate proteoglycan that is transported in motor axons and released from nerve terminals, where it associates with the synaptic basal lamina. Neuronal Agrin acts to organize postsynaptic differentiation by anchoring postsynaptic proteins, including AChRs, in the muscle membrane, and by enhancing transcription of genes encoding postsynaptic proteins in subsynaptic nuclei. The binding of agrin and LRP4 is a key step in MuSK activation and initiation of downstream signaling. Ligand-induced activation of MuSK creates a positive-feedback loop that induces further MuSK clustering. This finding is consistent with the observation that overexpression of ectopic MuSK induces agrin-independent autophosphorylation and non-synaptic AChR clustering.

Neuromuscular synapses initially form during development in the absence of Agrin. These nascent synapses, however, are labile and susceptible to an acetylcholine (ACh)-mediated pathway that disassembles synapses that are not stabilized by neuronal Agrin. As such, in the absence of neuronal Agrin, ACh extinguishes the nascent synapses, causing lethal respiratory failure at birth. Extracellular interactions with LRP4 are mediated by MuSK Ig domains. Residues Asp38 (blue) and IIe96 (white), on the exposed surfaces of the Ig1 domains, are respectively involved in congenital myasthenia and LRP4 binding. LRP and MUSK are expressed in well differentiated myotubes, but not myoblasts.

“Agrin deficiency” as used herein may mean a lower expression of agrin or agrin with a reduced activity. Such activity includes the activity of agrin to bind to LRP4, to induce LRP4-binding to MuSK, and/or to activate MuSK dimerization and/or MuSK phosphorylation.

A disease or condition resulting from agrin deficiency may be caused by a genetic defect in the AGRIN gene, deficiency in regulators of AGRIN expression and/or AGRIN activity, abnormal AGRIN metabolism, and/or disruption/intervention of AGRIN binding to its receptors. Such agrin deficiency may lead to reduced expression and/or function of agrin (protein) at NMJs.

A neuromuscular disorder may be caused by a genetic defect. A disease or condition resulting from a genetic defect is caused in whole or in part by a change in the DNA sequence away from the normal sequence. Genetic defect can be caused by a mutation in one gene (monogenic disorder), by mutations in multiple genes (multifactorial inheritance disorder), by a combination of gene mutations and environmental factors, or by damage to chromosomes (changes in the number or structure of entire chromosomes, the structures that carry genes). Types of genetic mutation include base substitutions, deletions and insertions.

In an embodiment, the genetic defect is caused by a mutation in the AGRIN gene. In an embodiment, the genetic defect in AGRIN results in no agrin protein expression, In an embodiment, the genetic defect in AGRIN results in reduced agrin expression.

In a first embodiment, the genetic defect is caused by a mutation in AGRIN, and the deficient agrin can no longer bind its receptor LRP4 In an embodiment, the MuSK antibody-based molecule for use in preventing, delaying, reversing or treating a disease or condition resulting from agrin deficiency: (i) binds to an epitope of human MuSK, preferably said epitope being preferably present in the MuSK Frizzled (Fz)-like domain sequence of SEQ ID NO: 130, and (ii) induces MuSK phosphorylation upon binding to its epitope. In such embodiment, the MuSK antibody-based molecule for use in preventing, delaying, reversing or treating a disease or condition resulting from agrin deficiency: (i) binds to an epitope of human MuSK, preferably said epitope being preferably present in the MuSK Frizzled (Fz)-like domain sequence of SEQ ID NO: 130, (ii) induces MuSK phosphorylation upon binding to its epitope and (iii) wherein the binding simulates the binding of the natural ligand of MuSK, being agrin or LRP/agrin.

In a second embodiment, the genetic defect is caused by a mutation in AGRIN, and the deficient agrin is still able to bind its natural receptor, LRP4, but with a deteriorated/decreased affinity and/or with a lower capacity to activate MuSK phosphorylation than its counterpart wild type agrin. In this embodiment, the MuSK antibody-based molecule for use in preventing, delaying, reversing or treating a disease or condition resulting from agrin deficiency may elicit a higher capacity to activate MuSK phosphorylation than deficient agrin.

In this context, affinity may be assessed using techniques known to the skilled person as earlier defined herein. Techniques for assessing the phosphorylation of MuSK are also known to the skilled person and have also been defined earlier herein.

In this embodiment, the deteriorated/decreased affinity of the deficient agrin to LRP4 may be at least 10%, 20%, 30%, 40%, 50%, 60%, 70%, 80%, 90% lower than the corresponding affinity of wild type agrin to LRP4.

In this embodiment, the capacity to activate MuSK phosphorylation by the deficient agrin is at least 10%, 20%, 30%, 40%, 50%, 60%, 70%, 80%, 90% less than the corresponding capacity of wild type agrin.

In this embodiment, the higher capacity to activate MuSK phosphorylation of the MuSK antibody-based molecule is at least 10%, 20%, 30%, 40%, 50%, 60%, 70%, 80%, 90%, 100%, 200%, 500%, 1000% higher than the corresponding capacity of deficient agrin.

In a third embodiment, the disease or condition resulting from agrin deficiency is such that the expression level of MuSK is lower than the expression level of MuSK in a subject who is not agrin deficient. In this embodiment, the MuSK antibody-based molecule for use in preventing, delaying, reversing or treating a disease or condition resulting from agrin deficiency may elicit a higher capacity to activate MuSK phosphorylation than deficient agrin.

Such properties allow to compensate for the lower expression level of MuSK.

Each of these features have been defined earlier herein.

In this embodiment, the lower expression of MuSK may at least 10%, 20%, 30%, 40%, 50%, 60%, 70%, 80%, 90% less than the corresponding expression of MuSK in a subject expressing wild type agrin.

In a fourth embodiment, the genetic defect is caused by a mutation in AGRIN, and the disease or condition resulting from agrin deficiency is such that the binding of LRP4 to MuSK is impaired/antagonized, which in its turn leads to an impaired activation of MuSK. A binding of LRP4 to MuSK may be considered to be impaired/antagonized when the binding affinity of LRP4 to MuSK is lower than the binding affinity of LRP4 to MuSK in subjects who are not deficient for agrin. In this embodiment, the MuSK antibody-based molecule for use in preventing, delaying, reversing or treating a disease or condition resulting from agrin deficiency may elicit a higher capacity to activate MuSK phosphorylation than deficient agrin.

Such properties allow to compensate for the impaired/antagonized binding of LRP4 to MuSK and/or for the impaired activation of MuSK.

Each of these features have been defined earlier herein.

In this embodiment, a lower binding affinity means the binding affinity is at least 10%, 20%, 30%, 40%, 50%, 60%, 70%, 80%, 90% lower than the corresponding binding affinity in a subject expressing wild type agrin. In this embodiment, the impaired activation of MuSK may be at least 10%, 20%, 30%, 40%, 50%, 60%, 70%, 80%, 90% lower than the corresponding activation of MuSK in a subject expressing wild type agrin.

Each of the first, third and/or fourth embodiments defined above may be combined. Each of the second, third and/or fourth embodiments defined above may be combined.

In an embodiment, the genetic defect is an AGRIN mutation. In some embodiments, the genetic defect in agrin is caused by bi-allelic mutations in the AGRN gene that reduce expression or function of AGRN at the NMJ. Such AGRN defects affect both formation and maintenance of the NMJ.

In an embodiment, the genetic defect in AGRIN is the sole genetic defect present in the subject. In other words, this subject does not have another genetic defect in a gene involved in the formation and maintenance of neuromuscular synapses.

In an embodiment, the AGRIN mutation is one of the mutations selected from deletion in the N-terminal laminin-binding domain, c.4744G>A-at the last nucleotide of exon 26 causing skipping of exon 26, p.S1180L, p.R1509W, p.G1675S, and p.Y1877D, c.5125G>C, Q353X and V1727F, p.L1176P and p.R1698C, V1727F, c.5023G>A, p.Glyl675Ser, C.5302G>C, p.R1671Q, p.R1698P, p.L1664P mutations, and amino acid deletion or deletions in AGRIN. In an embodiment, the agrin deficiency is caused by inflammation, infection, radiation or chemical toxicity. In an embodiment, an anti-MuSK antibody or the antigen-binding fragment thereof can be used to prevent, delay, reverse or treat a disease or condition resulting from AGRIN deficiency or mutation. In this context, the genetic defect or agrin deficiency is detected by genetic screening or diagnostic tests known to the skilled person in the art.

In an embodiment, the AGRIN deficiency is caused by deficiency in the regulators of AGRIN expression and/or AGRIN activity. In an embodiment, the agrin deficiency is caused by abnormal agrin metabolism. In an embodiment, the agrin deficiency is caused by disruption and/or intervention of AGRIN binding to its receptors. In an embodiment, the agrin deficiency is caused by poly-GA. Poly-GA is the most abundant form of DPR in C9-ALS, which sequesters soluble Agrin ligands and inhibits Agrin-MuSK signaling. In a preferred embodiment, the subject has ALS. In another embodiment, the agrin deficiency is caused by autoimmune antibodies that neutralize/binds to agrin, thereby interfere the binding of agrin to its receptors.

The antibody-based molecule of the present invention specifically acts on MUSK, thereby restoring disrupted acetylcholine signalling pathway or other signalling pathways important for neuromuscular junction formation, and the disrupted signalling pathway is linked to or due to blocked, deactivated, attenuated or disrupted MuSK signalling pathway in a cell, preferably a muscle cell. A known target for antibody-based molecule are molecules presenting an antigen or epitope for binding of an antibody-based molecule, which are known to be a target for treatment. In some embodiments, the antibody-based molecule is an antibody. In an embodiment, the antibody-based molecule is anti-MuSK agonists or antigen-binding fragment thereof, and the known target in the human subject is MuSK.

In an embodiment, the invention provides an antibody-based molecule for use in preventing, delaying, reversing or treating a disease or condition resulting from agrin deficiency in a subject, wherein the subject is characterized by abnormal development, abnormal growth, displaying poor hindlimb motor control and atrophy, abnormal/decreased fully innervated NMJ in the subject, abnormal motor performance and/or grip strength, abnormal contractile properties of a muscle at the NMJ, reduced/impaired resistance to fatigue of a muscle (at the NMJ), decreased muscle weight at the NMJ, abnormal muscle mass, abnormal muscle size, abnormal or impaired nerve terminal area and complexity, abnormal acetylcholine perimeter, abnormal endplate area, abnormal myofiber size, abnormal NMJ morphology, abnormal muscle strength, abnormal body/muscle weight, decreased survival rate and/or early postnatal death of the subject, absent the administration of the antibody-based molecule. The antibody-based molecule of this invention may reverse, delay or prevent the developmental abnormalities of the treated subject. Growth may be measured by muscle fiber size and/or muscle mass of the subject.

Hindlimb motor control and atrophy may be measured by techniques known to the skilled person in the art. The subject may be considered to have development abnormalities or abnormal growth of the subject, when at least one of such measurements deviates at least 10%, 20%, 30%, 40%, 50%, 60%, 70%, 80%, 90% from a normal/healthy/control subject absence of agrin deficiency at the same age. The development or growth or muscle strength of a subject may be assessed using assays known to the skilled person. In this context, the subject may be a human or an animal.

The subject may be considered to have early postnatal death, when the newborn die within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 days or within 1, 2, 3, 4, 5, 6, 7, 8, 9, 10, 11, 12 months, or within 1, 2, 3, 4, 5, 6, 7, 8, 9, 10, 11, 12, 13, 14, 15 years after birth.

In an embodiment, the subject is further characterized by abnormal neuromuscular junction formation, abnormal synapse development, and/or deficient motor function. The abnormal neuromuscular junction formation, abnormal synapse development, and/or deficient motor function may be assessed using assays (for example immunostaining, imaging techniques etc.) known to the skilled person, for example by assessing the number of synapses, the synaptic size or area, AChR density at the synapse, colocalization of synapse and AChR, the amount of denervated synapses, and/or fragmented synapses. The subject may be considered to have abnormal neuromuscular junction formation, abnormal synapse development, and/or deficient motor function, when at least one of such synapse measurements deviates at least 30%, 40%, 50%, 60%, 70%, 80%, 90% from a normal/control/healthy subject of the same age.

In an embodiment, the invention provides an antibody-based molecule for use in preventing, delaying, reversing or treating a disease or condition resulting from agrin deficiency in a subject wherein the disease or condition is a neuromuscular disease or condition. In an embodiment, a neuromuscular disease or condition is characterized by an impaired neuromuscular transmission and/or an NMJ denervation. In an embodiment, an impaired neuromuscular transmission or impaired NMJ denervation may be characterized by a deficient or absent agrin-MuSK binding, a deficient MuSK signaling, a deficient MuSK phosphorylation, and/or deficient acetylcholine receptor clustering.

In an embodiment, the subject is treated for an impaired neuromuscular transmission and/or impaired NMJ. In a preferred embodiment, the subject is treated for an impaired neuromuscular transmission by treatment with the MuSK antibodies or antigen binding fragment thereof as defined above. The activation of MuSK is expected to transduce signal at the NMJ of the subject and therefore to restore at least to some extent an impaired neuromuscular transmission. The MuSK agonist antibody and related aspects of the invention (i.e. polynucleotide, expression vector as earlier defined herein) are said to restore at least to some extent an impaired neuromuscular transmission when they are able to elicit an agonistic MuSK activity. In the context of the application, an agonistic MuSK activity may be replaced by the triggering of a MuSK-induced signal in a muscle cell at the NMJ. A MuSK-induced signal may be at least one of the induction of MuSK dimerization, the induction of MuSK tyrosine phosphorylation, the induction of AChR clustering at the NMJ (or the clustering is assessed in vitro in myotubes AChR patches), the increase of the number of fully innervated NMJ, the decrease of the number of fully denervated NMJ, an improvement of the reliability of synapse release, a restoration of synapse in the presence of axons, a prevention of altered synapse formation, a stabilization of formed synapses, a prevention/stabilization or even a reduction/decrease of motor neuron death, prolonged postnatal survival, or extension of the lifespan of a treated subject, an increase in muscle mass or size of the subject, an increase in muscle size of the subject, an increase of nerve terminal area and complexity, acetylcholine perimeter, and/or endplate area. In an embodiment, the impaired NMJ or the impaired formation of the NMJ of the treated subject is prevented, diminished, reversed, delayed or postponed. Within this embodiment, said MuSK agonist antibody or related aspects of the invention defined herein are able to restore at least to some extent an impaired neuromuscular transmission when they are able to elicit an agonistic MuSK activity. The restoration of at least to some extent the impaired neuromuscular transmission has been earlier defined herein.

In an embodiment, the invention provides an antibody-based molecule for use in preventing, delaying, reversing or treating a disease or condition resulting from agrin deficiency in a subject, wherein the subject is characterized by increase in type I, or type II myofibers and abnormal NMJ morphology, decreased fiber size (in muscle), decrease in nerve terminal area and complexity, decreased acetylcholine perimeter, decreased axon diameter, abnormal/decreased fully innervated NMJ in the subject, abnormal contractile properties of a muscle at the NMJ, reduced/impaired resistance to fatigue of a muscle (at the NMJ), decreased muscle weight at the NMJ, abnormal or impaired nerve terminal area and complexity, abnormal acetylcholine perimeter, abnormal endplate area, abnormal myofiber size, and/or abnormal NMJ morphology measured by assays known to the skilled person, for example by measuring nerve terminal area (μm2), nerve terminal perimeter (μm), number of terminal branches, number of branch points, total length branches, postsynaptic absent AChR staining (%), AChR area, AChR perimeter, endplate area, endplate perimeter, endplate diameter, derived Variables, presynaptic average length of branches complexity, postsynaptic, average area of AChR clusters, fragmentation, compactness (%), overlap (%), area of synaptic contact, associated nerve variables, axon diameter (μm), and number of axonal inputs. The subject may be considered to have development abnormalities or abnormal growth of the subject, when at least one of such measurements deviates at least 10%, 20%, 30%, 40%, 50%, 60%, 70%, 80%, 90% from a normal/control/healthy subject absence of agrin deficiency at the same age. The measurements may be assessed using techniques known to the skilled person. In this context, the subject may be a human or an animal.

In an embodiment, the MuSK antibody-based molecule, polynucleotide, expression vector, or composition for use according to the present invention, wherein the disease or condition is characterized by an impaired neuromuscular transmission and/or a denervation at the NMJ (neuromuscular junction). In an embodiment, the disease is a neuromuscular disease.

An impaired neuromuscular transmission may be characterized by at least one of:
a. motor neuron death,
b. NMJ denervation,
c. impaired synaptic transmission
d. abnormal development and abnormal growth of the subject
e. abnormal/decreased fully innervated NMJ,
f. abnormal motor performance and/or grip strength,
g. abnormal contractile properties of a muscle at the NMJ,
h. reduced/impaired resistance to fatigue of a muscle (at the NMJ),
i. decreased muscle weight at the NMJ,
j. abnormal muscle mass or size of the subject,
k. abnormal or impaired nerve terminal area and complexity,
l. abnormal acetylcholine perimeter, abnormal endplate area,
m. abnormal myofiber size,
n. abnormal NMJ morphology, and
o. abnormal muscle strength, and abnormal body/muscle weight of the subject.

In an embodiment, an impaired neuromuscular transmission or impaired synaptic transmission may be characterized by a deficient MuSK signaling, deficient MuSK dimerization, deficient MuSK phosphorylation, deficient MuSK signaling and/or deficient acetylcholine receptor clustering.

In an embodiment, an impaired neuromuscular transmission or impaired synaptic transmission may be characterized by a poor motor performance, a decreased grip strength, the poor contractile properties of a muscle at the NMJ, the poor resistance to fatigue of the muscle, a decreased muscle weight, abnormal development, abnormal growth of the subject, abnormal/decreased fully innervated NMJ, abnormal muscle mass of the subject, abnormal muscle size of the subject, abnormal or impaired nerve terminal area and complexity, abnormal acetylcholine perimeter, abnormal endplate area, abnormal myofiber size, abnormal NMJ morphology, abnormal muscle strength of the subject, abnormal body/muscle weight of the subject, abnormal survival of the subject, absent the administration of the antibody-based molecule.

In an embodiment, a neuromuscular disorder is analyzed or assessed or diagnosed via electrophysiological assessment; pharmacodynamic assessment; the level of neurofilaments (e.g. neurofilament light chain (NFL)) in blood serum, plasma and/or cerebrospinal fluid (CSF); or NMJ biopsies.

In an embodiment, there is provided an antibody-based molecule for use in preventing, delaying, reversing or treating a disease or condition resulting from agrin deficiency in a subject, wherein the disease is neuromuscular disease. In an embodiment, the neuromuscular disease may be selected from the group consisting of: amyotrophic lateral sclerosis (ALS), spinal muscular atrophy (SMA), myasthenia gravis (MG), congenital myasthenia, congenital myasthenic syndrome (CMS), Lambert-Eaton myasthenic syndrome (LEMS), Lyme disease, poliomyelitis, post-poliomyelitis, heavy metal intoxication, Kennedy syndrome, adult-onset Tay-Sachs disease, hereditary spastic paraplegia, multifocal neuropathy, cervical spondylosis, extramedullary tumor with compressive radiculopathy and myelopathy, inclusion body myositis, progressive bulbar palsy, progressive muscular atrophy, motor neuron syndrome and thyrotoxic myopathy. In an embodiment, the neuromuscular disease may be selected from the group consisting of: amyotrophic lateral sclerosis (ALS), spinal muscular atrophy (SMA), myasthenia gravis (MG), congenital myasthenia, congenital myasthenic syndrome (CMS), Lambert-Eaton myasthenic syndrome (LEMS), post-poliomyelitis, heavy metal intoxication, Kennedy syndrome, hereditary spastic paraplegia, multifocal neuropathy, and motor neuron syndrome. A preferred neuromuscular disease is CMS, more preferably the disease is presynaptic CMS or agrin induced CMS. Another preferred neuromuscular disease is ALS or SMA. In an embodiment, the subject is treated in combination with genetic treatment or genetic therapy for agrin deficiency. In an embodiment, the genetic therapy may be performed using antisense oligonucleotide, a gene replacement therapy, or small molecule to increase or restore the target gene which comprises a defect to cause the genetic disease.

In an embodiment, the neuromuscular disease is congenital myasthenia (CM). In an embodiment, the neuromuscular disease is congenital myasthenia (CM), and the CM is caused by AGRIN deficiency. In an embodiment, the neuromuscular disease is congenital myasthenia (CM) and the genetic defect is AGRIN mutation. In an embodiment, the genetic defect is caused by one of the genetic mutations selected from the deletion in the N-terminal laminin-binding domain, c.4744G>A-at the last nucleotide of exon 26 causing skipping of exon 26, p.S1180L, p.R1509W, p.G1675S, and p.Y1877D, c.5125G>C, Q353X and V1727F, p.L1176P and p.R1698C, V1727F, c.5023G>A, p.Glyl675Ser, C.5302G>C, p.R1671Q, p.R1698P, p.L1664P mutations.

In an embodiment, the neuromuscular disease is amyotrophic lateral sclerosis (ALS). In an embodiment, the neuromuscular disease is amyotrophic lateral sclerosis (ALS), and the ALS is caused by AGRIN deficiency. In an embodiment, the neuromuscular disease is amyotrophic lateral sclerosis (ALS) and the genetic defect is AGRIN mutation. In an embodiment, the neuromuscular disease is amyotrophic lateral sclerosis (ALS) and the AGRIN deficiency is caused by poly-GA.

In an embodiment, the neuromuscular disease is congenital myasthenic syndrome (CMS). Congenital myasthenic syndromes (CMS) are a heterogeneous group of early-onset genetic neuromuscular transmission disorders due to mutations in proteins involved in the organization, maintenance, function, or modification of the motor endplate (also called endplate myopathies). CMS are clinically characterized by abnormal fatigability, or transient or permanent weakness of extra-ocular, facial, bulbar, truncal, respiratory, or limb muscles. Onset of endplate myopathy is prenatal, congenital, in infancy, or childhood, and rarely in adolescence. Severity ranges from mild, phasic weakness, to disabling, permanent muscle weakness, respiratory insufficiency, and early death. All subtypes of CMS share the clinical features of fatigability and muscle weakness. There are 35 known causative genes for CMS, and one of them is Agrn, while some treatments exist for Agrin-CMS, there can come with side effects, therefore there is a need to develop more therapies. In an embodiment, the neuromuscular disease is congenital myasthenia syndrome (CMS), and the CMS is caused by AGRIN deficiency (Agrin CMS or Agrn CMS). In an embodiment, the neuromuscular disease is congenital myasthenia syndrome (CMS) and the genetic defect is AGRIN mutation. In an embodiment, the neuromuscular disease is presynaptic congenital myasthenia syndrome (CMS). In an embodiment, the neuromuscular disease is agrin induced CMS. In this context, “agrin induced CMS” is synonymous with “Agrin-CMS”, and can be used interchangeably. In an embodiment, the genetic defect is caused by one of the genetic mutations selected from the deletion in the N-terminal laminin-binding domain, c.4744G>A-at the last nucleotide of exon 26 causing skipping of exon 26, p.S1180L, p.R1509W, p.G1675S, and p.Y1877D, c.5125G>C, Q353X and V1727F, p.L1176P and p.R1698C, V1727F, c.5023G>A, p.Glyl675Ser, C.5302G>C, p.R1671Q, p.R1698P, p.L1664P mutations.

In an embodiment, the neuromuscular disease is Myasthenia Gravis (MG). In an embodiment, the neuromuscular disease is Myasthenia Gravis (MG), and the MG is caused by AGRIN deficiency. In an embodiment, the neuromuscular disease is Myasthenia Gravis (MG) and the deficiency in AGRIN is caused by auto-Abs against Agrin.

In an embodiment, the neuromuscular disease is spinal muscular atrophy (SMA). In preferred embodiment, the neuromuscular disease is spinal muscular atrophy (SMA) and the SMA is associated with AGRIN deficiency. In preferred embodiment, the neuromuscular disease is spinal muscular atrophy (SMA), and the genetic defect is in the SMN1 gene. In a preferred embodiment, the subject is treated with an antibody-based molecule against MuSK in combination with gene therapy, wherein the gene therapy is to correct the genetic defect in the AGRIN or SMN1 gene. In an even more preferred embodiment, an antisense oligonucleotide is used for the gene therapy. In an embodiment, the genetic defect is caused by one of the genetic mutations selected from the deletion in the N-terminal laminin-binding domain, c.4744G>A-at the last nucleotide of exon 26 causing skipping of exon 26, p.S1180L, p.R1509W, p.G1675S, and p.Y1877D, c.5125G>C, Q353X and V1727F, p.L1176P and p.R1698C, V1727F, c.5023G>A, p.Glyl675Ser, C.5302G>C, p.R1671Q, p.R1698P, p.L1664P mutations.

In one embodiment, the invention provides an antibody-based molecule of the present invention is administered parenterally. The phrases “parenteral administration” and “administered parenterally” as used herein denote modes of administration other than enteral and topical administration, usually by injection, and include epidermal, intravenous, intramuscular, intraarterial, intracardiac, intradermal, intraperitoneal, intratendinous, subcutaneous, subcuticular, and infusion. In one embodiment that antibody-based molecule is administered by intraperitoneal, intravenous, intramuscular or subcutaneous injection. In some methods, such molecules are administered as a sustained release composition or device, such as a Medipad™ device (Elan Pharm. Technologies, Dublin, Ireland).

In an embodiment, the antibody-based molecule is administered to the subject in an amount therapeutically effective for the treatment of said subject. The term “therapeutically effective amount” is intended to mean the quantity or dose of an antibody-based molecule that is sufficient to produce a therapeutic effect, for example, the quantity or dose of antibody-based molecule required to elicit an antigen binding activity. In an embodiment, such antibody-based molecule is able to eradicate or at least alleviate the symptoms associated with a disease or condition resulting from an agrin deficiency in a subject. An appropriate amount or dose can be determined by a physician, as appropriate.

Effective doses of the provided therapeutic molecules of the present invention for the treatment of the above-described conditions may vary depending upon many different factors, including means of administration, target site, physiological state of the subject, and/or other medications administered. Treatment dosages are typically titrated to optimize their safety and efficacy. On any given day that a dosage is given, the dosage of the antibody-based molecules (e.g. MuSK antibody or antigen binding fragment thereof) as described herein may range from about 0.0001 to about 100 mg/kg, and more usually from about 0.01 to about 20 mg/kg, of the subject's body weight. For example, dosages can be 0.5 mg/kg, 1 mg/kg, or 10 mg/kg of the subject's body weight or within the range of 0.5-10 mg/kg of the subject's body weight. Exemplary dosages thus include: from about 0.1 to about 10 mg/kg of the subject's body weight, from about 0.1 to about 5 mg/kg of the subject's body weight, from about 0.1 to about 2 mg/kg of the subject's body weight, from about 0.1 to about 1 mg/kg of the subject's body weight, for instance about 0.1 mg/kg of the subject's body weight, about 0.2 mg/kg of the subject's body weight, about 0.5 mg/kg of the subject's body weight, about 1 mg/kg of the subject's body weight, about 1.5 mg/kg of the subject's body weight, about 2 mg/kg of the subject's body weight, about 5 mg/kg of the subject's body weight, 10 mg/kg of the subject's body weight or about 20 mg/kg of the subject's body weight. In general, the dosing may be selected to obtain a serum titer of the therapeutic molecule in the subject that is known or expected to be effective in the treatment of a subject.

A physician or veterinarian having ordinary skill in the art may readily determine and prescribe the effective amount of the pharmaceutical composition required. For example, the physician or veterinarian could start doses of antibody-based molecule in the pharmaceutical composition at levels lower than that required in order to achieve the desired therapeutic effect and gradually increase the dosage until the desired effect is achieved. In general, a suitable daily dose of a composition of the present invention will be that amount of the compound which is the lowest dose effective to produce a therapeutic effect. Such an effective dose will generally depend upon the factors described above. Administration may e.g. be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible the antibody-based molecule of the present invention to be administered alone, it is preferable to administer the antibody-based molecule as a pharmaceutical composition as described.

For therapeutic purposes, the antibody-based molecules (e.g. MuSK antibody-based molecules) of the present invention are usually administered on multiple occasions. Intervals between single dosages (e.g., a bolus or infusion) can be daily, weekly, or monthly. In some methods, dosage is adjusted to achieve a plasma concentration of 1 ng/ml to 1000 μg/mL, preferably 1-1000 μg/mL, more preferably 25-300 μg/mL. Alternatively, the therapeutic molecules of the present invention can be administered as a sustained release formulation, in which case less frequent administration is required. Dosage and frequency vary depending on the half-life of the antibody in the subject to be treated. In general, human antibodies show the longest half-life, followed by humanized antibodies, chimeric antibodies, and non-human antibodies. scFv molecules generally have short serum half-lives.

In an embodiment, the antibody-based molecule for use in preventing, delaying, reversing or treating a disease or condition resulting from agrin deficiency in a subject is administered to the subject immediately or within 1, 2, 3, 4, 5, 6 or 7 days, or 1 or 2 weeks following a positive diagnosis of agrin deficiency, for example from a genetic test. “Immediately” in this context may mean within 1, 2, 3, 4, 5, 6, 7, 8, 9, 10, 11, 12, 13, 14, 15, 16, 17, 18, 19, 20, 21, 22, 23, 24 hours.

In an embodiment, the subject having agrin deficiency is treated for 1, 2, 3, 4, 5, 6, 7, 8, 9, 10, 11, 12, 13, 14, 15, 16, 17, 18, 19, 20, 30, 40, or 50 times. For therapeutic purpose, the antibody-based molecule is usually administered several times. Intervals between single dosage can be weekly, monthly or yearly. In some embodiments, the antibody-based molecule of the present invention is administered to the subject at least 1, at least 2, at least 3, at least 4, at least 5 times. Depending on the growth or development of the treated subject, the skilled person may decide to modify the frequency and/or dose of administration of the antibody-based molecule. In an embodiment, the subject is being treated only after birth (or postnatally).

In the context of the present invention, the subject has never been exposed or treated with said antibody-based molecules prior to birth (prenatal or in utero). In an embodiment, the antibody-based molecule is a MuSK agonist antibody.

In some embodiments, the doses administered to the subject are separated by 1, 2, 3, 4, 5, 6 or 7 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks. In some instances, the first dose is administered to the subject having disease or condition resulting from agrin deficiency at earliest at day 1. In some instances, the doses are administered to the subject having disease or condition resulting from agrin deficiency every 1, 2, 3, 4, 5, 6, 7, 8, 9, 10 weeks from the beginning of 1, 2, 3, 4 weeks or 1, 2, 3, 4, 5, 6, 7 8, 9, 10, 11, 12 months or 1, 2, 3, 4, 5 years after birth (e.g., for 1 month, 2 months, three months, four months, five months, six months, seven months, eight months, nine months, ten, eleven, twelve months).

In some embodiments, the antibody-based molecule for use reverses, delays and/or prevents the development of said disease or condition resulting from agrin deficiency. The delay may be of 1, 2, 3, 4, 5, 6 or 7 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 weeks or 1, 2, 3, 4, 5, 6, 7, 8, 9, 10, 11, 12, month or at least 1, 2, 3, 4 years. In this context, the development of said disease or condition resulting from agrin deficiency may be assessed by the function in respiratory, energy expenditure, activity, water intake, food intake, motor deficit, and/or body weight compared to a control or healthy or normal subject. Such functions may be assessed by measuring the corresponding metabolic parameters (for example O₂ consumption, CO₂ production, energy expenditure, total activity, water intake, food intake, latency to fall, and/or body weight measurements) known to a skilled person. In this context, the subject may be an animal.

In an embodiment, the subject is at the age of 1, 2, 3, or 4 weeks, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 years old when an antibody-based molecule or a maintenance dose is administered to said subject, for preventing, delaying, reversing or treating a disease or condition resulting from agrin deficiency.

In an embodiment, the invention provides an antibody-based is for use in preventing, delaying, reversing or treating or treating a disease or condition resulting from agrin deficiency in a subject, wherein said treatment results in the reversion, delay and/or prevention of abnormal synaptic development, abnormal synapse maturation, abnormal synapse generation, abnormal synapse regeneration and/or deficient motor function, wherein the treated subject is assessed by characterization of neuromuscular synapse, synapses counting, synapse size estimation, and/or acetylcholine receptor (AChR) density estimation at the synapse. In this context, synaptic size may mean the size of the acetylcholine receptor (AChR) rich area, assessed by AChR density estimation for example. Synapses generation or synapses regeneration or synapses maturation may be characterized from the change of its shape, from example from a plaque-like shape to a complex, pretzel-like shape, using imaging techniques known to a skilled person. The synaptic development, abnormal synapse maturation and/or deficient motor function may be considered to be reversed, delayed and/or prevented, if the value of such assessment has been improved at least 10%, 20%, 30%, 40%, 50% or 60% in an experiment administering the antibody-based molecule of the invention to the subject by comparison with the same experimental setting without administering a subject. In this context, the synaptic development, abnormal synapse maturation, abnormal synapse generation, abnormal synapse regeneration and/or deficient motor function may be considered to be reversed, delayed or prevented, if such assessment may represent at least 30%, 40%, 50%, 60%, 70% of the corresponding measurements in a normal/control/healthy subject (for example a healthy/normal/control subject at the same or similar age). In an embodiment, the number of synapses can be restored to at least 40%, 45%, 50%, 55%, 60%, 65% or 70% of normal levels in a healthy/normal/control subject. In an embodiment, the synaptic size can be restored to at least 40%, 45%, 50%, 55%, 60%, 65% or 70% of normal levels in a healthy/control/normal subject. In an embodiment, the density of synaptic AChRs can be restored to at least 40%, 45%, 50%, 55%, 60%, 65% or 70% of normal levels in a healthy/normal/control subject.

In an embodiment, the invention provides an antibody-based molecule for use in preventing, delaying, reversing or treating a disease or condition resulting from agrin deficiency in a subject, wherein said treatment results in a moderate function in respiratory, energy expenditure, activity, water intake, food intake, and/or body weight compared to a control or healthy or normal subject. Such functions may be assessed by measuring the corresponding metabolic parameters (for example O₂ consumption, CO₂ production, energy expenditure, total activity, water intake, food intake, and/or body weight measurements) known to a skilled person. In this context, the subject may be an animal. In an embodiment, the treated subject maintains a function in respiratory, energy expenditure, activity, water intake, food intake, and/or body weight at a level of at least 10%, 15%, 20%, 25%, 30%, 35%, 40%, 45%, 50%, 55%, 60%, 65%, 70%, 75%, 80% of the level of a control. Such levels may be maintained or gradually decrease until the disease or condition relapses. The decrease of the levels of any of said functions or body weight may be rescued by a maintenance dose or maintenance dosages.

The term “treatment” or “treating” as used herein means ameliorating, slowing or reversing the progress or severity of a disease or disorder, or ameliorating, slowing or reversing one or more symptoms or side effects of such disease or disorder. The term “delaying” as used herein means slowing the progress or severity of a disease or disorder, or ameliorating, slowing one or more symptoms or side effects of such disease or disorder. The term “reversing” as used herein means reversing the progress or severity of a disease or disorder, or ameliorating or reversing one or more symptoms or side effects of such disease or disorder.

For purposes of this invention, “treatment”, “treating”, “delaying” or “reversing” further means an approach for obtaining beneficial or desired clinical results, where “beneficial or desired clinical results” include, without limitation, alleviation of a symptom, diminishment of the extent of a disorder or disease, stabilized (i.e., not worsening) disease or disorder state, delay, prevention or slowing of the progression a disease or disorder state, amelioration or palliation of a disease or disorder state, and remission of a disease or disorder, whether partial or total, detectable or undetectable.

An antibody-based molecule may correct the abnormal weight gain of the treated subject. The postnatal body weight gain after treatment is at least 50%, 60%, 70%, 80%, 90% or 100% identical compared to the body weight gain of a normal new-born of the same age. The weight gain of a treated subject may be considered to have been improved when such weight gain is improved by at least 10%, 20%, 30%, 40%, 50%, 60%, 70%, 80%, 90%, or 100% in an experiment administering the antibody-based molecule of the invention to the subject by comparison with the same experimental setting without administering a subject. The correction may be seen as a reversal of at least part of the weight loss induced by the disease. In an embodiment, the reversal of at least part of the body weight loss is a reversal of at least 10%, 20%, 30%, 30%, 40%, 50%, 60%, 70%, 80%, 90% or 100% of the body weight loss. The weight gain of a treated subject may be assessed using assays known to the skilled person. In this context, the subject may be an animal.

An antibody-based molecule may induce a prolonged postnatal survival of the treated subject. The postnatal survival rate of treated subject may have been considered to have been increased when the lifespan of the treated subject extends at least 1, 2, 3, 4, 5, 6, 7, 8, 9, 10, 11, 12 weeks or at least 1, 2, 3, 4, 5, 6, 7, 8, 9, 10, 11 or 12 months or at least 1, 2, 3 or more years compared to the expected lifespan of a subject having agrin deficiency and who has not been administered with an antibody-based molecule of the invention. In this context, the subject may be an animal.

An antibody-based molecule may induce an enhanced muscle strength of the treated subject. The muscle strength of treated subject may have been considered to have been increased when the muscle strength of the treated subject is improved by at least 50%, 60%, 70%, 80%, 90%, or 100% in an experiment administering the antibody-based molecule of the invention to the subject by comparison with the same experimental setting without administering a subject. Muscle strength may be measured by tube test, hanging wire test, and/or grip strength meter.

An antibody-based molecule may induce an increased or steady growth of muscle mass and/or weight gain in the treated subject. The muscle mass and/or weight gain may have been considered to have been improved when the measurements are improved by at least 50%, 60%, 70%, 80%, 90%, or 100% in an experiment administering the antibody-based molecule of the invention to the subject by comparison with the same experimental setting without administering a subject.

An antibody-based molecule may induce an increased muscle size in the treated subject. The muscle size may have been considered to have been improved when the measurements are improved by at least 50%, 60%, 70%, 80%, 90%, or 100% in an experiment administering the antibody-based molecule of the invention to the subject by comparison with the same experimental setting without administering a subject.

An antibody-based molecule may induce an improvement in myofiber size and NMJ morphology in the treated subject. The myofiber size and NMJ morphology may have been considered to have been improved when the measurements are improved by at least 50%, 60%, 70%, 80%, 90%, or 100% in an experiment administering the antibody-based molecule of the invention to the subject by comparison with the same experimental setting without administering a subject. The methods of measuring myofiber size and NMJ morphology are exemplified in the examples.

Each of the therapeutic effects characterized herein could be seen as a stabilization of the disorder. In an embodiment, the use of an antibody-based exhibits a therapeutic effect on the treated subject defined herein. In a preferred embodiment, the use of an antibody-based molecule exhibits a therapeutic effect on the subject having a disease or condition resulting from an agrin deficiency, wherein the disease or condition is a neuromuscular disease.

The use of a compound results in activating, inducing a mechanism that promotes NMJ (neuromuscular junction) stability and/or repair is attractive for the prevention and treatment of any disease or symptoms, especially wherein such NMJ is affected.

Accordingly, in an embodiment, the use of an anti-MuSK antibody or antigen binding fragment according to the invention or a composition according to the invention in preventing, delaying, reversing or treating a disease or condition resulting from agrin deficiency in a human subject result in a stabilization of said disorder. The stabilization may be for at least 1, 2, 3, 4, 5, 6, 7, 8, 9, 10, 11 or 12 months or at least 1, 2 or 3 years. Each of the therapeutic effects further characterized herein could be seen as a stabilization of the disorder.

In an embodiment, the use of an anti-MuSK antibody or antigen-binding fragment (or polynucleotice, expression vector, composition) exhibits a therapeutic effect on the treated human subject defined herein.

By binding to an epitope of MuSK, the anti-MuSK antibody or antigen binding fragment of the invention are able to elicit an agonistic MuSK activity. Within the context of the application “elicit an agonistic MuSK activity” may be replaced by “activate MuSK”. An agonistic MuSK activity or an activation of MuSK may be triggered at the molecular and/or at the cellular level and/or in a more biological complex system as a NMJ, a synapse, a living organism. In the context of the application, an agonistic MuSK activity may be replaced by the triggering of a MuSK-induced signal or by the induction of MuSK activation in a muscle cell at the NMJ. A MuSK-induced signal (or MuSK activation or MuSK activity) may be at least one of the induction of MuSK dimerization, the induction of MuSK tyrosine phosphorylation, the induction or increase of induction of AChRs clustering at the NMJ (or clustering in vitro in myotubes AChR patches), the increase of the number or percentage of fully innervated NMJ, the decrease of the number or percentage of fully denervated NMJ, maintenance of the number or percentage of fully innervated NMJ (disease stabilization/disease progression stabilization), an improvement of the reliability of synaptic transmission, an improvement of motor performance, a prevention/stabilization or even a reduction/decrease of motor neuron death, an extension of the lifespan of a treated subject.

A MuSK-induced signal by the anti-MuSK antibody of the invention may be the induction of MuSK dimerization, which may be assessed by western blotting. In the context of the invention, an agonistic activity of MuSK may have been assessed when the induction of MuSK dimerization is increased of at least 10%, 20%, 30%, 40%, 50%, 60%, 70%, 80%, 90%, or 100% or more in an experiment using the antibody of the invention by comparison with the same experimental setting without any antibody or with a negative control or with a negative control antibody. Alternatively, in the context of the invention, an agonistic activity of MuSK antibody may have been assessed when the induction of MuSK dimerization is the same or about the same (20% less, 10% less or the same or 10% more or 20% more) in an experiment using the antibody of the invention by comparison with the same experimental setting with a positive control. A positive control in the assessment of MuSK dimerization may be wt agrin.

A MuSK-induced signal by the anti-MuSK antibody of the invention may be the induction of MuSK tyrosine phosphorylation and such phosphorylation may be assessed by western blotting using an antibody specific for tyrosine phosphorylation. In the context of the invention, an agonistic activity of MuSK may have been assessed when the induction of MuSK tyrosine phosphorylation is increased of at least 10%, 20%, 30%, 40%, 50%, 60%, 70%, 80%, 90%, 100%, 120%, 150%, 180%, 200% or more in an experiment using the antibody of the invention by comparison with the same experimental setting without any antibody. Alternatively, in the context of the invention, an agonistic activity of MuSK may have been assessed when the induction of MuSK tyrosine phosphorylation is the same or about the same (20% less, 10% less or the same or 10% more or 20% more) in an experiment using the antibody of the invention by comparison with the same experimental setting with a positive control. Such a MuSK tyrosine phosphorylation may be assessed without agrin. A positive control in the assessment of MuSK tyrosine phosphorylation may be wt agrin.

A MuSK-induced signal by the anti-MuSK antibody of the invention may be the induction of acetylcholine receptor (AChR) clustering at the NMJ and such clustering may be assessed by staining of AChR using an antibody specifically binding to AChR and visualising such staining in fluorescent microscopy using techniques known to the skilled person. Alternatively, the clustering may be assessed in vitro in myotubes AChR patches. A preferred antibody used to visualise AChR clustering is an antibody specific for AChR. More preferred antibody is AlexaFluor488 conjugated a-bungarotoxin (B13422, ThermoFisher). Usually the region to be analyzed is fixed in paraformaldehyde and incubated at room temperature with the relevant antibody of the invention or with a positive or negative control and subsequently each region is washed with PBS and observed under an epi-fluorescent microscopy. In the context of the invention, an agonistic activity of MuSK may have been assessed when the induction of AChR clustering at the NMJ is the same or about the same (i.e. 20% less, 10% less or the same or 10% more or 20% more) in an experiment using the antibody of the invention by comparison with the same experimental setting with a positive control, or is increased of at least 10%, 20%, 30%, 40%, 50%, 60%, 70%, 80%, 90%, or 100% in an experiment using the antibody of the invention by comparison with the same experimental setting without any antibody. Such a AchR clustering may be assessed without agrin. A positive control in the assessment of AchR clustering may be wt agrin.

In a preferred embodiment, the anti-MuSK antibody of the invention exhibits an induction or increase of induction of acetylcholine receptor clustering at the NMJ and such clustering may be assessed by visualizing a staining or an increased staining for AchRs at the NMJ of diaphragms of mice compared to the staining obtained without MuSK agonist antibody. In an embodiment, this induction or increase of clustering of AchRs at the NMJ results in a more normal/physiological NMJ morphology maintaining synaptic innervation and/or pre- and post-synaptic alignment. For example, a normal/physiological NMJ can be characterized by plaque-like shape.

A MuSK-induced signal by the anti-MuSK antibody of the invention in a muscle cell at the NMJ may be the increase of the number or percentage of fully innervated NMJ, the decrease of the number or percentage of fully denervated NMJ, maintenance of the number or percentage of fully innervated NMJ (disease stabilization/disease progression stabilization), an improvement of the reliability of synaptic transmission, a prevention/stabilization or even a reduction/decrease of motor neuron death. Each of these features could be assessed using techniques known to the skilled person such as staining of AchR using the a-bungarotoxin antibody as earlier defined herein, presynaptic labelling and quantifying innervation by fluorescent confocal microscopy, EMG single fibre EMG, electrophysiology of single synapses, staining of motor neuron cell bodies in bone marrow specific regions. All these assays have been described in Cantor S et al 2018 (Elife, 2018; 7: e34375).

An anti-MuSK antibody or antigen-binding fragment may improve nerve terminal area and complexity of the treated subject. The nerve terminal area and complexity may have been considered to have been improved when such nerve terminal area and complexity may have been increased of at least 10%, 20%, 30%, 40%, 50%, 60%, 70%, 80%, 90%, or 100% in an experiment using the anti-MuSK antibody of the invention by comparison with the same experimental setting without any antibody. Each of these features could be assessed using techniques known to the skilled person such as staining with H&E (Haematoxylin and Eosin), MHC (myosin heavy chain), confocal microscope imaging, Mira Vision analysis, immunofluorescent labeling and FIJI analysis.

An anti-MuSK antibody or antigen-binding fragment may improve acetylcholine perimeter of the treated subject. The acetylcholine perimeter may have been considered to have been improved when such acetylcholine perimeter may have been increased of at least 10%, 20%, 30%, 40%, 50%, 60%, 70%, 80%, 90%, or 100% in an experiment using the anti-MuSK antibody of the invention by comparison with the same experimental setting without any antibody. Each of these features could be assessed using techniques known to the skilled person such as staining with H&E (Haematoxylin and Eosin), MHC (myosin heavy chain), confocal microcrope imaging, Mira Vision analysis, immunofluorescent labeling and FIJI analysis.

An anti-MuSK antibody or antigen-binding fragment may improve endplate area, perimeter or diameter of the treated subject. The endplate area, perimeter or diameter may have been considered to have been improved when such endplate area, perimeter or diameter may have been increased of at least 10%, 20%, 30%, 40%, 50%, 60%, 70%, 80%, 90%, or 100% in an experiment using the anti-MuSK antibody of the invention by comparison with the same experimental setting without any antibody. Each of these features could be assessed using techniques known to the skilled person such as staining with H&E (Haematoxylin and Eosin), MHC (myosin heavy chain), confocal microcrope imaging, Mira Vision analysis, immunofluorescent labeling and FIJI analysis.

An anti-MuSK antibody or antigen-binding fragment may improve the motor performance and/or grip strength of the treated subject. The motor performance and grip strength of a treated subject may have been considered to have been improved when such motor performance or grip strength may have been increased of at least 10%, 20%, 30%, 40%, 50%, 60%, 70%, 80%, 90%, or 100% in an experiment using the anti-MuSK antibody of the invention by comparison with the same experimental setting without any antibody (i.e. control diseased subject). The motor performance (or grip strength) of a (treated) subject may be assessed using assays known to the skilled person. The experimental part discloses some exemplary methods.

An anti-MuSK antibody or antigen-binding fragment may improve the contractile properties of a muscle at the NMJ of the treated subject. The contractile properties of a muscle of a treated subject may have been considered to have been improved when such contractile properties may have been increased of at least 10%, 20%, 30%, 40%, 50%, 60%, 70%, 80%, 90%, or 100% in an experiment using the anti-MuSK antibody of the invention by comparison with the same experimental setting without any antibody (i.e. control diseased subject). The contractile properties of the muscle of a (treated) subject (at the NMJ) may be assessed using assays known to the skilled person. The experimental part discloses some exemplary methods. In this context, the subject may be an animal.

An anti-MuSK antibody or antigen-binding fragment may improve the resistance to fatigue of a muscle at the NMJ of the treated subject. The resistance to fatigue of a muscle of a treated subject may have been considered to have been improved when such fatigue properties may have been improved of at least 10%, 20%, 30%, 40%, 50%, 60%, 70%, 80%, 90%, or 100% in an experiment using the anti-MuSK antibody of the invention by comparison with the same experimental setting without any antibody (i.e. control diseased subject). The fatigue properties of the muscle of a (treated) subject (at the NMJ) may be assessed using assays known to the skilled person. The experimental part discloses some exemplary methods. In this context, the subject may be an animal.

An anti-MuSK antibody or antigen-binding fragment may induce an increase of the muscle weight at the NMJ of the treated subject. The muscle weight of a treated subject may have been considered to have been improved when such weight may have been increased of at least 10%, 20%, 30%, 40%, 50%, 60%, 70%, 80%, 90%, or 100% in an experiment using the anti-MuSK antibody of the invention by comparison with the same experimental setting without any antibody (i.e. control diseased subject). The experimental part discloses some exemplary methods. In this context, the subject may be an animal.

A MuSK-induced signal or effect by the anti-MuSK antibody of the invention may be characterized by the improvement of the quality of life or the delay in the apparition of the deterioration of the quality of life of a treated subject. The quality of life may be quantified by the weight of the subject. The improvement of the quality of life or the delay in the apparition of the deterioration of the quality of life may be of at least 1 day, 1 week, 2 weeks, 3 weeks, 4 weeks, 1 month, 2 months, 3 months, 4 months, 5 months, 6 months, 7 months, 8 months, 9 months, 10 months, 11 months, 1 year or more. This is assessed in comparison with the expected quality of life (or the expected apparition of the deterioration of the quality of life) of a subject suffering from the same condition and having not been treated with an antibody of the invention (i.e. control diseased subject). In this context, the subject may be an animal.

A MuSK-induced signal or effect by the anti-MuSK antibody of the invention may be characterized by the lifespan of a treated subject. The extension may be of at least 1 day, 1 week, 2 weeks, 3 weeks, 4 weeks, 1 month, 2 months, 3 months, 4 months, 5 months, 6 months, 7 months, 8 months, 9 months, 10 months, 11 months, 1 year or more. This is assessed in comparison with the expected lifespan of a subject suffering from the same condition and having not been treated with an antibody of the invention (i.e. control diseased subject). In this context, the subject may be an animal.

The properties of the anti-MuSK antibody described herein may be measured in accordance with the assays described herein. An activating activity of a MuSK agonist antibody may be measured relative to a control, for example a negative control antibody (such as an isotype control) that may not bind MuSK. A preferred control antibody not binding to MuSK is Motavizumab which targets RSV (Review, MAbs, 1 (5), 439-442, Sept-Octo 2009, DOI: 10.4161/mabs.1.5.9496). A preferred positive control agonist MuSK antibody is mAb #13 from Genentech. Another preferred positive control molecule for evidencing an activating MuSK activity is wt agrin (rat agrin from R&D systems, 550-AG).

NMJ repair may be the induction or increase of nerve sprouting and/or the increase of the innervation status of the NMJ. Each of these effects may be assessed using techniques known to the skilled person.

Accordingly, in an embodiment, the use of an anti-MuSK antibody or antigen-binding fragment (or polynucleotide, expression vector, composition), exhibit one or more of the following therapeutic effects:
an increase of the number or percentage of fully innervated NMJ in the subject, maintenance of the number or percentage of fully innervated NMJ in the subject, the decrease of the number or percentage of fully denervated NMJ in the subject, an improvement of the reliability of synaptic transmission, a prevention, stabilization or reduction of motor neuron death in the subject; and/or
an improvement of the motor performance and/or grip strength of the subject; and/or
an improvement of the contractile properties of a muscle at the NMJ of the subject; and/or
an improvement of the resistance to fatigue of a muscle at the NMJ of the subject; and/or
an induction of an increase of the muscle weight at the NMJ of the subject; and/or
an improvement of the quality of life or the delay in the apparition of the deterioration of the quality of life of the subject.

In an embodiment, the use of an anti-MuSK antibody or antigen-binding fragment (or polynucleotide, expression vector, composition) results in a minimum or acceptable level of at least one of the parameters listed hereafter: respiratory function, energy expenditure, activity, water intake, food intake, and/or body weight in the treated subject compared to the level of the same parameter in a control or healthy subject. Such parameters may be assessed by measuring the corresponding metabolic parameters (for example O₂ consumption, CO₂ production, energy expenditure, total activity, water intake, food intake, and/or body weight measurements) known to a skilled person. In this context, the subject may be an animal. In this context, “minimum or acceptable level” may mean a level of at least 50% of the level of the same parameter in a control subject. A control subject may be a normal/healthy subject or a subject at the onset of the disease. A “minimum or acceptable level” may be at least 55%, 60%, 65%, 70%, 75%, 80%, 85% or 90% of the level of a control subject.

In another embodiment, the use of an anti-MuSK antibody or antigen-binding fragment (or polynucleotide, expression vector, composition) results in an increase of the level of at least one of the parameters listed hereafter: respiratory function, energy expenditure, activity, water intake, food intake, and/or body weight in the treated subject compared to the level of the same parameter in the subject at the onset of the treatment. In this context, the subject may be an animal. In this context, “an increase of the level” may mean an increase of at least 5% of the level of the same parameter in the subject at the onset of the treatment. An “increase of the level” may be an increase of at least 10%, 15%, 20%, 25%, 30%, 35%, 40%, 45%, 50%, 55%, 60%, 65%, 70%, 75%, 80%, 85% or 90%.

In the context of the present invention, the onset of a disease resulting from agrin deficiency in a subject may be assessed, for example, using each of these parameters/features: body weight, O₂ consumption, CO₂ production, energy expenditure, synaptic size and AChR density, muscle strength, grip strength, and/or muscle performance. Each of these parameters/features may be assessed using techniques known to the skilled person. As an example, the disease onset may be defined when O₂ consumption is reduced by at least 5%, 10%, 15%, or 20%, CO₂ production and energy expenditure are reduced by at least 5%, 10%, 15%, 20%, 25%, or 30%, the synaptic size and synaptic AChR density are reduced by at least 20%, 30%, 40%, 50%, or 60%, and/or grip strength and performance on an accelerating rotarod are diminished by at least 10%, 20%, 30%, 40%, or 50% compared to a normal/healthy control.

These effects include the improvement/increase of the following parameters/symptoms: locomotor function and grip strength, the contractile properties of a muscle at the NMJ, resistance to fatigue of the muscle, muscle weight, impact on the general condition of life such as body weight.

An “effective amount,” of the antibody-based molecule refers to an amount sufficient, at dosages and for periods of time necessary, to achieve an intended biological effect or a desired therapeutic result including, without limitation, clinical results. The phrase “therapeutically effective amount” when applied to an antibody-based molecule of the invention is intended to denote an amount of the antibody that is sufficient to ameliorate, palliate, stabilize, reverse, slow or delay the progression of a disorder or disease state, or of a symptom of the disorder or disease. In an embodiment, the method of the present invention provides for administration of the antibody-based molecule in combinations with other compounds. In such instances, the “effective amount” is the amount of the combination sufficient to cause the intended biological effect.

In an embodiment, the invention provides an antibody-based molecule for use in the treatment of a disease or condition resulting from agrin deficiency in a human subject wherein the antibody-based molecule is administered to the subject intraperitonially (IP), intravenously, subcutaneously, or intramuscularly.

Intraperitonially (IP), Intramuscular injection (for example, into the arm or leg muscles) and intravenous infusion are preferred methods of administration of the antibody-based molecules of the present invention. In some methods, such molecules are administered as a sustained release composition or device, such as a Medipad™ device (Elan Pharm. Technologies, Dublin, Ireland). In some methods, the antibodies disclosed herein are injected directly into a particular tissue, for example μmbilical vein injection.

In one embodiment, the invention provides an antibody-based molecule of the present invention is administered parenterally. The phrases “parenteral administration” and “administered parenterally” as used herein denote modes of administration other than enteral and topical administration, usually by injection, and include epidermal, intravenous, intramuscular, intraarterial, intracardiac, intradermal, intraperitoneal, intratendinous, subcutaneous, subcuticular, and infusion. In one embodiment that antibody-based molecule is administered by intraperitoneal, intravenous or subcutaneous injection.

### Method of Treatment

The present invention also provides methods of preventing, delaying, reversing or treating a disease or condition resulting from agrin deficiency in a human subject, wherein the antibody-based molecule, the polynucleotide, the expression vector, or the pharmaceutical composition defined above is administered to the human subject. In an embodiment, the method comprises administering to the human subject a therapeutically effective amount of an antibody-based molecule, a polynucleotide, an expression vector, or a pharmaceutical composition. Each of an antibody-based molecule, a polynucleotide, an expression vector, and a pharmaceutical composition has been extensively defined in earlier sections of the description. In such methods of treatment, the antibody-based molecules are typically formulated as pharmaceutical compositions. In an embodiment, the polynucleotide, the expression vector or the composition is administered to the subject only after birth.

As used herein, the term “therapeutically effective amount” is intended to mean the quantity or dose of antibody-based molecule that is sufficient to trigger reversed, delayed and/or prevented postnatal developmental abnormalities, improved postnatal development, body weight gain, body muscle gain, prolonged postnatal survival, increase and maintenance of the number or percentage of fully innervated NMJ in the subject, improvement of the motor performance and/or grip strength of the subject, improvement of the contractile properties of a muscle at the NMJ of the subject, improvement of the resistance to fatigue of a muscle at the NMJ of the subject, induction of an increase of the muscle weight at the NMJ of the subject, increase in muscle mass of the subject, increase in muscle size of the subject, increase of nerve terminal area and complexity, acetylcholine perimeter, and/or endplate area, and/or a stabilization of said disorder relative to a human subject who is not administered with the antibody-based molecule, wherein the treated subject is assessed by techniques known to the skilled person, for example postnatal survival, body weight gain, muscle strength, muscle mass, muscle size, NMJ formation, and/or AChR size and morphology.

In an embodiment, the disease or condition resulting from agrin deficiency is a neuromuscular disease. In an embodiment, the antibody-based molecule is an anti-MuSK antibody or antigen binding fragment thereof. The anti-MuSK antibody or antigen binding fragment thereof is able to elicit a MuSK agonistic activity in the human subject. In an embodiment, such agonistic MuSK activity is able to eradicate or at least alleviate the symptoms associated with a disease or condition associated with agrin deficiency. An appropriate amount or dose can be determined by a physician, as appropriate.

As used herein, a method of “preventing” a disease or condition means preventing the onset of the disease, preventing the worsening of symptoms, preventing the progression of the disease or condition or reducing the risk of a subject developing the disease or condition. As used herein, a method of “treating” a disease or condition means curing a disease or condition and/or alleviating or eradicating the symptoms associated with the disease or condition such that the patient's suffering is reduced.

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. Each embodiment described herein may be combined together with any other embodiment described herein, unless otherwise indicated.

The present invention is further described by the following examples which should not be construed as limiting the scope of the invention.

### REFERENCES

1. Bogdanik, L.P., Burgess, R. W., 2011. A valid mouse model of AGRIN-associated congenital myasthenic syndrome. Hum Mol Genet 20, 4617-4633. https://doi.org/10.1093/hmg/ddr396
2. Jones, R.A., Reich, C.D., Dissanayake, K.N., Kristmundsdottir, F., Findlater, G.S., Ribchester, R.R., Simmen, M.W., Gillingwater, T. H., 2016. NMJ-morph reveals principal components of synaptic morphology influencing structure-function relationships at the neuromuscular junction. Open Biol 6, 160240. https://doi.org/10.1098/rsob.160240
3. Mira Vision, n.d. URL https://www.mira.vision/(accessed 6.29.23).
4. Oury, J., Zhang, W., Leloup, N., Koide, A., Corrado, A.D., Ketavarapu, G., Hattori, T., Koide, S., Burden, S. J., 2021. Mechanism of disease and therapeutic rescue of Dok7 congenital myasthenia. Nature 595, 404-408. https://doi.org/10.1038/s41586-021-03672-3
5. Schindelin, J., Arganda-Carreras, I., Frise, E., Kaynig, V., Longair, M., Pietzsch, T., Preibisch, S., Rueden, C., Saalfeld, S., Schmid, B., Tinevez, J.-Y., White, D.J., Hartenstein, V., Eliceiri, K., Tomancak, P., Cardona, A., 2012. Fiji: an open-source platform for biological-image analysis. Nat Methods 9, 676-682. https://doi.org/10.1038/nmeth.2019
6. Slater, C. R., 2020. “Fragmentation” of NMJs: a sign of degeneration or regeneration? A long journey with many junctions. Neuroscience 439, 28-40. https://doi.org/10.1016/j.neuroscience.2019.05.017
7. Spendiff, S., Howarth, R., McMacken, G., Davey, T., Quinlan, K., O'Connor, E., Slater, C., Hettwer, S., Mader, A., Roos, A., Horvath, R., Lochmüller, H., 2020. Modulation of the Acetylcholine Receptor Clustering Pathway Improves Neuromuscular Junction Structure and Muscle Strength in a Mouse Model of Congenital Myasthenic Syndrome. Front Mol Neurosci 13, 594220. https://doi.org/10.3389/fnmol.2020.594220
8. Ferns, M., et al., RNA splicing regulates agrin-mediated acetylcholine receptor clustering activity on cultured myotubes. Neuron, 1992. 8 (6): p. 1079-1086.
9. Gesemann, M., A.J. Denzer, and M.A. Ruegg, Acetylcholine receptor-aggregating activity of agrin isoforms and mapping of the active site. The Journal of cell biology, 1995. 128 (4): p. 625-636.
10. Hoch, W., et al., Developmental regulation of highly active alternatively spliced forms of agrin. Neuron, 1993. 11 (3): p. 479-490.
11. Ruegg, M.A., et al., The agrin gene codes for a family of basal lamina proteins that differ in function and distribution. Neuron, 1992. 8 (4): p. 691-699.
12. McMahan, U. The agrin hypothesis. in Cold Spring Harbor symposia on quantitative biology. 1990. Cold Spring Harbor Laboratory Press.
13. Tintignac, L.A., H.-R. Brenner, and M.A. Rüegg, Mechanisms regulating neuromuscular junction development and function and causes of muscle wasting. Physiological reviews, 2015. 95 (3): p. 809-852.
14. Sanes, J.R. and J.W. Lichtman, Induction, assembly, maturation and maintenance of a postsynaptic apparatus. Nat Rev Neurosci, 2001. 2 (11): p. 791-805.
15. Burden, S.J., Synapse-specific gene expression. Trends in Genetics, 1993. 9 (1): p. 12-16.
16. Moore, C., et al., Induction of multiple signaling loops by MuSK during neuromuscular synapse formation. Proceedings of the National Academy of Sciences, 2001. 98 (25): p. 14655-14660.
17. Kim, N., et al., Lrp4 is a receptor for Agrin and forms a complex with MuSK. Cell, 2008. 135 (2): p. 334-342.
18. Zhang, B., et al., LRP4 serves as a coreceptor of agrin. Neuron, 2008. 60 (2): p. 285-297.
19. Zhang, W., et al., Agrin binds to the N-terminal region of Lrp4 protein and stimulates association between Lrp4 and the first immunoglobulin-like domain in muscle-specific kinase (MuSK). Journal of Biological Chemistry, 2011. 286 (47): p. 40624-40630.
20. Zong, Y., et al., Structural basis of agrin-LRP4-MuSK signaling. Genes & development, 2012. 26 (3): p. 247-258.
21. Engel, A.G. and K. Ohno, Congenital myasthenic syndromes. Adv Neurol, 2002. 88: p. 203-15.
22. Hezareh, M., et al., Effector function activities of a panel of mutants of a broadly neutralizing antibody against human immunodeficiency virus type 1. J Virol, 2001. 75 (24): p. 12161-8.
23. Oury, J., et al., Mechanism of disease and therapeutic rescue of Dok7 congenital myasthenia. Nature, 2021.
24. Schneider, C.A., W.S. Rasband, and K.W. Eliceiri, NIH Image to ImageJ: 25 years of image analysis. Nat Methods, 2012. 9 (7): p. 671-5.

### Examples

### Example 1: MuSK Agonist Antibodies Rescue Agrin Deficiency in Agrn^{nmf380} Mice

### SUMMARY

The effects of a muscle specific kinase (MuSK) agonist antibody (3B2: represented by full length heavy chain SEQ ID NO: 282 and full length light chain SEQ ID NO: 283) on a mouse model of congenital myasthenic syndromes (CMS) caused by mutations in the AGRN gene was investigated. We used the Agrn^{nmf380} mouse generated through N-ethyl N-nitrosourea (ENU) chemical mutagenesis that induced a mutation in Agrn (nmf380-F1061S), resulting in a partial loss of function of the protein and so agrin deficient. Mice homozygous for the mutation are smaller than littermates, display poor hindlimb motor control and atrophy, and typically die within a few weeks to months of birth. They also have an increase in type I myofibers and abnormal NMJ morphology, closely recapitulating the human form of the disease and with shared features of motor neuron disease models (Bogdanik and Burgess, 2011). We injected mice at days P5 (20 mg/kg), P15 (10 mg/kg) and P35 (10 mg/kg) with either 3B2 or a control antibody (mota-hIgG1LALAdelk (Mot), represented by full length heavy chain SEQ ID NO:272 and full length light chain SEQ ID NO:273). Animals were weighed four times a week as a health monitoring tool, to determine the amount of drug to give, and as an outcome measure. In addition, animals underwent strength assessments at P7-8 (Tube Test), P30 & 48 (Hanging wire), and P23 & 40 (Grip strength meter). Animals were sacrificed at P50 and tissues collected. Injection of 3B2 rescued survival and body weight in Agrn^{nmf380} animals back to wild type (WT) levels, with improvements in strength also noted. Cellular analysis revealed an improvement in myofiber size and NMJ morphology in Agrn^{nmf380} (3B2) mice. We conclude that 3B2 may be a valid treatment for patients with CMS caused by mutations in AGRN.

### Materials

TABLE 1
test item information

| | Manufacturer | | Concentration | | Volume | Quantity |
|---|---|---|---|---|---|---|
| Antibody | code | Batch | (mg/mL) | Buffer | (mL) | (mg) |
| 3B2- | Evitria | 16155- | 7.55 | 50 mM | 6.63 | 50 |
| hIgG1LALAdelK | 907046.1 | SEC | | histidine/ | | |
| | | | | histidine—HCl with | | |
| | | | | 0.02% polysorbate 80 | | |
| Formulation | Evitria | 16155- | NA | 50 mM | 48 | NA |
| buffer | 907046.1 | FBB | | histidine/ | | |
| | | | | histidine—HCl with | | |
| | | | | 0.02% polysorbate 80 | | |
| Mota- | UPX | 4751 | 5.70 | 1xPBS 0.02% | 4.4 | 25.08 |
| hIgG1LALAdelK | #2020_311 | | | Tween80 | | |

### Methods

### Animal Husbandry

The Agrn^{nmf380} mouse with a point mutation in the Agrn gene on the C57BL/6J background was obtained from the Burgess lab (Bogdanik and Burgess, 2011). These mice were successfully rederived at the University of Ottawa by the animal care and veterinary service department (ACVS) transgenic core under the Lochmüller lab breeding protocol (#3089). All animal procedures were performed following the approval of the University of Ottawa Animal Care Committee (experimental protocol #3120) and complied with the guidelines of the Canadian Council on Animal Care and the Animals for Research Act. Animals were housed under 12 h light/dark cycles and had ad libitum access to standard chow and water. In addition, animals had access to wet food on the cage floor. Animals were weighed frequently, and any animal found to have lost >17% of body weight or showing a severe phenotype determined as specified on our ‘in house’ NMD monitoring sheet was humanely culled.

### Treatment Protocol and Tissue Collection

Animals were assigned at P5 to either drug treatment (3B2: represented by full length heavy chain SEQ ID NO: 282 and full length light chain SEQ ID NO: 283) or antibody (Mot: represented by full length heavy chain SEQ ID NO: 272 and full length light chain SEQ ID NO:273) control, with WT animals being used for comparison. Mice assigned to 3B2, or Mot groups received an IP injection (Table 1 Section 12) on days P5, P15, and P35. Animals underwent five strength assessments. At P7-9 mice underwent a Tube Test (aka Hindlimb suspension test), followed by grip strength measurements using a grip strength meter at P22, and P40, and a hanging wire test at P30 and P48 (FIG. 1, Section 12). For pharmacokinetic analysis, blood samples processed to serum were collected on the same day before the injection at P35 and P50 (end of study) when possible. These were sent to Argenx for further analysis. At the end of the study (P50) animals were euthanised (CO₂ followed by cervical dislocation), tissues collected and weighed.

Muscle for labelling was mounted in OCT and then frozen in isopentane pre-cooled in liquid nitrogen before being stored at −80° C. Muscle was cryosectioned on a Leica CM1860 at 12 μm and mounted on slides. Muscle for protein analysis was snap frozen in liquid nitrogen and stored at −80° C.

### NMJ Structural Analysis

NMJ labelling was performed on soleus muscle. Muscles were washed in ice-cold PBS for 2×10 mins and then separated out into small bundles of about 5 fibres using tweezers under a stereo-microscope. They were fixed overnight at 4° C. in 2% paraformaldehyde (PFA). The following morning, they were washed 2×1 hr with ice-cold PBS to remove the PFA. They were treated for 10 mins with Analar Ethanol followed by 10 mins with Analar Methanol, both at −20° C. Tissues were then incubated with blocking/permeabilization solution (5% horse serum, 5% BSA, 2% Triton X-100 in PBS) for 4 hours at room temperature with gentle agitation. Muscle bundles were incubated with antibodies, diluted in blocking buffer without triton, against neurofilament (mouse monoclonal IgG1, Cell Signalling, 1:100) and synaptophysin (rabbit polyclonal, ThermoFisherScientific, 1:100), overnight at 4° C. with agitation and then for a further 2 hrs at RT the next morning. Muscles were then washed in blocking buffer 4×1 hr at RT. They were incubated with Alexa 488-Conjugated a-Bungarotoxin (ThermoFisherScientific 1:250), Alexa Fluor 594 goat anti-rabbit IgG, and Alexa Fluor 594 goat anti-mouse IgG1 (ThermoFisher 1:200) for 4 hrs at RT. Samples were washed 4×1 hr in PBS and then mounted using Vectashield hardset mounting medium. Images were captured using Olympus FV1000c scanning confocal microscope using FV1000 application software (FV10-ASW) software. Parameters were kept constant for all samples and Z-stack images were acquired with a x63 oil immersion objective at 1 μm intervals. Analysis was performed blinded according to the NMJ_Morph protocol (Jones et al., 2016). In addition to the 19 variables calculated as part of the NMJ_Morph package, we also quantified the NMJs that completely lacked either synaptophysin or a-bungarotoxin staining.

Muscle Labelling Haematoxylin and Eosin (H&E)

Slides for H & E staining were thawed at room temperature for 20 mins. Sections were placed in tap water for 30 seconds then into haematoxylin for 2 mins. They were then washed in tap water until the water ran clear and placed in eosin for 30 seconds. They were again washed in tap water until the water ran clear before dehydrating through a graded ethanol series that finished with two clears with xylene. Sections were mounted with a cover slip and DPX. Tiled images were taken on a Zeiss Axio Imager at x20 magnification. Fibre area was then determined using an automated system (“Mira Vision,” n.d.).

### Myosin Heavy Chain (MHC)

Sections for labelling of MHCs (fibre type) and laminin were washed 1×5 min in PBS, then blocked in 10% normal goat serum (NGS) for 1 hour at RT. Primary antibodies were obtained from Developmental Studies Hybridoma Bank; BA-F8 (mouse anti-MHC1 IgG2b, 1:25), Sc-71 (mouse anti-MHC2a IgG1, 1:200), and BF-F3 (mouse anti-MHC2b 1:200). These were applied to one section, and on a second serial section 6H1 (mouse anti-MHC2×IgGM, 1:20) was labelled. Both sections were also reacted against rabbit anti-laminin IgG (1:750, Sigma). Muscles were incubated for 1 hour at RT and then washed 3×5 mins in PBS. Secondary antibodies (ThermoFisher Scientific): Alexa Fluor 350 IgG2b (y2b) goat anti-mouse (A-21140, 1:500), Alexa Fluor 594 IgG1 (y1) goat anti-mouse (A-21125, 1:100), Alexa Fluor 488 IgM goat anti-mouse (A-21042, 1:500), and Alexa Fluor 488 IgG goat anti-rabbit (A-11008, 1:500) were applied for 1 hour at RT. Following this, sections were given a final wash of 3×5 mins and mounted using Vectashield hardset mounting medium (Vector Laboratories). Tiled images of the whole muscle were captured on a Zeiss Axio Imager microscope with Zen software and analysed using FIJI (Schindelin et al., 2012) for MHC type. Fibre type grouping was recorded through the identification of ‘enclosed myofibres’, i.e. those that were completely surrounded by myofibres of the same MHC type.

### Data Analysis

All analysis was performed using Graphpad prism V.9.4.1. Data was first tested for normal distribution and then the appropriate test performed. Normally distributed data is presented as mean±sd, while data that does not follow a normal distribution is presented as box plots 5-95th percentile, with +denoting the mean.

### Protocol

Three groups of animals have been studied, an untreated wild type control group and 2 groups with Agrn^{nmf380} mice treated at days P5, P15, and P35 with an IP (intraperitoneal) injection of either the MuSK agonist antibody (represented by full length heavy chain SEQ ID NO:282 and full length light chain SEQ ID NO:283) or an isotope matched negative control antibody (Mota-hIgG1LALAdelk, represented by full length heavy chain SEQ ID NO:272 and full length light chain SEQ ID NO:273). Between P5 and P50 mice underwent several functional tests, at P50 mice were culled and tissues collected for further processing (Table 2, FIG. 1).

TABLE 2
Injection protocol for Agrn^{nmf380} mice.

| | | | | Dose Level | Dose | | Start of |
|---|---|---|---|---|---|---|---|
| Group | N | Genotype | Treatment | (mg/kg) | Route | Regimen | dosing |
| 1 | 6 | Agrn^{nmf380} | 3B2 | 20 mg/kg at P5 | IP | P5, P15, | P5 |
| 2 | 6 | | Mot | 10 mg/kg at P15 | | P35 | |
| | | | | and P35 | | | |
| 3 | 6 | Wild type | None | N/A | N/A | N/A | N/A |

### Results

### Functional Measures

Mice were followed for 50 days. While all WT mice completed the full 50 days only one Agrn^{nmf380} mouse treated with isotype control Ab (Mot) completed the study, however all Agrn^{nmf380} mice treated with the MuSK agonist Ab, 3B2 survived to the end of the study (FIG. 2A). Mice were weighed frequently to determine how much drug to administer, as a health monitoring tool, and as an outcome measure. Both WT mice and Agrn^{nmf380} (3B2) mice demonstrated a steady increase in body weight, however Agrn^{nmf380} (Mot) mice plateaued after 15 days of growth and those that survived longer had a very slow increase in body weight (FIG. 2B).

At P7-9 mice were subjected to the hindlimb suspension test, whereby they were inverted into a 50 mL falcon tube so that they could grip with their hindlimbs. Measures of holding time, number of pulls, and leg position were taken. This was repeated three times with a rest period between trials, and the average values used. Agrn^{nmf380} (3B3) animals held on for significantly longer than Agrn^{nmf380} (Mot) animals and were indistinguishable from WT animals (FIG. 3A). There were no significant differences between animals in terms of number of pulls or hindlimb suspension score (FIG. 3B-C).

Once the mice had been weaned, they were able to undergo further behavioral testing. Fore and hindlimb grip strength was measured using a grip strength meter. Mice underwent three trials with rest periods between each trial and the values were averaged. Mice were tested shortly after weaning at P23 and later in the study at P42. It should be noted that comparisons for Agrn^{nmf380} (Mot) mice are impacted by low survival and are thus made with a low n number. WT animals displayed a significantly stronger grip strength than Agrn^{nmf380} (Mot) at both P23 and P42, however they were only stronger than Agrn^{nmf380} (3B2) animals at P43. Agrn^{nmf380} (3B2) animals had a partially rescued grip strength, being significantly stronger than Agrn^{nmf380} (Mot) animals at both time points, however by the end of the study they were weaker than WT animals (FIG. 4A). Hindlimb grip strength also revealed differences between WT and Agrn^{nmf380} (Mot) mice at both P23 and P42. Like forelimb grip strength, WT mice were only stronger than Agrn^{nmf380} (3B2) mice at P42. Agrn^{nmf380} (3B2) mice were significantly stronger than Agrn^{nmf380} (Mot) mice at P42 (FIG. 4B). To determine grip strength ratios the strength in grams was divided by the body weight recorded on the same day. WT mice were again significantly stronger than Agrn^{nmf380} (Mot) mice at both time points in forelimb grip strength, suggesting that these differences were not completely attributable to an increase in muscle mass. While Agrn^{nmf380} (3B2) mice were significantly stronger than Agrn^{nmf380} (Mot) mice at P23, this difference did not persist by the end of the study (FIG. 4C). Hindlimb ratio was slightly different in that WT animals were not significantly stronger than Agrn^{nmf380} animals treated with Mot or 3B2 until P43. In addition, there were no differences between Agrn^{nmf380} (3B2) and Agrn^{nmf380} (Mot) animals at either time point (FIG. 4D).

In addition to the grip strength meter, animals were also tested using the inverted hanging wire test, where the holding time on an upturned grid was recorded. At P31 WT animals held on for an average of 170 seconds, Agrn^{nmf380} (mot) animals did not hold on at all while Agrn^{nmf380} (3B2) animals held on for an average of 51 second, however there were no significant differences between groups due to the large variability in data. By P48 WT animals held on for significantly longer than both Agrn^{nmf380} (Mot) and Agrn^{nmf380} (3B2) animals. At this stage only one Agrn^{nmf380} (Mot) animal was still alive, which held on for 1.3 seconds compared to the Agrn^{nmf380} (3B2) animals which held on for an average of 51 seconds (FIG. 5A). To determine the holding ratio the time in seconds was divided by the weight of the animal. As with the holding time, WT animals were not significantly stronger than Agrn^{nmf380} (Mot) or Agrn^{nmf380} (3B2) animals until P48, and there were no differences between the treated or untreated Agrn^{nmf380} groups (FIG. 5B).

At the end of the study animals were culled, and tissues collected and weighed. Muscles between WT and Agrn^{nmf380} (3B2) animals were not significantly different in weight (FIG. 6A). However, both WT and Agrn^{nmf380} (3B2) animals had significantly heavier quadriceps (Quads), gastrocnemius (GAS), and tibialis anterior (TA) muscles than Agrn^{nmf380} (Mot) animals. However, 5 of the 6 Agrn^{nmf380} (Mot) animals were considerably younger than WT and Agrn^{nmf380} (3B2) animals when they were culled. Although these differences persisted in the quads and gas muscles following normalization to body weight (FIG. 6B) suggesting the differences observed are not completely attributable to the differences in body weight of the animals.

### Cellular Measures

To determine muscle fibre size in the quadriceps muscle, cryosections were stained with H & E (FIG. 7A-1), imaged, and analysed using Mira Vision, while myosin heavy chain (fibre type) was determined using immunofluorescent labeling and analysed using FIJI. While there was a significant reduction in average fibre size in the quads of Agrn^{nmf380} (Mot) mice, this was rescued back to WT levels in the mice given 3B2 (FIG. 7A-2). This supports the above data (FIG. 6A-B) and suggests some of this increase in muscle mass is attributed to an increase in muscle fibre size.

In the soleus muscle there was a decrease in fibre size in the Agrn^{nmf380} (Mot) mice that was normalized in the Agrn^{nmf380} (3B2) mice, however this failed to reach statistical significance (FIG. 7A-3).

When we examined MHC type in the soleus muscle (FIG. 7B-1) we observed a decrease in the proportion of TIIx fibres and an increase in TI fibres (FIG. 7B-2) suggesting a fast to slow fibre type shift, as previously observed in this model (Spendiff et al., 2020). However, with the application of the drug, this shift was reversed in Agrn^{nmf380} (3B2) mice such that there were no significant differences between WT and Agrn^{nmf380} (3B2) mice in fibre type.

To determine if 3B2 had impacted the phosphorylation status of MuSK we performed immunolabelling of muscle cryosections of the gas muscle. While Agrn^{nmf380} mice had reduced levels of MuSK compared to WT animals (FIG. 7C-1), the levels of p-MuSK were restored in Agrn^{nmf380} (3B2) animals (FIG. 7C-2). Overall, the ratio of MuSK to p-MuSK was highest in the Agrn^{nmf380} (3B2) animals (FIG. 7C-3).

We also examined this through western blot (WB) (FIG. 7D-1) and again saw a reduction in p-MuSK in Agrn^{nmf380} (Mot) samples compared to WT. The rescue in Agrn^{nmf380} samples following 3B2 administration was striking (FIG. 7D-2).

To determine changes in NMJ morphology, analysis was performed on maximum intensity projections of NMJs taken using confocal microscopy (Table 3). There were many significant changes between WT and Agrn^{nmf380} (Mot) mice, especially in the presynaptic variables. These include a decrease in nerve terminal area and complexity, acetylcholine perimeter, and axon diameter. Administration of the MuSK agonist Ab in Agrn^{nmf380} (3B2) mice increased a number of these variables including nerve terminal area and complexity, acetylcholine perimeter, and endplate area. However, there were some variables where Agrn^{nmf380} (3B2) mice did not recover, including percentage overlap, however the importance of this measure is debated (Slater, 2020).

TABLE 3
Morphological variables of NMJs.
Initial analysis of NMJ structure in the soleus revealed no
sex related differences in variables and therefore data has
been combined for males and females.

| Variable | WT | Agrn^{nmf380} Mot | Agrn^{nmf380} 3B2 |
|---|---|---|---|
| Core Variables | | | |
| Presynaptic | | | |
| Absent Synaptophysin staining (%) | 0 | 0 | 0 |
| Nerve terminal area (μm²) | 184.3 ± 69.5(71) | 105.3 ± 92.9(28) ** | 167.1 ± 110.8(48) ^{#} |
| Nerve terminal perimeter (μm) | 216.6 ± 65.8(72) | 147 ± 105.2(28) * | 282.8 ± 176.2(49) ^{####} |
| Number of terminal branches | 27 ± 13.3(72) | 19.5 ± 9.8(28) | 49.0 ± 63(49) ^{##} |
| Number of branch points | 15.8 ± 7.8(72) | 8.4 ± 6.4(28) ** | 9.6 ± 8.1(49) * |
| Total length branches | 104.3 ± 33.1(72) | 67.0 ± 48.5(28) * | 114.2 ± 73.1(49) ^{###} |
| Postsynaptic | | | |
| Absent AChR staining (%) | 16.42 ± 13.4 (7) | 50 ± 46.4 (3) | 18.56 ± 16.67(5) |
| AChR area | 160.8 ± 82(60) | 65.2 ± 61.3(9) | 70.6 ± 54.4(40) *** |
| AChR perimeter | 240.4 ± 96(60) | 156.6 ± 124.3(9) ** | 203.2 ± 134.8(40) ^{#} |
| Endplate area | 381.5 ± 158.5(60) | 219.6 ± 96.3(9) | 585.6 ± 462.7(40) ^{#} |
| Endplate perimeter | 89.6 ± 17.4(60) | 81.5 ± 35(9) | 177.3 ± 249.7(40) |
| Endplate diameter | 39.8 ± 23.4(60) | 47.2 ± 24.7(9) | 55.9 ± 25.0(40) ** |
| Derived Variables | | | |
| Presynaptic | | | |
| Average length of branches | 4.5 ± 2.0(72) | 3.8 ± 1.8(28) | 2.9 ± 1.1(49) * |
| Complexity | 4.5 ± 0.5(72) | 3.7 ± 0.9(28) ** | 4.4 ± 0.9(49) ^{#} |
| Postsynaptic | | | |
| Average area of AChR clusters | 131.5 ± 97.9(59) | 48.2 ± 70.6(9) | 11.2 ± 10.7(40) **** |
| Fragmentation | 0.2 ± 0.3(61) | 0.6 ± 0.4(9) | 0.8 ± 0.1(40) **** |
| Compactness (%) | 42.5 ± 14(60) | 29.1 ± 18.5(9) | 14.2 ± 10.6(40) **** |
| Overlap (%) | 64.6 ± 15.3(60) | 37.1 ± 30.3(9) | 11.9 ± 14.3(40) **** |
| Area of synaptic contact | 101.4 ± 51.7(60) | 25.5 ± 37.6(9) * | 10.8 ± 14.8(40) **** |
| Associated Nerve Variables | | | |
| Axon diameter (μm) | 2.1 ± 0.8(47) | 1.7 ± 0.5(26) ** | 2.1 ± 0.7(43) |
| Number of axonal inputs | 1.0 ± 0.1(55) | 1.1 ± 0.3(27) | 1.0 ± 0.1(46) |

Analysis was performed using previously published method (Jones, Reich et al. 2016). Data presented as mean ± s.d. (no. of NMJs analysed). Most variables were not normally distributed and were analysed using Kruskal-Wallis test followed by Dunn's multiple comparisons test.
WT n = 7,
Agrn^{nmf380} (Mot) n = 3,
Agrn^{nmf380} (3B2) n = 5
* Significantly different from WT,
^{#} significantly different from Mot.
*^{/#} P < 0.05,
**^{/##} P < 0.005,
***^{/###} P < 0.001,
****^{/####} P < 0.0001.

### CONCLUSION

In conclusion, administration of the MuSK agonist Ab, 3B2, to a mouse model with agrin deficiency, (Agrn^{nmf380} mice) rescues survival and body weight to the point that Agrn^{nmf380} (3B2) mice were indistinguishable from WT on the graphs. In addition, there was an improvement in hanging wire time in Agrn^{nmf380} (3B2) mice only 3-4 days after the first administration of the drug. There was also improvement in forelimb ratio with 3B2 MuSK agonist Ab treatment though this was not a full rescue and Agrn^{nmf380} (3B2) remained significantly weaker than WT animals. The hanging wire test clearly separates the WT and Agrn^{nmf380} (Mot) mice despite considerable variation in the WT values. While there is no significant difference between Agrn^{nmf380} (Mot) and Agrn^{nmf380} (3B2) (mice, although a clear separation between groups can be seen. At P31 no Agrn^{nmf380} (Mot) mouse could hold onto the wire, however Agrn^{nmf380} (3B2) mice held on for between 35 to 73 seconds. By the final testing period only one Agrn^{nmf380} (Mot) remained which held on for 1.3 seconds compared to an average of 51 seconds for Agrn^{nmf380} (3B2) mice.

To explain the phenotypic changes observed we performed a number of cellular measures on the tissues collected. An improvement in many aspects of NMJ morphology was noted which likely resulted in an increase in muscle fibres size and over all muscle mass. The molecular mechanisms of this improvement are still being determined.

### Example 2: MuSK Agonist Antibodies Rescue Agrin^{ΔZ/ΔZ} Mutant Mice from Neonatal Lethality

Postnatal injection of MuSK agonist antibodies 3B2 (represented by full length heavy chain SEQ ID NO: 282 and full length light chain SEQ ID NO:283) shows a moderate effect on survival or weight gain of Agrin^{ΔZ/ΔZ} mice. Agrin^{ΔZ/ΔZ} C57BL/6 progeny, who were born from 3B2-injected Agrin^{ΔZ/+}C57BL/6 female mice during pregnancy ultimately shows disease relapse around P30 (FIG. 8).

Agrin^{ΔZ/ΔZ} C57BL/6 progeny, who were born from an 3B2-injected Agrin^{ΔZ/+}C57BL/6 female mouse during pregnancy ultimately shows disease relapse around P30. When they lost weight (~10% weight loss), they were re-injected with 3B2 (10 mg/kg), and monitored. Re-injection of 3B2 prolongs the survival and maintain body weight of the Agrin^{ΔZ/ΔZ} mice. These data suggest 3B2 can partially rescue Agrin^{ΔZ/ΔZ} mutant mice from neonatal lethality. P40 Agrin^{ΔZ/ΔZ} C57BL/6 mice, who were born from 3B2-injected Agrin^{ΔZ/+}C57BL/6 female mice during pregnancy, were placed in metabolic chambers and monitored 24 hours a day for 4 days. O₂ consumption, CO₂ production, energy expenditure, total activity, water intake, food intake, and body weight measurements were taken. The data shows that AgrinΔZ/ΔZ mice display respiratory, energy expenditure, activity, water intake, food intake, and body weight deficits compared to wildtype mice when disease relapses (FIG. 9). Agrin^{ΔZ/ΔZ} mice in a C57BL/6 background, who were born from 3B2-injected Agrin^{ΔZ/+}C57BL/6 female mice during pregnancy, display motor deficits at disease onset (10% weight loss) as assessed by the latency to fall from a rotating rotarod compared to non-injected wildtype mice. As shown in FIG. 10, Agrin^{ΔZ/ΔZ} mice display motor deficits when disease relapses. 3B2 partially restores synapse development in Agrin^{ΔZ/ΔZ} progeny born from Agrin^{ΔZ/+}C57BL/6 female, injected with 3B2 during pregnancy (FIG. 11).

1.-32. (canceled)

33. A method of increasing muscle fiber size in a subject with agrin deficiency comprising administering to the subject a therapeutically effective amount of an anti-MuSK antibody.

34. The method of claim 33, wherein the anti-MuSK antibody specifically binds the MuSK Frizzled (Fz)-like domain sequence of SEQ ID NO: 130.

35. The method of claim 33, wherein the anti-MuSK antibody is an agonist anti-MuSK antibody and/or has reduced or eliminated effector function.

36. The method of claim 33, wherein the anti-MuSK antibody induces MuSK phosphorylation.

37. The method of claim 33, wherein administering the anti-MuSK antibody results in one or more of the following therapeutic effects:
an increase and maintenance of the number or percentage of fully innervated neuromuscular junctions (NMJs) in the subject,
an improvement of motor performance and/or grip strength of the subject,
an improvement of the contractile properties of a muscle at the NMJ of the subject,
an improvement of the resistance to fatigue of a muscle at the NMJ of the subject,
an induction of an increase in muscle weight at the NMJ of the subject,
an increase in muscle mass of the subject,
an increase in muscle size of the subject,
an increase of nerve terminal area and complexity, acetylcholine perimeter, and/or endplate area, and/or
a stabilization of a disease or condition.

38. The method of claim 33, wherein the anti-MuSK antibody comprises a heavy chain variable domain (VH) comprising a CDR-H1, a CDR-H2, and a CDR-H3 comprised in the amino acid sequence of SEQ ID NO: 234, and a light chain variable domain (VL) comprising a CDR-L1, a CDR-L2, and a CDR-L3 comprised in the amino acid sequence of SEQ ID NO: 235.

39. The method of claim 38, wherein:
CDR-H1 comprises the amino acid sequence of SEQ ID NO: 147,
CDR-H2 comprises the amino acid sequence of SEQ ID NO: 153,
CDR-H3 comprises the amino acid sequence of SEQ ID NO: 156,
CDR-L1 comprises the amino acid sequence of SEQ ID NO: 159,
CDR-L2 comprises the amino acid sequence of SEQ ID NO: 172, and
CDR-L3 comprises the amino acid sequence of SEQ ID NO: 195.

40. The method of claim 39, wherein the VH comprises an amino acid sequence at least 80% identical to the amino acid sequence of SEQ ID NO: 234, and the VL comprises an amino acid sequence at least 80% identical to the amino acid sequence of SEQ ID NO: 235.

41. The method of claim 39, wherein the VH comprises the amino acid sequence of SEQ ID NO: 234, and the VL comprises the amino acid sequence of SEQ ID NO: 235.

42. The method of claim 41, wherein the anti-MuSK antibody comprises a heavy chain comprising an amino acid sequence at least 80% identical to the amino acid sequence of SEQ ID NO: 268, and a light chain comprising an amino acid sequence at least 80% identical to the amino acid sequence of SEQ ID NO: 269.

43. The method of claim 41, wherein the heavy chain comprises the amino acid sequence of SEQ ID NO: 268, and the light chain comprises the amino acid sequence of SEQ ID NO: 269.

44. A method of treating a disease or condition resulting from agrin deficiency in a human subject in need thereof, comprising administering to the subject a therapeutically effective amount of an anti-MuSK antibody.

45. The method of claim 44, wherein the disease or condition resulting from agrin deficiency is caused by a genetic defect in the agrin gene, deficiency in regulators of agrin expression and/or agrin activity, abnormal agrin metabolism, and/or disruption/intervention of agrin binding to its receptors.

46. The method of claim 45, wherein the disease or condition resulting from agrin deficiency is caused by a genetic defect in the agrin gene, wherein the genetic defect is caused by a mutation in the agrin gene, and the mutated agrin can no longer bind LRP4.

47. The method of claim 45, wherein the disease or condition resulting from agrin deficiency is caused by a genetic defect in the agrin gene, wherein the genetic defect is caused by a mutation in the agrin gene, and the mutated agrin binds LRP4 with decreased affinity and/or a lower capacity to activate MuSK phosphorylation than wild-type agrin.

48. The method of claim 44, wherein the disease or condition is characterized by impaired neuromuscular transmission and/or denervation at the NMJ.

49. The method of claim 48, wherein the disease is a neuromuscular disease selected from the group consisting of: amyotrophic lateral sclerosis (ALS), spinal muscular atrophy (SMA), myasthenia gravis (MG), congenital myasthenia, congenital myasthenic syndrome (CMS), Lambert-Eaton myasthenic syndrome (LEMS), Lyme disease, poliomyelitis, post-poliomyelitis, heavy metal intoxication, Kennedy syndrome, adult-onset Tay-Sachs disease, hereditary spastic paraplegia, multifocal neuropathy, cervical spondylosis, extramedullary tumor with compressive radiculopathy and myelopathy, inclusion body myositis, progressive bulbar palsy, progressive muscular atrophy, motor neuron syndrome, and thyrotoxic myopathy.

50. The method of claim 49, wherein the disease is agrin-induced CMS, MG caused by anti-agrin antibodies, or C9-ALS.

51. A method of treating a disease or condition resulting from agrin deficiency in a human subject in need thereof, comprising administering to the subject a therapeutically effective amount of a pharmaceutical composition comprising an antibody-based molecule that specifically binds MuSK, and at least one pharmaceutically acceptable carrier or excipient.

52. A method of treating a disease or condition resulting from agrin deficiency in a human subject in need thereof, comprising administering to the subject a therapeutically effective amount of a polynucleotide comprising a nucleotide sequence encoding an antibody-based molecule that specifically binds MuSK, an expression vector comprising said polynucleotide, or a host cell or cell-free expression system containing said expression vector.


SEQUENCE LISTING (289 sequences)

SEQ ID NO: 1 - protein, 5 aa, synthetic construct
  REGION 1..5: note = X1 HCDR1
SSSIH

SEQ ID NO: 2 - protein, 5 aa, synthetic construct
  REGION 1..5: note = X2 HCDR1
SSSIH

SEQ ID NO: 3 - protein, 5 aa, synthetic construct
  REGION 1..5: note = X3 HCDR1
SSSIH

SEQ ID NO: 4 - protein, 5 aa, synthetic construct
  REGION 1..5: note = X4 HCDR1
SSSIH

SEQ ID NO: 5 - protein, 5 aa, synthetic construct
  REGION 1..5: note = X5 HCDR1
SSSIH

SEQ ID NO: 6 - protein, 5 aa, synthetic construct
  REGION 1..5: note = X6 HCDR1
SSSIH

SEQ ID NO: 7 - protein, 5 aa, synthetic construct
  REGION 1..5: note = X7 HCDR1
SSSIH

SEQ ID NO: 8 - protein, 5 aa, synthetic construct
  REGION 1..5: note = X8 HCDR1
SSSIH

SEQ ID NO: 9 - protein, 5 aa, synthetic construct
  REGION 1..5: note = X9 HCDR1
SSSIH

SEQ ID NO: 10 - protein, 5 aa, synthetic construct
  REGION 1..5: note = X10 HCDR1
SSSIH

SEQ ID NO: 11 - protein, 5 aa, synthetic construct
  REGION 1..5: note = X11 HCDR1
SSSIH

SEQ ID NO: 12 - protein, 5 aa, synthetic construct
  REGION 1..5: note = X12 HCDR1
SSSIH

SEQ ID NO: 13 - protein, 5 aa, synthetic construct
  REGION 1..5: note = X13 HCDR1
SSSIH

SEQ ID NO: 14 - protein, 5 aa, synthetic construct
  REGION 1..5: note = X14 HCDR1
SSSIH

SEQ ID NO: 15 - protein, 5 aa, synthetic construct
  REGION 1..5: note = X15 HCDR1
SSSIH

SEQ ID NO: 16 - protein, 5 aa, synthetic construct
  REGION 1..5: note = X16 HCDR1
SSSIH

SEQ ID NO: 17 - protein, 17 aa, synthetic construct
  REGION 1..17: note = X1 HCDR2
SISSSSGSTSYADSVKG

SEQ ID NO: 18 - protein, 17 aa, synthetic construct
  REGION 1..17: note = X2 HCDR2
SISSSYGSTSYADSVKG

SEQ ID NO: 19 - protein, 17 aa, synthetic construct
  REGION 1..17: note = X3 HCDR2
SISSSSGYTYYADSVKG

SEQ ID NO: 20 - protein, 17 aa, synthetic construct
  REGION 1..17: note = X4 HCDR2
SISSSSGSTYYADSVKG

SEQ ID NO: 21 - protein, 17 aa, synthetic construct
  REGION 1..17: note = X5 HCDR2
SISSSSGSTSYADSVKG

SEQ ID NO: 22 - protein, 17 aa, synthetic construct
  REGION 1..17: note = X6 HCDR2
SISSYSGYTYYADSVKG

SEQ ID NO: 23 - protein, 17 aa, synthetic construct
  REGION 1..17: note = X7 HCDR2
SISSSYSSTYYADSVKG

SEQ ID NO: 24 - protein, 17 aa, synthetic construct
  REGION 1..17: note = X8 HCDR2
SISSSSGSTSYADSVKG

SEQ ID NO: 25 - protein, 17 aa, synthetic construct
  REGION 1..17: note = X9 HCDR2
SIYSSSGSTYYADSVKG

SEQ ID NO: 26 - protein, 17 aa, synthetic construct
  REGION 1..17: note = X10 HCDR2
SISSSYSSTSYADSVKG

SEQ ID NO: 27 - protein, 17 aa, synthetic construct
  REGION 1..17: note = X11 HCDR2
SISSYSGSTYYADSVKG

SEQ ID NO: 28 - protein, 17 aa, synthetic construct
  REGION 1..17: note = X12 HCDR2
YISPSSGYTSYADSVKG

SEQ ID NO: 29 - protein, 17 aa, synthetic construct
  REGION 1..17: note = X13 HCDR2
SISSSSGSTSYADSVKG

SEQ ID NO: 30 - protein, 17 aa, synthetic construct
  REGION 1..17: note = X14 HCDR2
SISSSSGYTYYADSVKG

SEQ ID NO: 31 - protein, 17 aa, synthetic construct
  REGION 1..17: note = X15 HCDR2
SISSSYGSTSYADSVKG

SEQ ID NO: 32 - protein, 17 aa, synthetic construct
  REGION 1..17: note = X16 HCDR2
SIYSSYGYTSYADSVKG

SEQ ID NO: 33 - protein, 17 aa, synthetic construct
  REGION 1..17: note = X1 HCDR3
KYWSQYYWAHYYGGLDY

SEQ ID NO: 34 - protein, 15 aa, synthetic construct
  REGION 1..15: note = X2 HCDR3
SEGDRYVSGYMGMDY

SEQ ID NO: 35 - protein, 15 aa, synthetic construct
  REGION 1..15: note = X3 HCDR3
SWYEMWMSGYFGFDY

SEQ ID NO: 36 - protein, 15 aa, synthetic construct
  REGION 1..15: note = X4 HCDR3
GEHDYYVFGYLGMDY

SEQ ID NO: 37 - protein, 20 aa, synthetic construct
  REGION 1..20: note = X5 HCDR3
SYTMFYYGGWYGSGYFGMDY

SEQ ID NO: 38 - protein, 15 aa, synthetic construct
  REGION 1..15: note = X6 HCDR3
TYGSYYVSSYTGMDY

SEQ ID NO: 39 - protein, 15 aa, synthetic construct
  REGION 1..15: note = X7 HCDR3
LAGLYHYPGYLGLDY

SEQ ID NO: 40 - protein, 19 aa, synthetic construct
  REGION 1..19: note = X8 HCDR3
SWSYHPWYYHVGWYTGLDY

SEQ ID NO: 41 - protein, 15 aa, synthetic construct
  REGION 1..15: note = X9 HCDR3
SGGEFYITSYYGMDY

SEQ ID NO: 42 - protein, 15 aa, synthetic construct
  REGION 1..15: note = X10 HCDR3
KYYRWRHNKYQGFDY

SEQ ID NO: 43 - protein, 15 aa, synthetic construct
  REGION 1..15: note = X11 HCDR3
SWGSYYVSGFVGFDY

SEQ ID NO: 44 - protein, 17 aa, synthetic construct
  REGION 1..17: note = X12 HCDR3
QYWVPQWWITQYFGMDY

SEQ ID NO: 45 - protein, 15 aa, synthetic construct
  REGION 1..15: note = X13 HCDR3
SSEHWYTIGYYGIDY

SEQ ID NO: 46 - protein, 15 aa, synthetic construct
  REGION 1..15: note = X14 HCDR3
GSHHWFLWIYSGLDY

SEQ ID NO: 47 - protein, 15 aa, synthetic construct
  REGION 1..15: note = X15 HCDR3
SEGDRYVSGYMGMDY

SEQ ID NO: 48 - protein, 16 aa, synthetic construct
  REGION 1..16: note = X16 HCDR3
NWGYYMYWGWYYALDY

SEQ ID NO: 49 - protein, 11 aa, synthetic construct
  REGION 1..11: note = X1 LCDR1
RASQSVSSAVA

SEQ ID NO: 50 - protein, 11 aa, synthetic construct
  REGION 1..11: note = X2 LCDR1
RASQSVSSAVA

SEQ ID NO: 51 - protein, 11 aa, synthetic construct
  REGION 1..11: note = X3 LCDR1
RASQSVSSAVA

SEQ ID NO: 52 - protein, 11 aa, synthetic construct
  REGION 1..11: note = X4 LCDR1
RASQSVSSAVA

SEQ ID NO: 53 - protein, 11 aa, synthetic construct
  REGION 1..11: note = X5 LCDR1
RASQSVSSAVA

SEQ ID NO: 54 - protein, 11 aa, synthetic construct
  REGION 1..11: note = X6 LCDR1
RASQSVSSAVA

SEQ ID NO: 55 - protein, 11 aa, synthetic construct
  REGION 1..11: note = X7 LCDR1
RASQSVSSAVA

SEQ ID NO: 56 - protein, 11 aa, synthetic construct
  REGION 1..11: note = X8 LCDR1
RASQSVSSAVA

SEQ ID NO: 57 - protein, 11 aa, synthetic construct
  REGION 1..11: note = X9 LCDR1
RASQSVSSAVA

SEQ ID NO: 58 - protein, 11 aa, synthetic construct
  REGION 1..11: note = X10 LCDR1
RASQSVSSAVA

SEQ ID NO: 59 - protein, 11 aa, synthetic construct
  REGION 1..11: note = X11 LCDR1
RASQSVSSAVA

SEQ ID NO: 60 - protein, 11 aa, synthetic construct
  REGION 1..11: note = X12 LCDR1
RASQSVSSAVA

SEQ ID NO: 61 - protein, 11 aa, synthetic construct
  REGION 1..11: note = X13 LCDR1
RASQSVSSAVA

SEQ ID NO: 62 - protein, 11 aa, synthetic construct
  REGION 1..11: note = X14 LCDR1
RASQSVSSAVA

SEQ ID NO: 63 - protein, 11 aa, synthetic construct
  REGION 1..11: note = X15 LCDR1
RASQSVSSAVA

SEQ ID NO: 64 - protein, 11 aa, synthetic construct
  REGION 1..11: note = X16 LCDR1
RASQSVSSAVA

SEQ ID NO: 65 - protein, 7 aa, synthetic construct
  REGION 1..7: note = X1 LCDR2
SASSLYS

SEQ ID NO: 66 - protein, 7 aa, synthetic construct
  REGION 1..7: note = X2 LCDR2
SASSLYS

SEQ ID NO: 67 - protein, 7 aa, synthetic construct
  REGION 1..7: note = X3 LCDR2
SASSLYS

SEQ ID NO: 68 - protein, 7 aa, synthetic construct
  REGION 1..7: note = X4 LCDR2
SASSLYS

SEQ ID NO: 69 - protein, 7 aa, synthetic construct
  REGION 1..7: note = X5 LCDR2
SASSLYS

SEQ ID NO: 70 - protein, 7 aa, synthetic construct
  REGION 1..7: note = X6 LCDR2
SASSLYS

SEQ ID NO: 71 - protein, 7 aa, synthetic construct
  REGION 1..7: note = X7 LCDR2
SASSLYS

SEQ ID NO: 72 - protein, 7 aa, synthetic construct
  REGION 1..7: note = X8 LCDR2
SASSLYS

SEQ ID NO: 73 - protein, 7 aa, synthetic construct
  REGION 1..7: note = X9 LCDR2
SASSLYS

SEQ ID NO: 74 - protein, 7 aa, synthetic construct
  REGION 1..7: note = X10 LCDR2
SASSLYS

SEQ ID NO: 75 - protein, 7 aa, synthetic construct
  REGION 1..7: note = X11 LCDR2
SASSLYS

SEQ ID NO: 76 - protein, 7 aa, synthetic construct
  REGION 1..7: note = X12 LCDR2
SASSLYS

SEQ ID NO: 77 - protein, 7 aa, synthetic construct
  REGION 1..7: note = X13 LCDR2
SASSLYS

SEQ ID NO: 78 - protein, 7 aa, synthetic construct
  REGION 1..7: note = X14 LCDR2
SASSLYS

SEQ ID NO: 79 - protein, 7 aa, synthetic construct
  REGION 1..7: note = X15 LCDR2
SASSLYS

SEQ ID NO: 80 - protein, 7 aa, synthetic construct
  REGION 1..7: note = X16 LCDR2
SASSLYS

SEQ ID NO: 81 - protein, 9 aa, synthetic construct
  REGION 1..9: note = X1 LCDR3
QQSSSSLIT

SEQ ID NO: 82 - protein, 9 aa, synthetic construct
  REGION 1..9: note = X2 LCDR3
QQSGVWLIT

SEQ ID NO: 83 - protein, 9 aa, synthetic construct
  REGION 1..9: note = X3 LCDR3
QQSSSSLIT

SEQ ID NO: 84 - protein, 11 aa, synthetic construct
  REGION 1..11: note = X4 LCDR3
QQSYKPGALIT

SEQ ID NO: 85 - protein, 9 aa, synthetic construct
  REGION 1..9: note = X5 LCDR3
QQSSSSLIT

SEQ ID NO: 86 - protein, 9 aa, synthetic construct
  REGION 1..9: note = X6 LCDR3
QQSSSSLIT

SEQ ID NO: 87 - protein, 10 aa, synthetic construct
  REGION 1..10: note = X7 LCDR3
QQSSRSSLLT

SEQ ID NO: 88 - protein, 9 aa, synthetic construct
  REGION 1..9: note = X8 LCDR3
QQSSSSLIT

SEQ ID NO: 89 - protein, 9 aa, synthetic construct
  REGION 1..9: note = X9 LCDR3
QQSSSSLIT

SEQ ID NO: 90 - protein, 9 aa, synthetic construct
  REGION 1..9: note = X10 LCDR3
QQSLWYPVT

SEQ ID NO: 91 - protein, 9 aa, synthetic construct
  REGION 1..9: note = X11 LCDR3
QQNSYYLIT

SEQ ID NO: 92 - protein, 9 aa, synthetic construct
  REGION 1..9: note = X12 LCDR3
QQSSSSLIT

SEQ ID NO: 93 - protein, 11 aa, synthetic construct
  REGION 1..11: note = X13 LCDR3
QQSYGSFSLIT

SEQ ID NO: 94 - protein, 9 aa, synthetic construct
  REGION 1..9: note = X14 LCDR3
QQGSYHLIT

SEQ ID NO: 95 - protein, 9 aa, synthetic construct
  REGION 1..9: note = X15 LCDR3
QQSGVWLIT

SEQ ID NO: 96 - protein, 11 aa, synthetic construct
  REGION 1..11: note = X16 LCDR3
QQWSSAQALIT

SEQ ID NO: 97 - protein, 126 aa, synthetic construct
  REGION 1..126: note = X1 VH
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARKYWSQYYWAHYYGGLDYWGQGTLVTVSS

SEQ ID NO: 98 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIK

SEQ ID NO: 99 - protein, 124 aa, synthetic construct
  REGION 1..124: note = X2 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYMGMDYWGQGTLVTVSS

SEQ ID NO: 100 - protein, 107 aa, synthetic construct
  REGION 1..107: note = X2 VL
DIQMTQSPSSLSASVGDRVTITCRASQSVSSAVAWYQQKPGKAPKLLIYSASSLYSGVPSRFSGSRSGTDFTLTISSLQPEDFATYYCQQSGVWLITFGQGTKVEIK

SEQ ID NO: 101 - protein, 124 aa, synthetic construct
  REGION 1..124: note = X3 VH
EVQLVESGGGLVQPGGSLRLSCAASGFTISSSSIHWVRQAPGKGLEWVASISSSSGYTYYADSVKGRFTISADTSKNTAYLQMNSLRAEDTAVYYCARSWYEMWMSGYFGFDYWGQGTLVTVSS

SEQ ID NO: 102 - protein, 107 aa, synthetic construct; identical to SEQ ID NO: 98
  REGION 1..107: note = VL

SEQ ID NO: 103 - protein, 124 aa, synthetic construct
  REGION 1..124: note = X4 VH
EVQLVESGGGLVQPGGSLRLSCAASGFTVSSSSIHWVRQAPGKGLEWVASISSSSGSTYYADSVKGRFTISADTSKNTAYLQMNSLRAEDTAVYYCARGEHDYYVFGYLGMDYWGQGTLVTVSS

SEQ ID NO: 104 - protein, 109 aa, synthetic construct
  REGION 1..109: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSYKPGALITFGQGTKVEIK

SEQ ID NO: 105 - protein, 129 aa, synthetic construct
  REGION 1..129: note = X5 VH
EVQLVESGGGLVQPGGSLRLSCAASGFTFYSSSIHWVRQAPGKGLEWVASISSSSGSTSYADSVKGRFTISADTSKNTAYLQMNSLRAEDTAVYYCARSYTMFYYGGWYGSGYFGMDYWGQGTLVTVSS

SEQ ID NO: 106 - protein, 107 aa, synthetic construct; identical to SEQ ID NO: 98
  REGION 1..107: note = VL

SEQ ID NO: 107 - protein, 124 aa, synthetic construct
  REGION 1..124: note = X6 VH
EVQLVESGGGLVQPGGSLRLSCAASGFTFSSSSIHWVRQAPGKGLEWVASISSYSGYTYYADSVKGRFTISADTSKNTAYLQMNSLRAEDTAVYYCARTYGSYYVSSYTGMDYWGQGTLVTVSS

SEQ ID NO: 108 - protein, 107 aa, synthetic construct; identical to SEQ ID NO: 98
  REGION 1..107: note = VL

SEQ ID NO: 109 - protein, 124 aa, synthetic construct
  REGION 1..124: note = X7 VH
EVQLVESGGGLVQPGGSLRLSCAASGFTLYSSSIHWVRQAPGKGLEWVASISSSYSSTYYADSVKGRFTISADTSKNTAYLQMNSLRAEDTAVYYCARLAGLYHYPGYLGLDYWGQGTLVTVSS

SEQ ID NO: 110 - protein, 108 aa, synthetic construct
  REGION 1..108: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSSRSSLLTFGQGTKVEIK

SEQ ID NO: 111 - protein, 128 aa, synthetic construct
  REGION 1..128: note = X8 VH
EVQLVESGGGLVQPGGSLRLSCAASGFTVSSSSIHWVRQAPGKGLEWVASISSSSGSTSYADSVKGRFTISADTSKNTAYLQMNSLRAEDTAVYYCARSWSYHPWYYHVGWYTGLDYWGQGTLVTVSS

SEQ ID NO: 112 - protein, 107 aa, synthetic construct; identical to SEQ ID NO: 98
  REGION 1..107: note = VL

SEQ ID NO: 113 - protein, 124 aa, synthetic construct
  REGION 1..124: note = X9 VH
EVQLVESGGGLVQPGGSLRLSCAASGFTFSSSSIHWVRQAPGKGLEWVASIYSSSGSTYYADSVKGRFTISADTSKNTAYLQMNSLRAEDTAVYYCARSGGEFYITSYYGMDYWGQGTLVTVSS

SEQ ID NO: 114 - protein, 107 aa, synthetic construct; identical to SEQ ID NO: 98
  REGION 1..107: note = VL

SEQ ID NO: 115 - protein, 124 aa, synthetic construct
  REGION 1..124: note = X10 VH
EVQLVESGGGLVQPGGSLRLSCAASGFTFSSSSIHWVRQAPGKGLEWVASISSSYSSTSYADSVKGRFTISADTSKNTAYLQMNSLRAEDTAVYYCARKYYRWRHNKYQGFDYWGQGTLVTVSS

SEQ ID NO: 116 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSLWYPVTFGQGTKVEIK

SEQ ID NO: 117 - protein, 124 aa, synthetic construct
  REGION 1..124: note = X11 VH
EVQLVESGGGLVQPGGSLRLSCAASGFTISSSSIHWVRQAPGKGLEWVASISSYSGSTYYADSVKGRFTISADTSKNTAYLQMNSLRAEDTAVYYCARSWGSYYVSGFVGFDYWGQGTLVTVSS

SEQ ID NO: 118 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQNSYYLITFGQGTKVEIK

SEQ ID NO: 119 - protein, 126 aa, synthetic construct
  REGION 1..126: note = X12 VH
EVQLVESGGGLVQPGGSLRLSCAASGFTISSSSIHWVRQAPGKGLEWVAYISPSSGYTSYADSVKGRFTISADTSKNTAYLQMNSLRAEDTAVYYCARQYWVPQWWITQYFGMDYWGQGTLVTVSS

SEQ ID NO: 120 - protein, 107 aa, synthetic construct; identical to SEQ ID NO: 98
  REGION 1..107: note = VL

SEQ ID NO: 121 - protein, 124 aa, synthetic construct
  REGION 1..124: note = X13 VH
EVQLVESGGGLVQPGGSLRLSCAASGFTISSSSIHWVRQAPGKGLEWVASISSSSGSTSYADSVKGRFTISADTSKNTAYLQMNSLRAEDTAVYYCARSSEHWYTIGYYGIDYWGQGTLVTVSS

SEQ ID NO: 122 - protein, 109 aa, synthetic construct
  REGION 1..109: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSYGSFSLITFGQGTKVEIK

SEQ ID NO: 123 - protein, 124 aa, synthetic construct
  REGION 1..124: note = X14 VH
EVQLVESGGGLVQPGGSLRLSCAASGFTFSSSSIHWVRQAPGKGLEWVASISSSSGYTYYADSVKGRFTISADTSKNTAYLQMNSLRAEDTAVYYCARGSHHWFLWIYSGLDYWGQGTLVTVSS

SEQ ID NO: 124 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQGSYHLITFGQGTKVEIK

SEQ ID NO: 125 - protein, 124 aa, synthetic construct; identical to SEQ ID NO: 99
  REGION 1..124: note = X15 VH

SEQ ID NO: 126 - protein, 107 aa, synthetic construct; identical to SEQ ID NO: 100
  REGION 1..107: note = VL

SEQ ID NO: 127 - protein, 125 aa, synthetic construct
  REGION 1..125: note = X16 VH
EVQLVESGGGLVQPGGSLRLSCAASGFTFSSSSIHWVRQAPGKGLEWVASIYSSYGYTSYADSVKGRFTISADTSKNTAYLQMNSLRAEDTAVYYCARNWGYYMYWGWYYALDYWGQGTLVTVSS

SEQ ID NO: 128 - protein, 109 aa, synthetic construct
  REGION 1..109: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQWSSAQALITFGQGTKVEIK

SEQ ID NO: 129 - protein, 869 aa, Homo sapiens
MRELVNIPLVHILTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMTVIISIMSSFAIFVLLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 130 - protein, 139 aa, synthetic construct
  REGION 1..139: note = Fz-like domain of MuSK
DNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARL

SEQ ID NO: 131 - protein, 124 aa, synthetic construct
  REGION 1..124: note = X17 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVFGYLGMDYWGQGTLVTVSS

SEQ ID NO: 132 - protein, 109 aa, synthetic construct; identical to SEQ ID NO: 104
  REGION 1..109: note = X17 VL

SEQ ID NO: 133 - protein, 126 aa, synthetic construct
  REGION 1..126: note = X18 VH
EVQLVESGGGLVQPGGSLRLSCAASGFTISSSSIHWVRQAPGKGLEWVASISSSSGYTSYADSVKGRFTISADTSKNTAYLQMNSLRAEDTAVYYCARKYSKRAYPDYYWRGLDYWGQGTLVTVSS

SEQ ID NO: 134 - protein, 107 aa, synthetic construct
  REGION 1..107: note = VL
DIQMTQSPSSLSASVGDRVTITCRASQSVSSAVAWYQQKPGKAPKLLIYSASSLYSGVPSRFSGSRSGTDFTLTISSLQPEDFATYYCQQSYWWPITFGQGTKVEIK

SEQ ID NO: 135 - protein, 5 aa, synthetic construct
  REGION 1..5: note = X17 HCDR1
YSSIH

SEQ ID NO: 136 - protein, 5 aa, synthetic construct
  REGION 1..5: note = X18 HCDR1
SSSIH

SEQ ID NO: 137 - protein, 17 aa, synthetic construct
  REGION 1..17: note = X17 HCDR2
SIYSSSGSTYYADSVKG

SEQ ID NO: 138 - protein, 17 aa, synthetic construct
  REGION 1..17: note = X18 HCDR2
SISSSSGYTSYADSVKG

SEQ ID NO: 139 - protein, 15 aa, synthetic construct
  REGION 1..15: note = X17 HCDR3
GDHGYYVFGYLGMDY

SEQ ID NO: 140 - protein, 17 aa, synthetic construct
  REGION 1..17: note = X18 HCDR3
KYSKRAYPDYYWRGLDY

SEQ ID NO: 141 - protein, 11 aa, synthetic construct
  REGION 1..11: note = X17 LCDR1
RASQSVSSAVA

SEQ ID NO: 142 - protein, 11 aa, synthetic construct
  REGION 1..11: note = X18 LCDR1
RASQSVSSAVA

SEQ ID NO: 143 - protein, 7 aa, synthetic construct
  REGION 1..7: note = X17 LCDR2
SASSLYS

SEQ ID NO: 144 - protein, 7 aa, synthetic construct
  REGION 1..7: note = X18 LCDR2
SASSLYS

SEQ ID NO: 145 - protein, 11 aa, synthetic construct
  REGION 1..11: note = X17 LCDR3
QQSYKPGALIT

SEQ ID NO: 146 - protein, 9 aa, synthetic construct
  REGION 1..9: note = X18 LCDR3
QQSYWWPIT

SEQ ID NO: 147 - protein, 5 aa, synthetic construct
  REGION 1..5: note = 14D10 HCDR1
DYGMS

SEQ ID NO: 148 - protein, 7 aa, synthetic construct
  REGION 1..7: note = 17H10 HCDR1
ARYYSWS

SEQ ID NO: 149 - protein, 5 aa, synthetic construct
  REGION 1..5: note = 16F11 HCDR1
LYYMN

SEQ ID NO: 150 - protein, 17 aa, synthetic construct
  REGION 1..17: note = 14D10 HCDR2
AIPWNGGSTYYKESVKG

SEQ ID NO: 151 - protein, 16 aa, synthetic construct
  REGION 1..16: note = 17H10 HCDR2
VIAYDGSTYYSPSLKS

SEQ ID NO: 152 - protein, 16 aa, synthetic construct
  REGION 1..16: note = 16F11 HCDR2
VIDTHSIAYYADSVKG

SEQ ID NO: 153 - protein, 17 aa, synthetic construct
  REGION 1..17: note = 3B2g1m1 HCDR2
AIPWSGGSTYYKESVKG

SEQ ID NO: 154 - protein, 17 aa, synthetic construct
  REGION 1..17: note = 3B2g1m2 HCDR2
AIPGSGGSTYYKESVKG

SEQ ID NO: 155 - protein, 17 aa, synthetic construct
  REGION 1..17: note = 3B2g1m4 HCDR2
AIPWQGGSTYYKESVKG

SEQ ID NO: 156 - protein, 12 aa, synthetic construct
  REGION 1..12: note = 14D10 HCDR3
RSGRIAFGALDA

SEQ ID NO: 157 - protein, 11 aa, synthetic construct
  REGION 1..11: note = 17H10 HCDR3
GSSRVAAAFDS

SEQ ID NO: 158 - protein, 7 aa, synthetic construct
  REGION 1..7: note = 16F11 HCDR3
GRTALVR

SEQ ID NO: 159 - protein, 14 aa, synthetic construct
  REGION 1..14: note = 14D10 LCDR1
GLSSGSVTSSNYPD

SEQ ID NO: 160 - protein, 14 aa, synthetic construct
  REGION 1..14: note = 3C4 LCDR1
GLSSGSVTASNYPD

SEQ ID NO: 161 - protein, 11 aa, synthetic construct
  REGION 1..11: note = 17H10 LCDR1
GGNRIGGKSVQ

SEQ ID NO: 162 - protein, 11 aa, synthetic construct
  REGION 1..11: note = 23B6 LCDR1
GGDNIGSKNAQ

SEQ ID NO: 163 - protein, 11 aa, synthetic construct
  REGION 1..11: note = 30E1 LCDR1
GGDNIGSKNTQ

SEQ ID NO: 164 - protein, 11 aa, synthetic construct
  REGION 1..11: note = 30A11 LCDR1
GGDNIASKNVQ

SEQ ID NO: 165 - protein, 17 aa, synthetic construct
  REGION 1..17: note = 16F11 LCDR1
KSSQSVVFGSNQKSYLN

SEQ ID NO: 166 - protein, 17 aa, synthetic construct
  REGION 1..17: note = 4C11 LCDR1
RSSQSVLYSSNQKNYLN

SEQ ID NO: 167 - protein, 15 aa, synthetic construct
  REGION 1..15: note = 7A12 LCDR1
ESSQSVLYNQKNYLN

SEQ ID NO: 168 - protein, 17 aa, synthetic construct
  REGION 1..17: note = 7G12 LCDR1
KSSQRVQLGSNQKSYLN

SEQ ID NO: 169 - protein, 15 aa, synthetic construct
  REGION 1..15: note = 7B8 LCDR1
KSSQSVLYNQKNYLA

SEQ ID NO: 170 - protein, 7 aa, synthetic construct
  REGION 1..7: note = 14D10 LCDR2
TTNSRHS

SEQ ID NO: 171 - protein, 7 aa, synthetic construct
  REGION 1..7: note = 7G4 LCDR2
STNSRHS

SEQ ID NO: 172 - protein, 7 aa, synthetic construct
  REGION 1..7: note = 3C4 LCDR2
STDSRHS

SEQ ID NO: 173 - protein, 7 aa, synthetic construct
  REGION 1..7: note = 31G2 LCDR2
STNSRLS

SEQ ID NO: 174 - protein, 7 aa, synthetic construct
  REGION 1..7: note = 17H10 LCDR2
ADSRRPS

SEQ ID NO: 175 - protein, 7 aa, synthetic construct
  REGION 1..7: note = 16F11 LCDR2
YASTQES

SEQ ID NO: 176 - protein, 7 aa, synthetic construct
  REGION 1..7: note = 4C11 LCDR2
WASARES

SEQ ID NO: 177 - protein, 7 aa, synthetic construct
  REGION 1..7: note = 7A12 LCDR2
WASTRQS

SEQ ID NO: 178 - protein, 7 aa, synthetic construct
  REGION 1..7: note = 7G12 LCDR2
YASTQQS

SEQ ID NO: 179 - protein, 7 aa, synthetic construct
  REGION 1..7: note = 7B8 LCDR2
WASTRES

SEQ ID NO: 180 - protein, 12 aa, synthetic construct
  REGION 1..12: note = 14D10 LCDR3
ALYMGGGSNVYV

SEQ ID NO: 181 - protein, 13 aa, synthetic construct
  REGION 1..13: note = 7G4 LCDR3
ALYMGRGSNKDYV

SEQ ID NO: 182 - protein, 12 aa, synthetic construct
  REGION 1..12: note = 3C4 LCDR3
ALYMYSDSKLYV

SEQ ID NO: 183 - protein, 12 aa, synthetic construct
  REGION 1..12: note = 3B2 LCDR3
GLYMYSGSKNYV

SEQ ID NO: 184 - protein, 12 aa, synthetic construct
  REGION 1..12: note = 3G3 LCDR3
ALYMGSDIRNYV

SEQ ID NO: 185 - protein, 12 aa, synthetic construct
  REGION 1..12: note = 31G2 LCDR3
ALYMGSGSRNYV

SEQ ID NO: 186 - protein, 12 aa, synthetic construct
  REGION 1..12: note = 31B7 LCDR3
ALYMGSESRNYV

SEQ ID NO: 187 - protein, 10 aa, synthetic construct
  REGION 1..10: note = 17H10 LCDR3
HVWGSTASAD

SEQ ID NO: 188 - protein, 10 aa, synthetic construct
  REGION 1..10: note = 23B6 LCDR3
HVWDSSTNAW

SEQ ID NO: 189 - protein, 11 aa, synthetic construct
  REGION 1..11: note = 30A11 LCDR3
QVWDSSTNVAV

SEQ ID NO: 190 - protein, 8 aa, synthetic construct
  REGION 1..8: note = 16F11 LCDR3
QQAYSAPT

SEQ ID NO: 191 - protein, 9 aa, synthetic construct
  REGION 1..9: note = 4C11 LCDR3
QQSYKPPYG

SEQ ID NO: 192 - protein, 9 aa, synthetic construct
  REGION 1..9: note = 7A12 LCDR3
QQAYNAPLT

SEQ ID NO: 193 - protein, 9 aa, synthetic construct
  REGION 1..9: note = 7G12 LCDR3
QQGYSAPFT

SEQ ID NO: 194 - protein, 9 aa, synthetic construct
  REGION 1..9: note = 7B8 LCDR3
QQGYSVPYT

SEQ ID NO: 195 - protein, 12 aa, synthetic construct
  REGION 1..12: note = 3B2g2m1 LCDR3
GLYSYSGSKNYV

SEQ ID NO: 196 - protein, 121 aa, synthetic construct
  REGION 1..121: note = 14D10 VH
ELQLVESGGGLVQPGGSLRLSCAASGFTFDDYGMSWVRQAPGKGLEWVSAIPWNGGSTYYKESVKGRFTISRDNAKKTLYLQMNSLKSEDTAVYYCAKRSGRIAFGALDAWGQGTLVTVSS

SEQ ID NO: 197 - protein, 112 aa, synthetic construct
  REGION 1..112: note = VL
QAVVTQEPSLSVSPGGTVTLTCGLSSGSVTSSNYPDWYQQTPGQAPRTLIYTTNSRHSGVPSRFSGSISGNKAALTITGAQPEDEADYYCALYMGGGSNVYVFGGGTKLTVL

SEQ ID NO: 198 - protein, 121 aa, synthetic construct; identical to SEQ ID NO: 196
  REGION 1..121: note = 7G4 VH

SEQ ID NO: 199 - protein, 113 aa, synthetic construct
  REGION 1..113: note = VL
QAVVTQEPSLSVSPGGTVTLTCGLSSGSVTSSNYPDWYQQTPGQAPRALIYSTNSRHSGVPSRFSGSISGNKAALTITGAQPEDEADYYCALYMGRGSNKDYVFGGGTKLTVL

SEQ ID NO: 200 - protein, 121 aa, synthetic construct; identical to SEQ ID NO: 196
  REGION 1..121: note = 3C4 VH

SEQ ID NO: 201 - protein, 112 aa, synthetic construct
  REGION 1..112: note = VL
QAVVTQEPSLSVSPGGTVTLTCGLSSGSVTASNYPDWYQQTPGQAPRGLIYSTDSRHSGVPSRFSGSISGNKAALTITGAQPEDEADYYCALYMYSDSKLYVFGGGTKLTVL

SEQ ID NO: 202 - protein, 121 aa, synthetic construct; identical to SEQ ID NO: 196
  REGION 1..121: note = 3B2 VH

SEQ ID NO: 203 - protein, 112 aa, synthetic construct
  REGION 1..112: note = VL
QAVVTQEPSLSVSPGGTVTLTCGLSSGSVTSSNYPDWYQQTPGQAPRGLIYSTDSRHSGVPSRFSGSISGNKAALTITGAQSEDEADYYCGLYMYSGSKNYVFGGGTKLTVL

SEQ ID NO: 204 - protein, 121 aa, synthetic construct; identical to SEQ ID NO: 196
  REGION 1..121: note = 3G3 VH

SEQ ID NO: 205 - protein, 112 aa, synthetic construct
  REGION 1..112: note = VL
QTVVTQEPSLSVSPGGTVTLTCGLSSGSVTSSNYPDWYQQTPGQAPRALIYSTNSRHSGVPSRFSGSTSGNKAALTITGAQPEDEADYYCALYMGSDIRNYVFGGGTKLTVL

SEQ ID NO: 206 - protein, 121 aa, synthetic construct; identical to SEQ ID NO: 196
  REGION 1..121: note = 31G2 VH

SEQ ID NO: 207 - protein, 112 aa, synthetic construct
  REGION 1..112: note = VL
QAVVTQEPSLSVSPGGTVTLTCGLSSGSVTSSNYPDWYQQTPGQAPRALIYSTNSRLSGVPSRFSGSFSGNKAALTITGAQPEDEADYYCALYMGSGSRNYVFGGGTKLTVL

SEQ ID NO: 208 - protein, 121 aa, synthetic construct; identical to SEQ ID NO: 196
  REGION 1..121: note = 31B7 VH

SEQ ID NO: 209 - protein, 112 aa, synthetic construct
  REGION 1..112: note = VL
QAVVTQEPSLSVSPGGTVTLTCGLSSGSVTSSNYPDWYQQTPGQAPRALIYSTNSRLSGVPSRFSGSFSGNKAALTITGAQPEDEADYYCALYMGSESRNYVFGGGTKLTVL

SEQ ID NO: 210 - protein, 121 aa, synthetic construct
  REGION 1..121: note = 17H10 VH
QVQVQESGPGLVKPSQTLSLTCTVSGGSITARYYSWSWIRQPPGKGLEWMGVIAYDGSTYYSPSLKSRTSISRDTSKNQFSLHLSSVTPDDTAVYYCARGSSRVAAAFDSWGQGTQVTVSS

SEQ ID NO: 211 - protein, 107 aa, synthetic construct
  REGION 1..107: note = VL
SYELTQSPSVSVALRQTAKITCGGNRIGGKSVQWYQQKPGQAPMLVIYADSRRPSGIPERFTGSNSGNTATLTITGAQAEDEADYYCHVWGSTASADFGGGTHLTVL

SEQ ID NO: 212 - protein, 121 aa, synthetic construct; identical to SEQ ID NO: 210
  REGION 1..121: note = 23B6 VH

SEQ ID NO: 213 - protein, 107 aa, synthetic construct
  REGION 1..107: note = VL
SYELTQSPSVSVALRQTAKITCGGDNIGSKNAQWYQQKPGQAPVMVLYADSRRPSGIPERFSGSNSGNTATLTISGAQAEDEADYYCHVWDSSTNAWFGGGTHLTVL

SEQ ID NO: 214 - protein, 121 aa, synthetic construct; identical to SEQ ID NO: 210
  REGION 1..121: note = 30E1 VH

SEQ ID NO: 215 - protein, 107 aa, synthetic construct
  REGION 1..107: note = VL
SYELTQSPSVSVALRRTAKITCGGDNIGSKNTQWYQQKPGQAPVLVIYADSRRPSGIPERFSGSNSGNTATLTISGAQAEDEADYYCHVWDSSTNAWFGGGTHLTVL

SEQ ID NO: 216 - protein, 121 aa, synthetic construct; identical to SEQ ID NO: 210
  REGION 1..121: note = 30A11 VH

SEQ ID NO: 217 - protein, 108 aa, synthetic construct
  REGION 1..108: note = VL
SYELTQSPSVTVALRQTAKITCGGDNIASKNVQWYQQKPGQAPSLVIWADSRRPSGIPVRFSGSNFGNTATLTISGAQAEDEADYYCQVWDSSTNVAVFGGGTHLTVL

SEQ ID NO: 218 - protein, 115 aa, synthetic construct
  REGION 1..115: note = 16F11 VH
EVQLVESGGGLVQPGGSLSLSCVASGFTFSLYYMNWVRQAPGKGLEWLSVIDTHSIAYYADSVKGRFTISRDNVKNTLYLQLNNLKPEDTALYYCVLGRTALVRWGQGTQVTVSS

SEQ ID NO: 219 - protein, 112 aa, synthetic construct
  REGION 1..112: note = VL
DIVMTQSPSSVTASVGEKVTINCKSSQSVVFGSNQKSYLNWYQQRPGQSPRLLIYYASTQESGIPDRFSGSGSTTDFTLTISSVQPEDAAVYYCQQAYSAPTFGSGTRLEIK

SEQ ID NO: 220 - protein, 115 aa, synthetic construct; identical to SEQ ID NO: 218
  REGION 1..115: note = 4C11 VH

SEQ ID NO: 221 - protein, 113 aa, synthetic construct
  REGION 1..113: note = VL
DIVMTQSPSSVTASAGERVTINCRSSQSVLYSSNQKNYLNWYQQRLGQSPRLLIYWASARESGVPDRFSGSGSTTNFTLTISSFQPEDAAVYYCQQSYKPPYGFGSGTRLEIK

SEQ ID NO: 222 - protein, 115 aa, synthetic construct; identical to SEQ ID NO: 218
  REGION 1..115: note = 7A12 VH

SEQ ID NO: 223 - protein, 113 aa, synthetic construct
  REGION 1..113: note = VL
EIVLTQSPSSVTASIGEKVTINCESSQSVLYNQKNYLNWYQQRPGQSPRLLIYWASTRQSGVPDRFSGSGSGSTTDFTLTISSFQPEDVAVYYCQQAYNAPLTFGPGTKVELK

SEQ ID NO: 224 - protein, 115 aa, synthetic construct; identical to SEQ ID NO: 218
  REGION 1..115: note = 7G12 VH

SEQ ID NO: 225 - protein, 113 aa, synthetic construct
  REGION 1..113: note = VL
EIVLTQSPNSVTASVGEKVTINCKSSQRVQLGSNQKSYLNWYQQRPGQSPRLLIYYASTQQSGIPDRFSGSGSATDFTLTINSVQPEDAAVYYCQQGYSAPFTFGQGTKVELK

SEQ ID NO: 226 - protein, 115 aa, synthetic construct; identical to SEQ ID NO: 218
  REGION 1..115: note = 7B8 VH

SEQ ID NO: 227 - protein, 111 aa, synthetic construct
  REGION 1..111: note = VL
EIVLTQSPSSVTASAGEKVTINCKSSQSVLYNQKNYLAWYQQRPGQSPRLLIYWASTRESGVPDRFSGSGSTTDFTLTISSFQPEDVAVYYCQQGYSVPYTFGSGTRLEIK

SEQ ID NO: 228 - protein, 121 aa, synthetic construct
  REGION 1..121: note = 3B2g1m1 VH
EVQLLESGGGLVQPGGSLRLSCAASGFTFSDYGMSWVRQAPGKGLEWVSAIPWSGGSTYYKESVKGRFTISRDNSKNTLYLQMNSLRAEDTAVYYCAKRSGRIAFGALDAWGQGTLVTVSS

SEQ ID NO: 229 - protein, 112 aa, synthetic construct
  REGION 1..112: note = VL
QTVVTQEPSFSVSPGGTVTLTCGLSSGSVTSSNYPDWYQQTPGQAPRTLIYSTDSRHSGVPDRFSGSILGNKAALTITGAQADDESDYYCGLYMYSGSKNYVFGGGTKLTVL

SEQ ID NO: 230 - protein, 121 aa, synthetic construct
  REGION 1..121: note = 3B2g1m2 VH
EVQLLESGGGLVQPGGSLRLSCAASGFTFSDYGMSWVRQAPGKGLEWVSAIPGSGGSTYYKESVKGRFTISRDNSKNTLYLQMNSLRAEDTAVYYCAKRSGRIAFGALDAWGQGTLVTVSS

SEQ ID NO: 231 - protein, 112 aa, synthetic construct; identical to SEQ ID NO: 229
  REGION 1..112: note = VL

SEQ ID NO: 232 - protein, 121 aa, synthetic construct
  REGION 1..121: note = 3B2g1m4 VH
EVQLLESGGGLVQPGGSLRLSCAASGFTFSDYGMSWVRQAPGKGLEWVSAIPWQGGSTYYKESVKGRFTISRDNSKNTLYLQMNSLRAEDTAVYYCAKRSGRIAFGALDAWGQGTLVTVSS

SEQ ID NO: 233 - protein, 112 aa, synthetic construct; identical to SEQ ID NO: 229
  REGION 1..112: note = VL

SEQ ID NO: 234 - protein, 121 aa, synthetic construct; identical to SEQ ID NO: 228
  REGION 1..121: note = 3B2g2m1 VH

SEQ ID NO: 235 - protein, 112 aa, synthetic construct
  REGION 1..112: note = VL
QTVVTQEPSFSVSPGGTVTLTCGLSSGSVTSSNYPDWYQQTPGQAPRTLIYSTDSRHSGVPDRFSGSILGNKAALTITGAQADDESDYYCGLYSYSGSKNYVFGGGTKLTVL

SEQ ID NO: 236 - protein, 121 aa, synthetic construct; identical to SEQ ID NO: 230
  REGION 1..121: note = 3B2g2m2 VH

SEQ ID NO: 237 - protein, 112 aa, synthetic construct; identical to SEQ ID NO: 235
  REGION 1..112: note = VL

SEQ ID NO: 238 - protein, 121 aa, synthetic construct; identical to SEQ ID NO: 232
  REGION 1..121: note = 3B2g2m4 VH

SEQ ID NO: 239 - protein, 112 aa, synthetic construct; identical to SEQ ID NO: 235
  REGION 1..112: note = VL

SEQ ID NO: 240 - protein, 15 aa, synthetic construct
  REGION 1..15: note = X2m1 HCDR3
SEGDRYVSGYFGFDY

SEQ ID NO: 241 - protein, 15 aa, synthetic construct
  REGION 1..15: note = X2m2 HCDR3
SEGDRYVSGYFGLDY

SEQ ID NO: 242 - protein, 15 aa, synthetic construct
  REGION 1..15: note = X2m3 HCDR3
SEGDRYVSGYSGFDY

SEQ ID NO: 243 - protein, 15 aa, synthetic construct
  REGION 1..15: note = X2m4 HCDR3
SEGDRYVSGYSGLDY

SEQ ID NO: 244 - protein, 15 aa, synthetic construct
  REGION 1..15: note = X2m5 HCDR3
SEGDRYVSGYFGMDY

SEQ ID NO: 245 - protein, 15 aa, synthetic construct
  REGION 1..15: note = X2m6 HCDR3
SEGDRYVSGYSGMDY

SEQ ID NO: 246 - protein, 15 aa, synthetic construct
  REGION 1..15: note = X2m7 HCDR3
SEGDRYVSGYMGFDY

SEQ ID NO: 247 - protein, 15 aa, synthetic construct
  REGION 1..15: note = X2m8 HCDR3
SEGDRYVSGYMGLDY

SEQ ID NO: 248 - protein, 15 aa, synthetic construct
  REGION 1..15: note = X17m1 HCDR3
GDHGYYVSGYLGMDY

SEQ ID NO: 249 - protein, 15 aa, synthetic construct
  REGION 1..15: note = X17m2 HCDR3
GDHGYYVYGYLGMDY

SEQ ID NO: 250 - protein, 15 aa, synthetic construct
  REGION 1..15: note = X17m3 HCDR3
GDHGYYVSGYLGFDY

SEQ ID NO: 251 - protein, 15 aa, synthetic construct
  REGION 1..15: note = X17m6 HCDR3
GEHGYYVSGYLGFDY

SEQ ID NO: 252 - protein, 124 aa, synthetic construct
  REGION 1..124: note = X2m1 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYFGFDYWGQGTLVTVSS

SEQ ID NO: 253 - protein, 124 aa, synthetic construct
  REGION 1..124: note = X2m2 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYFGLDYWGQGTLVTVSS

SEQ ID NO: 254 - protein, 124 aa, synthetic construct
  REGION 1..124: note = X2m3 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYSGFDYWGQGTLVTVSS

SEQ ID NO: 255 - protein, 124 aa, synthetic construct
  REGION 1..124: note = X2m4 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYSGLDYWGQGTLVTVSS

SEQ ID NO: 256 - protein, 124 aa, synthetic construct
  REGION 1..124: note = X2m5 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYFGMDYWGQGTLVTVSS

SEQ ID NO: 257 - protein, 124 aa, synthetic construct
  REGION 1..124: note = X2m6 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYSGMDYWGQGTLVTVSS

SEQ ID NO: 258 - protein, 124 aa, synthetic construct
  REGION 1..124: note = X2m7 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYMGFDYWGQGTLVTVSS

SEQ ID NO: 259 - protein, 124 aa, synthetic construct
  REGION 1..124: note = X2m8 VH
EVQLVESGGGLVQPGGSLRLSCAASGFTVSSSSIHWVRQAPGKGLEWVASISSSYGSTSYADSVKGRFTISADTSKNTAYLQMNSLRAEDTAVYYCARSEGDRYVSGYMGLDYWGQGTLVTVSS

SEQ ID NO: 260 - protein, 124 aa, synthetic construct
  REGION 1..124: note = X17m1 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVSGYLGMDYWGQGTLVTVSS

SEQ ID NO: 261 - protein, 124 aa, synthetic construct
  REGION 1..124: note = X17m2 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVYGYLGMDYWGQGTLVTVSS

SEQ ID NO: 262 - protein, 124 aa, synthetic construct
  REGION 1..124: note = X17m3 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGDHGYYVSGYLGFDYWGQGTLVTVSS

SEQ ID NO: 263 - protein, 124 aa, synthetic construct
  REGION 1..124: note = X17m6 VH
EVQLVESGGGLVQPGGSLRLSCAASGFTISYSSIHWVRQAPGKGLEWVASIYSSSGSTYYADSVKGRFTISADTSKNTAYLQMNSLRAEDTAVYYCARGEHGYYVSGYLGFDYWGQGTLVTVSS

SEQ ID NO: 264 - DNA, 1432 bp, synthetic construct
  misc_feature 1..1432: note = VH_3B2g2m1 coding sequence
  source 1..1432: mol_type = other DNA
aagcttgccgccaccatggaatggtcctgggtgttcctgttcttcctgtccgtgaccaccggcgtgcactctgaagttcagctgctggaatctggcggcggattggttcaacctggcggctctctgagactgtcttgtgccgcttctggcttcaccttctccgactacggcatgtcctgggtccgacaggctcctggcaaaggactggaatgggtgtccgccattccttggagcggcggctccacctactacaaagaatccgtgaagggccgctttaccatcagccgggacaactccaagaacaccctgtacctgcagatgaactccctgagagccgaggacaccgccgtgtactactgcgctaagagatccggcagaatcgccttcggcgctctggatgcttggggacagggaacactggtcaccgtgtcctccgcttctaccaagggaccctctgtgttccctctggctccttccagcaagtctacctctggcggaacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattctttctgtactccaagctgacagtggacaagtccagatggcagcagggcaacgtgttctcctgcagcgtgatgcacgaggccctgcacaatcactacacacagaagtccctgtctctgtcccctggctgatgaattc

SEQ ID NO: 265 - DNA, 739 bp, synthetic construct
  misc_feature 1..739: note = VL_3B2g2m1 coding sequence
  source 1..739: mol_type = other DNA
aagcttgccgccaccatgtctgtgcctacacaggttctgggactgctgctgctgtggctgaccgatgctagatgccagaccgtcgtgacccaagagccttccttctctgtgtctcctggcggcaccgtgacactgacatgtggactgtcctctggctccgtgacctccagcaactaccccgactggtatcagcagacccctggacaggctcctcggacactgatctactccaccgactccagacactctggcgtgcccgatagattctccggctctatcctgggcaacaaggccgctctgacaatcactggcgcccaggccgatgacgagtccgattactactgcggcctgtactcctactccggctccaagaactacgtgttcggcggaggcaccaagctgacagtgctgggacaacctaaggctgccccttccgtgactctgttccctccatcctctgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctcctgatgaattc

SEQ ID NO: 266 - protein, 220 aa, synthetic construct
  REGION 1..220: note = Fcc with K (human domain)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 219 aa, synthetic construct
  REGION 1..219: note = Fcc without K (human domain)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 268 - protein, 450 aa, synthetic construct
  REGION 1..450: note = ARGX-119 hIgG1, full length heavy chain, reduced effector function, delK
EVQLLESGGGLVQPGGSLRLSCAASGFTFSDYGMSWVRQAPGKGLEWVSAIPWSGGSTYYKESVKGRFTISRDNSKNTLYLQMNSLRAEDTAVYYCAKRSGRIAFGALDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 269 - protein, 218 aa, synthetic construct
  REGION 1..218: note = ARGX-119 hIgG1, full length light chain, reduced effector function, delK
QTVVTQEPSFSVSPGGTVTLTCGLSSGSVTSSNYPDWYQQTPGQAPRTLIYSTDSRHSGVPDRFSGSILGNKAALTITGAQADDESDYYCGLYSYSGSKNYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 270 - protein, 451 aa, synthetic construct
  REGION 1..451: note = ARGX-119 hIgG1, full length heavy chain, reduced effector function
EVQLLESGGGLVQPGGSLRLSCAASGFTFSDYGMSWVRQAPGKGLEWVSAIPWSGGSTYYKESVKGRFTISRDNSKNTLYLQMNSLRAEDTAVYYCAKRSGRIAFGALDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 218 aa, synthetic construct; identical to SEQ ID NO: 269
  REGION 1..218: note = ARGX-119 hIgG1, full length light chain, reduced effector function

SEQ ID NO: 272 - protein, 449 aa, synthetic construct
  REGION 1..449: note = Mota-hIgG1, full length heavy chain, reduced effector function, delK
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 273 - protein, 213 aa, synthetic construct
  REGION 1..213: note = Mota-hIgG1, full length light chain, reduced effector function, delK
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 450 aa, synthetic construct
  REGION 1..450: note = Mota-hIgG1, full length heavy chain, reduced effector function
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - protein, 213 aa, synthetic construct; identical to SEQ ID NO: 273
  REGION 1..213: note = Mota-hIgG1, full length light chain, reduced effector function

SEQ ID NO: 276 - DNA, 1350 bp, synthetic construct
  misc_feature 1..1350: note = coding DNA for HC_ARGX-119
  source 1..1350: mol_type = other DNA
gaagttcagctgctggaatctggcggcggattggttcaacctggcggctctctgagactgtcttgtgccgcttctggcttcaccttctccgactacggcatgtcctgggtccgacaggctcctggcaaaggactggaatgggtgtccgccattccttggagcggcggctccacctactacaaagaatccgtgaagggccgctttaccatcagccgggacaactccaagaacaccctgtacctgcagatgaactccctgagagccgaggacaccgccgtgtactactgcgctaagagatccggcagaatcgccttcggcgctctggatgcttggggacagggaacactggtcaccgtgtcctccgcttctaccaagggaccctctgtgttccctctggctccttccagcaagtctacctctggcggaacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaagctgctggcggcccatccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattctttctgtactccaagctgacagtggacaagtccagatggcagcagggcaacgtgttctcctgcagcgtgatgcacgaggccctgcacaatcactacacacagaagtccctgtctctgtcccctggc

SEQ ID NO: 277 - DNA, 363 bp, synthetic construct
  misc_feature 1..363: note = coding DNA for VH_ARGX-119
  source 1..363: mol_type = other DNA
gaagttcagctgctggaatctggcggcggattggttcaacctggcggctctctgagactgtcttgtgccgcttctggcttcaccttctccgactacggcatgtcctgggtccgacaggctcctggcaaaggactggaatgggtgtccgccattccttggagcggcggctccacctactacaaagaatccgtgaagggccgctttaccatcagccgggacaactccaagaacaccctgtacctgcagatgaactccctgagagccgaggacaccgccgtgtactactgcgctaagagatccggcagaatcgccttcggcgctctggatgcttggggacagggaacactggtcaccgtgtcctcc

SEQ ID NO: 278 - DNA, 654 bp, synthetic construct
  misc_feature 1..654: note = coding DNA for LC_ARGX-119
  source 1..654: mol_type = other DNA
cagaccgtcgtgacccaagagccttccttctctgtgtctcctggcggcaccgtgacactgacatgtggactgtcctctggctccgtgacctccagcaactaccccgactggtatcagcagacccctggacaggctcctcggacactgatctactccaccgactccagacactctggcgtgcccgatagattctccggctctatcctgggcaacaaggccgctctgacaatcactggcgcccaggccgatgacgagtccgattactactgcggcctgtactcctactccggctccaagaactacgtgttcggcggaggcaccaagctgacagtgctgggacaacctaaggctgccccttccgtgactctgttccctccatcctctgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccaccggtcctacagctgccaagtgacccatgagggctccaccgtggaaaagaccgtggctcctaccgagtgctcc

SEQ ID NO: 279 - DNA, 336 bp, synthetic construct
  misc_feature 1..336: note = coding DNA for VL_ARGX-119
  source 1..336: mol_type = other DNA
cagaccgtcgtgacccaagagccttccttctctgtgtctcctggcggcaccgtgacactgacatgtggactgtcctctggctccgtgacctccagcaactaccccgactggtatcagcagacccctggacaggctcctcggacactgatctactccaccgactccagacactctggcgtgcccgatagattctccggctctatcctgggcaacaaggccgctctgacaatcactggcgcccaggccgatgacgagtccgattactactgcggcctgtactcctactccggctccaagaactacgtgttcggcggaggcaccaagctgacagtgctg

SEQ ID NO: 280 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 281 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 282 - protein, 450 aa, synthetic construct
ELQLVESGGGLVQPGGSLRLSCAASGFTFDDYGMSWVRQAPGKGLEWVSAIPWNGGSTYYKESVKGRFTISRDNAKKTLYLQMNSLKSEDTAVYYCAKRSGRIAFGALDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 283 - protein, 218 aa, synthetic construct
QAVVTQEPSLSVSPGGTVTLTCGLSSGSVTSSNYPDWYQQTPGQAPRGLIYSTDSRHSGVPSRFSGSISGNKAALTITGAQSEDEADYYCGLYMYSGSKNYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 284 - DNA, 363 bp, synthetic construct
  source 1..363: mol_type = other DNA
gagttgcagctggtggagtctgggggaggcttggtgcagcctggggggtctctgagactctcctgtgcagcttctggattcacttttgatgattatggcatgagctgggtccgacaggctccagggaaggggctggaatgggtctcagctataccctggaatggtggtagcacatactataaagagtccgtgaagggccgattcaccatctccagagacaacgccaagaaaacgctgtatctgcaaatgaacagtctgaaatctgaggacacggccgtgtattactgtgcaaagcgatcaggccgaattgccttcggggctttggacgcatggggccaggggaccctggtcactgtctcctca

SEQ ID NO: 285 - DNA, 336 bp, synthetic construct
  source 1..336: mol_type = other DNA
caggctgtggtgacccaggagccgtccctgtcagtgtctccaggagggacggtcacactcacctgcggcctcagctctgggtctgtcacttccagtaactaccctgattggtaccagcagacaccaggccaggctccccgaggtcttatctacagcacagacagccgccactctggggtccccagtcgcttctccggatccatctctgggaacaaagccgccctcaccatcacgggggcccagtccgaggacgaggccgactattactgtggtctgtacatgtatagtggtagtaagaattatgtgttcggcggagggaccaagctgaccgtcctc

SEQ ID NO: 286 - DNA, 987 bp, synthetic construct
  source 1..987: mol_type = other DNA
gccagtacaaaaggtccaagtgtgttccctcttgctccctcatccaagagtaccagtggaggcaccgccgctcttggctgcttggttaaggattatttcccagagcctgtcactgtttcatggaactccggcgccttgacatctggtgtgcatacctttccagccgtgctgcagtcaagtggcctctacagcctcagtagcgtggtcactgtgcccagcagctctctcggcacacaaacttatatctgtaatgtgaatcataagccttcaaataccaaggtggataagaaagtggaaccaaaatcatgtgacaagacacacacctgccctccttgtccagcccccgaagcggccggtgggcccagcgtgttcctgtttcctcctaaacccaaagacactctgatgattagtaggaccccagaagtcacttgcgtggtggttgacgtgtcacatgaagatcccgaggtcaagttcaattggtatgttgacggggtcgaagttcacaacgctaaaactaaaccaagagaggaacagtataactctacctaccgggtggtgagtgttctgactgtcctccatcaagactggctgaatggcaaagaatacaagtgtaaggtgagcaacaaagccctgcccgctcctatagagaaaacaatatccaaagccaaaggtcaacctcgcgagccacaggtgtacaccctcccaccaagccgcgatgaacttactaagaaccaagtctctcttacttgcctggttaaggggttctatccatccgacattgcagtcgagtgggagtctaatggacagcctgagaacaactacaaaaccacccctcctgttctggattctgacggatctttcttcctttattctaaactcaccgtggataaaagcaggtggcagcagggcaacgtgttcagctgttccgttatgcatgaggccctgcataaccattatacccagaagtctttgtccctcagtccagga

SEQ ID NO: 287 - DNA, 318 bp, synthetic construct
  source 1..318: mol_type = other DNA
ggtcaacctaaagcagcaccttcagttactctgtttccacctagttcagaggaactgcaggccaataaagccacactcgtctgcctcatcagtgacttctacccaggagccgtgaccgtggcctggaaagccgacagtagccccgtgaaggccggggtggagacaacaactcctagtaaacagagtaataacaaatatgccgctagtagttatctctccctcactcccgagcagtggaagtctcacagaagttactcttgtcaggttactcacgagggttccacagtggaaaagactgtggcccctactgaatgtagt

SEQ ID NO: 288 - DNA, 1350 bp, synthetic construct
  source 1..1350: mol_type = other DNA
gagttgcagctggtggagtctgggggaggcttggtgcagcctggggggtctctgagactctcctgtgcagcttctggattcacttttgatgattatggcatgagctgggtccgacaggctccagggaaggggctggaatgggtctcagctataccctggaatggtggtagcacatactataaagagtccgtgaagggccgattcaccatctccagagacaacgccaagaaaacgctgtatctgcaaatgaacagtctgaaatctgaggacacggccgtgtattactgtgcaaagcgatcaggccgaattgccttcggggctttggacgcatggggccaggggaccctggtcactgtctcctcagccagtacaaaaggtccaagtgtgttccctcttgctccctcatccaagagtaccagtggaggcaccgccgctcttggctgcttggttaaggattatttcccagagcctgtcactgtttcatggaactccggcgccttgacatctggtgtgcatacctttccagccgtgctgcagtcaagtggcctctacagcctcagtagcgtggtcactgtgcccagcagctctctcggcacacaaacttatatctgtaatgtgaatcataagccttcaaataccaaggtggataagaaagtggaaccaaaatcatgtgacaagacacacacctgccctccttgtccagcccccgaagcggccggtgggcccagcgtgttcctgtttcctcctaaacccaaagacactctgatgattagtaggaccccagaagtcacttgcgtggtggttgacgtgtcacatgaagatcccgaggtcaagttcaattggtatgttgacggggtcgaagttcacaacgctaaaactaaaccaagagaggaacagtataactctacctaccgggtggtgagtgttctgactgtcctccatcaagactggctgaatggcaaagaatacaagtgtaaggtgagcaacaaagccctgcccgctcctatagagaaaacaatatccaaagccaaaggtcaacctcgcgagccacaggtgtacaccctcccaccaagccgcgatgaacttactaagaaccaagtctctcttacttgcctggttaaggggttctatccatccgacattgcagtcgagtgggagtctaatggacagcctgagaacaactacaaaaccacccctcctgttctggattctgacggatctttcttcctttattctaaactcaccgtggataaaagcaggtggcagcagggcaacgtgttcagctgttccgttatgcatgaggccctgcataaccattatacccagaagtctttgtccctcagtccagga

SEQ ID NO: 289 - DNA, 654 bp, synthetic construct
  source 1..654: mol_type = other DNA
caggctgtggtgacccaggagccgtccctgtcagtgtctccaggagggacggtcacactcacctgcggcctcagctctgggtctgtcacttccagtaactaccctgattggtaccagcagacaccaggccaggctccccgaggtcttatctacagcacagacagccgccactctggggtccccagtcgcttctccggatccatctctgggaacaaagccgccctcaccatcacgggggcccagtccgaggacgaggccgactattactgtggtctgtacatgtatagtggtagtaagaattatgtgttcggcggagggaccaagctgaccgtcctcggtcaacctaaagcagcaccttcagttactctgtttccacctagttcagaggaactgcaggccaataaagccacactcgtctgcctcatcagtgacttctacccaggagccgtgaccgtggcctggaaagccgacagtagccccgtgaaggccggggtggagacaacaactcctagtaaacagagtaataacaaatatgccgctagtagttatctctccctcactcccgagcagtggaagtctcacagaagttactcttgtcaggttactcacgagggttccacagtggaaaagactgtggcccctactgaatgtagt